(19) United States
(12) Patent Application Publication
(10) Pub. No.: US 2022/0325330 A1
(43) Pub. Date: Oct. 13, 2022

(54) MOLECULAR ELECTRONIC SENSOR FOR PRECISION TELEMEDICINE DIAGNOSTICS AND PERSONAL VIROMETER

(71) Applicant: Roswell Biotechnologies, Inc., San Diego, CA (US)

(72) Inventors: Barry Merriman, San Diego, CA (US); Carl Fuller, San Diego, CA (US); Paul Mola, San Diego, CA (US)

(73) Assignee: Roswell Biotechnologies, Inc., San Diego, CA (US)

(21) Appl. No.: 17/557,028

(22) Filed: Dec. 20, 2021

Related U.S. Application Data
(63) Continuation-in-part of application No. PCT/US21/53030, filed on Sep. 30, 2021.
Application No. PCT/US21/53030 is a continuation of application No. 17/476,427, filed on Sep. 15, 2021.
(60) Provisional application No. 63/085,874, filed on Sep. 30, 2020.
(60) Provisional application No. 63/128,300, filed on Dec. 21, 2020.

(51) Int. Cl. C12Q 1/6825; G01N 27/414; C12Q 1/70; C12Q 1/6837

(52) U.S. Cl. CPC C12Q 1/6825; G01N 27/4146; C12Q 1/701; G01N 27/4145; C12Q 1/6837

(57) ABSTRACT

Described herein is a portable virometer for detecting viral targets. An exemplary virometer has a molecular electronics sensor with a first electrode, a second electrode spaced-apart from the first electrode by a nanogap, a bridge molecule having a first end and a second end, the first end coupled to the first electrode and the second end coupled to the second electrode, a hybridization probe having an oligonucleotide sequence from or related to a viral target is conjugated to the bridge molecule, a sample applicator for acquiring a sample and transferring it to the chip, and data processing software and hardware for providing a report of detection of viral targets. Methods of using the virometer for testing in the home, in schools, in workplaces, in hotels, in restaurants, or in public places are also described.

### CROSS-REFERENCE TO RELATED APPLICATIONS

This application claims priority to, and the benefit of, U.S. Provisional Patent Application Ser. No. 63/128,300 by Carl Fuller et al., entitled ‘Molecular Electronic Sensors for Precision Telemedicine Diagnostics’ filed on Dec. 21, 2020, U.S. Provisional Patent Application Ser. No. 63/085,874 by Carl Fuller et al., entitled ‘System, Method and Apparatus for Personal Virometer’ filed on Sep. 30, 2020, U.S. Ser. No. 17/476,427 by Carl Fuller et al., entitled ‘Molecular Electronic Sensors for Detecting Sars-Cov-2 Virus’, filed on Sep. 15, 2021, and is a continuation-in-part of PCT/US2021/053,030 by Carl Fuller et al., entitled ‘System, Method and Apparatus for Personal Virometer’ filed on Sep. 30, 2021, the disclosures of which are incorporated herein by reference in their entireties for all purposes.

### SEQUENCE LISTING

The instant application contains a Sequence Listing which has been submitted electronically in ASCII format and is hereby incorporated by reference in its entirety. Said ASCII copy, created on Mar. 23, 2022, is named ROS-0600-CIP SL.txt and is 19,285 bytes in size.

### FIELD

The disclosure generally relates to the field of virology and detection. More particularly, an embodiment of the invention generally relates to systems, methods and devices to provide a portable, handheld virometer for personal use.

### BACKGROUND

The COVID-19 pandemic created a need to greatly increase the amount and accessibility of testing for the SARS-CoV-2 virus, as well as cold and flu viruses that may produce similar symptoms. All the initial diagnostic tests for COVID-19, as well as all of those used for diagnosing Flu and cold viruses, require a centralized clinical lab to perform the test, or a point-of-care testing device operated by a clinical professional. To increase test accessibility during the Pandemic, various efforts were made by the FDA and test providers to extend such clinical testing to the home.

In one approach, which is used for the LabCorp Pixel Test, and approved by the FDA under Emergency Use Authorization (EUA), the at-home user takes a sample at home, such as a nasal swab, using a provided sample collection kit, and sends in the sample for clinical lab processing, and gets results reported via an app or email. However, this still involves a 24-to-48-hour delay in receiving test results.

To get the full benefit of rapid results at home, another approach to home testing that retains clinical oversight, and which is used for the Lucira Health “COVID-19 All-In-One Test Kit”, also approved under FDA EUA, is for a Physician to prescribe the test to the home-user, at which point a testing kit is procured from a Pharmacy, by prescription, and otherwise administered and read-out entirely at home. In the case of the Lucira test, this home use process involves the home user taking a nasal swab, and inserting it into the test cartridge, and reading the result with the help of a mobile phone app. The home test itself takes under 30 minutes from sample to results. However, the home user must still first receive a prescription, and moreover only then obtain the test from a Pharmacy, through some pick-up or delivery process that itself requires substantial time and effort. This lengthens the time from need-to-answer, possible by hours, and adds a substantial burden on the user, who may themselves be sick and not have means or assistance to go and retrieve a test. In one embodiment, the disclosure relates to medical devices used at home for in vitro diagnostics. In one specific embodiment, the disclosure relates to detection and/or diagnosis of infectious disease. In another embodiment, the disclosure relates o personal diagnosis of viral diseases. This may include viral pandemic disease detection, such as the detection of COVID-19 virus or its mutations. In certain embodiments, molecular electronic sensors are used to detect the presence or absence of a virus or its mutations. In another application, molecular diagnostics is used for direct detection of DNA, RNA, proteins, antibodies or antigens which are then used to identify infectious disease agents, infections or exposures. An exemplary embodiment is directed to a personal virometer device for personal screening for viral disease, including pandemic viral diseases such as COVID-19 and the compositions, methods, applications and manufacture and of such virometer devices.

### SUMMARY

The virus SARS-Cov-2 emerged in the human population in 2019 and spread rapidly to produce a global pandemic of the associated disease, known as Coronavirus Disease 2019 (COVID-19). Within a year, it is estimated that the global pandemic infected nearly 30 million people, resulted in nearly a million deaths, and caused tens of trillions of dollars in economic losses.

The COVID-19 viral pandemic highlighted the critical need to greatly improve global response capabilities to pandemic viruses, across the entire spectrum of countermeasures, including vaccines, therapeutics, and diagnostics. In particular, in the area of diagnostics, there is a critical need for diagnostics that are rapid, low cost, deployable in the field for personal use, and capable of highly informative, multiplex measurements. For example, in response to COVID-19, the most widely deployed screening diagnostic was a simple temperature measurement, taken by thermometer. This meets all these criteria, except for multiplex, highly informative measurements. Elevated temperature provides a very crude indicator of potential COVID-19 infection. It is severely lacking in both specificity and sensitivity. What is ideally needed therefore is a device that has the practical advantages of the thermometer, but that can be highly informative for viral infection. Ideally, the information provided would not be just a yes/no answer on a single target virus, but instead such information across many possible viruses, and also information on the precise genetic strain or variant of such viruses, as well as information on the viral load or concentration of the infectious agent. The object of this invention, the Personal Virometer, provides this solution, as disclosed below.

For the purpose of pathogen detection and disease diagnosis in the area of infectious disease, including viral pathogens, but also more generally encompassing virus, bacteria, fungus or parasites, a number of categories of detection assays have been developed. These broadly include detection of pathogen DNA, RNA, proteins, antibodies, antigens, or of the infectious organism itself (e.g. viral particle, bacterial cell, fungal spore or hyphae, or parasite organism). There are classical detection methods that are well established for these different detection modalities. For DNA or RNA, detection methods using hybridization (binding to a complementary strand) or Polymerase Chain Reaction (PCR) are well known to those skilled in molecular diagnostics. For proteins or antibodies, detection methods based on Enzyme Linked Immunosorbent Assays (ELISA) or antibody-protein interactions are well known. For antigens, detection methods based on ELISA or antibody-antigen interactions are well known. For direct detection of the infectious agent, methods based on microscopy and imaging are well known, as are methods of culturing and observing the morphology or staining properties of cultures.

A variety of specific measurement techniques and technologies have been established to carry out versions of these general assays. Hybridization assays are often carried out using DNA microarray technology. PCR-based detection is commonly carried out using quantitative PCR instruments (q-PCR) or Real-time PCR instruments (RT-PCR). Protein or Antigen binding assays are carried out using Immunoprecipitation Assays or ELISA assays and optical detectors or plate-readers or lateral flow strip readouts. Detection of the pathogen organisms is carried out by using a microscope for imaging, or by methods of cell culture, and characterization of culture morphology or staining, also typically read out by imaging systems.

The technology foundation for the instant disclosure is molecular electronics. This is a general field of technology in which a single molecule is placed as a component in an electrical circuit, to perform some useful electrical function, such as transducing chemical or molecular events into an electrical signal. Such devices may be particularly powerful as biosensors. This general concept is illustrated in FIG. 1, which shows a cartoon depiction of a single molecule that has been bound to two nano-scale electrodes, and where a surrounding circuit is used to apply a voltage, V, across the electrodes, and to measure the current, i, that flows through the electrodes and molecule, over time. When such a molecule interacts with other molecules, these events may be transduced into signals or detectible signatures present in the measured current versus time trace. The resulting system has the potential to be used as a sensor for a great variety of molecular interaction processes. The scale of the gap between electrodes is set based on the length of the molecules of interest. As a result, molecular electronic devices readily scale to the smallest possible dimensions for electrical circuits—the scale of molecules. Molecular electronics therefore offers the potential to make semiconductor chip-based, all-electronic sensor devices that are both maximally scalable and maximally sensitive, with single molecule detection capabilities. This is an emerging field of technology, with much promise, but it remains challenging both to devise and to fabricate such systems that actually perform applications of interest.

The following presents certain exemplary and non-limiting of the disclosure.

In one embodiment, the disclosure relates to the Personal Virometer, which can replace the limited effectiveness of a thermometer for detecting viral disease, by a device that has the advantage of being highly informative for detecting viral infections, while retaining the desirable attributes of being rapid, low cost, and field deployable for personal use. The personal Virometer disclosed here further has the advantage that it is not limited to providing a yes/no answer for the presence of a single target virus, it can also survey across many possible viruses, and also provide information on the precise strain or genetic variant of such viruses. It can also provide information on the degree of infection, based on quantitative measures of the various possible biomarker indicators of viral infection, such as the abundance of viral DNA or RNA, viral proteins, viral antigens, or viral particles.

In another embodiment, the disclosure relates to a personal device for the screening, detection, or diagnosis of viruses and viral disease, that has the advantages of being rapid, low-cost, deployable for field use or personal use, and providing multiplex, precision measurements. Such device can detect different types of disease-causing viruses, as well as different strains or genetic variants of such viruses, within a single test. In another embodiment, the disclosure relates to a test can be done for the COVID-19 virus, SARS-CoV-2, as well as other viruses of interest, such as influenza viruses, common cold viruses, common corona viruses, measles, mumps, rubella, chicken pox, and other well-known viral epidemic infectious agents, such as MERS, SARS, West Nile, Zika or Ebola. Certain embodiments allow a symptomatic individual to take one such test, and survey across many such possible causal viruses, in a single, personal, point-of-use or at home test.

In another embodiment, the disclosure relates to a personal virometer device suitable for individual screening at home or in other point-of-use settings, and in portable, mobile or handheld device formats. It is within the disclosed principles to provide high throughput personal virometers suitable for screening larger numbers of samples, such as from groups of people or many environmental swabs, in a point-of-use format.

In another embodiment, the disclosure relates to a Personal Pathometer device for the detection of infectious disease pathogens, and the related personal screening or diagnosis of infectious diseases, that has the advantages of being rapid, low-cost, deployable for field use or personal use, and providing multiplex, precision measurements. This is similar to the virometer, but extended to a broader class of pathogens. Such pathogens include viruses, bacteria, fungi, and parasites. In the special case of bacterial diseases, it is the object to disclose such a Personal Bactometer.

In still another embodiment, the disclosure relates to devices that use molecular electronics sensors to provide the fundamental multiplex detection measurements. This includes measurements of the viral or pathogen DNA or RNA, including measurements that provide information on the strain or genetic variant of the virus or pathogen, and the severity, load or strength of infection. This includes measurements of identifying viral or pathogen proteins or antigens. This includes measurements that directly detect viral particles, or the pathogen organism (such as bacterial cells or fungal spores or fungal hyphae).

In still another embodiment, the disclosure relates to compositions and the Personal Virometer or Personal Pathometer that utilize specific molecular electronic sensor array chip embodiments, including binding sensors for DNA or RNA targets, protein targets, or antigen or antibody targets.

In another embodiment, the disclosure relates to methods of using the Personal Virometer or Personal Pathometer for the purpose of individual monitoring of infectious disease status or exposure, that of other subjects, or for screening the environment for signs of infectious agents.

In yet another aspect, the disclosure provides methods manufacture of the Personal Virometer or Personal Pathometer, in the form of various system designs diagrams, that those skilled in the art of commercial medical device design and manufacturing could readily reduce to practice.

In certain other embodiments, the disclosure provides methods that allow Personal Virometer to perform multiplex measurements, include multiplexing of up to 10, up 100, up to 1000, up to 10,000, up to 100,000 or more multiplex target measurements.

In another embodiment, the disclosure relates to methods that allow Personal Virometer or Pathometer devices which are field-programmable, such that the basic platform and sensor chip can be rapidly programmed via a reaction performed in the field, to have new content (detection targets). This provides advantages of more rapid response to new infectious agents, both in terms of the manufacturing chain, and in terms of regulatory approval, by such field-programmable changes being minimal alterations of the general Virometer or Pathometer device and consumables.

In another embodiment, the disclosure provides a Personal Virometer which can be combined with big data collection methods and Artificial Intelligence (AI) methods to enable creation of regional, national or global rapid response systems. This provides new ways to monitor, predict or react to outbreaks of infectious disease, and to control the spread of such diseases.

In another implementation, the disclosure relates to methods for use of the Personal Virometer or Pathometer to detect the presence or absence of sexually transmitted disease and food-born disease.

In another embodiment, the disclosure relates to methods in which the personal virometer or pathometer can also collect a DNA fingerprint or identifying set of genotypes from the test subject, within the same test, which can be used to automatically identify or trace the identity of each tested individual over time, for the purpose of affiliating results of testing with specific individuals in fully automated ways. This provides advantages of providing a chain of custody of test results, proper affiliation of test results with subjects, and the ability to reliably affiliate multiple tests over time with the same individual for precision tracking of personal health status. This also provides advantages in fully enabling tracing of infections in a population and information-rich contact tracing. This also provides advantages for biosecurity, such as in monitoring and tracking of individuals have may have come into contact with bioweapon pathogens. This provides the ability of the virometer to produce digital certificates of individual test status results, including verification of time, location, and personal identity based on measured DNA fingerprint. Such digital certificates can provide an advantageous way of conveniently ensuring individuals have been recently screened as free from infection, prior to admission into public or commercial places or venues, or prior to admission into various forms of public transportation, such as airplanes, trains, buses or taxi or rideshare services.

In yet another implementation, the disclosure is directed to extracting rich information from personal virometer. This resource can be used to promote personal and public health, and help control the spread of pandemic viruses at both the personal and societal level. This includes automated affiliation of GPS coordinates or other timestamp and location information on location and time of testing with the test results, as well as other metadata such as personal identifiers.

It is also an object of this disclosure to provide a method by which at-home user or user in the field can utilize a test regulated by Physician prescription, but in which there is the benefit of no time delay needed to acquire the test after the prescription is provided, and in particular when this is provided via telemedicine. In particular, a full telemedicine solution is realized, in which a prescription can be had by telemedicine, and the test immediately preformed in its entirety at home or in the field.

It is also the object of this disclosure to provide a method by which such a telemedicine prescription and engagement can be used to prescribe entirely new tests, with new detection targets, directly to an at-home user or a user in the field. Thus entirely new diagnostics, such as for a new strain of flu or novel pandemic virus, or a new strain of pandemic virus, can be provided via telemedicine, immediately, to households across the country or world, or even to test takers located in earth orbit, the moon or outer space, or to users in the field in any locations accessible by telemedicine connections, such as aboard ships, submarines, aircraft, on trains, busses or cars, at remote work sites, or at schools, or while transit when travelling nationally or globally, or at diverse military deployment sites or battle theaters.

### BRIEF DESCRIPTION OF THE DRAWINGS

FIG. 1 Shows the general concept of a molecular electronic circuit with applied voltage and measured current.

FIG. 2.1 Illustrates the general processes of single stranded DNA hybridization to form a double stranded duplex DNA. FIG. discloses SEQ ID NOS 13, 14, 13, and 14, respectively, in order of appearance.

FIG. 2.2 Shows the general concept of engaging a DNA primer into a molecular electronics circuit. FIG. discloses SEQ ID NO: 15.

FIG. 3 Shows the concept of primer hybridization binding events generating sensor signals in the measured current. FIG. discloses SEQ ID NOS 15-16, respectively, in order of appearance.

FIG. 4 Shows the concept that when exposed to a complex pool of DNA targets, the hybridization sensor will generate stronger signals from proper hybridization binding events, and weaker signals from incomplete or off-target interactions. FIG. discloses SEQ ID NOS 15, 16, 16, and 16, respectively, in order of appearance.

FIG. 5. Shows the concept that a single sensor bridge molecule could comprise multiple hybridization probes. FIG. discloses SEQ ID NOS 15, 15, and 15, respectively, in order of appearance.

FIG. 6. Shows the architecture of a CMOS chips sensor array device and pixel that can provide for chip-based deployment of multiple molecular electronic hybridization sensors.

FIG. 7 Shows the concept of a CMOS chips sensor array device where each measurement pixels could provide for the monitoring of multiple molecular electronic sensor elements, allowing multiple sensors per pixel.

FIG. 8 Shows the concept of how molecular electronics hybridization sensors can be fully deployed in applications in pathogen detection and monitoring.

FIG. 9 Shows a specific sensor embodiment used in demonstration experiments of primer concentration effects. FIG. discloses SEQ ID NOS 2-3, respectively, in order of appearance.

FIG. 10 Shows an examples signal trace from one pixel of a 16k sensor pixel chip on which the sensor embodiment of FIG. 9 is deployed

FIG. 11 Shows a closeup view of the signal trace in the Primer binding phase of the sensor data shown in FIG. 10, and a histogram of the measurement values that indicates the relative time spent in on and off states.

FIG. 12 Shows a signal trace from a sensor on a 16k pixel chip, for an experiment in which the concentration of target primer is serially raised from 10 nM (nano-Molar) to 100 nM to 1000 nM.

FIG. 13 Shows close-up signals traces from the three concentration phases of the experiment of FIG. 12, along with histograms that indicate the relative time spent in on versus off states.

FIG. 14 Shows the measured fraction of time spent in the on/bound state (X icons), versus primer concentration, for the experiment of FIG. 12, illustrating the relationship between target concentration and fraction of time bound. Also shown is a fit to the expected exponential decay curve.

FIG. 15 Shows a specific sensor embodiment used in demonstration experiments on the impact of perfect matches versus mismatches in the primers.

FIG. 16 Shows the sequences of the hybridization probe used in experiments on primer length, location and mismatches, and of the various different length and target site primers used in these experiments. FIG. discloses SEQ ID NOS 4, 4, 17, 4, 18, 4, 19, 4, 20, 4, 21, 4, 22, 4, 23, 4, 18, 4, 24, 4, 25, 4, 26, 4, 27, 4, and 28, respectively, in order of appearance by column.

FIG. 17 Shows an example sensor signal pixel trace from a 16k pixel chip, from an experiment that serially exposed the sensor to different lengths of target primer.

FIG. 18 Shows close-ups of the signal from the data of FIG. 17, along with calculated values of primer length, melting point (Tm), duration of on events, and off-rate.

FIG. 19 Shows the perfect match primer for the hybridization probe, and various mismatch primers with from 1 to 7 mismatches to the hybridization probe. FIG. discloses SEQ ID NOS 18, 29-47, 18, 48-63, respectively, in order of appearance by graph.

FIG. 20 Shows close-up signal traces from perfect match, single mismatch and triple mismatch primers, and the related measured on-times and off-rates. FIG. discloses SEQ ID NOS 18, 51, and 38, respectively, in order of appearance.

FIG. 20.1 Shows the S (Spike) protein of the SARS-CoV-2 virus, and an aptamer for binding the S protein. FIG. discloses SEQ ID NOS 7, 7, 64, 65, and 64, respectively, in order of appearance.

FIG. 20.2 Shows a depiction of a molecular electronics aptamer sensor for the S protein of the SARS-CoV-2, and the measured signal from an embodiment of this sensor as the S protein is applied to the sensor in increasing concentrations.

FIG. 21 Shows different example embodiments for linking decoding probe hybridization targets to the primary hybridization probe.

FIG. 22 Shows alternative embodiments for conjugating the hybridization probe into a molecular electronics sensor, representing diverse attachment options.

FIG. 23 Shows alternative embodiments for conjugating the hybridization probe into a molecular electronics sensor, by use of a complexing molecule.

FIG. 24 Shows the use of probe secondary structure, optionally in conjugation with a signal enhancing group, in a molecular electronics hybridization sensor.

FIG. 25 Shows the use of probe hairpin secondary structure, optionally in combination with a signal enhancing group, in a molecular electronics hybridization sensor.

FIG. 26 Shows the use of probe hairpin secondary structure, with mismatches, as well as a bridge oligo secondary structure, optionally in combination with a signal enhancing group, in a molecular electronics hybridization sensor.

FIG. 27 Shows the use of probe secondary structure with a mismatched protection strand, to create a parallel bridge structure, optionally in combination with a signal enhancing group, in a molecular electronics hybridization sensor.

FIG. 28 Shows the use of probe secondary structure with a mismatched protection strand, to create the primary bridge structure, optionally in combination with a signal enhancing group, in a molecular electronics hybridization sensor which opens the circuit upon hybridization.

FIG. 29 Shows the use of probe secondary structure with a mismatched protection strand, to create the primary bridge structure, optionally in combination with a signal enhancing group, in a molecular electronics hybridization sensor.

FIG. 30 Shows the use of the probe as a primary bridge structure in a molecular electronics hybridization sensor, in which hybridization produced a double-stranded bridge.

FIG. 31 Shows the use of a hybridization probe in an electronic hybridization sensor, in which the target strand is labeled with a signal enhancing group.

FIG. 32 Illustrates the process of a primer binding to a template. FIG. discloses SEQ ID NOS 66, 67, 66 and 67, respectively, in order of appearance.

FIG. 33 Illustrates the process of a primer extension by a polymerase. FIG. discloses SEQ ID NOS 66, 67, 66, 67, 68, and 67, respectively, in order of appearance.

FIG. 34 Illustrates the selective extension of properly bound primers versus mis-matched primers. FIG. discloses SEQ ID NOS 66, 67, 69, 67, 67, 66, 67, 69, and 67, respectively, in order of appearance.

FIG. 35 Shows the measurement of signals related to primer binding and extension on a molecular electronics sensor.

FIG. 35.1 Illustrates the Single Base Sequencing Primer Extension Reaction. FIG. discloses SEQ ID NOS 70, 71, and 70, respectively, in order of appearance.

FIG. 36 Illustrates the detection of primer extension by incorporating bases with signal enhancing groups to enhance signals produced by a molecular electronics sensor monitoring a primer extension reaction, in order to enhance the detection during extension or post-extension.

FIG. 37 Illustrates the detection of primer extension by incorporating bases with a signal group binding site, labeling the extension product with the signal group, and detecting the signal group post extension.

FIG. 38 Illustrates different configurations for attaching a primer to a molecular electronics sensor, with conjugation at different locations along the length of the primer oligo.

FIG. 39 Illustrates the potential to extend in both directions for a target bound to a hybridization probe, as well as the potential to selectively block each such extension.

FIG. 40 Shows an embodiment of a primer extension probe with segments for sample encoding and probe encoding.

FIG. 41 Shows an embodiment in which blocking oligos are used for signal generation via strand displacing extension.

FIG. 42 Shows embodiments in which hybridization is used to detect the target, and primer extension is used to decode the identity of the hybridization probe on an array of probes.

FIG. 43 Shows embodiments in which an independent primer binding and extension with a universal primer is used to decode the probe identity on an array of probes.

FIG. 44 Shows an embodiment in which two selective extensions are used to read unknown sequence and read probe identity on an array of probes.

FIG. 45 Shows the catalytic cycle of polymerase enzyme extending a DNA strand.

FIG. 46 Shows an alternative embodiment for primer extension, with template strand down on the sensor, and primer incoming from solution.

FIG. 47 Shows an experimental signal trace from a molecular electronics sensor, with phases of primer binding, polymerase binding, and extension in various buffers.

FIG. 48 Shows an experimental signal trace from a molecular electronics sensor, with phases of primer binding, polymerase binding, and extension in various buffers.

FIG. 49. Shows an experimental signal trace from a molecular electronics sensor, with phases of primer binding, polymerase binding, and extension in various buffers.

FIG. 50. Shows the chemical structure of the four modified dNTPs used in extension experiments.

FIG. 51 Shows the concept of using primer extension for decoding and mapping of primers or hybridization probes on an array.

FIG. 52 Illustrates major external functional elements of the personal virometer.

FIG. 53 Illustrates major internal functional elements of the personal virometer.

FIG. 53.1 Shows a visualization of a CAD design for one embodiment of a handheld personal virometer, showing the base and disposable sample collection tip.

FIG. 53.2 Shows a visualization of a CAD design for one embodiment of a handheld personal virometer, illustrating the disposable tip docking to the base.

FIG. 53.3 Shows a visualization of a CAD design for one embodiment of a handheld personal virometer, illustrating the device in assembled processing mode.

FIG. 53.4 Shows an exploded view visualization of a CAD design for the disposable tip cartridge for one embodiment of a handheld personal virometer.

FIG. 54 Illustrates major features of the chip module of the personal virometer.

FIG. 55 Shows a cross-sectional view of one exemplary embodiment of a sensor chip board for a virometer device, with a detailed view of the chip flow cell and socket.

FIG. 56 Illustrates an embodiment of the personal virometer in which the sample wand is used independently from the base, and then used to directly transfer sample into the base.

FIG. 56.1 Shows a visualization of a CAD design for one embodiment of a personal virometer with a separate base module.

FIG. 56.2 Shows a visualization of a CAD design for one embodiment of a personal virometer with a separate base module, annotating key elements of the design.

FIG. 56.3 Shows a visualization of a CAD design for one embodiment of a personal virometer with a separate base module, depicted in its processing mode.

FIG. 56.4 Shows a visualization of a CAD design for one embodiment of a personal virometer with a separate base module, annotating the major functional elements of the design.

FIG. 56.5 Shows a visualization of a CAD design for the companion information app for one embodiment of a personal virometer, illustrating reporting of results and other analytics.

FIG. 56.6 Shows a visualization of a CAD design for one embodiment of a personal virometer with a separate base module, showing how multiple devices can be compactly deployed.

FIG. 57 Illustrates an embodiment of the personal virometer in which there is a separate sample preparation module.

FIG. 58 Illustrates the major series of operations in one exemplary embodiment of a method of using the personal virometer.

FIG. 59 Illustrates applications various exemplary use cases for the personal virometer.

FIG. 60 Illustrates a portable embodiment of the personal virometer.

FIG. 61 Illustrates an exemplary method of using a portable personal virometer to test sample from many subjects.

FIG. 62 Illustrates an exemplary method of using a portable personal virometer to test sample from environmental sampling sources.

FIG. 62.1 Shows a visualization of a CAD design for one embodiment of a personal virometer meant for cart-based usage, annotating major external elements.

FIG. 62.2 Shows a visualization of a CAD design for one embodiment of a personal virometer meant for cart-based usage, annotating elements exposed by the open lid.

FIG. 63 Illustrates a portable embodiment of the personal virometer, in which the sample prep module is a separate instrument.

FIG. 63.1 Shows a visualization of a CAD design for one embodiment of a personal virometer in which the sample prep and chip reader are in separate instruments.

FIG. 63.2 Shows a visualization of a CAD design for one embodiment of the environmental sample wand for the portable virometer.

FIG. 63.3 Shows a visualization of different use cases for one embodiment of the sample wand for environmental samples.

FIG. 63.4 Shows a visualization of a CAD design for one embodiment of a personal virometer, showing how the major steps of processing a sample wand through the sample prep and chip reader instruments

FIG. 63.5 Shows a visualization of a CAD design for one embodiment of a personal virometer, showing how the sample prep and reader instruments are deployed in desktop or cart-based formats.

FIG. 63.6 Shows a visualization of a CAD design for one embodiment of a personal virometer, showing a sample prep instrument configured to process multiple samples simultaneously.

FIG. 64 Shows detailed CAD designs for one embodiment of a portable personal virometer.

FIG. 65 Shows detailed system architecture schematics for one embodiment of a portable personal virometer.

FIG. 66 Shows a detailed instrument control architecture for one embodiment of a portable personal virometer.

FIG. 67 Shows an instrument electrical wiring diagram for one embodiment of a portable personal virometer.

FIG. 68 Shows a fluidic drive architecture for one embodiment of a portable personal virometer.

FIG. 69 Shows an instrument microcontroller architecture for one embodiment of a portable personal virometer.

FIG. 70 Shows the CAD design of the fluidic system and an associated test bench for testing the fluidics subsystem for one embodiment of a portable personal virometer.

FIG. 71 Shows cross sections of the instrument and flow cell and socket for one embodiment of a portable personal virometer.

FIG. 72 Shows a detailed cross-section of the flow cell and socket for one embodiment of a portable personal virometer.

FIG. 73 Shows the chip flow cell-to-instrument docking mechanism for one embodiment of a portable personal virometer.

FIG. 74 Shows a detailed design for the chip flow cell-to-instrument docking mechanism and action of this mechanism for one embodiment of a portable personal virometer.

FIG. 75 Shows cross-sectional design details for the chip flow cell for one embodiment of a portable personal virometer.

FIG. 76 Shows an exploded view of the chip flow cell-to-instrument docking mechanism for one embodiment of a portable personal virometer.

FIG. 77 Shows the chip flow cell internal fluidics design one embodiment of a portable personal virometer.

FIG. 78 Shows an exploded view of the design of the chip flow cell for one embodiment of a portable personal virometer.

FIG. 79 Shows one embodiment of the sample collection wand and sample processing module that utilizes bead-based processing, for one embodiment of a portable personal virometer.

FIG. 80 Shows one embodiment of the sample collection wand and sample processing module that utilizes filter-based processing, for one embodiment of a portable personal virometer.

FIG. 81 Shows an exploded view of the design for one embodiment of the sample collection wand for one embodiment of a portable personal virometer.

FIG. 82 Shows methods for transferring the sample to the flow cell using the wand directly, or using a prepared sample, for one embodiment of a portable personal virometer.

FIG. 83 Illustrates a sensor that can be programmed via hybridization to have a particular desired probe.

FIG. 84 Illustrates a sensor that can be programmed via hybridization to have a particular desired probe, in which a conjugation reaction is used to stabilize the probe attachment.

FIG. 85 Illustrates a field-programmable sensor chip, which can have a new probe set deployed by use of oligo-programmable sensors.

FIG. 86 Illustrates major types of probes, and their potential types of targets, which can be deployed on a virometer chip.

FIG. 87 Illustrates sensors based on alternative embodiments of the bridge molecule.

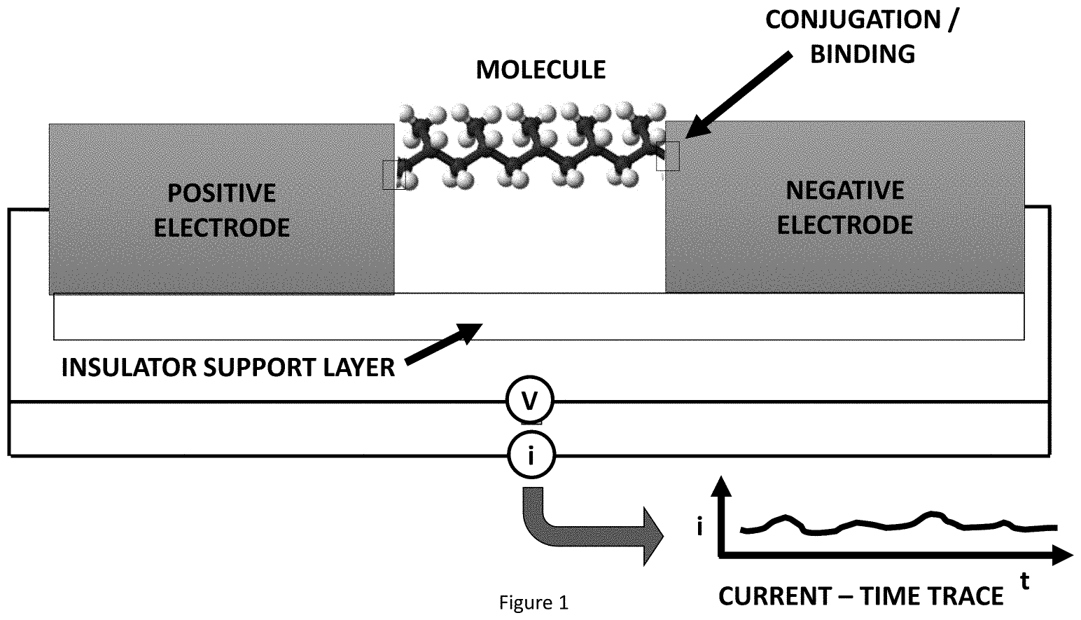

### DETAILED DESCRIPTION

The invention disclosed here consists of a device for testing personal biosamples on a molecular electronics sensor array chip, with molecular electronic sensor pixels configured to detect targets that may be any of viral DNA, RNA, proteins, antibodies, antigens or particles. The personal virometer and methods of use comprise methods of biosample collection, sample preparation for reading by the sensors, the readout of samples on a sensor array chip on a supporting device, the processing of this information for immediate report out of test results, and the subsequent storage of such test results, in affiliation with metadata related to the test, such as individual identifier, time and location information. In some embodiments the virometer also include informatics infrastructure that supports the databasing, aggregation, post-processing and retrieval of such information, to support diverse information applications related to promoting personal and public health.

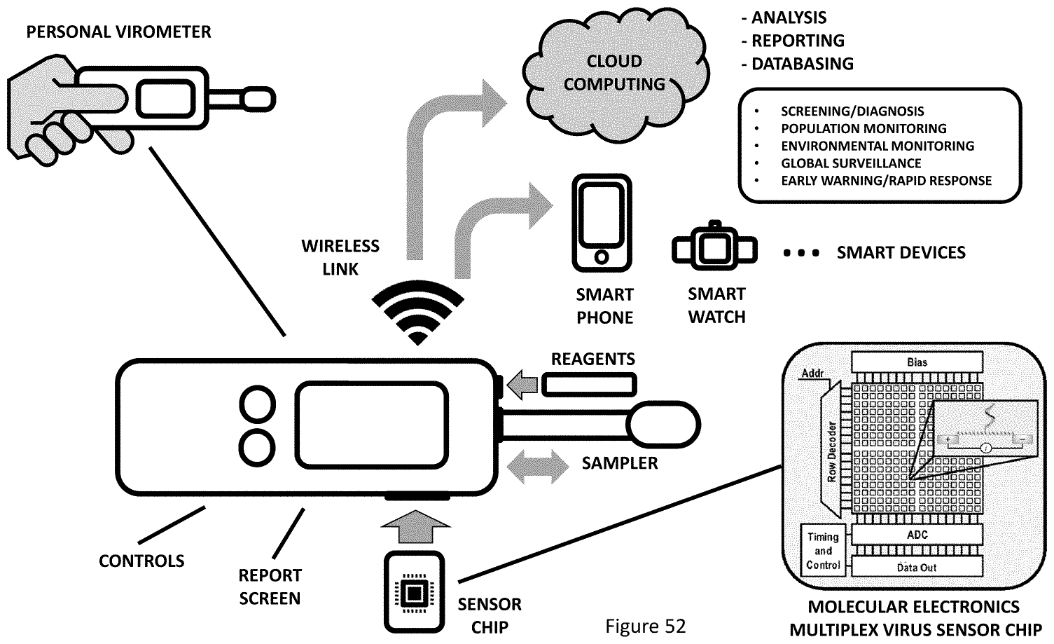

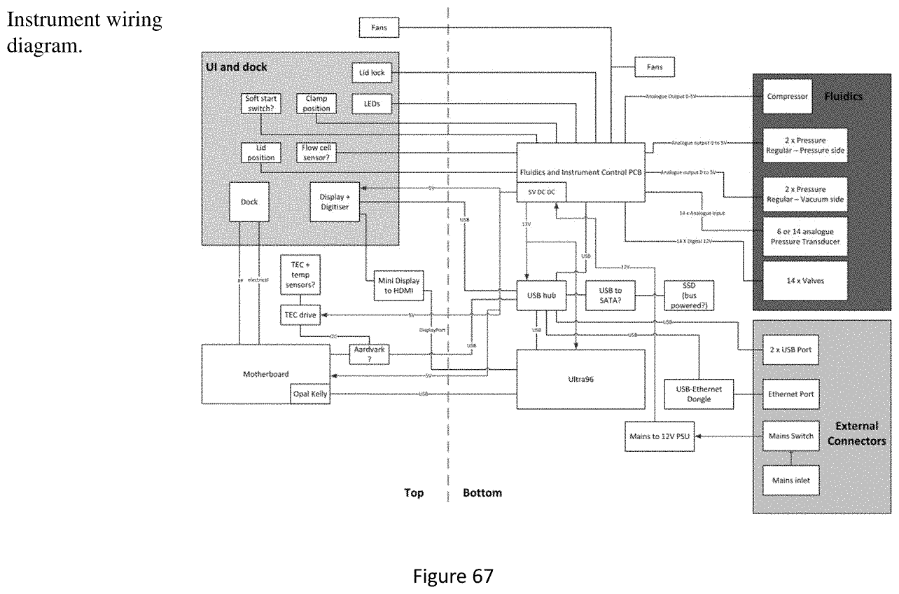

In the exemplary embodiments, as shown in FIG. 52, the virometer has the form factor a handheld device. This device has control buttons for power on/off, and selection of testing modes or reporting modes, as well as a read-out screen. In the exemplary embodiments, these functions may be integrated into a touch screen. The device has ports for inputting a disposable reagent cartridge, a sample acquisition tip, and a sensor chip. The sensor chip is a molecular electronics sensor array chip as indicated, with an array of pixels, each pixel providing a signal readout for a suitable molecular electronics sensor. The device has wireless communication links that can be further used to connect to a cell phone, smart device, or the cloud. This enables software updates, as well as connections to centralized reporting databases that can be used to provide monitoring of infectious disease outbreaks at a local, regional, national or global level. In other some embodiments, the Personal Virometer has the form factor of a portable device, as shown in FIG. 67.1. This form is suitable for higher throughout applications, for processing a greater number of samples, greater diversity of sample types, or greater diversity of assay types.

An exemplary virometer device may include an electronic instrument capable of running the molecular electronics sensor chip, a means to apply a sample to the sensor chip, and a means of processing this data to produce a report out on which if any of a set of viruses is detected. In one implementation, the output of the measurement may be transmitted to a centralized or cloud-based database. In another embodiment, this transmission occurs by wireless connections, including direct cellular connections, W-Fi or Bluetooth connections, or by connecting to another such connected device, such as a portable computer, cellphone, smart watch or smart ring, either by a local wireless connection, or by direct physical docking to such devices.

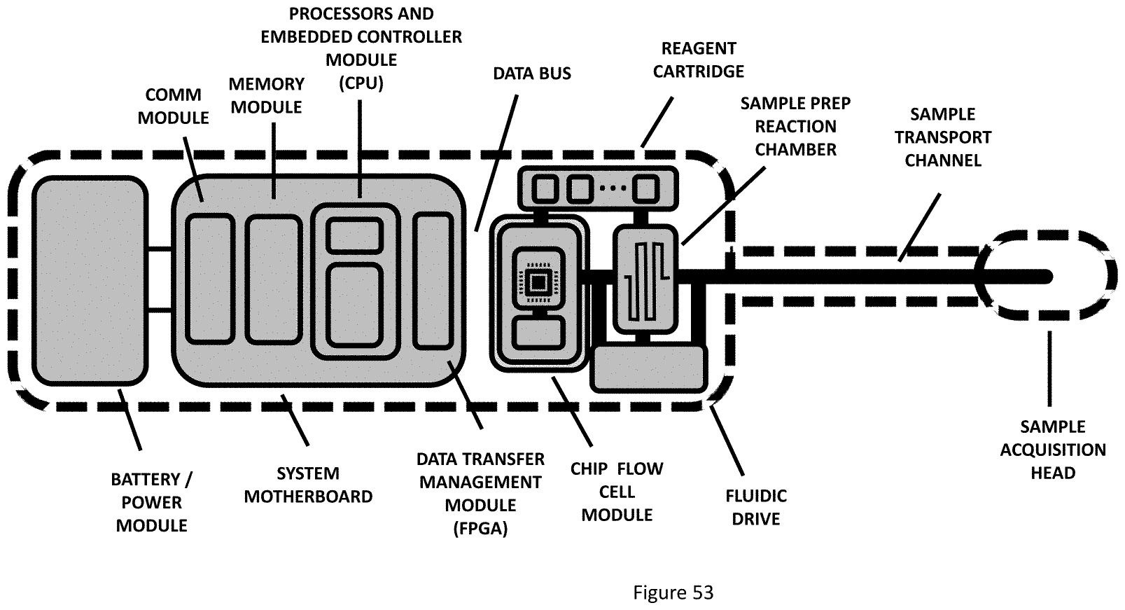

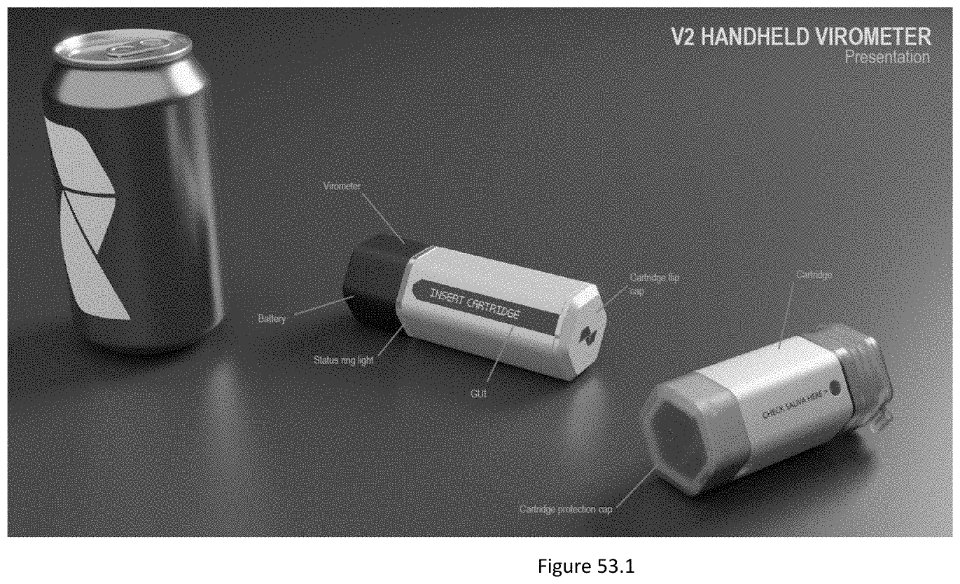

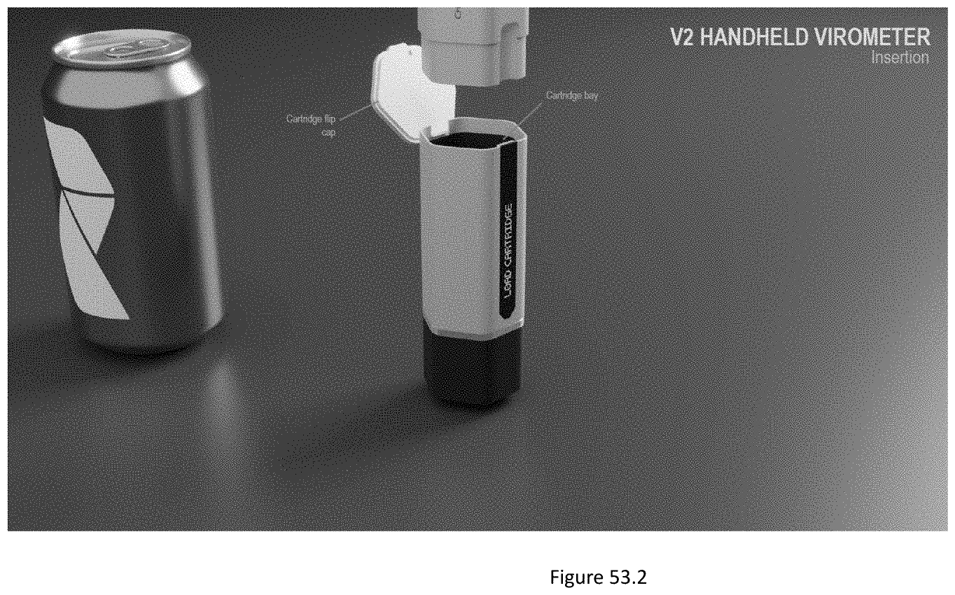

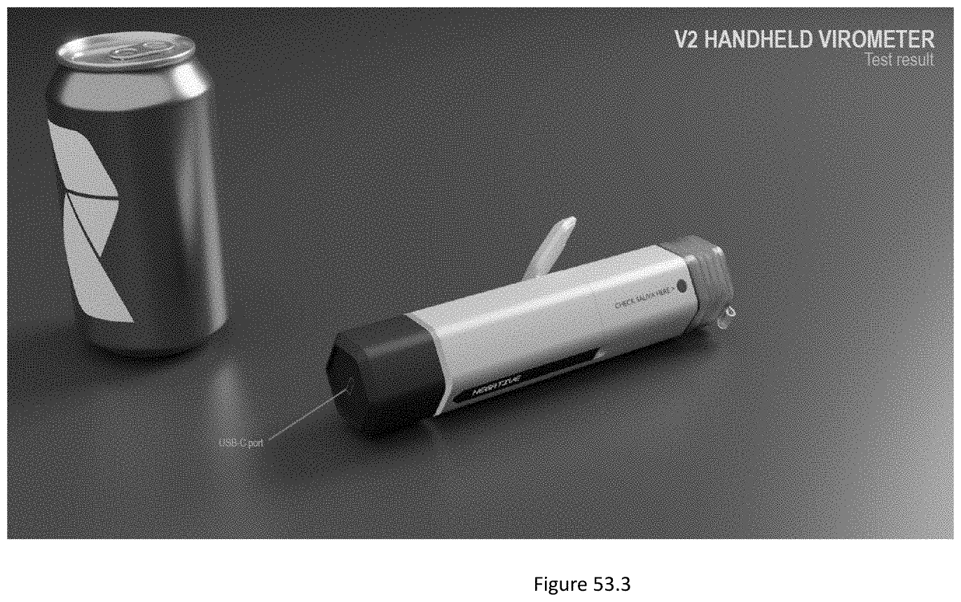

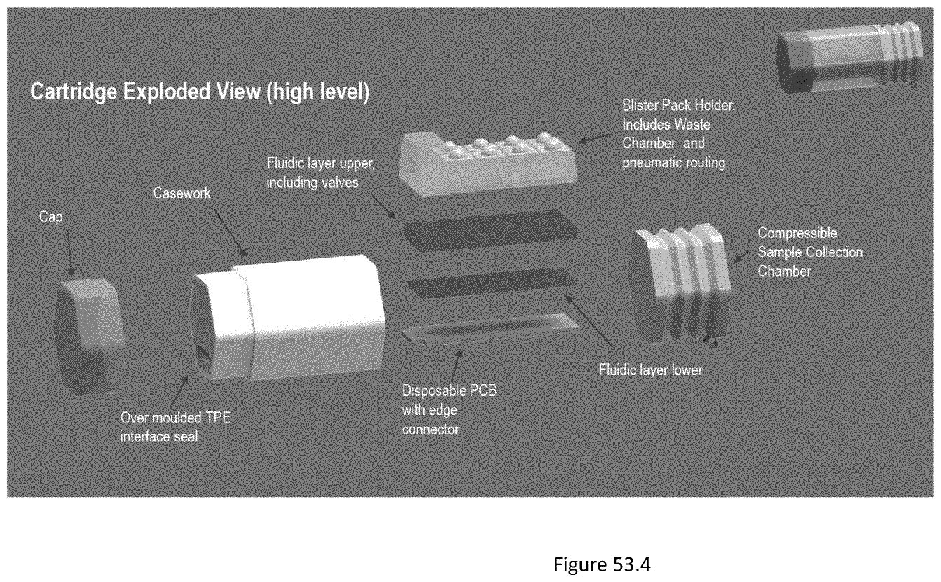

FIG. 53 an exemplary system for a Personal Virometer architecture. There is a sample acquisition head on the disposable sample, and a channel and fluidics drive for transporting an acquired sample from the head to a first reaction chamber. This reaction chamber performs reactions needed to prepare the sample to go on chip, which may include lysis, extraction, purification, buffer exchange, stabilization, or amplification reactions such as PCR reactions or isothermal PCR. The reagents to support these exemplary functions are supplied by the reagent cartridge. The prepared sample then goes into the chip flow cell, under the action of the fluidic drive. Data coming of the sensor chip travels on a data bus to a system motherboard. In some embodiments this is done by a data transfer module comprising a Field Programmable Gate Array (FPGA). This module may also perform a first phase of data analysis or reduction. This module moves the sensor data to a Processor module, for final processing, from where it is transferred to a memory module, such as DRAM or SSD or a Hard drive. From there it may also be transferred, all or in part, by the comms module, off device, either by a hard connection or a wireless link. The device generally has a power module, or battery module, used to provide system power. The overall operations of the system are controlled by an embedded microcontroller residing on the system board.

FIGS. 53.1, 53.2, 53.3, and 53.4 show renderings from CAD designs for the Personal Virometer illustrated in FIG. 53. These figures depict, for example, a standard size can of soda, at left, as a scale reference. FIG. 53.1 shows the virometer dissembled, with the hand-held end of the virometer (left), and the disposable sample cartridge end (right). In this embodiment, the consumable chip and reagents reside in the sample cartridge portion of the system, so that they can all be disposed of in a unit. FIG. 53.2 shows how the sample cartridge fits into the hand-held base. FIG. 53.3 shows the cartridge and base assembled into testing configuration, while the test is in process. FIG. 53.4 shows an exploded view of the disposable sample cartridge end, which shows the reagents in a blister pack packaging, the fluids control layer, and the chip board layer. In this embodiment, the saliva collection end is a compressible “accordion” chamber, that is manually compressed to drive the saliva into the fluidics.

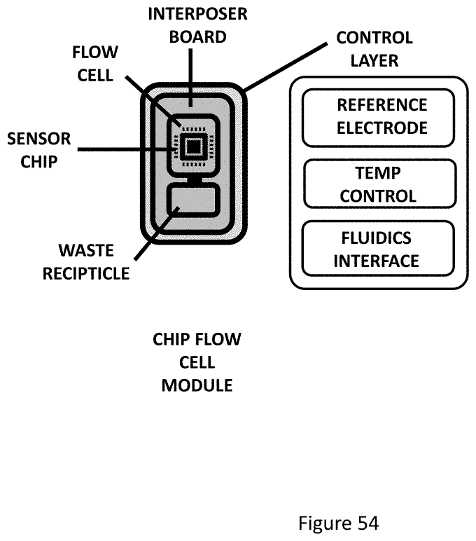

FIG. 54 shows a schematic view of an exemplary embodiment of the chip flow cell module. The chip is mounted on a substrate, which may be wire bonding to a PCB in some embodiments. In some embodiments, this may be mounter on an interposer board that facilitates docking into the system board. The chip is covered by a flow cell that contains the fluids applied to the chip, as well as an outlet to a fluid waste repository in some embodiments. All this is mounted on a control layer, that also houses a reference electrode for setting fluid potential, a temperature control device, which in some embodiments is a Peltier device temperature controller, and a fluidics interface.

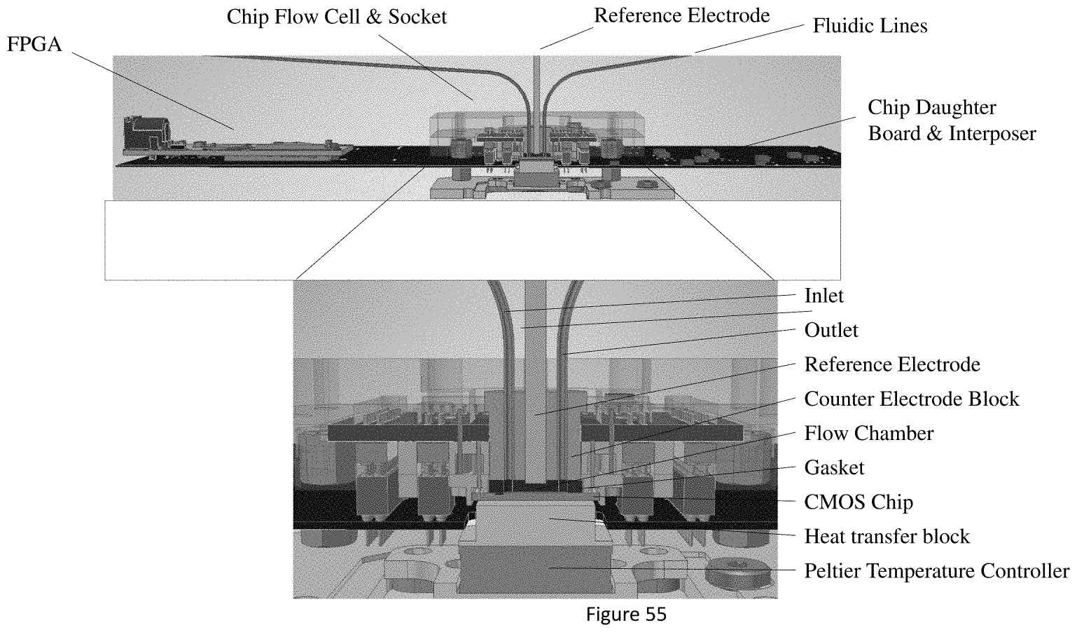

FIG. 55 provides a detailed CAD design for an exemplary embodiment of the chip flow cell module of FIG. 54. The upper panel shows the context on the system board, with the FPGA data transfer device shown, as well as fluid inlet and outlet lines going into the flow cell, a reference electrode going into the cell, and the chip daughter board and interposer. The lower panel shows an expanded view of the flow cell and socket. In this embodiment, the electrical connection is via pogo-pin connectors to the chip. Details of the fluidic design, reference electrode design, and temperature control design are shown there.

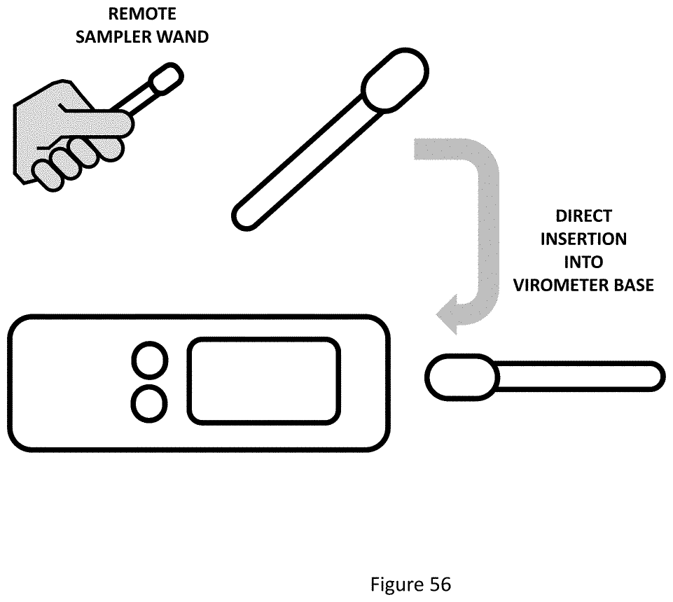

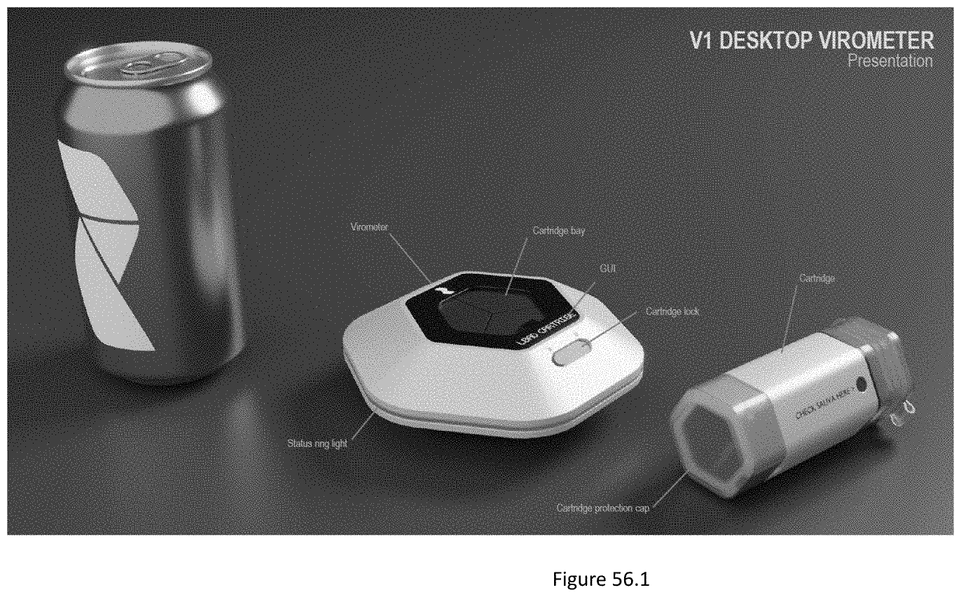

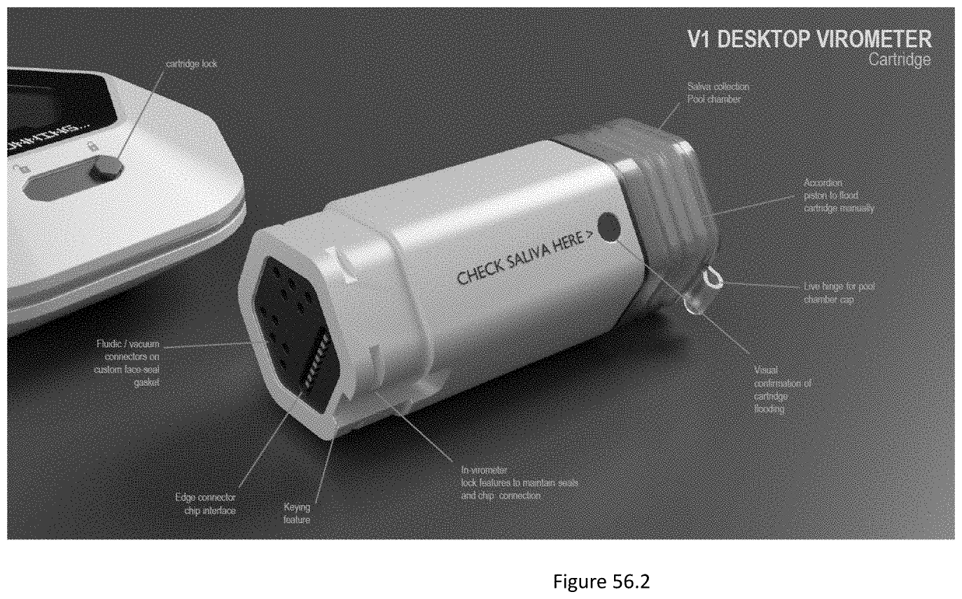

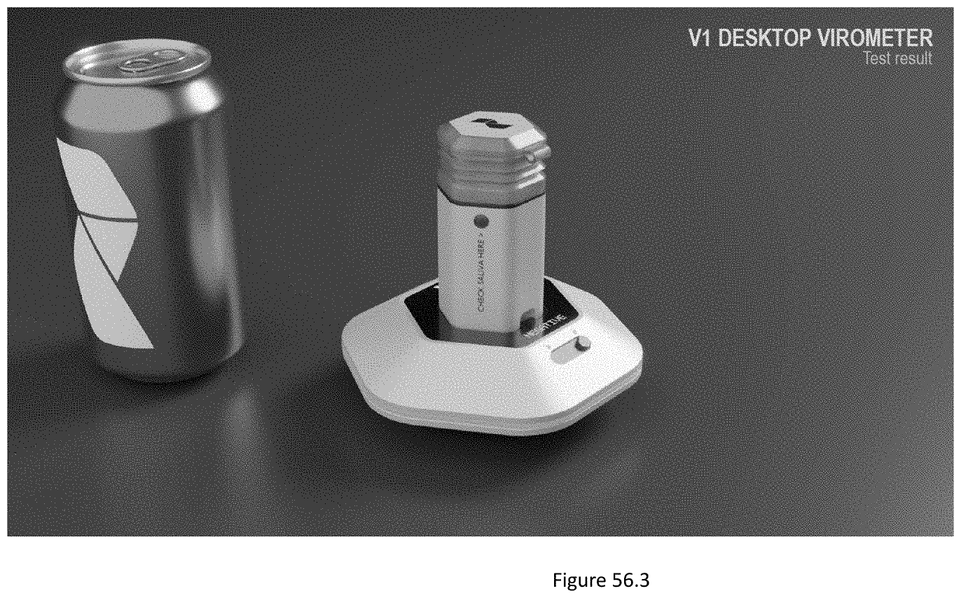

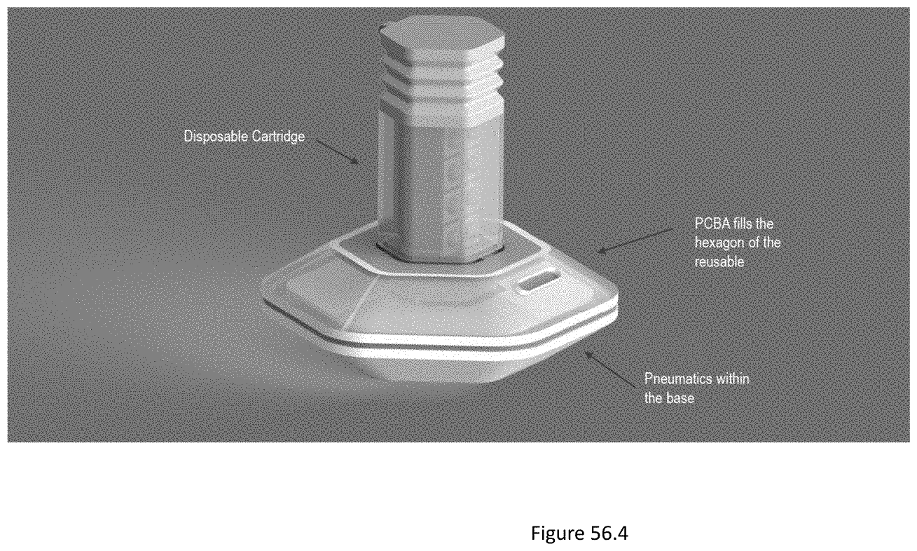

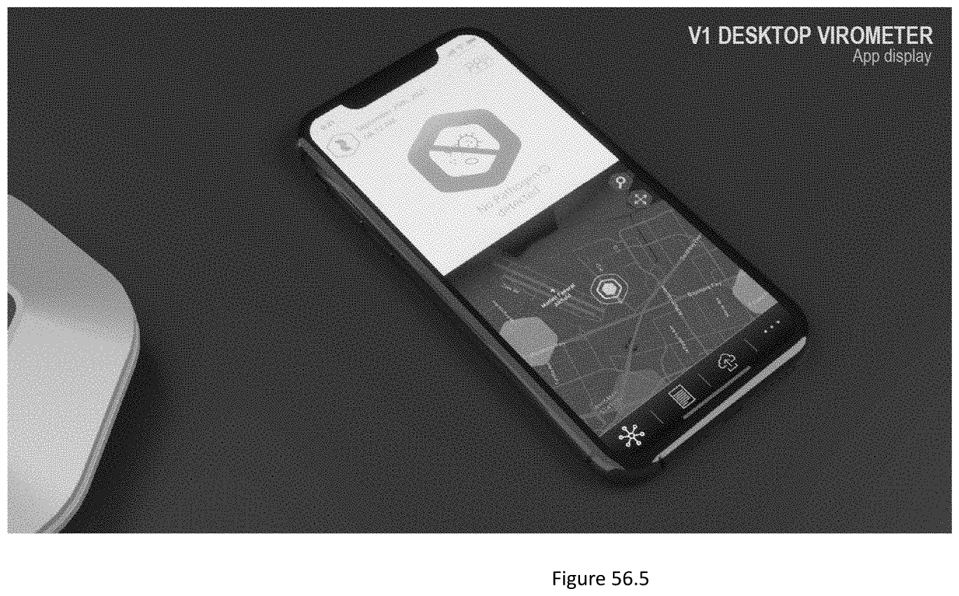

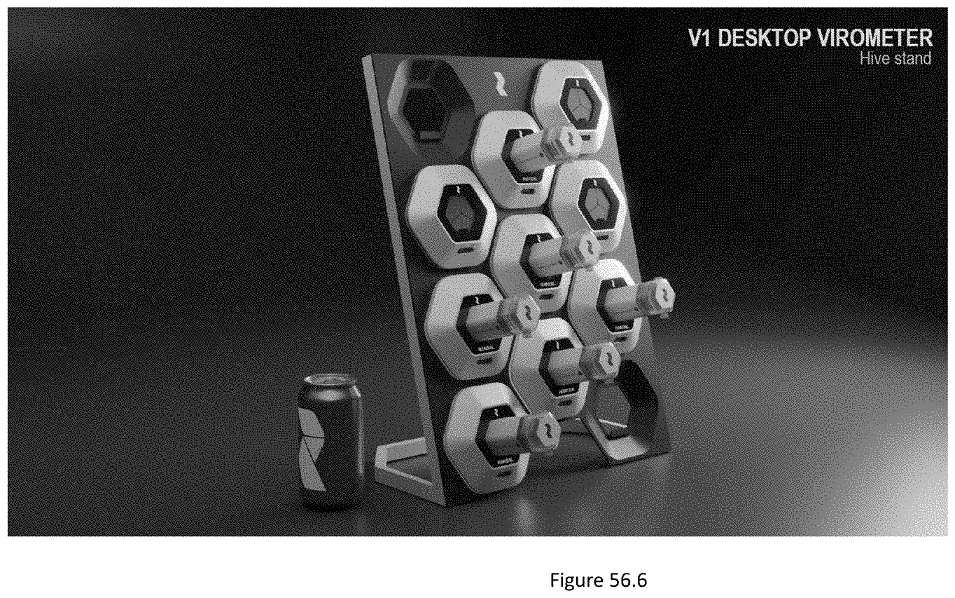

FIG. 56 illustrates another exemplary embodiment of the Personal Virometer where the sample is meant to be used as a handheld sample collection wand, which is used to collect a sample, and then is then manually inserted into the virometer base and interfaced with the sample prep reaction chamber, bypassing the need for sample transport from the collection head to the primary reaction chamber.

FIGS. 56.1, 56.2, 56.3, and 56.4 show renderings from CAD designs for the virometer illustrated in FIG. 56. These figures depict a standard size can of soda, at left, as a scale reference. FIG. 56.1 shows the hexagonal base station, and the disposable sample cartridge. In this embodiment, the consumable chip and reagents reside in the sample cartridge portion of the system, so that they can all be disposed of in a unit. FIG. 56.2 shows the sample cartridge features in more detail, as does the exploded view in FIG. 53.4. The detail shows a saliva collection chamber on one end, which may receive one or a pool of individual samples. Such a pool may be convenient for screening all family members in a single test, for example. FIG. 56.3 shows the cartridge and base in its testing configuration, while the test is in process. FIG. 56.4 shows the same view, with transparency applied to the outer surfaces of the rendering, so that the internal reagent cartridge is visible (blister pack format), and the circuit board and fluidics drive is visible within the base. FIG. 56.5 shows the associated informatic app for the virometer, running on a smart phone device. As indicated, the test results are displayed on the app, and test results along with GPS location data are also optionally uploaded to a cloud-based or web-based central monitoring site, either for personal monitoring, family monitoring, or larger scale group or population monitoring applications. FIG. 56.6 show multiple Personal Virometers mounted on a compact “hive” station. This station allows for convenient management of multiple virometers, for high throughout testing. The hive station may also supply power or communications support, such as internet or cloud access, for the virometers.

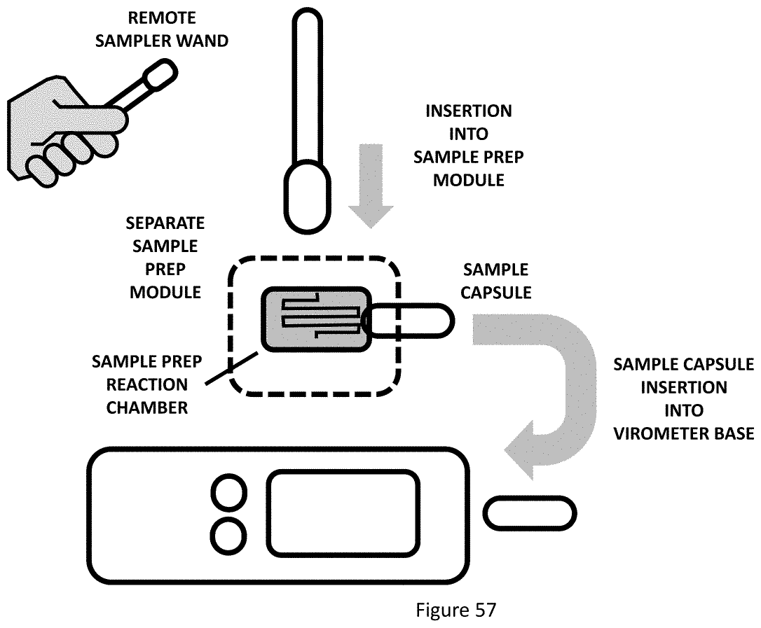

FIG. 57 illustrates another exemplary embodiment of the Personal Virometer where the sample is meant to be used as a handheld sample collection wand, which is used to collect a sample, and then is then manually inserted into a separate sample prep base, where the primary sample prep reaction takes place, instead of such a reaction occurring inside the virometer base. The sample prep module produces a capsule loaded with prepared sample, which can then be inserted manually into the virometer base. This further eliminates the need to integrate the sample prep reaction chamber into the handheld base, thereby putting less constraints on the instrument integration.

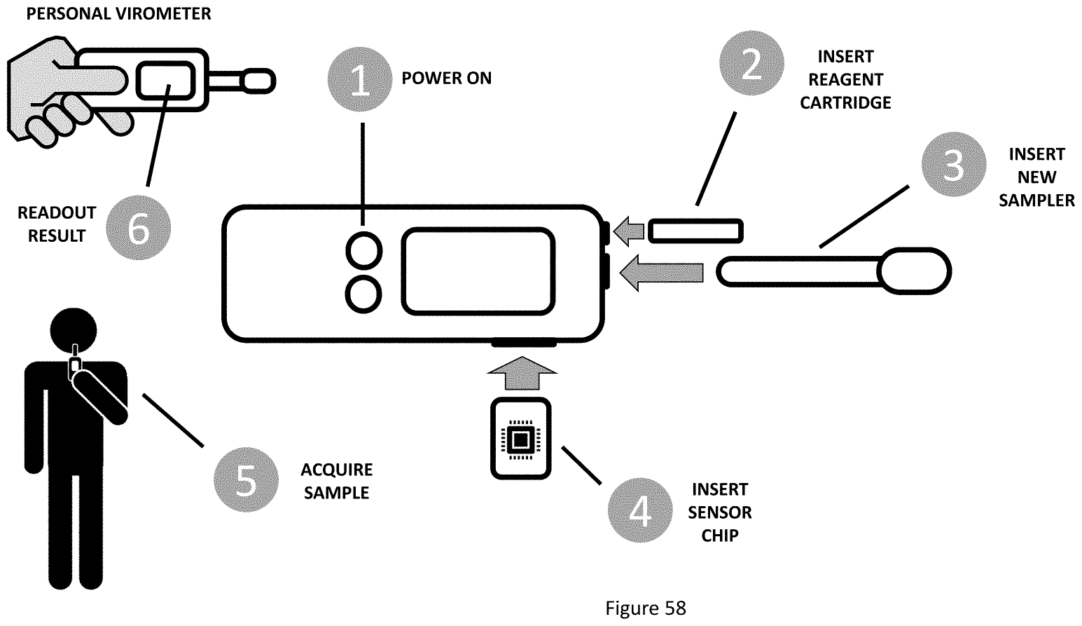

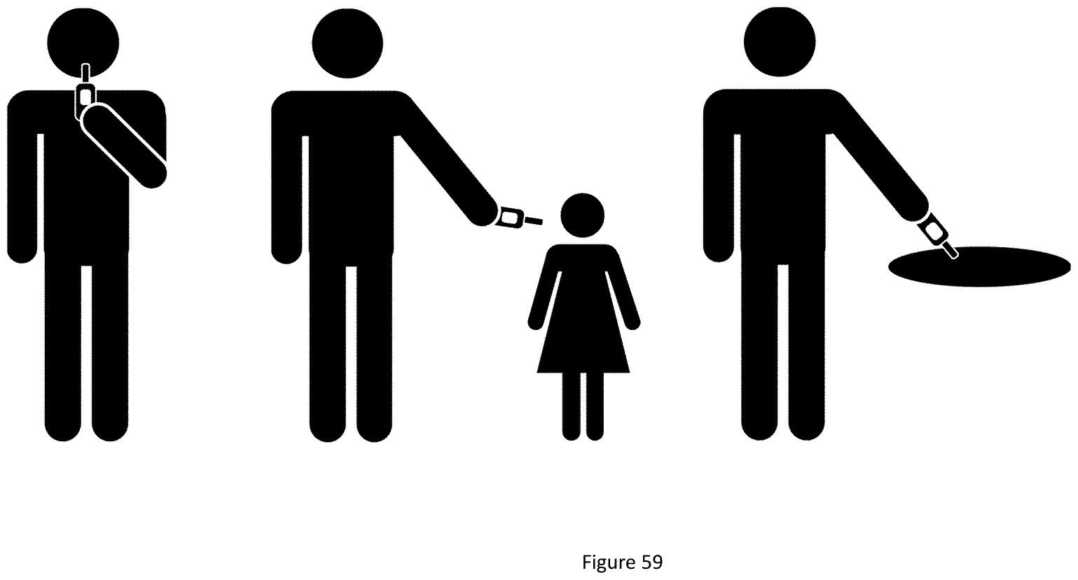

FIG. 58 illustrates an exemplary embodiment of a method of use for the Personal Virometer. Initially the device is turned on, all consumables are inserted and logged by the system: reagents, sampler wand, and chip. Then the device testing mode is selected, and a sample is acquired. Finally, after the required waiting time, the readout is available on the screen.

FIG. 59 illustrates three different some embodiments of use cases and methods of use: At left, an individual may take their own sample, for self-testing. In the middle, the sample for testing may be acquired from another person, such as a child. At right, the sample may be an environmental sample acquired by swabbing a surface, or from filtrate acquired from water or air.

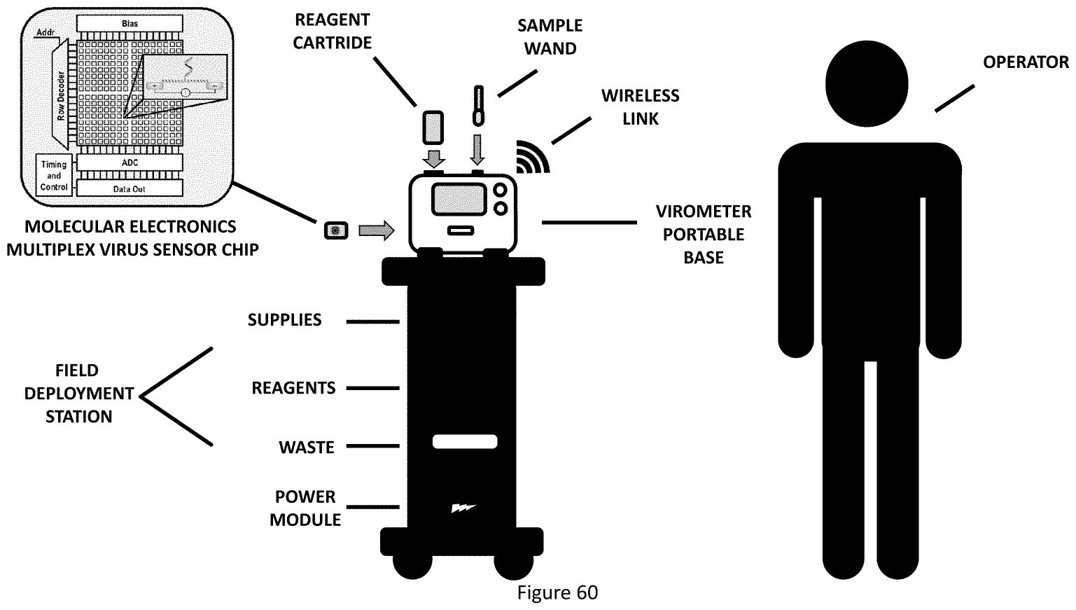

FIG. 60 illustrates an exemplary embodiment of the Personal Virometer in which the virometer has a portable form factor. In some embodiments, this may include an auxiliary mobile cart, which includes a battery or power converter module to power the virometer and any auxiliary powered equipment, a waste receptacle to receive all biological and biohazard waste, including used chips, reagent cartridges and sample wands, a reagent repository for the chips, reagent cartridges and sample wands, which may be a refrigerated or temperature controlled repository in some embodiments, and a supply repository, which may hold additional supplies, such as gloves, masks or personal protective equipment for the operator, or may also hold samples that are awaiting further processing. The portable format of the virometer instrument has a base with legs, suitable for sitting on a surface, and such legs may be made of materials such as rubber or plastic, to reduce vibration of the instrument, or prevent slipping. The instrument has a slot to take in the chip, a slot to take in a reagent cartridge for the reagents that support the sample prep or detection assay, a wireless link for the virometer communications, and front control buttons and a user screen, as indicated in FIG. 60. In exemplary embodiments, an operator attends to the virometer and cart, to facilitate collection and testing of multiple samples, according to defined protocols and procedures.

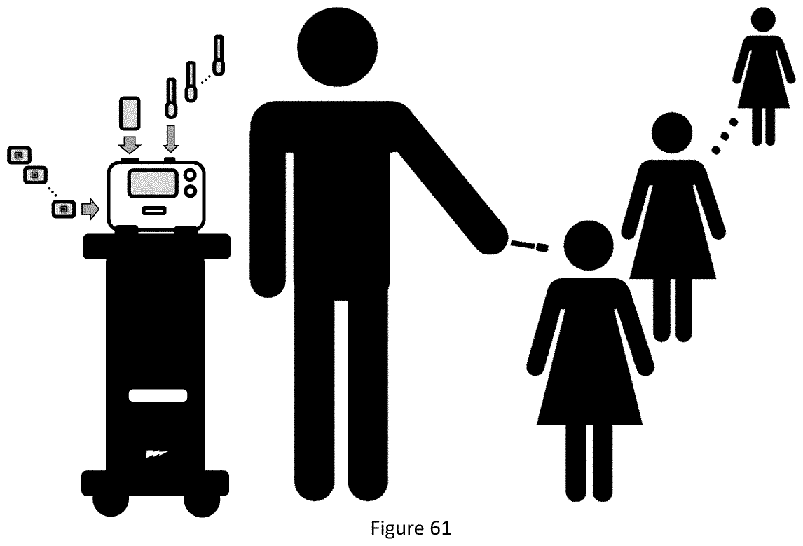

FIG. 61 illustrates exemplary methods of use of the portable virometer, in which multiple subjects are tested. In the course of this, the operator uses multiple sample wands to acquire samples from the test subjects, and the virometer is used to process this series of samples, using a series of chips. The virometer may in some embodiments process one sample and chip at a time, while in other some embodiments, the virometer can be loaded with multiple chips and multiple samples for simultaneous automated processing. In some embodiments, the identity of each sampled individual (which may include personal identifiers such as name or government issued ID codes, as well as a time-stamp and GPS coordinates or location indicator) is affiliated with a barcode or RFID for subsequent sample tracking, and such tracking codes are transferred to the instrument, for affiliation with reports or uploads of data to cloud or remote data repositories.

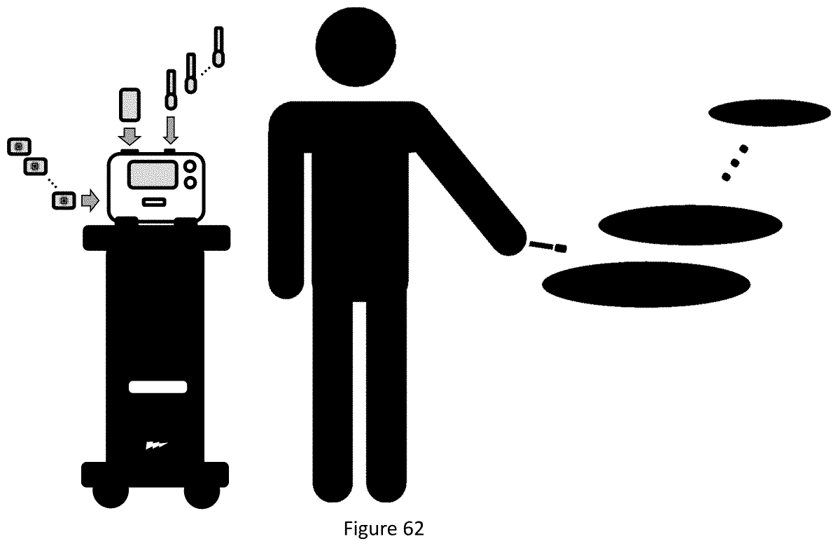

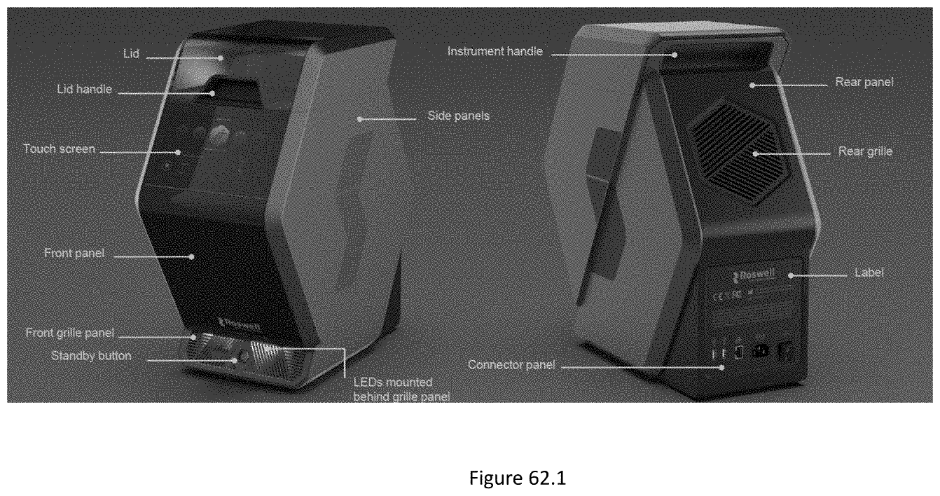

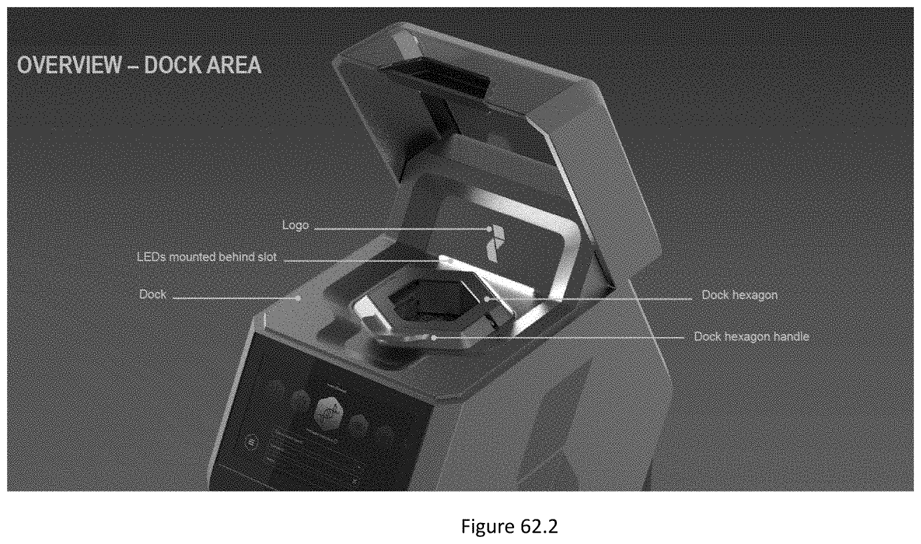

FIG. 62 illustrates exemplary methods of use of the portable virometer, in which multiple environmental samples are tested. In the course of this, the operator uses multiple sample wands to acquire samples from surface swabs, or air or water, or filtrates of air or water, and the virometer is used to process this series of samples, using a series of chips. The virometer may in some embodiments process one sample and chip at a time, while in other some embodiments, the virometer can be loaded with multiple chips and multiple samples for simultaneous automated processing. In some embodiments, the identity of each sample (which may include location identifiers addresses, room numbers, and landmarks, well as a time-stamp and GPS coordinates or location indicator) is affiliated with a barcode or RFID for subsequent sample tracking, and such tracking codes are transferred to the instrument, for affiliation with reports or uploads of data to cloud or remote data repositories.

FIG. 62.1 shows a rendering of a CAD design for of one exemplary embodiment of a portable virometer appropriate for cart-based portable use. Major elements of the external instrument are annotated, including the lid, user interface screen, and rear connectors. FIG. 62.2 shows the features with the lid open, showing the docking site for the chip and sample module.

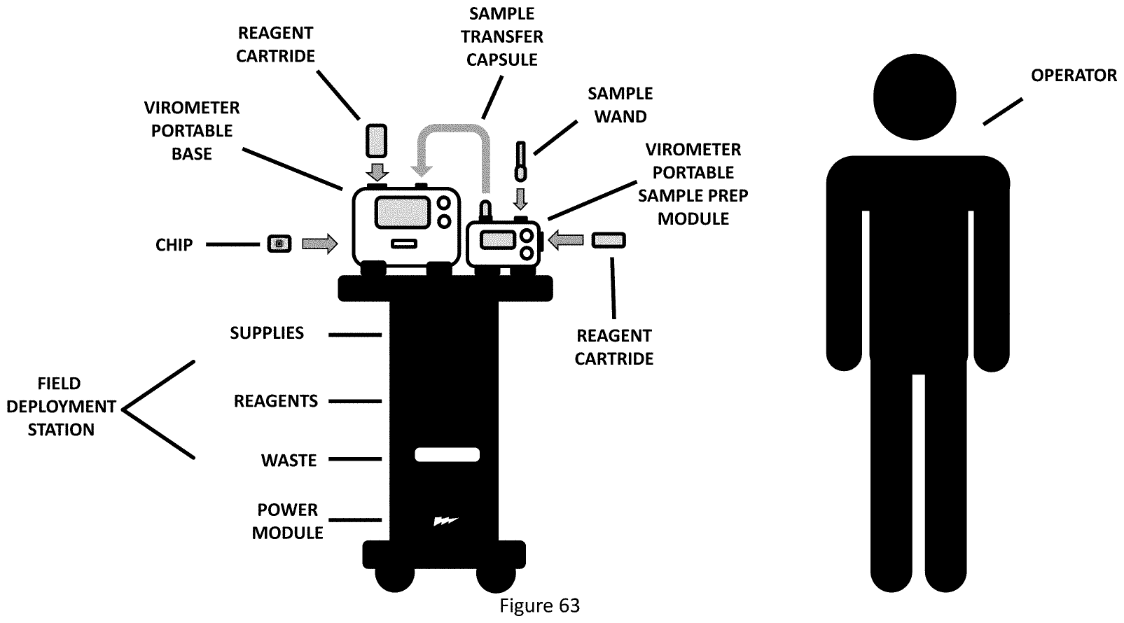

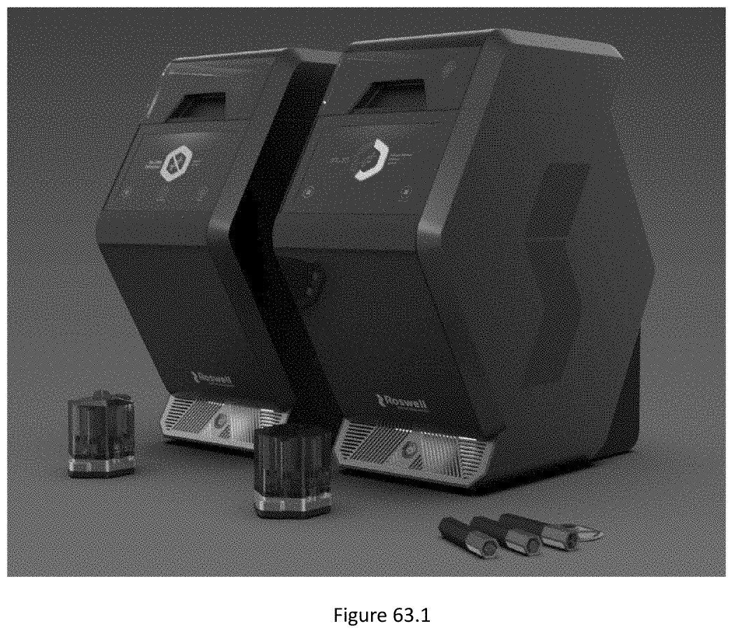

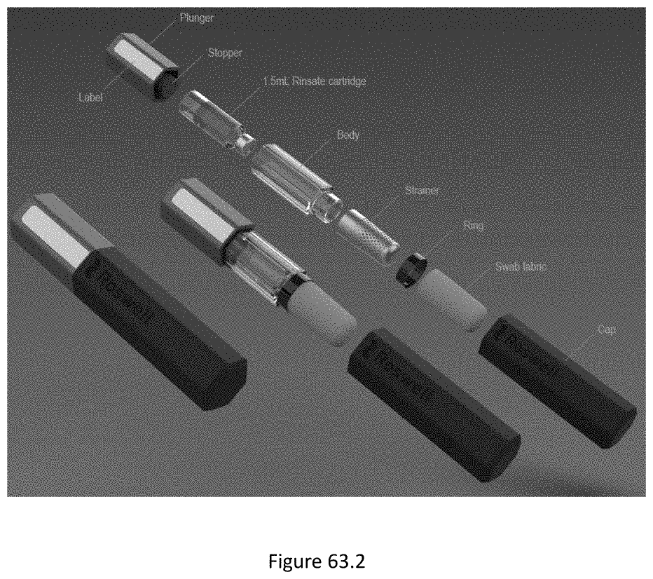

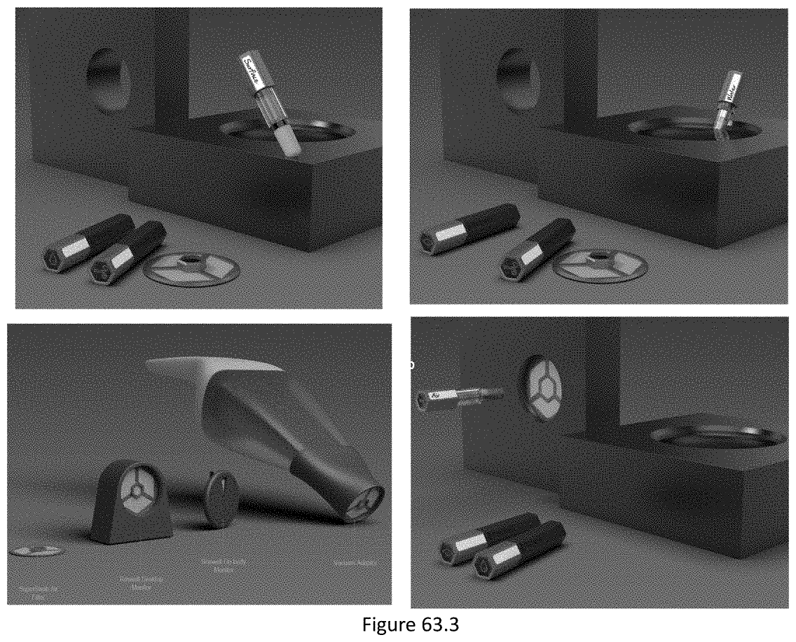

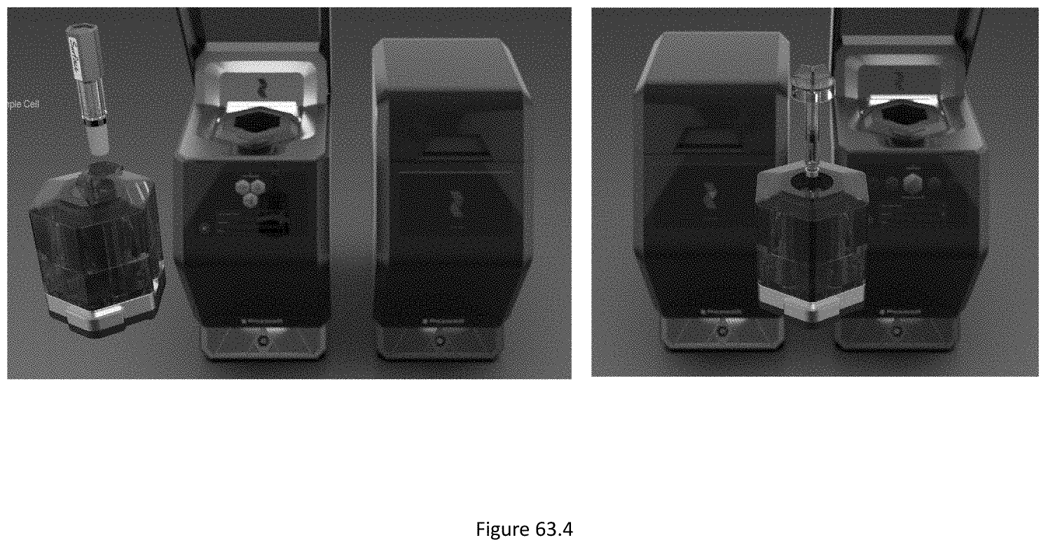

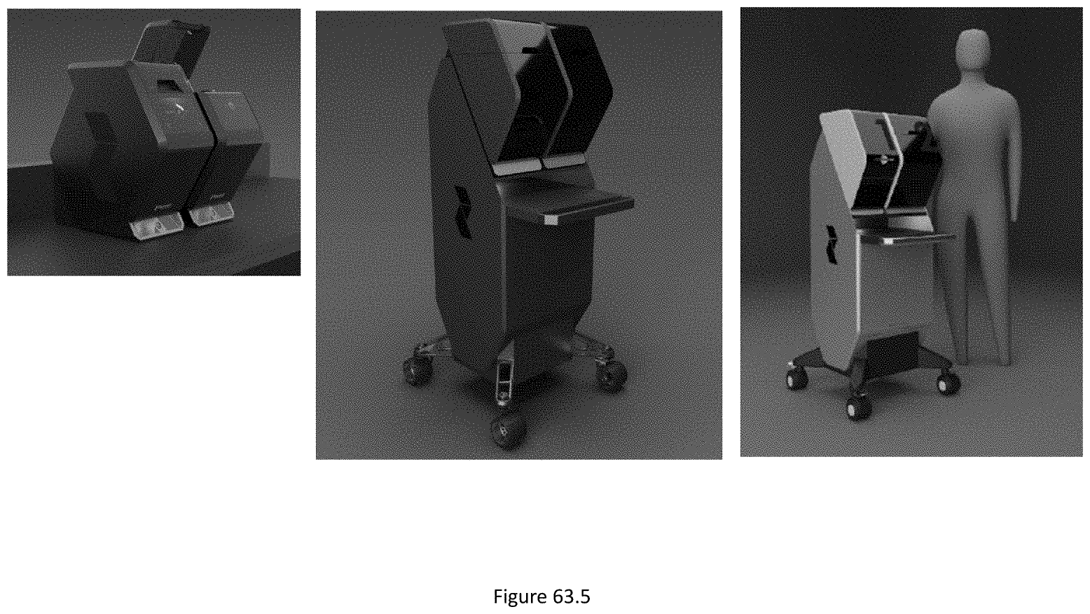

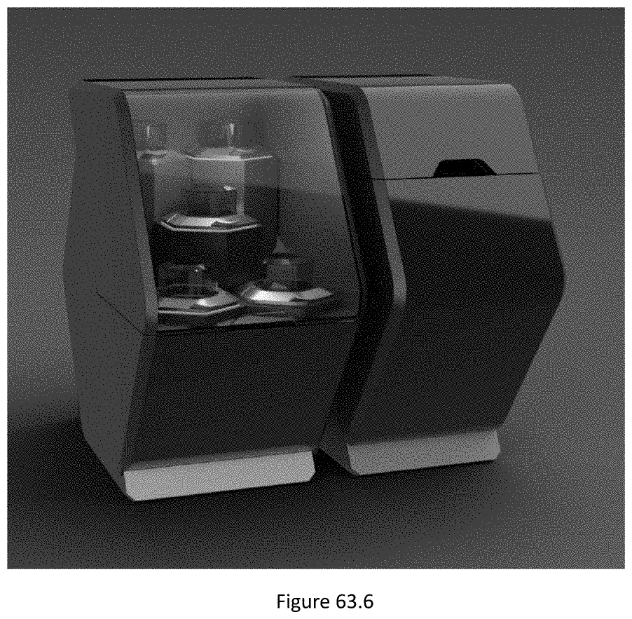

FIG. 63 illustrates an embodiment of the portable virometer in the sample processing module is resident in a separate instrument, which processes a sample collection wand, and produces a capsule of prepared sample, for subsequent on-chip analysis in the virometer base.

FIG. 63.1 show a rendering of a CAD design of one exemplary embodiment in which the sample processing module (left) and sensor chip reading module (right) reside in separate instruments. Shown with these are the sample insertion modules for each instrument (small hexagonal blocks) and one exemplary embodiment of the sample collection wands (foreground).

FIG. 63.2 show a rendering of a CAD design of one exemplary embodiment for the sample collection wand, for environmental sampling or other swab-based sampling. The exploded view illustrates major components of the wand, which provides for a plunger to push a rinsate through the primary sample collection material which is a swab fabric as shown.

FIG. 63.3 show a rendering of a CAD design of one exemplary embodiment for the sample collection wand, illustrating different use cases for environmental sampling applications: swabbing from a surface (upper left), sampling from a pooled water source (upper right), filters and vacuum or fan driven attachments for collecting primary samples from air (lower left), and the sample wand positioned for punch-through and capture of the air filter material (lower right).

FIG. 63.4 show a rendering of a CAD design of one exemplary embodiment in which the sample processing module (left) and sensor chip reading module (right) are separate instruments, and illustrates how the primary sample collection wand fits into a fluidics-enabled carrier module for mounting in the instrument to prepare a sample (left), and then the resulting prepared sample (right) is loaded into a fluidics-enabled carrier module, including the chip, for loading into the reader instrument.

FIG. 63.5 show a rendering of a CAD design of one embodiment, in which the separate instruments for sample prep and chip reading may be deployed on a desktop (left), or on a mobile field deployment cart (center and right), as previously indicated in FIG. 63.

FIG. 63.6 show a rendering of a CAD design of one embodiment in which the sample processing module (left) and sensor chip reading module (right) reside in separate instruments, and in which the sample prep instrument is configured to process multiple samples in parallel, as also indicated in FIG. 62. In the embodiment shown, multiple of sample transfer blocks with fluidic drives as indicated in FIG. 63.4 (left) fit into the sample prep instrument. The chip reader may be similarly configured to support multiple blocks, and in some embodiments the numbers matched to match the throughput of sample prep and reader. For example, if the reader is 5 times faster than the sample prep, for the system shown in FIG. 63.6, the chip reader may be configured to have a single block, so that in a continuous workflow, the rate of producing samples for reading equals the rate of reading such samples.

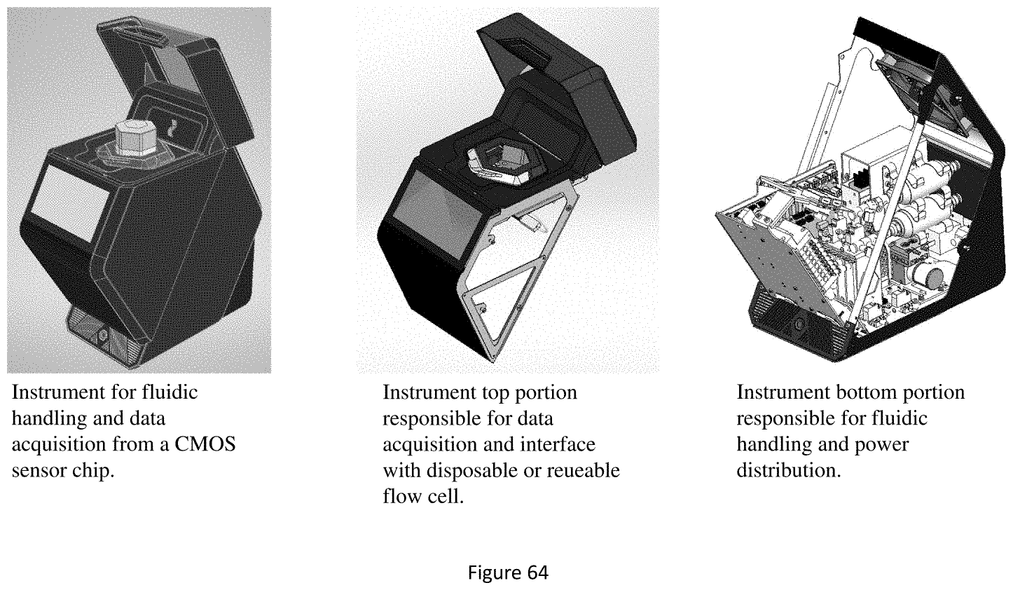

FIG. 64 shows detailed CAD designs of one exemplary embodiment of the Personal Virometer in the portable form factor. The inset figures show the external form, with chip flow cell in place, the upper portion of the instrument without the flow cell in place, and the internal subsystems for fluidic drive and power distribution.

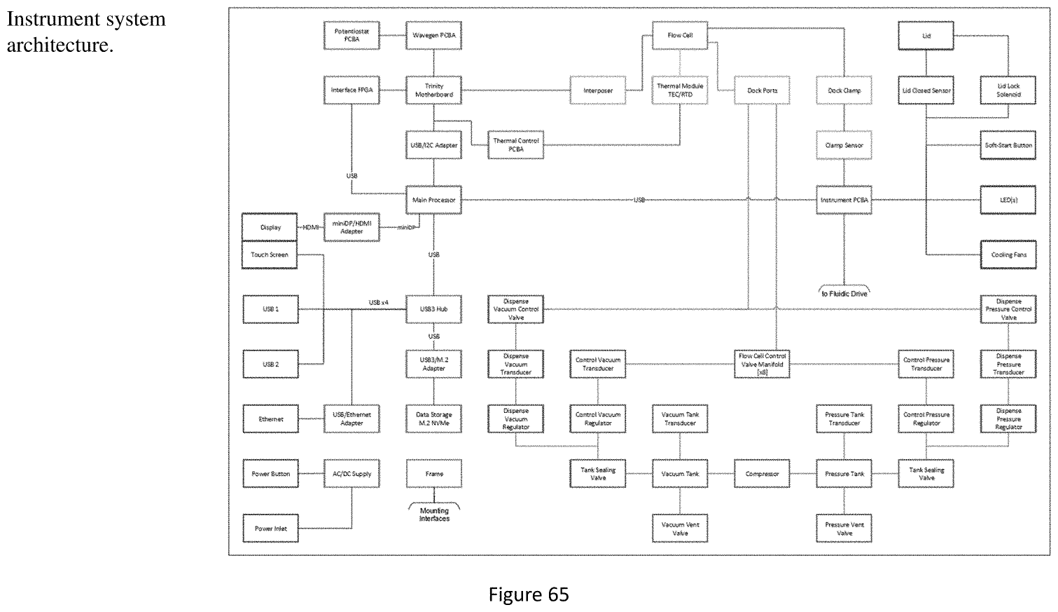

FIG. 65 shows the detailed system architecture schematic of an exemplary embodiment of the Personal Virometer which may have a portable form factor. The schematic shows essential system connections between various components and subsystems of the exemplary embodiment, including electrical and fluidic connections, and control connections.

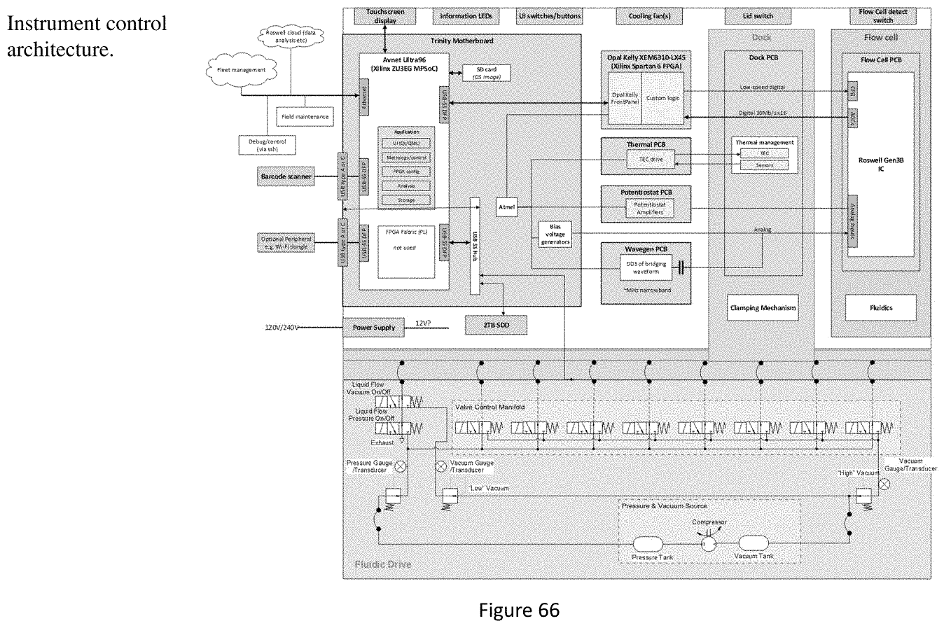

FIG. 66 shows the detailed system control architecture schematic of one exemplary embodiment of the Personal Virometer which may have a portable form factor. In this schematic, the master microcontroller is the Avnet Ultra96 FPGA module on the left. The sensor chip is indicated as the Roswell Gen3B IC on the far right. Connections indicate the flow of control signals or digital data signals coming off chip (ADCs).

FIG. 67 shows the detailed system wiring diagram schematic of one exemplary embodiment of the Personal Virometer which may have a portable form factor. Connections indicate all electrical wiring connections between components and subsystems.

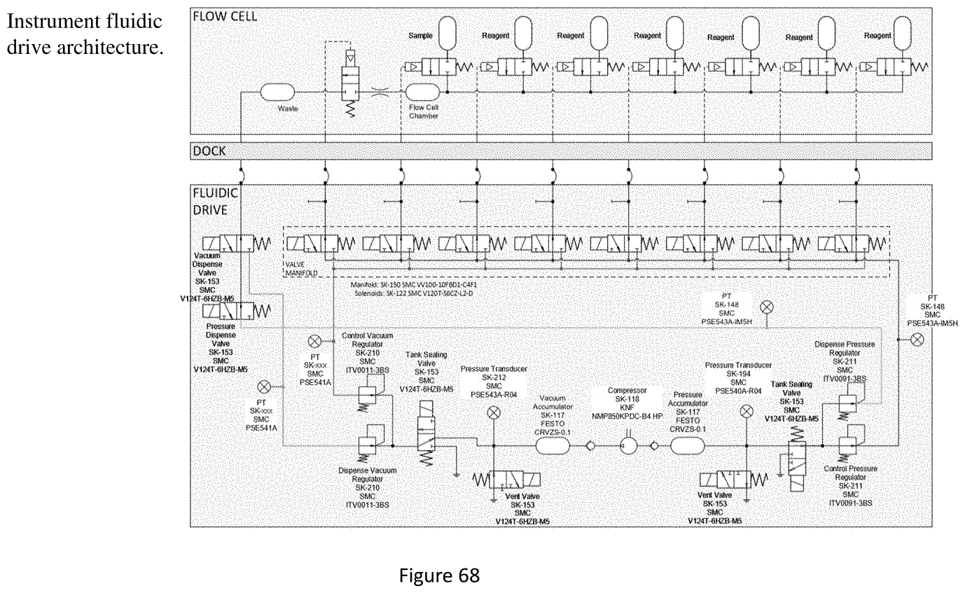

FIG. 68 shows the detailed fluidic drive schematic of an embodiment of the Personal Virometer which may have a portable form factor. The flow cell is indicated on top, with drives for transfer of sample and reagents. The dock layer provides interfaces to system vacuum and pressure sources, under the regulation of various valves. In this manner, the system is provided with pressure to push fluids, and vacuum to pull fluids, as needed in various modes and phases of operation.

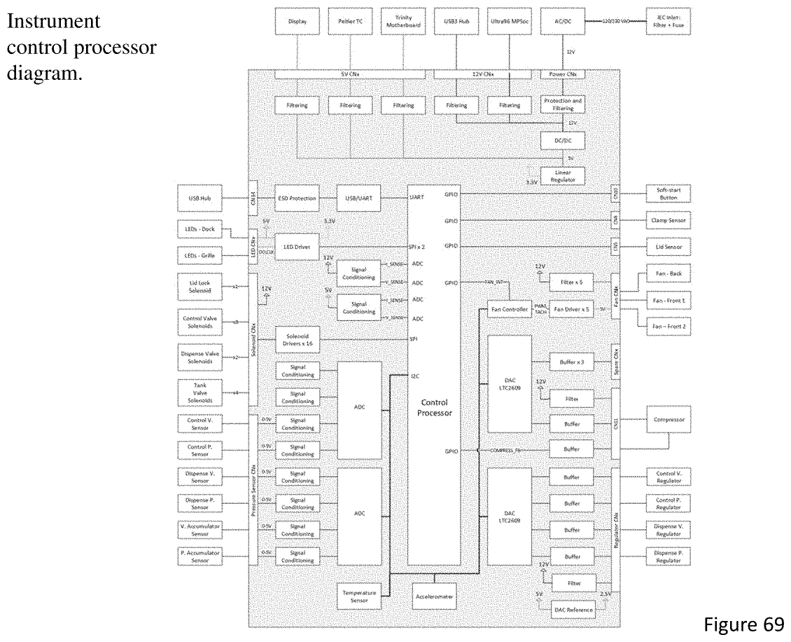

FIG. 69 shows the detailed system control schematic of one embodiment of the Personal Virometer which may have a portable form factor. The primary control processor (microcontroller) is indicated by the central block, and the diagram shows all the control signal signals and lines emanating from this.

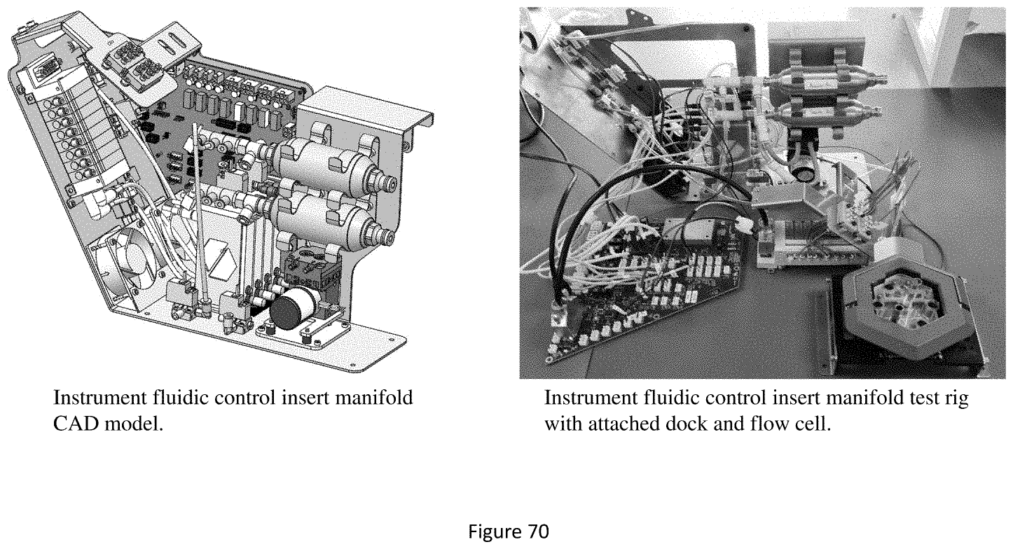

FIG. 70 shows the fluidic control insert manifold of one embodiment of the Personal Virometer which may have a portable form factor. At left is shown a CAD model of the manifold, and at right is shown a physical form of the manifold, integrated into a fluidic test bench for system testing.

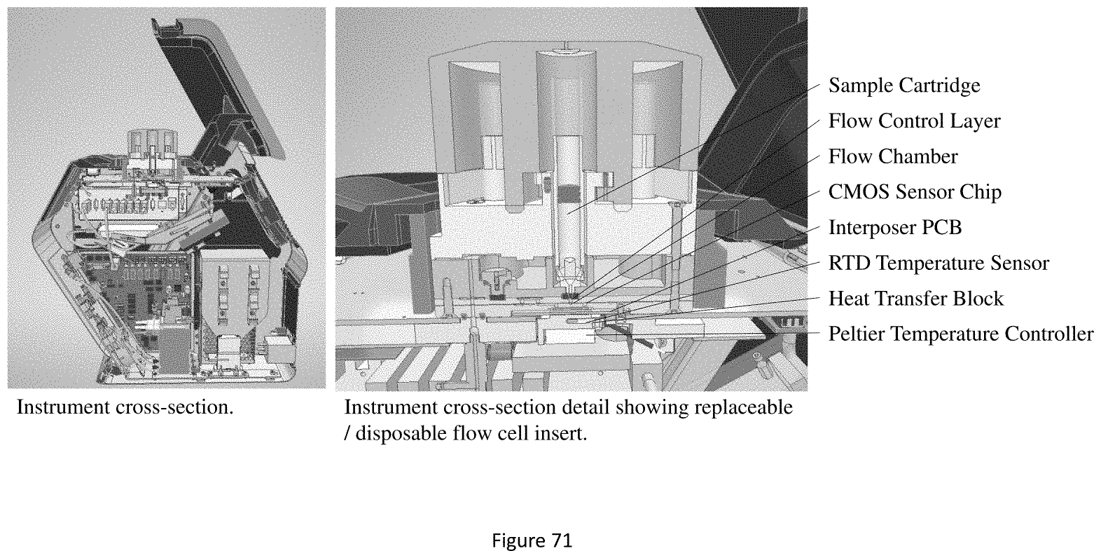

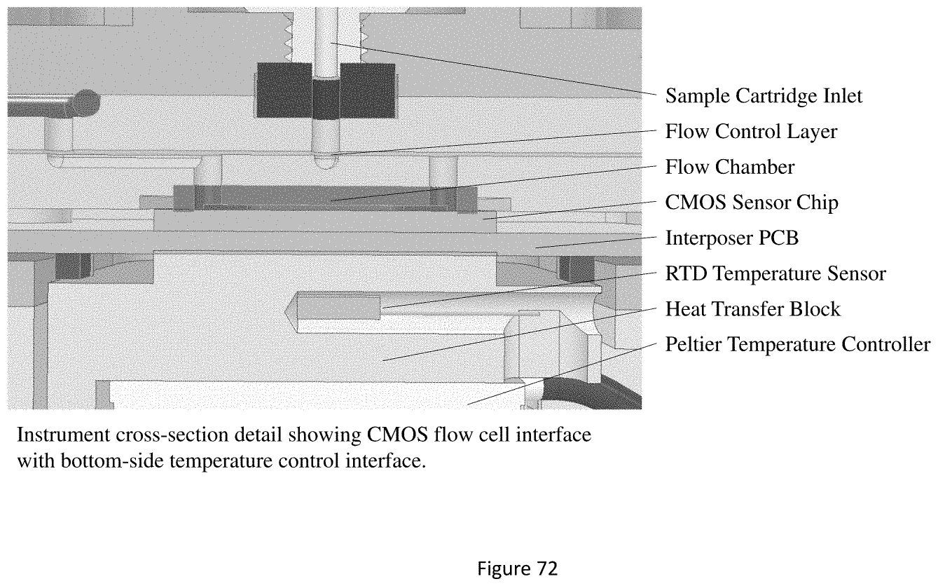

FIG. 71 shows detailed CAD designs of an exemplary embodiment of the Personal Virometer which may have a portable form factor. At left is a CAD model of a full-instrument cross section. At right is a close-up of the replaceable or disposal elements of the chip flow cell region, including the sample injection cartridge, flow cell and CMOS chip, and also highlighting elements of the temperature control system. FIG. 72 shows a further close-up of this region.

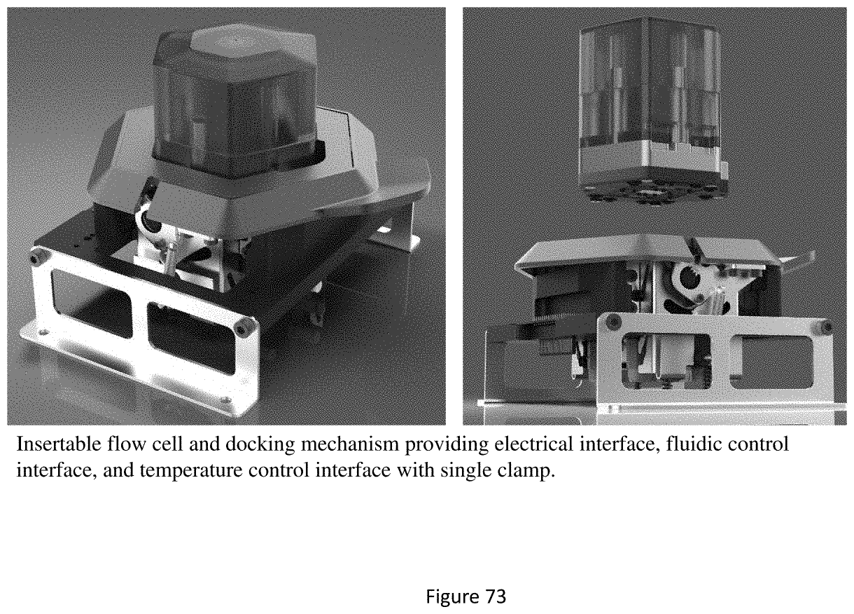

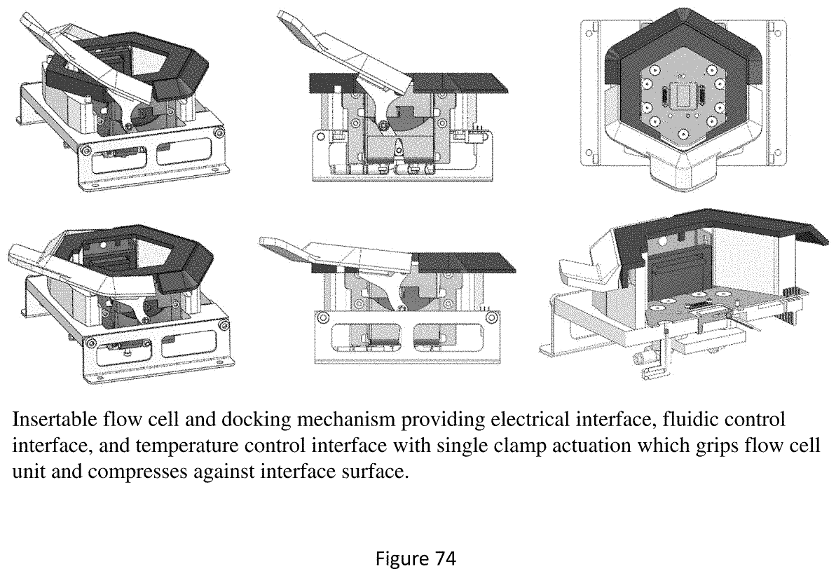

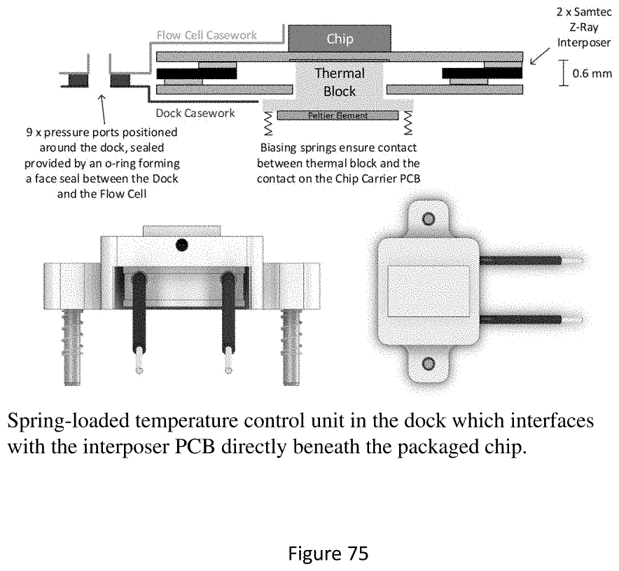

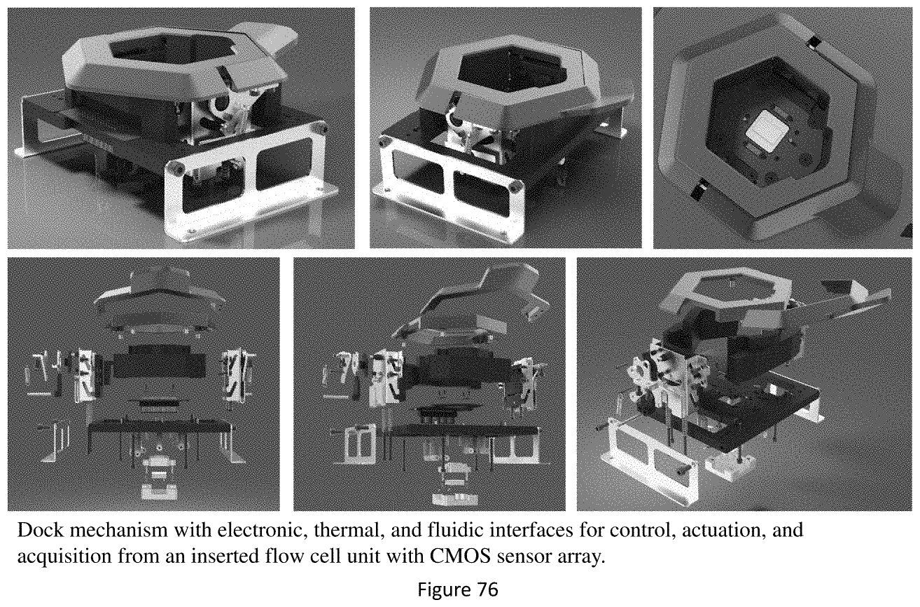

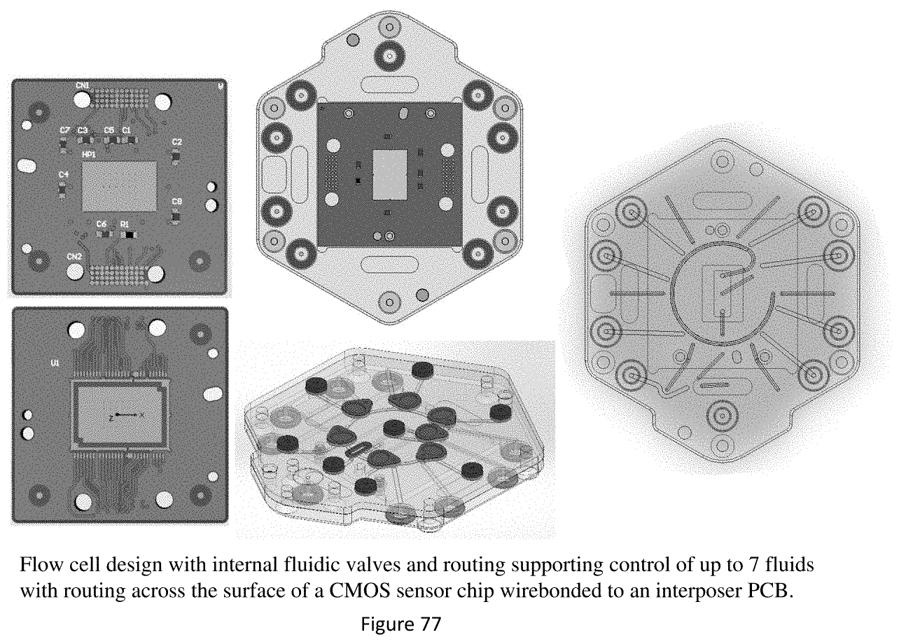

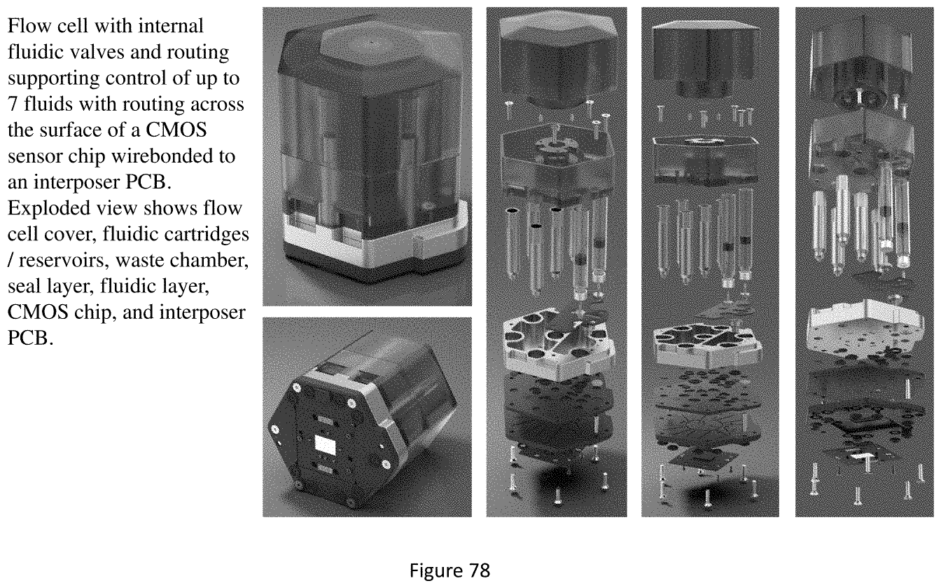

FIG. 73 shows CAD renderings of the flow cell docking module of one embodiment of the Personal Virometer which may have a portable form factor. At left is shown the flow cell docked into the fluidic/electrical connection socket. At right is shown the flow cell undocked, and the clamp mechanism used to engage and disengage the flow cell. FIG. 74 shows detailed drawing of the operation of the flow cell docking mechanism. FIG. 75 shows detailed drawing of the temperature control unit that the docking process places against the bottom of the chip. FIG. 76 shows CAD renderings of exploded views of the docking mechanism, showing the major component parts of the mechanism. FIG. 77 shows detailed designs of the routing and fluidic control layer that supports routing up to 7 different liquid reagent sources to apply them to the CMOS sensor chip in the flow cell. FIG. 78 shows CAD renderings of the exploded view of the reagent reservoirs, fluidic control layer, flow cell, CMOS chip and temperature controller.

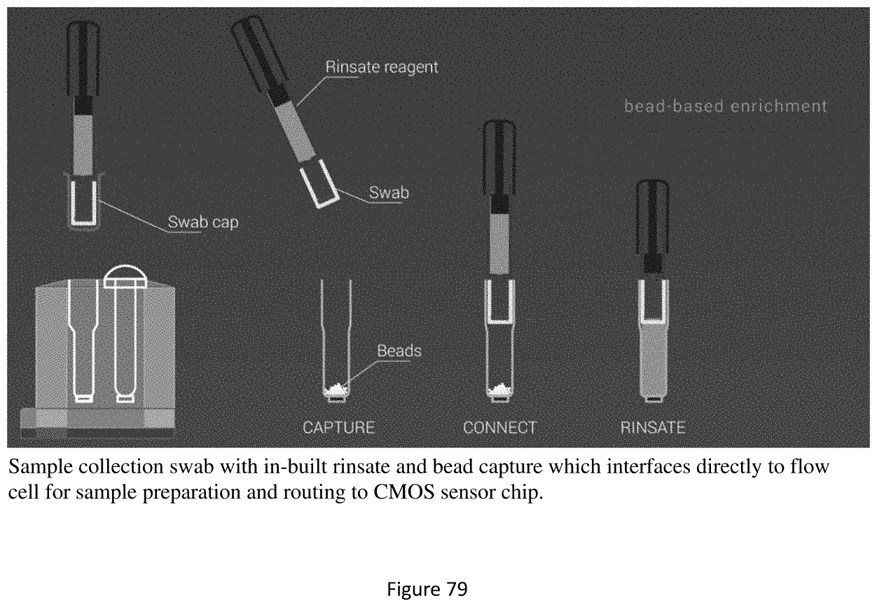

FIG. 79 shows an illustration of another embodiment of a sample collection wand with a swab tip and with built in rinsate, for use with collection beads, to enable bead-based sample purification and enrichment protocols. In this embodiment, the swab is used to collect a primary sample (such as saliva or a surface swab), the wand is used to inject rinsate through the sample, and into a chamber that incudes capture beads. Capture beads allow for bead-based purification and enrichment protocols, and can then be used to transfer the prepare sample to a second capsule, for delivery to the instrument or chip. The base module shown could be part of a separate sample prep module in some preferred embodiment, or integrated into the primary instrument flow cell in other embodiments.

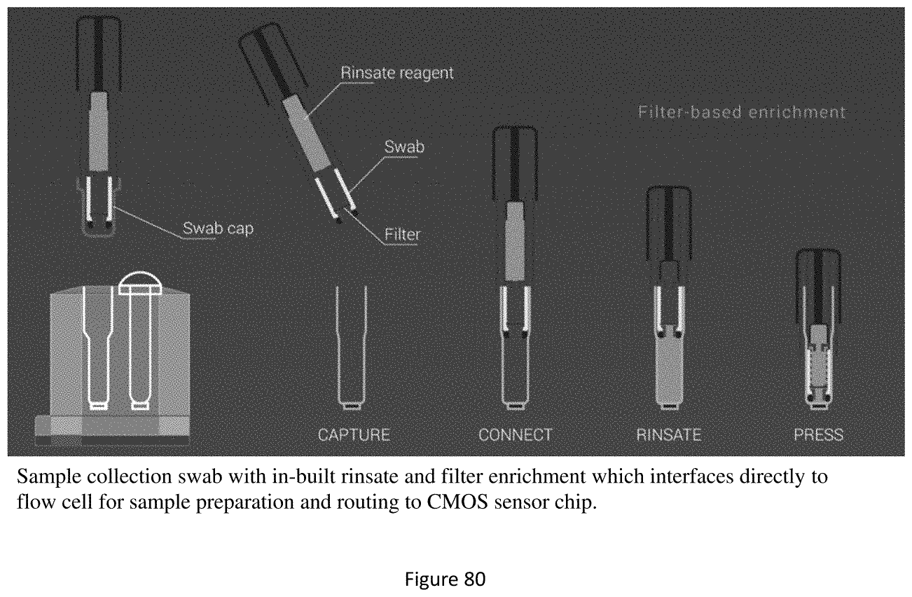

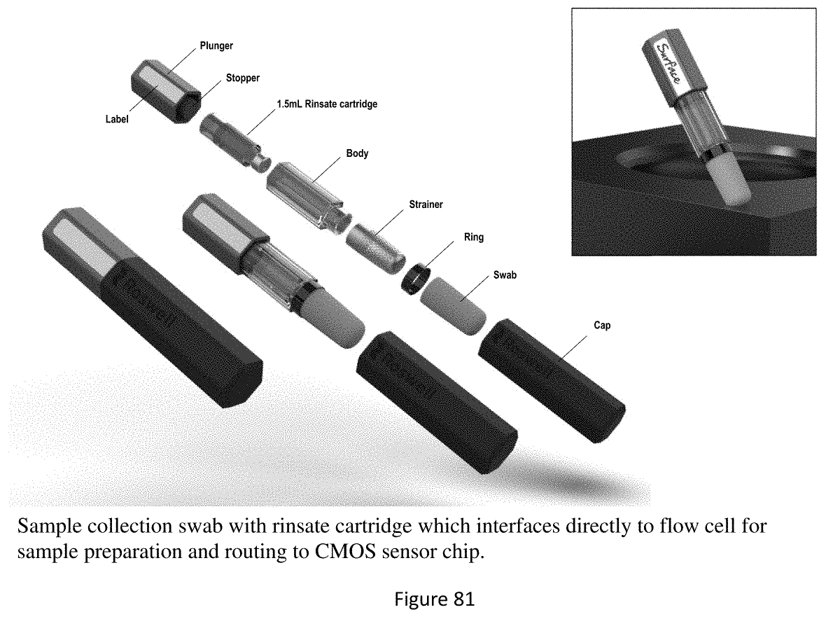

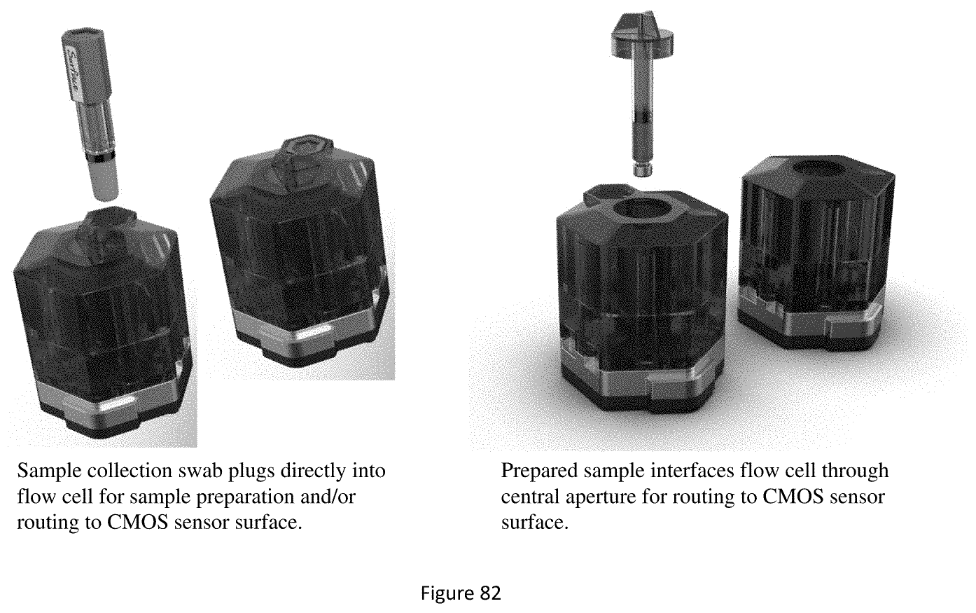

FIG. 80 shows an illustration of one exemplary embodiment of a sample collection wand with a swab tip and with built in rinsate, for use with a filter, to enable filter-based sample purification and enrichment protocols. In this embodiment, the swab is used to collect a primary sample (such as saliva or a surface swab), the wand is used to inject rinsate through the sample, and pass a filter through the sample and rinsate. In one embodiment, the filter will collect the desired component of the sample, and be passed on for subsequent processing. In another embodiment, the filter will remove unwanted components of the sample, and the filtered solution will be passed on for further processing. The base module shown could be part of a separate sample prep module in some preferred embodiment, or integrated into the primary instrument flow cell in other embodiments. FIG. 81 shows a CAD rendering of an exploded view of a sample collection wand with swab and rinsate, in an exemplary embodiment that can interface directly to the flow cell of a portable virometer. FIG. 82 shows an exemplary embodiment at left where the sample wand interfaces directly to the flow cell. At right is shown an alternative embodiment where a prepared sample from the wand is transferred to an injection capsule that interfaces to the flow cell.

In some embodiments of a method of use of the personal virometer, the virometer (or pathometer) can simultaneously read a DNA fingerprint of the test subject, for affiliation with the virometer test results. This DNA fingerprint can then be used for subsequent verification, security, chain of custody, data integrity, affiliation and databasing purposes. In some embodiments, this includes automatic affiliation of test results from multiple tests with the individual subject, such as for precision monitoring of an individual's health state over time, or for monitoring the health state of a population of individuals over time. In some embodiments, for a virometer in use in the home, on a recurring basis, this may be used to affiliate readings of taken across the entire family with individual family members, without the need for any other means of tracking sample sources. In some embodiments where the virometer is used at a place of employment, these may be used to reliably and automatically track the virometer health state of individuals, without the need for other means of tracking samples. In some embodiments, this virometer test result with affiliated human identifier can be used a secure and tamper-proof verification that an individual has been screened and is free from viral infection, and be used in this manner as a digital certificate of authenticity that a person is presenting their virometer test result, for themselves, and performed at a time that is also timestamped on the result. It is obvious that this can be used in many contexts to verify that individuals have tested as virus free on specific dates. In some embodiments, such a digital certificate could be required before travel, entering hotels, public or commercial venues, or places of work, or schools. In some embodiments, this DNA fingerprint, preferably combined with time stamp and GPS stamp of tests also acquired by the virometer instrument, can be using in methods of population scale contact tracing and infection tracing, such that field deployed virometers widely used in the population could have an independent means of identifying and tracking individuals, with the associated viral profile status, from the collective of virometer readings in the population, without the need for an other means of entering or acquiring such information that allows the identification of individuals across multiple tests, distributed in space, time and across the population. In other some embodiments, for biosecurity applications, such identified profiles may be used to monitor or identify or clear individuals who may have potentially been in contact with bioweapon pathogens.

In some embodiments, the DNA fingerprint used with the personal virometer or pathometer is comprised of human genetic markers that can be read by hybridization probes. In some embodiments, this is a set of Single Nucleotide Polymorphism (SNP) Genotypes, which are a well-known type of markers for use in human identification, and which are well-known to be typeable by hybridization assays. In some embodiments, a set of up to 10, up to 20, up to 30, or up to 100, or more than 100 such markers are used to reliably identify the human subject of the test. In tests that must distinguish closely related family members, such as home use involving distinguishing parents and children, or siblings, it is preferred to use 20 or more highly informative SNP markers for reliable, robust identification, and preferably 30 or more. In tests that must distinguish large numbers of people, such as millions or hundreds of millions, as in national scale viral screening, or up to billions of people, as in the case of viral screening for global air travel, in some embodiments such marker sets would use at least 40 markers, and preferably up to 100 markers or more.

In some embodiments of acquiring a DNA fingerprint with the virometer, this assay will use the same primary biosample acquired from the individual for purposes of testing a viral or pathogen profile, such as saliva, nasal swab, buccal swab, blood, urine, or bodily excretions, or skin swabs. Such samples contain genomic DNA of the test subject, and would undergo a suitable extraction method, and targeted PCR or target amplification method, to produce the set of amplified DNA markers suitable for human identification. In some embodiments, the same sensor chip that performs the viral profiling also includes probes suitable for typing these markers, such as hybridization probes for typing SNP markers. Running the chip on the pool of targets that includes the virus or pathogen targets, as we as the genetic marker targets, simultaneously producing the viral profile data, and the genetic fingerprint data, so that these data can be affiliated with each other, as well as with other data, and most preferably automatically acquired data not requiring user input, such as the time of test, or GPS location of the test.

In some embodiments, (FIG. 59) this Virometer is used by individuals, in field settings such as at home, in schools, at workplaces, at hotels, at transportation hubs, on mass transportation, or in public places, to determine whether a test subject or test sample of interest has specific indicators of viral infection, or viral exposure. The test subject in some embodiments is the individual themselves, or a family member, or test subject wishing to or required to have such an assessment (FIG. 59, left). The test sample may in other some embodiments be an environmental sample, such as a surface swab, and filter applied to an air or water sample (FIG. 59, right).

It is a general feature that, in some embodiments, the molecular electronic format can provide for rapid, low-cost, field-deployed, high multiplex and precise testing for viral exposure or infection. In some embodiments, such tests take less than 1 hour, less that 15 minutes, less than 10 minutes, less than 5 minutes, less than three minutes, less than 1 minute, or less than 30 seconds. In preferred embodiment, such tests cost less than $50 USD, less than $20 USD, less than $10 USD, less than $5 USD, less than $1 USD, or less than $0.5 USD. In some embodiments, the test can be applied to a sample from one individual, or to a pool of samples from multiple individuals, such as, for example, members of a family. In some embodiments, the device format may be portable, mobile, handheld, wearable, or embedded in a smartphone, smartwatch or smart ring. In some embodiments, the level of multiplex testing may be from 1-10 viral targets, 10-100 viral targets, 100-1000 viral targets, 1000 to 10,000 viral targets, or more than 10,000 viral targets.

In some embodiments, the device may be powered by batteries, solar power, mechanical power (including ambient motion, or mechanical winding mechanisms), or by plugging into an auxiliary device such as a smart phone, tablet or laptop, or power sockets as available in cars, homes, trains or planes. In some embodiments, the device may have a wireless or wired charging dock.

In some embodiments, the device may report out the abundance of the detection target, to provide a measure of severity of exposure, contamination or infection.

In some embodiments, the molecular electronics chip may utilize a molecular electronics hybridization sensor or primer extension sensor, to assess DNA or RNA in a sample for presence of target viral DNA or RNA, and thereby determine which virus is represented in the sample, as well as provide information on which strain or genetic variants are present. In some embodiments, the sensor chip is a multiplex sensor chip, capable of detecting a multitude of such targets, across multiple virus types, strains and variants.

In some embodiments, the molecular electronics chip may utilize a binding sensor comprising antibodies or aptamers as specific binding probe that detect a target viral protein or antigen. In some embodiments, the sensor chip is a multiplex sensor chip, capable of detecting a multitude of such targets, across multiple virus types, strains and variants.

In some embodiments, the molecular electronics chip may utilize a binding sensor comprising antibodies or aptamers that are used to directly bind the viral particles, and thereby directly detect viral particles in a sample.

In some embodiments, the entire device may be handheld, and include a disposable probe (FIG. 52, 53, 53.1-53.3, 58). In some embodiments, the probe may comprise a separate swab or applicator that is used to acquire the primary sample, and transfer it to the device. In some embodiments, this transference may comprise a step of an intermediate device that transforms the primary swab into a format suitable for introduction to the chip device.

In some embodiments, the device may also have a separate base for docking, which may perform functions such as charging, cleaning, data transfer or computation support for processing the data generated by the chip running device (FIG. 56, 56.1-56.6, 57)

In some embodiments, the molecular electronics chips used may be field programmable with new target detection content, through a reaction with suitable programming reagents. In some embodiments, this consists of a solution of probe molecules, that have addressable binding to the sensors on the array. In some embodiments, this addressable binding is via DNA oligos, with oligos resident on the sensors on chip, and the complimentary oligos on the programmable content probes, such that probes coupled to unique oligos will become conjugated to the complimentary oligos on the respective sensors. In some embodiments where detection involved hybridization to a DNA oligo probe, the programming reagent simply consists of the desired oligo probes, extended to have oligos that target the oligo-defined binding sites on the array. (FIG. X). This has the advantage that arbitrary target content can be deployed simply by deploying an updated oligo reagent, combined with a software update to specify the new targets mapping to the sensor chip, and any changes to the analysis required, with no change to the sensor chip or hardware platform. This greatly reduces the time, cost and complexity of making such changes, and also reduces the regulatory time, cost and complexity of validating the platform with the new content.

In some embodiments, individuals will use the virometer at home, to check viral status of family members before they go off to work, school, or to perform daily duties, or when they return from such.

In some embodiments, individuals will use the virometer to test environmental samples, such as surface swabs, or filtrates of water or air, acquired from any of a great diversity suspect samples. For example, this may include samples taken from door handles, car seats, groceries, mail or packages, takeout food containers or contents, household water sources, or household air sources. Many more such potential target samples are clearly possible, and in generally anything that may be subject to swabbing or filtering could be sampled for such testing.

In some embodiments, samples taken from individuals may include saliva, mouth or throat swabs, nasal or nasopharyngeal swabs, sputum or expectorate, or material ejected by coughing or sneezing, or swabs of hands or skin or hair. In some embodiments, the same may be a breath sample.

It is obvious to those skilled in infectious disease that the considerations above for the virometer and viral detection, apply similarly other infectious disease pathogens, including bacteria, fungi and parasites. In such cases, sample types may extend, as appropriate for the specific pathogens and diseases, to include other types of biospecimens, such as blood, sweat, tears, fecal matter, urine, or anal, vaginal or urethral swabs or secretions.

In particular, in some embodiments the applications of the virometer or pathometer extend to individual testing for sexually transmitted diseases. They also extend to individual testing for food born illness pathogens. In the latter case, the sample types naturally extend to include samples of the solid or liquid foods in questions.

Methods and Applications for Infectious Disease Testing—The personal pathometer device disclosed above has diverse applications to testing for or monitoring infectious disease pathogens. In some embodiments, testing or monitoring applications including pathogens that are parasites, fungi, bacteria or viruses. Such parasites include Malaria, Giardia, and Toxoplasmosis. Such bacterial pathogens include Salmonella, E. coli. Such viral pathogens includes influenza, flu viruses, cold viruses—including rhinovirus, adenovirus, and human corona virus, HIV, Ebola, Dengue, Hanta, Zika and West Nile viruses, SARS, MERS, and COVID-19 virus, and novel viruses of DNA or RNA type related to or unrelated to these, that have a known genetic sequence to provide for defining hybridization probes. In other embodiments, the detection targets may proteins or antigens, an any such pathogens where these are available can be used to define aptamer probes or antibody probes.

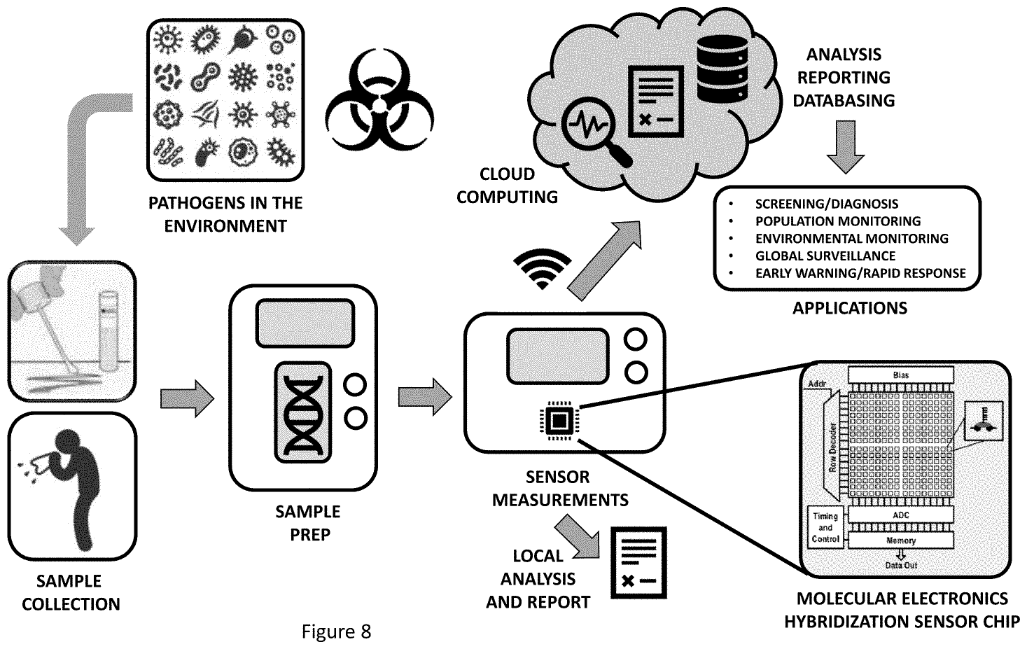

The major elements for infectious disease pathogen detection applications are disclosed here, as illustrated in FIG. 8. As indicated there, Infectious disease pathogens exist in the environment. A primary biosample is obtained, which in some embodiments may be material from a human subject, or a swab or other material collection from the environment, including possibly from plants or animals in the environment. This primary biosample is provided in some fashion to a sample preparation sub-system, which extracts and purifies DNA contained within the primary sample (or RNA, or in other some embodiments, proteins or antigenic material), and puts it into a form suitable for application to the sensor chip device disclosed herein. This sample is provided in some fashion to sub-system instrumentation that applies the sample to the chip, and controls the chip operations, and collects the sensor signal data from the chip. This instrument in some embodiments may locally analyze the data, record it locally, and produce a report on the pathogen content of the sample, such as on a screen on the instrument. In other some embodiments, this instrument transmits the data to a remote cloud, where analysis, reporting out on the pathogen content of the sample, and databasing of results can occur. Such cloud-enabled embodiments, in conjunction with many deployed instruments, are well suited to large scale efforts, such as national or international or global scale screening of samples for pathogens, for diagnosis of disease caused by pathogens, for monitoring the occurrence or spread of such disease at the population scale, or for monitoring such pathogens in the environment, and for global surveillance and early warning/rapid response efforts to contend with outbreaks of such infectious diseases.

In some embodiments for testing methods and applications, a primary biosample is acquired directly from a test subject or the environment, and then some form of sample prep is required to prepare materials to the proper state to apply to the sensor device for measurement. The primary sample in some embodiments could be tissue, saliva, mucous, buccal swab, blood, sweat, urine, stool, out bodily fluids, or exhaled air, or material filtered from air or water, or material swabbed from a surface. It could also be such samples acquired from plants or animals in the environment, or from food, or from known vectors in the environment that carry such pathogens, such as bats, rodents, mosquitoes or snails. The sample prep could in some embodiments be a crude cell lysate extract containing DNA, or could be DNA further purified from the sample by standard purification column or filter paper purifications, or other extraction such as phenyl-chloroform. In some embodiments the purified sample could be the results from applying any of the many forms of PCR amplification reaction to the sample, which could in some embodiments be thermocycling or isothermal forms of PCR. In some embodiments, such sample prep is done by a self-contained sample prep device, or in other some embodiments, such a device integrated with the sensor platform, such as in the case of fully integrated point-of-us testing devices.

In some embodiments for the testing method using such devices and systems, the test system is deployed at a testing site, a primary biosample is collected and delivered to the testing site, to be tested for presence of a given pathogen or pathogen strain, a sample preparation process is applied to the primary sample to produce a product suitable to be applied to the molecular electronic sensor array chip device, which comprises a multiplicity of hybridization probe sensors that target a pathogen or pathogen strain of interest, and the device signals are readout, undergo primary local signal processing, and these data are then transferred to a centralized or cloud-based server for subsequent additional analysis or testing outcome report generation.

In some embodiments, the testing site could be a centralized site of high capacity, for a business, hospital or other organizations, or for a region such as a city, county, state, our country. In other some embodiments, the testing site could be a field deployment site, or a point-of-contact or point-of-use site, such as at an airport, transportation hub, or major gathering site such as an arena or stadium, or at an immigration control checkpoint or temporary monitoring point set up by the military, police, or government officials. In other embodiments, the testing site could be a mobile van that is deployed to sites as needed. In other some embodiments, the testing site may be in the home for private individuals. In other some embodiments, the testing site could be autonomous environmental monitoring stations deployed into the field, stationary or mobile, including driving, flying or aquatic drones, that monitor samples acquired locally from the environment, such as through filtering of air, or water, or trapping of known disease vectors or carriers in the environment, such as insects, rodents, bats or birds, or aquatic snails. In some embodiments, mosquitoes are one such vector.

In some embodiments, the primary biosample could be obtained as a swab of a surface that collects material deposited on the surface, as filtered material collected from air or water, or a water sample, or as a bodily fluid sample or buccal swab or saliva or excrement or tissue sample provided from a person or animal, or as a sample of a food item, or agricultural product.

In some embodiments, the sample collection may be done in close proximity to the test system, such as within 1 foot, 10 feet, or 100 feet, and in some embodiments such samples are rapidly delivered to the test system, such as within 10 seconds, one minute, 10 minutes or 1 hour, in order to have the benefit of distributed sample collection combined with rapid testing and test results. In some embodiments, the sample collection includes the assignment of a unique ID to the sample, such as an alpha-numeric code, serial number, barcode or QR code, to be used for sample tracking, and affiliation of final report back to the sample. In some embodiments, other identifying information may be collected and attached to the sample or affiliated with the sample ID, such as personal identifier, such as a personal name, social security number, government issued ID number, employee number, or date of birth, facial image or fingerprint.

In some embodiments, the sample preparation process comprises a PCR-based amplification method applied to the sample to produce amplified DNA material for detection. In other some embodiments, the sample preparation process is a process to extract and purify DNA or RNA without any amplification to produced purified material for detection. In preferred embodiment, this sample prep process is performed in a separate instrument from the sensor chip instrument, and is transferred to that instrument. In other some embodiments, the sample prep processes are performed on a subsystem integrated into the same instrument that runs the sensor chip device.

Application to Viral Pandemics and COVID-19—In some embodiments, the pathogen of interest is a virus, such as influenza, flu viruses, cold viruses—including rhinovirus, adenovirus, and human corona virus, HIV, Ebola, SARS, MERS, and COVID-19, and novel viruses of DNA or RNA type related to or unrelated to these, that have a known genetic sequence to provide for defining hybridization probes. In such cases the hybridization probes include specific DNA probes common to many strains of such a virus of interest. In other some embodiments, the pathogen includes the specific strains of such viruses, and the corresponding hybridization probes include strain-specific DNA probes.

In some embodiments, the primary data analysis performed on system includes data reduction algorithms that reduce the amount of data needed to be transferred off-system. Such methods may include discarding uninformative portions of the signal trace, subsampling or parameterization of parts of the signal trace, and general data compression algorithms known to those skilled in data compression, such as methods utilized in zip, gzip, bzip, and other common compression utilities. In some embodiments, the primary analysis also includes analysis of traces to produce a net hybridization intensity score for each probe on the sensor chip, and in some embodiments, a final call of detection, non-detection, or indeterminate measurement for each probe on the sensor chip. In other some embodiments, such analysis is done in the off-instrument phase of an analysis. In other some embodiments, the off-instrument analysis includes the generation of a final report that affiliates sample identifiers with the outcome of the test for the presence of pathogens of interest. Such identifies may include a subject name or assigned ID or other identifier provided at the point of sample collection, as well as sample identifiers such as the time and place of sample collection, and time and place of sample processing on the sensor chip system.

In some embodiments, the test is performed rapidly, with the time from providing the primary biosample, to completion of analysis and report generation being less than 1 hour, or less than 30 minutes, or less than 15 minutes, or less than 10 minutes, or less than 5 minutes, or less than 1 minute.

In an exemplary embodiment, the system disclosed above is applied to the monitoring of the pandemic disease COVID-19, a viral disease outbreak in 2019 originating in Wuhan, China. In this application, the hybridization probes are selected to be complements to segments from the genome of the underlying virus, the Severe Acute Respiratory Syndrome Coronavirus 2, also designated SARS-CoV-2. This SARS-CoV-2 virus has a single stranded RNA genome, of size approximately 30,000 bases. One exemplar sequence for this genome is available at the Genbank database as accession ID LC528232. Thus, in some embodiments where a DNA probe hybridization probe directly detects the genomic material by hybridization, this will be DNA-RNA hybridization, and the sample prep must extract and purify RNA from the primary biosample. In some embodiments where the sample prep comprises a PCR amplification of the genome, this would be a reverse-transcriptase mediated PCR that produces amplified DNA product, either of specific target segments, or non-specific segments of the entire genome, and the resulting hybridization detection is DNA-DNA hybridization. By taxonomy, this virus a specific strain of the Severe Acute Respiratory Syndrome-related Coronavirus (SARS-Cov), which is a species of coronavirus that infects humans, bats and certain other mammals. There are hundreds of known strains of this virus, and hybridization probes must be chosen for sequence segments that distinguish the COVID-19 strain from other harmless strains, or other disease-causing strains, such as the strain designated SARS-CoV, which caused SARS disease outbreak in 2002 in Guangdong Province, China. There are numerous sequence differences between these strains, providing many candidates for distinguishing target sequences.

In other disclosed embodiment of testing for SARS-CoV-2, the targets for detection are viral proteins. These may include any of the viral proteins, in particular the Spike (S) protein, or the Nucleocapsid (N) protein may be such targets of detection. The probes for these may be based on aptamers or antibodies targeting these proteins. The targets may be other antigens from SARS-CoV-2, such as components of the viral capsule, and the probes for such may be based on aptamers or antibodies targeting these antigens.

In some embodiments for COVID-19 testing, primary samples would be environmental surface swabs, or air filters, and such testing provides for monitoring of the presence of the virus in a target location where such samples are collected. In other some embodiments for COVID-19 testing, primary samples would be saliva, buccal swab or mucus samples from individuals, and such testing provides for detection or diagnosis of subjects with active viral infections. In some embodiments of such testing, the present device provides for rapid, distributed testing. In some embodiments for this, the system provides for a test in less than 1 hour, or less than 30 minutes, or less than 15 minutes, or less than 10 minutes, or less than 5 minutes, or less than 1 minute.

Other Infection Disease Applications—In another preferred embodiment, the personal pathometer (or bactometer) disclosed above can be applied to testing for and response to outbreaks of bacterial disease, such as when the food supply is contaminated by E. coli or Salmonella. For example, in some embodiments, this could be an outbreak where lettuce is contaminated by E. coli, or where ground beef is contaminated by Salmonella. such cases, in some embodiments testing platforms are deployed in point-of-use format to sites of food production, such as farms, fields, and processing plants. In some embodiments such testing platforms are also deployed in point-of-use form, as well as mobile or distributed permanent or temporary monitoring installations, to points of distribution, such as warehouses, shipping centers, or grocery stores and restaurants. In some embodiments, such testing platforms are deployed to the end point of consumption, such as in the home, for home-based testing. In some embodiments, the aggregated cloud-based analysis, including Big Data, AI and machine learning techniques, can be used to track the outbreak, and pinpoint the source.

In some embodiments, the personal pathometer systems disclosed above can be applied to testing for sexually transmitted diseases (STDs). In this application, it is an advantage of the invention disclosed that such molecular electronic hybridization sensor systems can be deployed for rapid, low-cost testing in highly distributed fashion, such as in community or field clinics, or for the privacy of home use. For STDs, the causal pathogens to be detected may be parasites, such as Trichomoniasis, or fungi, such as Candidiasis (yeast infection), or bacteria such as Syphilis, Gonorrhea, or Chlamydia, or viruses such as Herpes, HPV, EBV, Hepatitis, and HIV. In such applications, the primary samples required are clinically well established, and may be a blood sample, or a swab of bodily fluids or of discharges, or from open sores. In some embodiments of such testing, the present device provides for rapid, distributed testing. In some embodiments for this, the system provides for a test in less than 1 hour, or less than 30 minutes, or less than 15 minutes, or less than 5 minutes, or less than 1 minute. In some embodiments, the system provides the advantage of extreme personal privacy, with systems and test kits that can be used entirely within the home.

Molecular Electronic Sensors and Sensor Array Chips for the Personal Virometer—It is object of this invention to disclose molecular electronic sensors and sensor chips for use in the personal virometer or personal pathometer. Such sensors make use of a variety of molecular binding systems. In the following, we disclose some embodiments of the targets of detection, the means of detecting such targets, and the molecular electronic sensor constructs and sensor array chips that perform such sensing.

Detection of Nucleic Acid Targets—In the field of genetic analysis, it is important to be able to determine if a given sample of biological material contains a target DNA or RNA segment of interest. An important example is species identification, where a sequence characteristic of a species, is searched for within the sample. This is, for example, important for the monitoring of, and diagnosis of, infectious disease. For example, a DNA segment that identifies a pathogen, such as a parasite, fungus, bacteria or virus, can be looked for within the sample taken from the environment, or from an animal or person that may be infected. This is especially important for the environmental surveillance and epidemiology of viral diseases with the potential for large scale, rapidly progressing infection or pandemics, such as COVID-19. It is also important in genetic analysis to look for known genetic variants that may occur, relative to a give segment of DNA. This type of measurement is known as genotyping in the context of looking for known variants in humans and animals. In the context of pathogens, this takes the form of identification of strains, which are defined by DNA or RNA variants relative to a reference genome sequence, or by the sequence differences between two genomes. It is also important to be able to determine the concentration level of a DNA or RNA segment of interest in a sample, rather than just presence or absence. In the context of infectious disease, this may be related to determining the severity of the infection in an individual, based on the amount of pathogen DNA detected, such as in the case of “viral load” in the case of viral infection. Determining quantity or concentration pf pathogen DNA is also important for determining the infectious potential in environmental samples, based on the concentration of the pathogen in the sample.

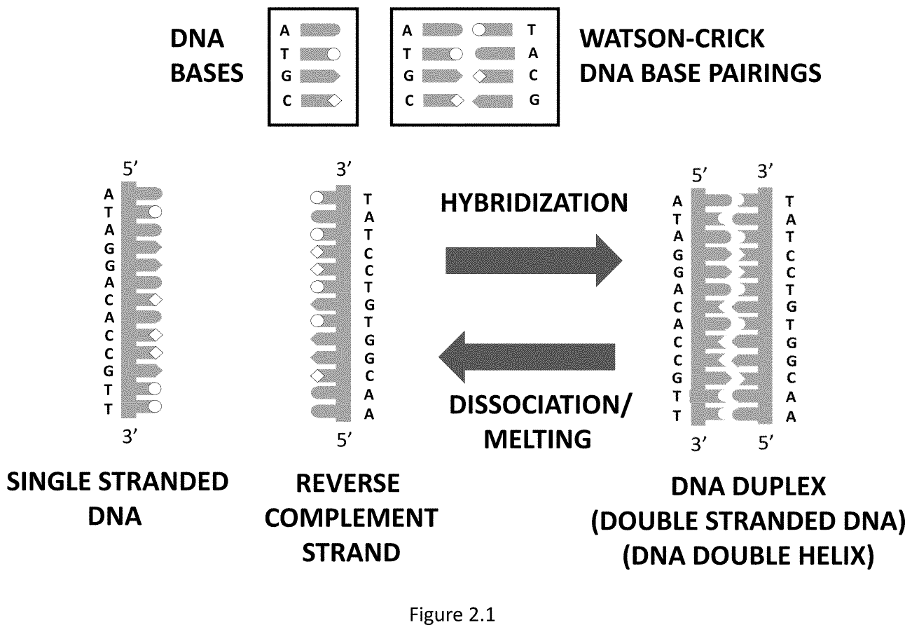

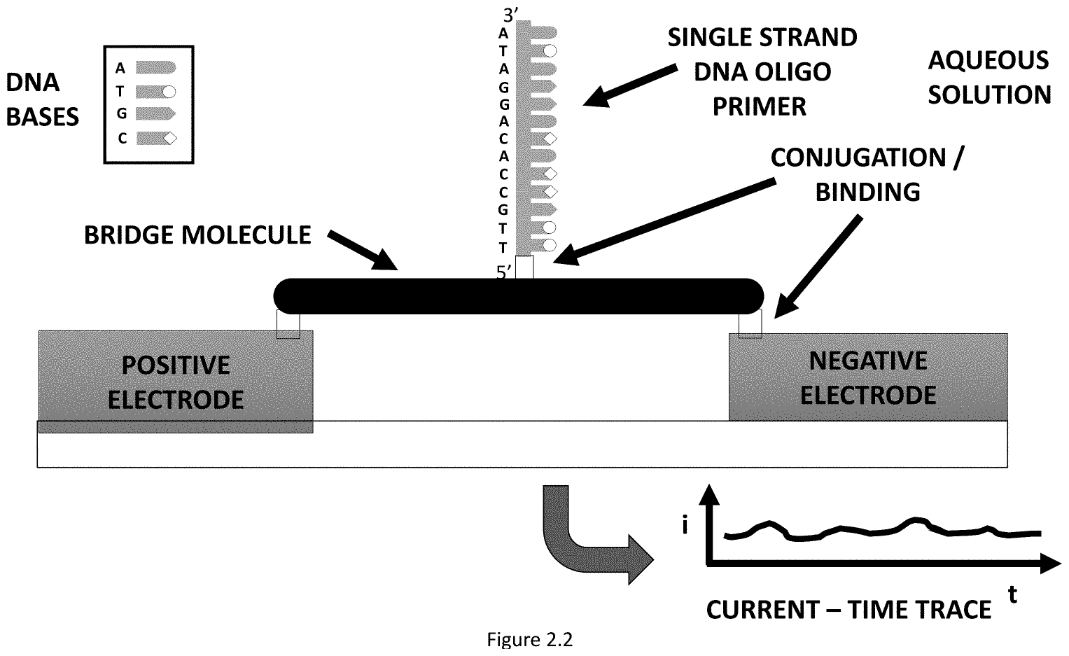

Hybridization Reactions—These general classes of genetic analysis problems—measuring the presence of or concentration of DNA segments of interest—are addressed using well known modern molecular biology tools such as PCR, DNA Microarrays, and DNA sequencing. Older techniques predating these, such as Southern Blots (for measuring DNA targets) and Northern Blots (for measuring RNA targets) have also been applied to these problems. All such assays, including Southern and Northern Blots, employ the process of DNA “hybridization” as a fundamental part of the detection scheme. As shown in FIG. 2.1, hybridization is the natural phenomena where, a single strand of DNA will, with high efficiency, bind to its specific reverse complementary strand in a solution. In FIG. 2.2, DNA is depicted in cartoon fashion to illustrate the logical structure as a sequence of bases, with the important backbone orientations (3′ and 5′ ends) indicated, and to indicate the logical Watson-Crick Base Pairings (A-T, G-C in DNA, as well as (not shown) U-T in RNA-RNA or RNA-DNA pairings), but the figure does not indicate the double-helix physical structure of the duplex, or the chemical hydrogen bonds involved in base pairing. The reverse process, where a duplex separates into independent single strands, is referred to as dissociation or melting, and it is a strongly temperature dependent process, the transition temperature being known as the melting temperature (Tm, which depends on the solution composition as well, and to a lesser extent on the strand concentration, and which can also depend on other chemical, steric or entropy effects like the proximity of other molecules, or surfaces, or the tethering of DNA to other molecules). For DNA free in solution, substantially below the melting temperature, the duplex form is stable and long lived, and substantially above the melting point, it is unstable and short lived. Within a few degrees C. of the melting point, the on and off reactions are both common. Generally, hybridization is a chemical reaction process obeying Boltzmann distribution kinetics, and as such it is a reversible process, for which the precise definition of melting point is the temperature where strands are equally likely to be bound and unbound for the given solution conditions. In common physiological solution conditions, melting point for duplexes shorter than 10 bases may be below room temperature (25 C), while for duplexes 50 bases or longer, or with higher G-C pair content, Tm may approach the boiling point of water (100 C). In typical buffer solutions used in molecular biology, the salt levels and divalent cation levels (such as mg++) have moderate effect on the melting point, and therefore the kinetics of hybridization and dissociation. At low concentration of complimentary strands, it may take a long time for duplex formation to occur, as the strands must meet first, primarily by diffusive transport.

Similar to what is shown in FIG. 2.1 for DNA, any combination of complementary DNA and RNA strands can pair by hybridization in this way, as can strands of DNA or RNA that contain various nucleotide analogs such as LNA or PNA, or various chemically modified nucleotide. Longer segments of DNA that match along a segment will form a duplex pair along that segment, even if the ends do not match, although the free ends may result in a slightly lower melting point. Strands that are not perfectly matched can bind, but the presence of mis-paired bases substantially lowers the melting point, and they may not be stable under many conditions of interest. This relatively lower Tm for mis-paired strands (sometimes called cross-hybridization) can be used advantageously to design detection assays using hybridization, such that an exact match is preserved and produces and enduring detectable signal, while all the great many possible mismatch interactions with off-target fragments can be eliminated from producing signals, or otherwise result in signals that are substantially below some form of detection threshold. The well-known Southern Blot assay relies on DNA-DNA hybridization, and the Northern Blot assay relies on DNA-RNA or RNA-RNA hybridization, for the critical part of the sequence-specific detection process. DNA Microarrays, which were a technological descendent of Southern and Northern Blots, also rely on DNA hybridization as the critical part of their sequence-specific detection process. Indeed, even PCR reactions rely in part on the hybridization process, as the single stranded DNA oligo primers used in PCR and related reactions must stably hybridize to a single stranded template in a sequence-specific manner, free of mismatches, in order for the polymerase to efficiently extend the primer. Similarly, many forms of DNA sequencing processes also utilize some form of priming and polymerase extension as a part of the overall process. In these particular contexts involving priming for polymerases, the hybridization is often referred to as primer binding, or primer-template binding.

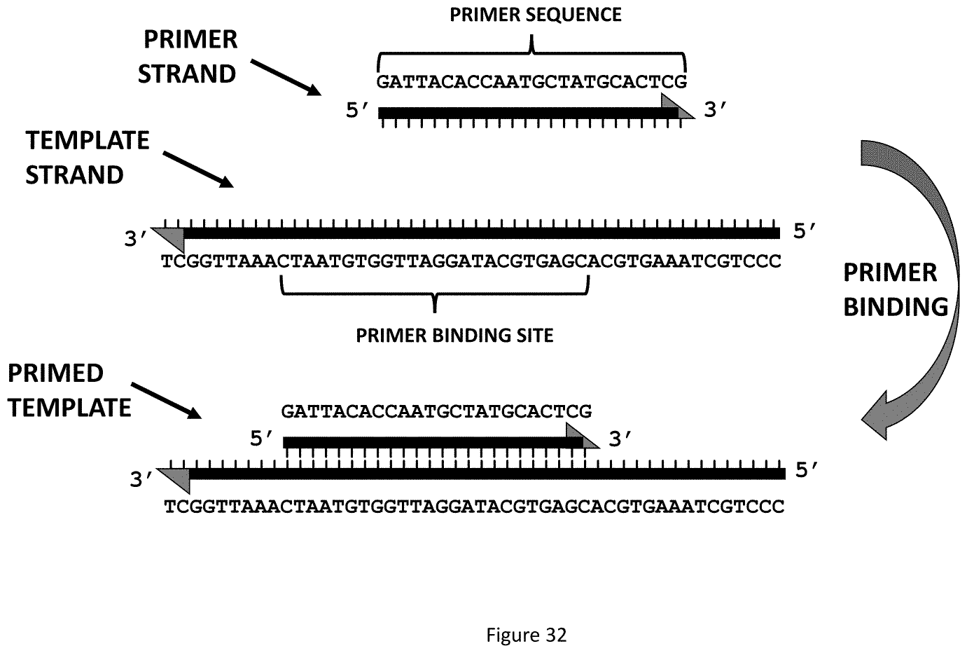

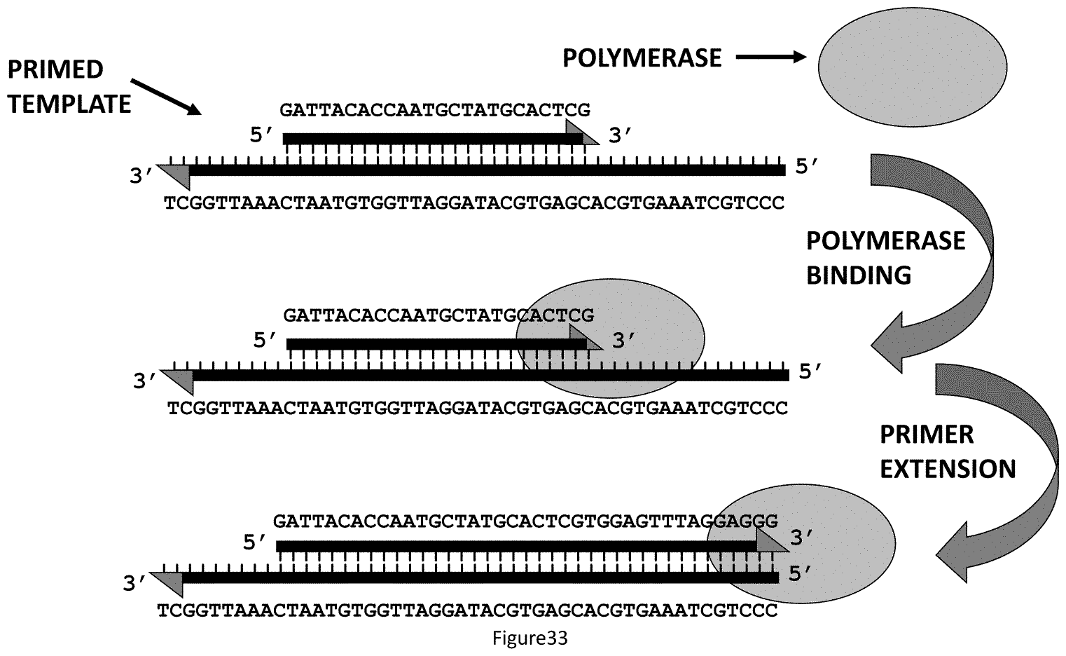

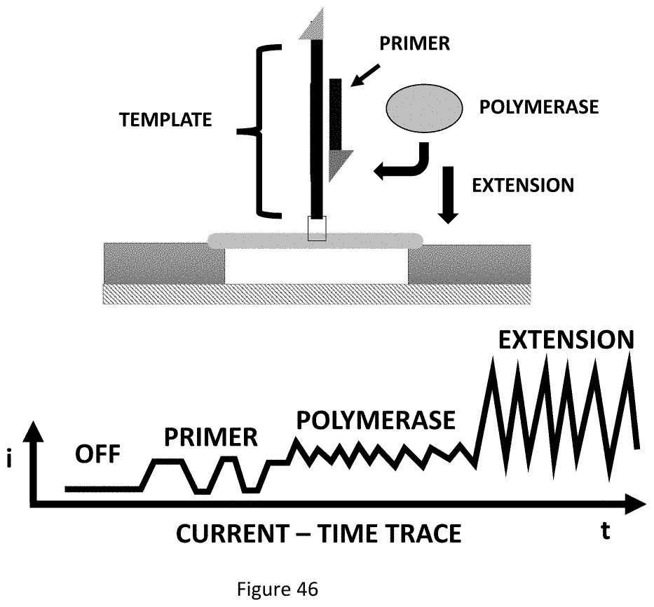

Primer Binding and Primer Extension Reactions—One particularly important process where hybridization plays an essential role is in the application of a polymerase to produce the complementary strand from a given single stranded template. For example, this is a critical component of common PCR assays and sequencing assays, as well as certain DNA microarray genotyping assays. The key steps of this process are illustrated in FIGS. 32 and 33. In this context, a so-called “primer” DNA oligo is hybridized to its target on a single stranded template, as shown in FIG. 32. A specific sequence is shown in FIG. 32, to illustrate the general principle that the primer hybridizes specifically to its complementary site on the template. The 3′ and 5′ ends are emphasized as well, since the action of the polymerase to synthesize DNA strongly distinguishes between these two ends. As shown in FIG. 33, a polymerase enzyme will bind specifically to the 3′ end of the bound primer. In this configuration, if supplied with the raw materials required to synthesize a new strand, deoxynucleotide triphosphates (dNTPs), and in a suitable solution environment, the polymerase will synthesize the complementary strand, as indicated in FIG. 33, sequentially incorporating dNTPs to form the growing strand. This is referred to as extension of the primer. The specific substrates for polymerase extension, used to build the growing strand, are the deoxynucleotide triphosphate forms of the A, C, G and T bases, these are collectively referred to as dNTPs, and are, explicitly, dATP, dCTP, dGTP, and dTTP, which incorporate opposite the T, G, C and A bases, respectively, in the template strand. The polymerase will typically eventually release from the primer-template complex, either after extending for some bases, or after it reaches the 5′ end of the template strand, as indicated in FIG. 33. In the case where the enzyme does not reach the 5′ end, the number of bases incorporated before the enzyme releases is generally a random variable in any particular single molecule extension, as there is a generally a small probability of release after each incorporation event. Some types polymerases, even under favorable conditions, typically extend only a few bases, whereas other types may extend for tens, hundreds or thousands of bases under favorable conditions. This action of polymerase extending a primer follows from their general biological function, which is either to replicate DNA directed by a template strand, and starting from a 3′ end of a duplex-single strand junction, or to repair single stranded breaks in DNA that leave free 3′ ends. This general catalytic cycle of a polymerase is shown in FIG. 46. Once a primer binds the template to form double stranded DNA of length n base pairs, with a free 3′ end, the polymerase binds to this, then a suitable dNTP binds. In the presence of a divalent metal cation cofactor (denoted M++, such as specific examples of Mg++ or Mn++ or others), an activated form of the polymerase, Pol*, forms and extends the DNA strand by one base DNA(n)→DNA(n+1), and in so doing, cleaves off two of the three phosphate groups of the dNTP, in the form of a released pyrophosphate PPi. This cycle can then repeat to further extend the strand.

Conditions for Primer Extension—In common molecular biology applications, the primer is typically an oligo in the range of 6-60 bases in length, and most commonly in the range of 14-30 bases in length for PCR reactions. It is important for the primer to be properly bound to the template in order for extension to occur. This need not be an exact match binding to the template, in general—some level of mismatches in a bound primer may still be compatible with primer extension occurring.

One key element of proper binding is the temperature of the extension reaction should be at or below the melting point, Tm, for the primer hybridization (including the case where there are mismatches), for efficient extension to occur. Substantially above this temperature, the primer does not remain bound long enough for the extension to occur with high probability, and in general the probability of extension drops off rapidly with temperature as temperature is increased beyond the melting point. This has the practical consequence that the rate of the extension reaction initiating drops dramatically at such elevated temperatures. Furthermore, at temperatures near or above Tm, even if extension of one or a few bases occurs, there is also the potential the extended primer may melt off during this process, leading to very limited extension products, and with such products not bound to the template.

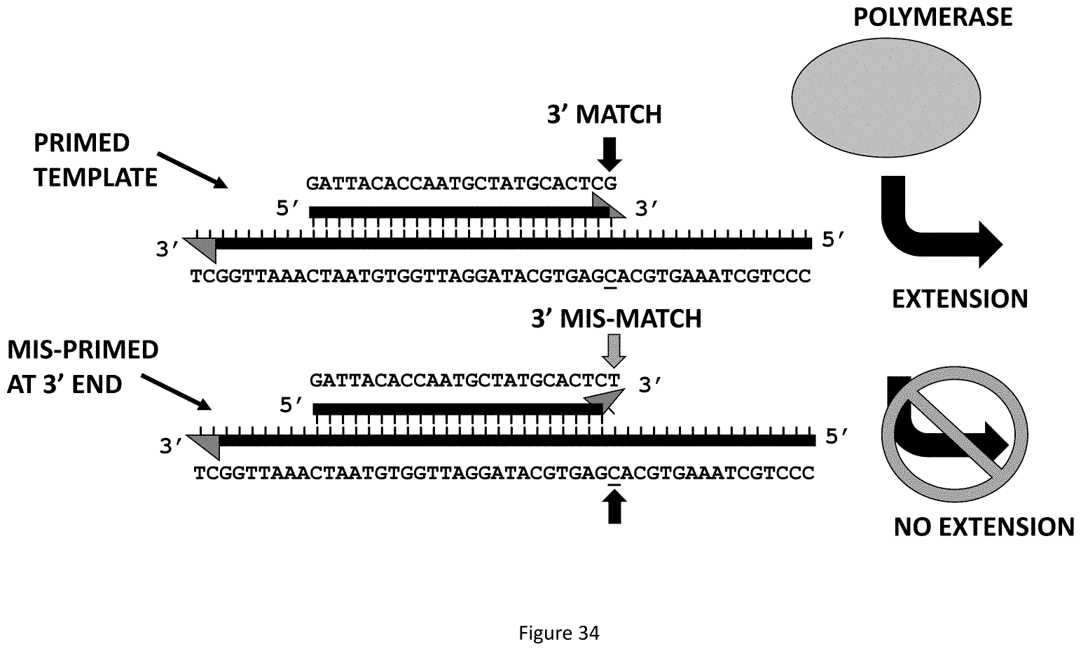

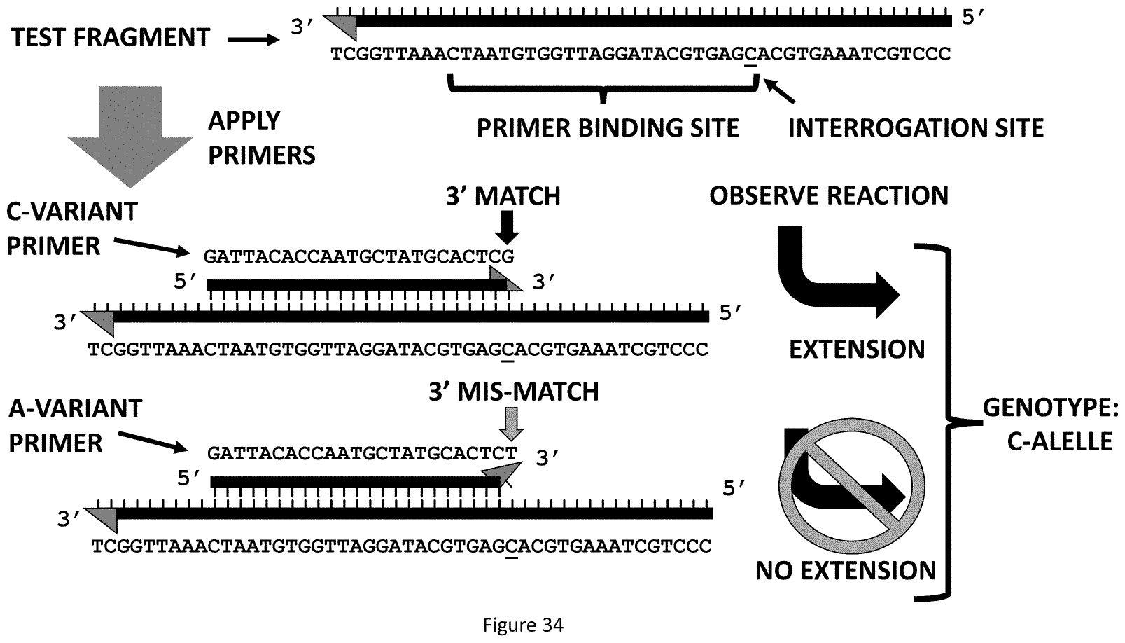

The other key element of proper binding is a match at the 3′ end. A primer does not in general have to be bound as a perfect match to the template for extension to occur—mismatches in the primer-template binding may be acceptable as long as the reaction temperature is not too high relative to the melting point, Tm, of the mismatched binding. However, the critical exception to this is, as shown in FIG. 34, that for extension to occur the 3′-most base of the primer must be properly matched to the template. Polymerase enzymes are very sensitive to this. As shown there in the lower figure, if the 3′ terminal base of the primer does not match—there the terminal T of the primer does not pair with the C in the template, in contrast to the upper figure where a terminal G on the primer is paired—the polymerase will not extend the template, even if the rest of the primer is perfectly matched and the temperature is well below the Tm. More precisely, if the 3′ end is mismatched, the probability of an extension is greatly reduced, typically by two orders of magnitude or more, even if the primer is otherwise very stably bound to the template, i.e. the temperature is well below the Tm.

Detection of Non-Nucleic Acid Targets—In the field of molecular diagnostics, it is important to be able to detect other biomarker targets other than DNA or RNA. In particular, such targets could be proteins, small molecules, or diverse components or substituents of pathogens. In particular, for detection of viral pathogens, targets may include viral proteins, or other components of the viral capsule or structure.

Antibody-Antigen Binding and Antigen Assays—One general method of detection is based on having a target antigen, and an antibody specific for binding the antigen. Such antigens can be proteins, fragments of proteins, small molecules, or potentially entire pathogens such as viral particles, or portions thereof, such as material from their capsules. Antibodies are generally generated in a host animal, as an immune response to the presence of the antigen in the bloodstream. Antibodies can then be used as the basis of detection assays for detecting the antigen in a complex sample. One common class of such assays is the Immunoprecipitation Assay (IPA), in which a surface-immobilized antibody is used to capture its antigen from solution, possibly with a secondary dye-label for optical detection. Another common class assays for doing this is the so-called Enzyme Linked Immunosorbent Assay (ELISA). In these assays, of which there are several common variations, the successful antibody-antigen binding is typically used to recruit to into a well an enzyme reporter construct that produces a detectible colorimetric signal in the well. Alternatively, the isolated antigen can similarly be used as the bound probe to look for a cognate antibody in a complex sample. Diagnostic assays based on an isolated antibody binding its target in a complex sample are commonly referred to as antigen assays. For viral pathogen detection, such assays may use antibodies against the viral coat proteins or viral coat materials to detect the presence of virus in a biosample, such as saliva or a nasal swab. Diagnostic assays that instead start from an isolated antigen and look for the presence of antibodies in a complex sample, are commonly referred to as antibody assays. In the context of viral pathogen assays, viral protein antigens may be used to screen a subject blood samples for presence of antibodies, indicating prior exposure to the virus—e.g. prior infection—that resulted in a prior immune response.

Receptor-Target Binding and Protein-Protein Binding—Other forms of biological binding pairs can be used for detection assays, in manners similar to general antibody-antigen binding described above. One common case is that of a cell surface receptor and its target, which bind selectively as part of some biochemical process or pathway. The receptor and target can be used for detection assays in manners similar to the antibody and antigen of an ELISA assay. Another common case is a more general protein-protein binding interaction from some biochemical process, which can similarly be deployed for relevant detection assays of either partner. An example of this is the context of viral pathogen detection is the case of the ACE2 receptor, present on human lung tissue cells, and the spike protein on the surface of the SARS-CoV-2 virus, which binds to the ACE2 receptor as part of the infection mechanism. The ACE2 receptor has been utilized in detection assays for the viral particle, exploiting this binding reaction.

Aptamer Binding Reactions and Aptamer Binding Assays—An aptamer is a single stranded DNA (or RNA) oligomer that engages in specific binding with a target which may be a protein, small molecule, or other molecular target. Aptamers can also be formed from peptides. The primary and secondary structure of such oligos determine the specific binding for such targets. Aptamers have a functional analogy to antibodies, in that they are a large family of biomolecules that can be selected upon to specifically bind a given target, and allowing for a very broad range of target types. This can be used as the basis for detection reactions or assays in ways analogous to the use of antibodies, such as in IPA-style or ELISA-style assays. For example, the context of viral detection, aptamers have been produced for the spike protein on the surface of the SARS-CoV-2 virus.

The disclosures that follow describe diverse some embodiments of molecular electronics sensors using hybridization probes, primer extension probes, and aptamers. It is obvious to those skilled in the art of molecular biology that these have examples have diverse and varied extension to sensors that using the other types of binding probes described above, such as antibody or protein probes, or antigen probes, and to any other molecular binding system in which one component could be attached to a bridge molecule, or could otherwise comprise a portion of such a bridge molecule connecting the nanoelectrodes. This disclosures below are therefore not meant to be limiting, and are meant to encompass such obvious alternative sensor embodiments.

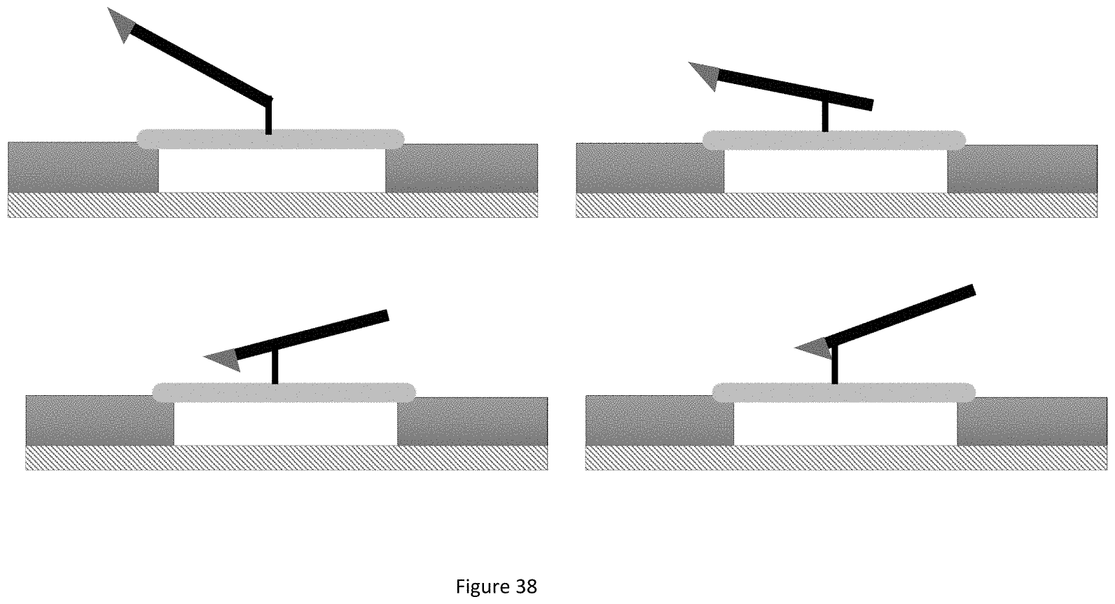

Hybridization and Aptamer Sensors—FIGS. 2 through 51 illustrate various some embodiments of hybridization sensors that may be used for the sensor array chip of the personal virometer (or, as indicated, aptamer sensors).

In various aspects of this disclosure, a DNA hybridization probe, which is a short piece of single stranded DNA, is attached by various means of conjugation to a bridge molecule that itself spans between two nano-electrodes and is suitably attached to each on either end, by some means of conjugation or binding. This configuration is further established within an aqueous solution. One exemplary embodiment of this configuration is illustrated in FIG. 2.2. Voltage is applied across the electrodes, which is accompanied by current flow through the bridge molecule. This current can be measured versus time, and this forms the primary measurement output of the device, as indicated in FIG. 2.2. Either end of the probe DNA (5′ or 3′) could in general be the end attached to the bridge, the FIG. 2,2 merely shows one of these embodiments, and is not limiting. In some embodiments, an internal base in the probe, rather than either ends, may also be the site for the conjugation. The DNA probe shown in FIG. 2.2 is a linear strand, but this is not meant to be limiting, and in other some embodiments it could be formed into a closed loop, have both ends attached to bridge, or comprise multiple linear segments joined by linkers, and in general need only be a molecular construct or complex that comprises a single stranded segment having the hybridization probe sequence of interest. Nor is it essential that the entirety of this segment engage in the specific hybridization of interest, in some embodiments it may be only a subsegment that engages in the hybridizing the target DNA. Nor is the length shown there (13 bases) meant to be limiting, the length could be in the range of 4-1000 bases, preferably 10-100 bases, and more preferably 12 to 50 bases, and in general the appropriate length range among these depends on the application. The gap spacing suggested by FIG. 2.2 is on the order of 10 nm, which is also not limiting, in various some embodiments, this gap could be in the range of 3-10 nm, 10-30 nm, 30-100 nm, or 100-1000 nm, or 1000 nm-10,000 nm. In various some embodiments, magnitude of applied differential voltage across the electrodes may be in the range of 0.01 Volts to 10 Volts, 0.05V to 3V, or 0.1V to 1.2V. In various some embodiments, this may be a DC voltage, or an AC or time varying voltage, either sinusoidal, periodic or varying according to some other applied waveform. In various some embodiments, there may also be a reference and working electrode provided to the solution phase, the set and control the potential of the solution relative to the potential of either electrode or to the ground of the circuit. Such electrodes may be platinum electrodes (or pseudo-electrodes as these are sometimes called), Silver/Silver Chloride electrodes, or other types of electrodes use to set solution potential known to those skilled in the art of electrochemistry. The resulting current measured in various some embodiments, may have baseline or typical magnitudes in the range of 1 pico-Amp (pA) to 10 pA, or 10 pA to 100 pA, or 100 pA to 1000 pA=1 nano-Amp (nA), or 1 nA to 10 nA, or 10 nA to 100 nA. In various some embodiments, there may also be a buried “back gate” electrode some distance underneath the insulating substrate, that may be used to apply an additional modulating voltage or field to the vicinity of the bridge and hybridization probe. In some embodiments, such an electrode may be buried 1 nm-10 nm below the surface, 10 nm-100 nm below, or 100 nm-1000 nm below. In some embodiments, such an electrode may also have lateral localization such that it is localize to be near the footprint of the bridge, in one or both lateral dimensions, such as extending no more than 10 nm or no more than 50 nm, or no more that 500 nm or no more than 1000 nm beyond the footprint of the bride, in one or both lateral dimensions.

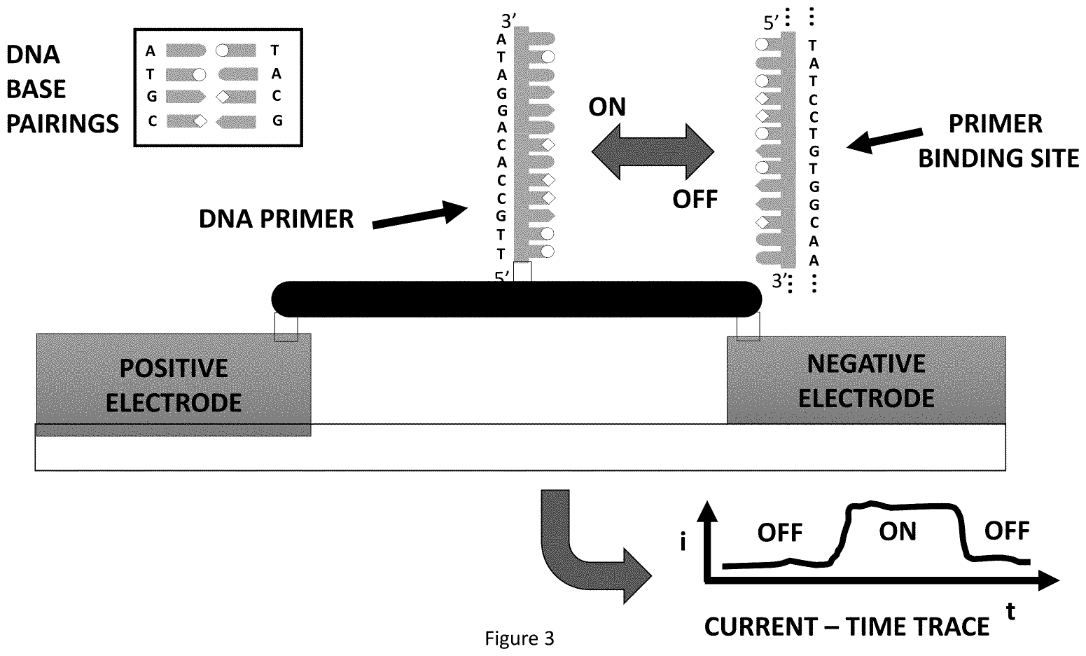

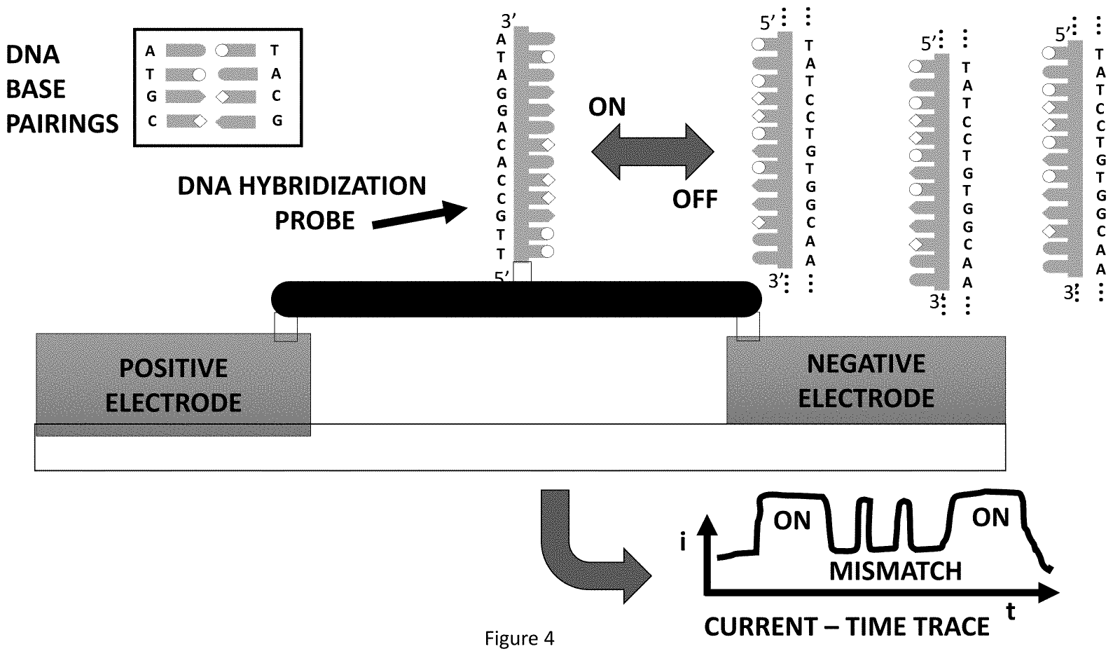

As stated, an embodiment of the disclosure is directed to provide a composition that can function as a molecular electronics sensor for hybridization events. When such a molecular electronic sensor is exposed to a solution that contains single stranded DNA material, if the hybridization probe encounters and binds by hybridization to its intended exact match target, a distinguishable signal is produced in the measured current. This is illustrated in FIG. 3, which shows the probe encountering a segment (of a longer DNA molecule as indicated) that is an exact match (i.e. reverse complement sequence), and the current changing—there shown as jumping up to a larger value—when the probe has hybridized to this target, indicated as the “on” state, and then the current reverting back when the probe dissociates to the “off” state again. This signal thereby provides the primary detection of the target being present in the sample. If the exact match target is not encountered, there is no such distinguishable signal. Furthermore, as indicated in Figure. 4, when the this sensor is exposed to complex pool of DNA fragments, possibly including hybridization targets, indicated by the two specific complementary sequences shown, but also containing many non-matching or off-target fragments, indicated by the shaded anonymous segments shown, the hybridization events with exact matches produce the long duration detectible signals, which the many interactions with non-matching segments may result in no detectable jump in current, or many result in transient, rapid or shorter duration pulses that are readily discriminated for the stronger (i.e. longer duration) correct match detection pulse or step. This different in signal between the exact match hybridization event, and diverse non-specific interactions with other fragments, include interactions with just one or a few mismatches, provides for the primary detection of targets of interest in a sample, relative to a background of many other off-target fragments, even if such fragments may differ by only a single base from the target. Thus, this provides a molecular electronic single molecule hybridization sensor that can detect the presence/absence of a target DNA of interest in a potentially complex pool of DNA as might be extracted from a biosample of interest by common sample preparation methods for extracting, purifying, or amplifying DNA (or RNA). As shown in FIG. 4, it is suggested that the target one events result in a long step up pulse, while the off-target events result in short step ups of similar magnitudes but shorter duration. These details of the Figure are not meant to be limiting, and in some embodiments, the exact target binding events might result in a step up or step down in magnitude of current, and this might be a step that does not return to baseline in conditions where the target stays bound. The off-target events in some embodiments may also make pulses of shorter duration and/or smaller amplitude, or that may otherwise have a signature in the current trace that differs from the exact match signatures. In some embodiments, the conditions of the operating temperature, buffer chemical composition, pH, applied voltage, back gate voltage (if any), solution potential, or other parameters of the system may be modified to enhance or optimize the ability to discriminate between exact match hybridization signals and various background interaction signals that the probe may engage in, or that are noise arising from other aspects of the molecular electronic system. In terms of buffer composition, there are many factors that may be used and that are known to those skilled in molecular biology that may impact hybridization reactions, such as concentrations of salts (such as NaCl, KCl), divalent cations (such as Mg++, Mn++, Ca++, Sr++), detergents and solvents (such as Triton or DMSO or betaine), and the primary buffering agent, such as Tris, HEPES or MOPS.

In some embodiments, the nature of the detectible signal produced by the presence of the target is a series of spikes in the current, that correspond to target DNA binding to the probe, and then coming off of the probe. This is in general an expected behavior, as the hybridization binding is reversible, and the rate of binding “on” is influenced by concentration of the target DNA in solution, as well as composition of the buffer, pH, temperature, etc., as is the rate of coming “off” for a bound target also dependent on temperature, and such properties of the solution.

In some embodiments, the properties of the observed spikes, such as the length of time from exposure to first observation an exact-match step up, the time between pulses, or the ratio of time on to time off, or other properties of the on/off rates, are relatable to the concentration of the target, and therefore provide a measure of concentration of the target of interest. Thus, by analyzing the data to extract and compute such measures, this provides a molecular electronic single molecule hybridization sensor that can detect concentration of a target DNA of interest in a sample, including samples with a background that may be a complex pool of off-target fragments.

In some embodiments, a perfect match hybridization between the probe and target DNA will produce a detectible signal, and a single based mismatch in an off-target DNA relative to the probe DNA in the sensor will produce a distinguishably altered signal, and further levels of mismatches will produce even more distinguishably diminished signals, or little or no detectible signals. In this way, the sensor signal can be used to distinguish targets that are a perfect match to the hybridization probe DNA from other fragments that have even a single base mismatch. This provides sufficient sensitivity and selectivity to perform the genetic analysis applications of genotyping and strain determination, which often require the ability to discriminate DNA targets that differ by as little as a single base mismatch, or otherwise may differ by just a few bases of mismatch, or by insertions or deletions of one or a few bases.

In particular, the identification of Single Nucleotide Polymorphism (SNP) genotypes becomes possible, as these require single base discrimination among different DNA segments. For this, we disclose a method for SNP genotyping, where in the organism or biosample of interest, two or more sequence variants may be present, differing between any two by as little as one base substitution, or one base insertion or deletion, and specific hybridization probes are made for each such sequence, and put into molecular electronic sensors of the type disclosed. From a primary bio-sample of interest, DNA or RNA is suitably purified, using any of various means well known to those skilled in molecular biology, such as using an extraction column or phenol-chloroform extraction, and the purified sample is applied to these sensors, either separately, in different reaction volumes, or within one reaction volume applied to a device containing all such sensors. Such a device could be a CMOS chip with all such sensors present on the chip. By monitoring the signal results from each sensor, it can then be determined which if any of the variant targets are present in the sample, and this information can be used to determine a genotype for subsequent interpretation, or to identify the presence of one or more specific strains of a pathogen. In some embodiments of this method, this could be determining the strain of a parasite, fungi, bacteria or virus.

Referring again to the general hybridization sensor disclosed, and as illustrated in FIG. 2.2, the bridge molecule may be any molecule that can serve as a conducting connection between the electrodes. Such a conducting bridge molecule could, in some embodiments, be a double stranded DNA segment, an alpha-helical protein, a carbon nanotube, a graphene nanoribbon, a multi-chain protein such as a bacterial pilin filament or bacterial nanowire, or a conducting polymer such as PDOT. In some embodiments, the bridge molecule is fabricated by bottom up chemical synthesis, and is made to have a defined chemical structure, and to have engineered in specific binding groups at precise locations in the chemical structure to provide the conjugation or binding sites for the hybridization probe and the nanoelectrodes. In some embodiments, such a bridge molecule has a specific conjugation site to which the hybridization probe DNA is conjugated to, either covalently or non-covalently, through any of many possible conjugation mechanisms known to those skilled in the art of bioconjugation. In some embodiments, this conjugation could be a click chemistry coupling, such as DBCO-azide or TCO-azide or other copper or non-copper click reactions, an APN-thiol coupling, an amine-NETS ester coupling, a biotin-avidin coupling, a peptide-tag based coupling such as Spy-Catcher, or an AviTag or an Aldehyde Tag. In some embodiments, the electrodes provide exposed metal contact targets for binding the bridge, and in preferred embodiment, the metals may be from among gold, platinum, palladium, or ruthenium. In some embodiments, the bridge may be conjugated to the electrodes by a connection comprising a thiol-metal bond, dithiol-metal bond, amine-metal bond, carbene-metal bond, or diazonium-metal bond, or other carbon-metal bonds, or may comprise a metal binding peptide, of which many are known to those skilled in biochemistry, such as the known Gold Binding Peptide (GBP) with amino acid sequence MHGKTQATSGTIQS (SEQ ID NO: 9), and which many also be repeated in tandem 2-6 times, separated by short GS rich linkers, or alternatively the known Palladium binding peptide QQSWPIS (SEQ ID NO: 10). The conjugation could also be achieved by applying a bifunction linker with any of these or other binding groups use to attach one end to the electrode, and have a short linker such as a PEG or carbon chain 1-3 nm long, presenting arbitrary second conjugation group as the head group, such as a click chemistry group, that can then be used to conjugated to a cognate group on the bridge molecule.

In some embodiments, the DNA hybridization probe may be between 4 and 200 bases in length, preferably between 10 and 100 bases in length, and preferably 12 to 60 bases in length. In applications requiring single base discrimination, such as SNP genotyping or pathogen strain determination, the probe length is preferably 10 to 35 bases in length, or more preferably 15 to 25 bases in length. The probe may also comprise other nucleic acids or nucleic acid analogs, such as RNA, PNA or LNA, which may provide stronger binding or great specificity of binding. Such probes can have reduced length. The probe DNA may also comprise a fluorescent group, such as a FAM dye molecule on the 5′ end, and groups can be used for quality control or characterization in the synthesis and purification of the probe-bridge conjugates, or the characterization of the assembled sensors, such as in optically assessing whether a nanoelectrode has received a probe-bridge complex.

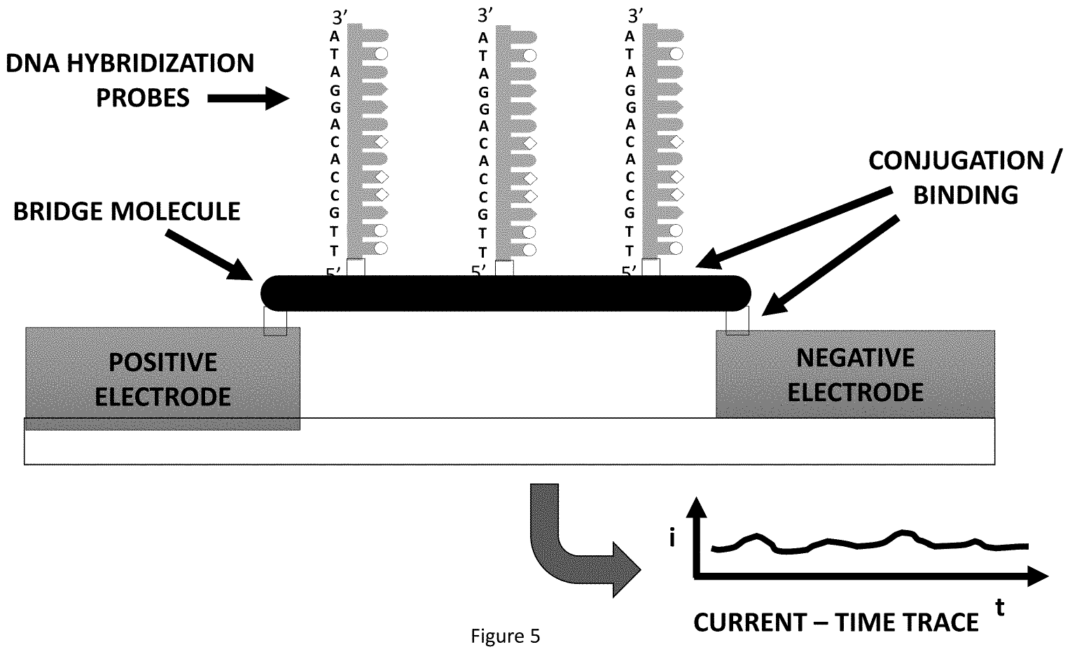

In some embodiments, the molecular bridge illustrated in FIG. 2.2 could have a length of 3-10 nm, or 10-100 nm, or 100-1000 nm, or 1000 nm-10000 nm. In some embodiments, as illustrated in FIG. 5, there could be more than one hybridization probe conjugated at specific sites on the molecular wire, as illustrated in FIG. 5. In preferred embodiment, such multiple probes may identical DNA probes, or distinct DNA probes—i.e. probes that target the same sequence, or different sequences. The benefits of multiple probes could include increased total signal, increased signal to noise, robustness against failures of probe conjugation, robustness against probes being in inaccessible configurations, or the ability to multiplex one sensor to detect multiple targets, for greater robustness or breadth of detection. Such multiple probes in referred embodiments would number between 2 and 10, between 10 and 100, or between 100 and 1000. Such probes may be located so as to point in similar orientations relative to the bridge, as suggested by the cartoon in FIG. 5, or they may be located so as to point in different orientations away from the bridge. This may provide a benefit of ensuring some probes are spatially accessible once the bridge is in place. The spacing between such probes along the molecular bridge would in some embodiments be in the range of 1-10 nm, or in the range of 10 nm-100 nm. In some embodiments, the bridge molecule is fabricated by bottoms up synthesis to have these multiple probe conjugation sites at precisely defined locations in the chemical structure of the bridge.

In some embodiments, the application could be gene expression analysis of any cellular samples, and in general could be any application where methods such as DNA microarrays have been used for gene expression. In some embodiments, this would include gene expression applied to tumor tissue as may be used in cancer diagnostics.

In some embodiments, the application could be SNP genotyping in human, animal, or other cellular samples, and in general any application where methods such as DNA microarrays have been used for genotyping. In some embodiments, this would be applied to SNP genotyping in humans.

In some embodiments, the application is massively multiplexed hybridization probe detection and/or concentration measurement of targets in a complex pool. The level of multiplexing could be up to 100 different probes, 1000 probes, 10,000 probes, 100,000 probes, 1 million probes, 10 million probes, 100 million probes, or 1 billion probes. The provides an alternative to DNA microarrays for such high levels of multiplex detection, with the advantages of all-electronic, chip-based system, single molecule sensitivity, speed, low cost consumables and instrument, and compact mobile, portable, or point-of-use instruments.

In some embodiments, the application could be species identification, in particular determining what species a given tissue sample is taken from, or in the identification of which pathogens, such as bacteria or viruses are present in a given sample. In some embodiments, this could be testing of environmental samples for the presence of a given virus, such as COVID-19. In an exemplary embodiment, the sample could be a tissue, or fluid, from any of the common vectors for viral transmission, such as bats, birds, rodents or mosquitoes. In preferred embodiment the sample could be material filtered from air or water, or material swabbed from a surface. In some embodiments the sample could be a biosample taken from a human or animal subject, such as saliva, mucous, buccal swab, blood, sweat, urine, stool, or exhaled air.

In some embodiments, the application could be strain identification, in particular determining what strain of a pathogen, such as a bacteria or virus, are present in a given sample. In some embodiments, this could be testing of environmental samples for the presence of a given strain of a virus, such as COVID-19. The samples for this, could be the same as for the previous species identification application.

Primer Extension Assays—In some embodiments, a primer extension reaction is used to detect the presence of a given DNA sequence target in a sample, by the following method. A primer for the target of interest is applied to the sample, polymerase and dNTP are provided to allow for extension, under conditions where if the primer is bound off target, it will not extend, and the reaction is monitored for a detectible signal of extension. If the extension is observed, the primer found its exact match, and thus the target is detected. This Primer Extension Assay has the advantage of greater specificity than hybridization assays, because it has the additional ability to discriminate between perfect match on-target hybridization, and off-target or mis-matched hybridization. It may also afford an advantage of a stronger signal or larger signal to noise ratio (SNR) because of the potentially larger signal of the extension process.

Allele Specific Primer Extension Assays—In some embodiments, allele specific primer extension can be used to discriminate with great specificity between alleles or variants that differ at a single base, by the following method. The fact that extension can only occur if the 3′ end of the primer matches the template can be used as the basis of an assay to interrogate what base is on the template at that site. In an exemplary embodiment, illustrated in FIG. 34.1, there is provided a test sample fragment, with a primer binding site, and the primer binding site defines an interrogation site at the position of the 3′ end of the primer, indicated by the underline. Two primers are provided that differ in the 3′ terminal base, and which are used to probe the interrogation site. As shown in the example, one ends in a 3′ G—which probes for a C in the test template, and the other ends in 3′ T—which probes an A in the test template. The test fragment is allowed to bind with each primer, and then an extension reaction is carried out, and the result of this is observed, as either extension or no extension. Which primer extends provides the information on what base was at the interrogation site in the test fragment—as shown in the example, the C-variant Primer extends, detecting the presence of the C variant form of the template. Put into the language of a genetic test, the primer would define the locus under consideration, these two variant forms of the test fragment would be considered as two alleles, which would be called the C allele and A allele, and the resulting observation of C would be the genotype measured at this locus, for the test sample. The primers in this application are referred to as “allele specific primers”, and the assay is referred to as an “allele specific extension” assay.

In general, the some embodiments disclosed above provide for a genotyping assay, that can be used to type for any of the (up to 4) bases occur at the single-base interrogation site defined by the 3′ end of the primer. In some embodiments, this can be applied to SNP genotyping. In other some embodiments, this method can also be used to type for insertion or deletion variants, as long as the primer binding site can be chosen such that the set of variants under consideration comprises a base change at that site, and do not alter the sequence at the rest of the primer site.

In more general embodiments of this method, for any given set of variant alleles at a locus of interest, there can be provided a corresponding set of primer extension detection primers, which perfectly bind their respective target allele, and otherwise do not bind properly for extension on any of the other alleles, either because of, in some embodiments, a mismatch at the 3′ end, and/or because there are substantial mismatches internally in such off-target bindings that lower the melting point well below the reaction temperature compatible with the perfect match extension reactions. In this case, by applying the different allele specific primers to a sample of test fragments—at a suitable reaction temperature compatible with extension of the perfect match primers—and observing the extension or non-extension, a sample can be typed for the presence or absence of any of these alleles. These more general assays, that go beyond SNP genotyping, are also referred to as allele specific extension assays, based on the respective allele specific primers.

In some embodiments of this method, for any given set of sequence-similar variants, the same method can be applied. In some embodiments, this can be applied to distinguishing similar strains of a pathogen, and the assay would then be a “strain specific primer extension” assay.

Molecular Electronics Sensors for Primer Extension Assays—In various aspects of this disclosure, a DNA primer is attached by various means of conjugation to a bridge molecule that itself spans between two nano-electrodes and is suitably attached to each electrode on either end, by some means of conjugation or binding. Voltage is applied across the electrodes, which is accompanied by current flow through the bridge molecule. This current can be measured versus time, and this forms the primary measurement output of the device, as indicated in FIG. 2.2.

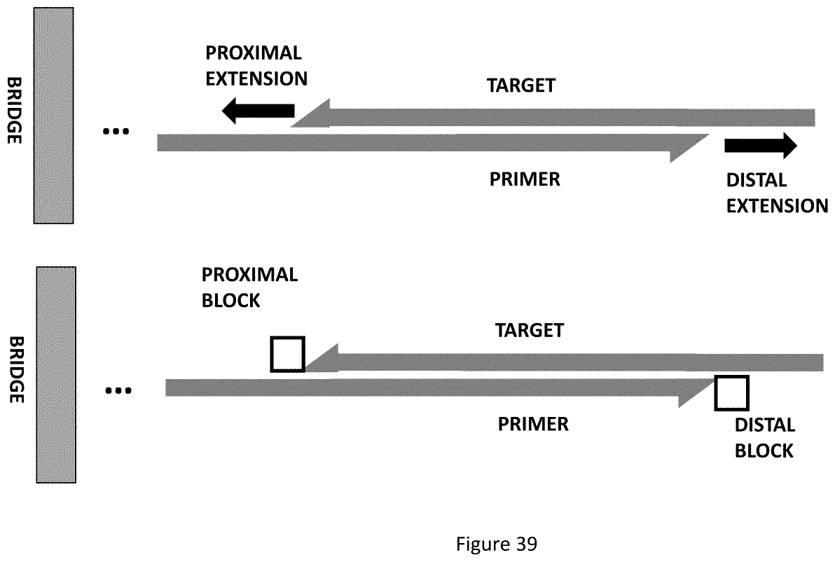

Either end of the primer DNA (5′ or 3′), could in general be the end attached to the bridge, as long as the primer is not inhibited from hybridizing to its target on a template, and undergoing extension by a polymerase. FIG. 2.2 merely shows one of these embodiments, where the 3′ end is distal to the bridge, and clearly availably for target binding and extension, and this is not meant to limit the disclosure. In some embodiments, an internal base in the probe, rather than at or near either ends, may also be the site for the conjugation, such as illustrated in FIG. 39. It is not essential that the entirety of this segment engage in the primer binding, in some embodiments it may be only a subsegment including the 3′ end that binds the primer binding site. Nor is the length shown there (13 bases) meant to be limiting, the length of the primer could be in the range of 4-100 bases, preferably 10-50 bases, and more preferably 12 to 40 bases, and in general the appropriate length range depends on the application and also on desired aqueous solution conditions, such as temperature and salt concentration and other additives. The electrode gap spacing suggested by FIG. 2.2 is on the order of 10 nm, which is also not limiting, in various some embodiments, this gap could be in the range of 3-10 nm, 10-30 nm, 30-100 nm, or 100-1000 nm, or 1000 nm-10,000 nm. In various embodiments, the magnitude of applied differential voltage across the electrodes may be in the range of 0.01 Volts to 10 Volts, 0.05V to 3V, or 0.1V to 1.2V. In various some embodiments, this may be a DC voltage, or an AC or time varying voltage, either sinusoidal, periodic or varying according to some other applied waveform. In various some embodiments, there may also be a reference and working electrode provided to the solution phase, the set and control the potential of the solution relative to the potential of either electrode or to the ground of the circuit. Such electrodes may be platinum electrodes (or pseudo-electrodes as these are sometimes called), Silver/Silver Chloride electrodes, or other types of electrodes use to set solution potential known to those skilled in the art of electrochemistry. The resulting current measured in various some embodiments, may have baseline or typical magnitudes in the range of 1 pico-Amp (pA) to 10 pA, or 10 pA to 100 pA, or 100 pA to 1000 pA=1 nano-Amp (nA), or 1 nA to 10 nA, or 10 nA to 100 nA. In various some embodiments, there may also be a buried “back gate” electrode some distance underneath the insulating substrate, that may be used to apply an additional modulating voltage or field to the vicinity of the bridge and hybridization probe. In some embodiments, such an electrode may be buried 1 nm-10 nm below the surface, 10 nm-100 nm below, or 100 nm-1000 nm below. In some embodiments, such an electrode may also have lateral localization such that it is localize to be near the footprint of the bridge, in one or both lateral dimensions, such as extending no more than 10 nm or no more than 50 nm, or no more that 500 nm or no more than 1000 nm beyond the footprint of the bride, in one or both lateral dimensions.

In an aspect, the composition can function as a molecular electronics sensor for determining whether the primer finds an exact match, or not, in a sample. In some embodiments, when such a molecular electronic sensor is exposed to a solution that contains single stranded DNA material, if the primer binds to a binding site, a signal may be produced in the measured current. This is illustrated in FIG. 3, which shows the probe encountering a segment (of a longer DNA molecule as indicated) that is an exact match (i.e. reverse complement sequence) to the primer, and the current changing—there shown as jumping up to a larger value—when the primer has hybridized to this target, indicated as the “on” state, and then the current reverting back when the probe dissociates to the “off” state again. In general, such primer binding could be a perfect base-pair match between the primer sequence and target sequence, or could in comprise mismatches. The binding signal in itself may not determine whether the primer formed a perfect match and therefore found its target, or has mismatches and therefore has not found its proper target. As indicated in FIG. 4, in some embodiments of this process, the primer is exposed to complex pool of DNA fragments, possibly containing any combination of off-target (indicated by the shaded anonymous segments shown) and on-target templates (illustrated by the complementary sequences shown), and the resulting primer binding events may produce diversity of signals (shown as longer and shorter pulses), which again may not be sufficient to fully distinguish on-target perfect binding of the primer to target, from off-target primer binding. It is an advantage of the primer extension process that, in this setting, it can add additional information that can be used to distinguish exact-match primer binding from mis-matched primer binding, especially when such a mismatch is at the 3′ end of the primer.

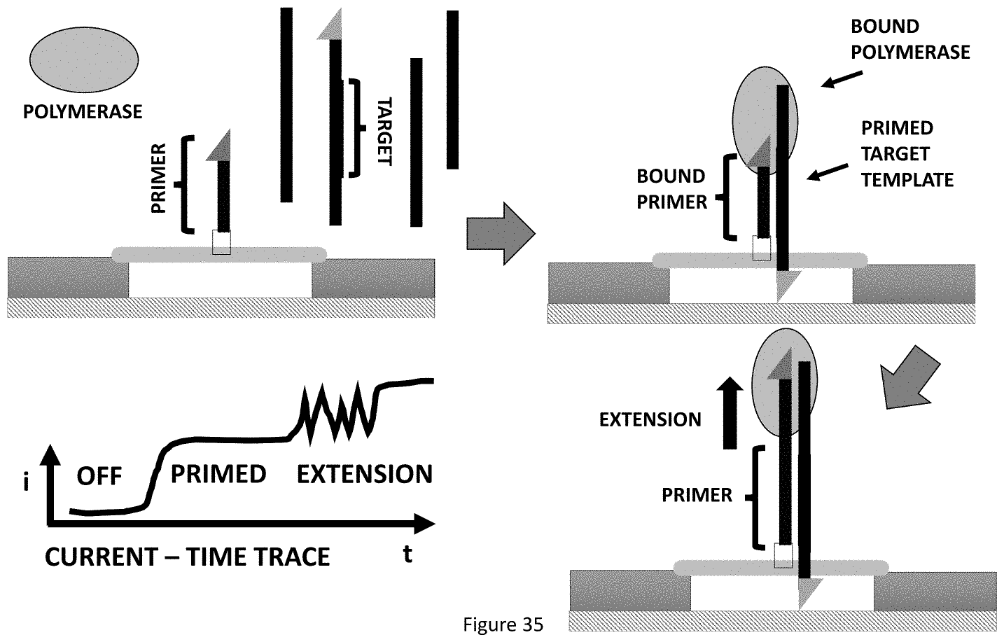

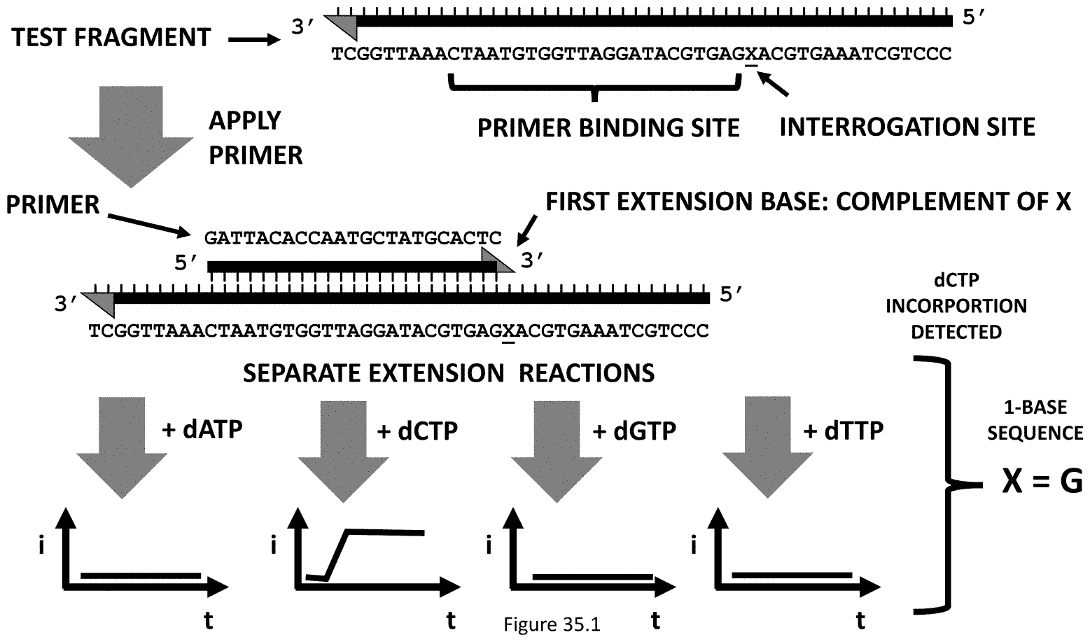

The sensor of FIG. 2.2 can be used for a primer extension assay as disclosed here. As illustrated in FIG. 35, first the primer is allowed to bind a target in a pool of sample fragments (upper left), which may produces distinguishable signal indicative of primer binding events (lower left, where, for example, the signal is shown elevated in the segment marked “primed”). However, such events may include both perfect match and mis-matched primer binding. There is further provided a supply of polymerase and dNTPs, and in the presence of a properly primed template the polymerase bind end extend properly primed template (upper right), which will in some embodiments produce a distinguishable signal coming from the primer extension, as shown in the signal trace (lower left, where, for example, there are large signal spikes and elevated signals observed in the segment marked “extension”). Such an outcome illustrates the sensor successfully detecting primer extension, and therefore that the primer was perfectly matched to the template. The lack of such an extension signal in contrast is a negative outcome, and would be interpreted that the primer did not properly match the template.

It is an advantage of the molecular electronic primer extension sensor disclosed that it can detect with high sensitivity and specificity a primer target sequence of interest within a test sample that contains a complex pool of diverse DNA sequence fragments. It is an advantage that is can do so with much higher specificity and sensitivity than pure hybridization assays based on a hybridization probe binding signal.

In some embodiments, such a primer extension assay can be used to accurately detect in a complex test sample, a target site of the primer that represents a particular genetic allele, for the purpose of genotyping, or a particular species or strain specific sequence, for purpose of identifying a species or strain. In some embodiments, this would be identification of a species or strain of pathogen responsible for an infectious disease, for the application of monitoring or diagnosis of infectious disease.

Thus, this provides a molecular electronic single molecule primer extension sensor that can detect the presence/absence of a target DNA of interest in a potentially complex pool of DNA as might be extracted from a biosample of interest by common sample preparation methods for extracting, purifying, or amplifying DNA (or RNA). In some embodiments, the conditions of the reaction temperature, buffer chemical composition, pH, applied voltage, back gate voltage (if any), solution potential, or other parameters of the system may be modified to enhance or optimize the ability to discriminate between primer extension signals and various background signals or noise arising from other aspects of the molecular electronic system. In terms of buffer composition, there are many factors that may be used and that are known to those skilled in molecular biology that may impact primer extension reactions, such as concentrations of salts (such as NaCl, KCl), divalent cations (such as Mg++, Mn++, Ca++, Sr++), detergents and solvents (such as Triton or DMSO or betaine), and the primary buffering agent, such as Tris, HEPES or MOPS.

In some embodiments, the nature of the detectible signal produced by primer extension is a series of spikes in the current, that may correspond to the polymerase engaging with template-matched dNTPs and incorporating template-matched dNTPs.

In some embodiments, the properties of the observed real-time signal, such as the length of time from first exposure to the sample to first observation a primer binding signal, or features of the primer binding signal such as pulse rate or on/off time fractions, or the time to start of the primer extension signal are relatable to the concentration of the target, and therefore provide a measure of concentration of the target of interest. Thus, by analyzing the data to extract and compute such measures, this provides a molecular electronic single molecule primer extension sensor that can detect concentration of a target DNA of interest in a sample, including samples with a background that may be a complex pool of off-target fragments.

In some embodiments, a perfect match hybridization between the probe and target DNA will produce a detectible signal, and a single based mismatch in an off-target DNA relative to the probe DNA in the sensor will produce a distinguishably altered signal, and further levels of mismatches will produce even more distinguishably diminished signals, or little or no detectible signals. In this way, the sensor signal can be used to distinguish targets that are a perfect match to the hybridization probe DNA from other fragments that have even a single base mismatch. This provides sufficient sensitivity and selectivity to perform the genetic analysis applications of genotyping and strain determination, which often require the ability to discriminate DNA targets that differ by as little as a single base mismatch, or otherwise may differ by just a few bases of mismatch, or by insertions or deletions of one or a few bases.

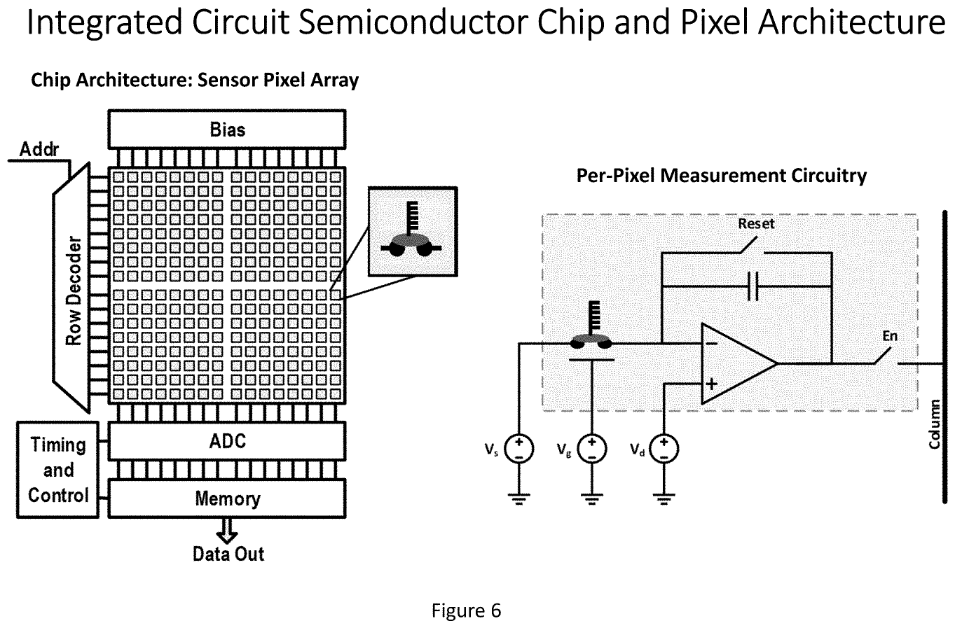

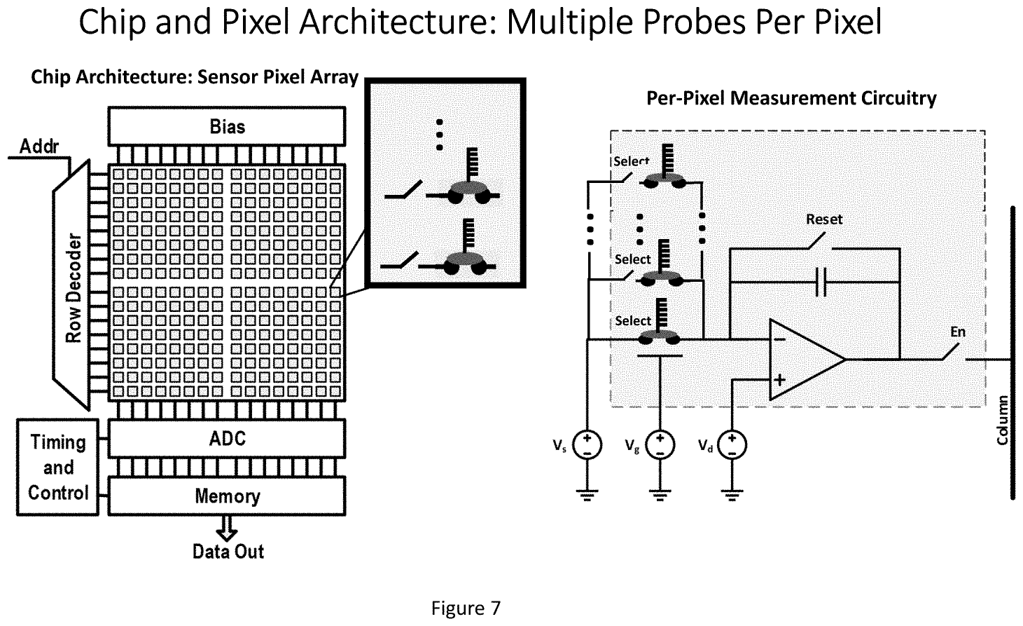

In some embodiments, there is a set of target sites {T1, T2, . . . , TN} that have similar sequences and that may be present in the test sample, and the primer of the sensor is designed to have a perfect match to one such target T of interest, and comprises a single base mismatch at the 3′ end if bound to any of the other sequence-similar sites Tj≠T, on the other possible segments that may be present, and in the setting, such a primer extension assay is used to detect the presence of the exact target sequence T in the sample, with specificity, even if the other similar segments Tj≠T are present. In some embodiments, there are distinct such primers, Pi, corresponding to each of the potential target sites, Ti, i=1, . . . , N. N such sensors are formed, and the test sample is applied to each such sensor, so that the presence or absence of each Ti in the sample can be assessed by this primer extension assay. In some embodiments, these N primers {P1, . . . , PN} will be deployed as a sensor array that can be exposed to a single sample solution volume. In some embodiments, this array of sensors will be deployed on the semiconductor chip sensor array device, as further disclosed in reference to FIGS. 6 and 7 below.

In some embodiments, such target sites {T1, T2, . . . , TN} are the different allele variants at a particular genetic locus, and such a multiplex test fully genotypes the sample for these alleles. In other some embodiments, the Ti may be different strain identifying sequences, for similar strains, and such as multiplex test fully characterizes the sample for which strains are present. In other some embodiments, the Ti may be different species identifying sequences, and such as multiplex test fully characterizes the sample for which species are present. In some embodiments, such species identifiers may variants from so-called “barcode of life” sequences, which are well-known and routinely used for the genetic identification of diverse species. In some embodiments, this would be segments of the Cytochrome C Oxidase subunit 1 (CO1) gene, which is used for species identification in animal species. In other some embodiments, this would be segments of the 16S ribosomal RNA gene, which is used for identification of bacteria and other diverse species.

SNP Genotyping—In some embodiments, such targets sites may represent the 4 possible variants of a SNP allele, corresponding to A, C, G, or T at a variable site of interest. In preferred embodiments, such targets sites may the two alleles of a bi-allelic SNP.

Single Base Sequencing Primer Extension Assay—In some embodiments for the application of typing SNPs, the primer 3′ end is located at the position adjacent to the SNP site, as illustrated in FIG. 35.1. The base immediately after the primer 3′ end, marked as “X” in the template, is the unknown base for the SNP site. Given such a primer, primer sensor for detection of extension, 4 different extension reactions are performed: The A-reaction, in which only dATP is provided to the polymerase, The C-reaction, in which only dCTP is provided to the polymerase, The G-reaction, in which only dGTP is provided to the polymerase, and The T-reaction, in which only dTTP is provided to the polymerase. Which of these 4 registers a detection of extension in the sensor—in this case, it is only a single base extension—determines which of the 4 bases is present at X. As shown in FIG. 35.1, only the dCTP reaction registers a signal, the other three reactions register no signal of extension, and therefore the unknown base must be X=G (the complement to dCTP). In an exemplary embodiment, the SNP is bi-allelic, with and only two of the four possible reactions are required to genotype the strand.

In an exemplary embodiment, primers for a set of N SNPs are put on a sensor array, so that N SNPs are assayed in the sample reaction. In one exemplary embodiment, a single such array is allowed to prime the sample, and then using this one array, in series the 4 respective A, C, G, and T reactions are performed, one after the other. In some embodiments, these may be performed by serial additions of the next trial dNTP to the solution, and after each such trial the sensor records the signal produced. In other some embodiments, four such identical arrays could be prepared, and the four reactions could be done in parallel on these, each on one of the arrays, to provide the benefit of reduce overall time to complete the reactions for rapid testing.

In an alternative preferred embodiment, for single molecule single bases extension reactions as are done on the sensors disclosed herein, the four reactions above, serially or in parallel, can be instead reduced to just three, with the outcome of the fourth reaction inferred, since at the single molecule level, as shown in FIG. 35.1, it is mutually exclusive that only one of the four reactions can result in extension, and therefore the fourth and final reaction is redundant. In this way, three reactions suffice.

In some embodiments, it is possible to reduce the need for 4 trial extension reactions above down to just 2 trial reactions, by the following method: Note that for the four possible bases {A, C, G, T} for a general unknown SNP base X as indicated in FIG. 35.1, in double stranded DNA form for the template, correspond to two possible bases pairs, either an A-T pair or G-C pair. Therefore, it is possible to choose primers such that only an exemplary pair of dNTPs need to be considered for single base extension, and with such choices made, the need for 4 reactions in the above methods can be reduced to just 2 reactions. Specifically, referring to the double-stranded form of the DNA near a SNP site X shown here (here N and n denote complementary bases, as do X and Y)

5′-[NNN . . . N]-3′ [X] 5′-[NNN . . . N]-3′

3′-[nnn . . . n]-5′ [Y] 3′-[nnn . . . n]-5′

the choice of 5′-[NNN . . . N]-3′ on the “upper strand” as a primer requires dXTP to extend, while the choice of 3′-[nnn . . . n]-5′ on the “lower strand” as a primer requires dYTP to extend, and either one of these could be used for single base sequencing determination of this SNP genotype. Therefore, if, for example, A-T SNPs are to be tested for dATP incorporation, and C-T SNPS are to be tested for dGTP incorporation, for each SNP of interest, by choosing the proper upper or lower primers, the trials of dATP addition and dGTP addition are sufficient to type the SNP (assuming the sample is prepared from double stranded DNA, so that both upper and lower strands are present for the SNP). In general, there are 4 possible choices for the preferred trial pairs of dNTPs: {dATP, dCTP}, {dATP, dGTP}, {dTTP, dCTP}, {dTTP, dGTP}. In some embodiments, one such choice of a trial pair is made, along with the constrained choice of primers, and then to type a total set of N SNPs, such primers are put onto an sensor array, and only two reactions need be performed, such as dATP and dGTP (or whichever of the 4 possible pairs of two are used to determine primers). These reactions may be performed serially in time on one sensor array device, by serial additions and measurement of extension, or on two identically prepared arrays, in two parallel single addition reaction experiments.

In an alternative preferred embodiment, for single molecule single bases extension reactions as are done on the sensors disclosed herein, the two reactions above, serially or in parallel, can be instead reduced to just a single reaction, with the outcome of the second reaction inferred, since at the single molecule level, as shown in FIG. 35.1, it is mutually exclusive that only one of the two reactions can result in extension, and therefore the reaction is redundant. In this way, in some embodiments, a single reaction suffices to type a set of N SNPs by single base sequencing primer extension, on a single molecule primer sensor array.

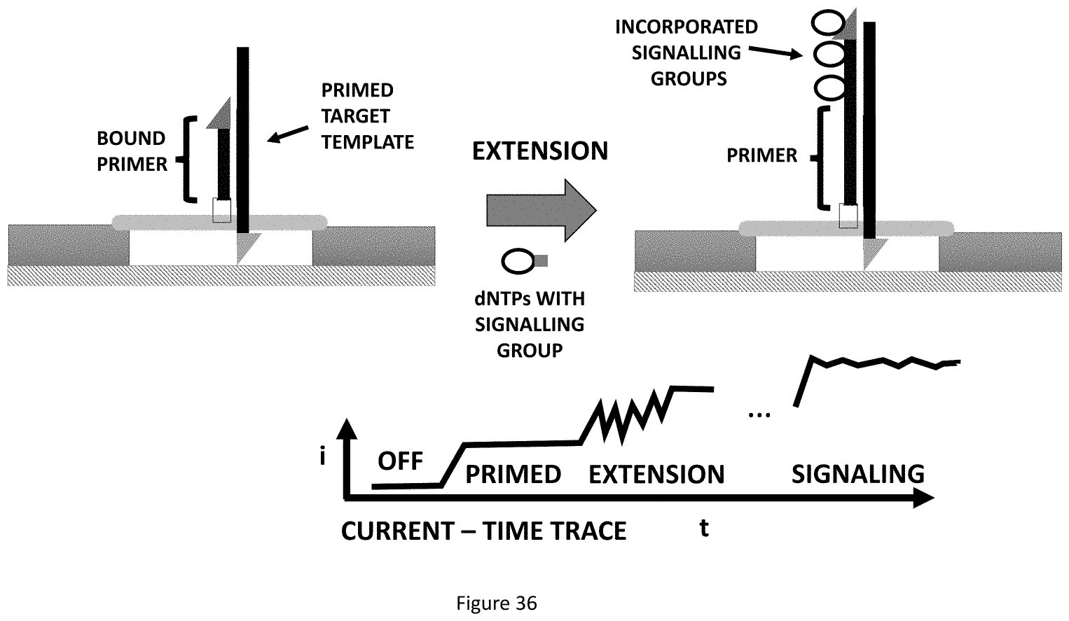

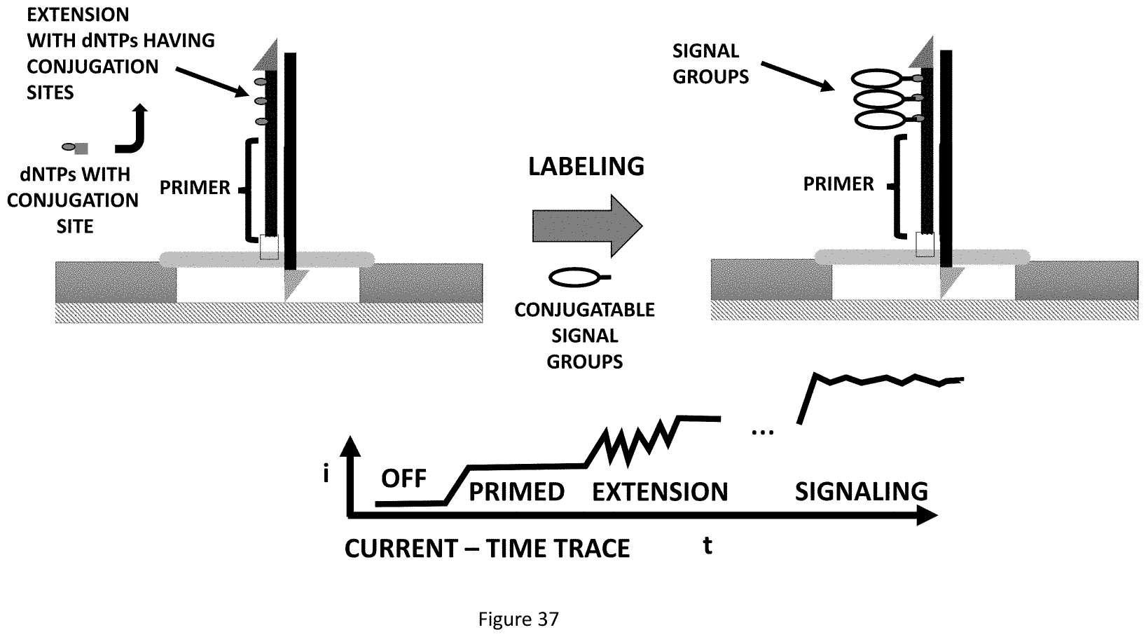

In some embodiments of single base sequencing by primer extension, the trial dNTPs will have attached signaling groups, as in FIG. 36, or have conjugation sites to be labeled with such, as in FIG. 37. In some embodiments, this produces an altered signaling that remains detectable after the single base extension is complete.

In another embodiment of single base extension, the 4 trial reactions indicated in FIG. 35.1 can instead be the addition of dBTP, dDTP, dHTP, or dVTP, where B, D, H, V are the IUAPAC codes for all bases other than A, other than C, other than G, and other than T, respectively. Thus, groups of 3 of the dNTPs are added in each reaction, leaving one out. In this case, the report out is inverted—non-extension determines uniquely which base is present on the template, as the complement of the missing dNTP in the group of three.

In particular, the identification of Single Nucleotide Polymorphism (SNP) genotypes becomes possible, as these require single base discrimination among different DNA segments. For this, we disclose a method for SNP genotyping, where in the organism or biosample of interest, two or more sequence variants may be present, differing between any two by as little as one base substitution, or one base insertion or deletion, and specific primers are made for each such SNP allele sequence, with a mismatch at the 3′ where the SNP is located on all other alleles, and put into molecular electronic sensors of the type disclosed. From a primary bio-sample of interest, DNA or RNA is suitably purified, using any of various means well known to those skilled in molecular biology, such as using an extraction column or phenol-chloroform extraction, and the purified sample is applied to these sensors, either separately, in different reaction volumes, or within one reaction volume applied to a device containing all such sensors. Such a device could be a CMOS chip with all such sensors present on the chip. By monitoring the signal results from each sensor during the primer extension reaction, it can then be determined which if any of the variant targets are present in the sample, and this information can be used to determine a genotype for subsequent interpretation, or to identify the presence of one or more specific species or strains of a pathogen. In some embodiments of this method, this could be determining the species or strain of a parasite, fungi, bacteria or virus.

Referring again to the general primer extension sensor disclosed, and as illustrated in FIG. 2.2, In some embodiments, the bridge molecule may be any molecule that can serve as a conducting connection between the electrodes. Such a conducting bridge molecule could, in some embodiments, be a double stranded DNA segment, an alpha-helical protein, a carbon nanotube, a graphene nanoribbon, a multi-chain protein such as a bacterial pilin filament or bacterial nanowire, or a conducting polymer such as PDOT. In some embodiments, the bridge molecule is fabricated by bottom up chemical synthesis, and is made to have a defined chemical structure, and to have engineered in specific binding groups at precise locations in the chemical structure to provide the conjugation or binding sites for the hybridization probe and the nanoelectrodes. In some embodiments, such a bridge molecule has a specific conjugation site to which the primer DNA is conjugated to, either covalently or non-covalently, through any of many possible conjugation mechanisms known to those skilled in the art of bioconjugation. In some embodiments, this conjugation could be a click chemistry coupling, such as DBCO-azide or TCO-azide or other copper or non-copper click reactions, an APN-thiol coupling, an amine-NETS ester coupling, a biotin-avidin coupling, a peptide-tag based coupling such as Spy-Catcher, or an AviTag or an Aldehyde Tag. In some embodiments, the primer could be conjugated to the bridge at any base along the primer, as indicated in FIG. 39, preferably in ways that does not interfere with the primer binding its target, or the polymerase extending the 3′ end. In some embodiments with conjugation comprises a spacer linker, which could be a PEG chain, preferably with a length in the range of 1-20 PEG units, or a Cn carbon chain, preferably of length n=1-20 carbon atoms. In some embodiments, the electrodes provide exposed metal contact targets for binding the bridge, and in preferred embodiment, the metals may be from among gold, platinum, palladium, or ruthenium. In some embodiments, the bridge may be conjugated to the electrodes by a connection comprising a thiol-metal bond, dithiol-metal bond, amine-metal bond, carbene-metal bond, or diazonium-metal bond, or other carbon-metal bonds, or may comprise a metal binding peptide, of which many are known to those skilled in biochemistry, such as the known Gold Binding Peptide (GBP) with amino acid sequence MHGKTQATSGTIQS (SEQ ID NO: 9), and which many also be repeated in tandem 2-6 times, separated by short GS rich linkers, or alternatively the known Palladium binding peptide QQSWPIS (SEQ ID NO: 10). The conjugation could also be achieved by applying a bifunction linker with any of these or other binding groups use to attach one end to the electrode, and have a short linker such as a PEG or carbon chain 1-3 nm long, presenting arbitrary second conjugation group as the head group, such as a click chemistry group, that can then be used to conjugated to a cognate group on the bridge molecule.

In some embodiments, the primer may be between 4 and 200 bases in length, preferably between 10 and 100 bases in length, and preferably 12 to 40 bases in length. In applications requiring single base discrimination, such as SNP genotyping or pathogen strain determination, the probe length is preferably 10 to 35 bases in length, or more preferably 15 to 25 bases in length. The probe may also comprise other nucleic acids or nucleic acid analogs, such as RNA, PNA or LNA, which may provide stronger binding or great specificity of binding. Such probes can have reduced length. The probe DNA may also comprise a fluorescent group, such as a FAM dye molecule on the 5′ end, and groups can be used for quality control or characterization in the synthesis and purification of the probe-bridge conjugates, or the characterization of the assembled sensors, such as in optically assessing whether a nanoelectrode has received a probe-bridge complex.

In some embodiments, the molecular bridge illustrated in FIG. 2.2 could have a length of 3-10 nm, or 10-100 nm, or 100-1000 nm, or 1000 nm-10000 nm. In some embodiments, as illustrated in FIG. 5, there could be more than one hybridization probe conjugated at specific sites on the molecular wire, as illustrated in FIG. 5. In preferred embodiment, such multiple probes may identical DNA probes, or distinct DNA probes—i.e. probes that target the same sequence, or different sequences. The benefits of multiple probes could include increased total signal, increased signal to noise, robustness against failures of probe conjugation, robustness against probes being in inaccessible configurations, or the ability to multiplex one sensor to detect multiple targets, for greater robustness or breadth of detection. Such multiple probes in referred embodiments would number between 2 and 10, between 10 and 100, or between 100 and 1000. Such probes may be located so as to point in similar orientations relative to the bridge, as suggested by the cartoon in FIG. 5, or they may be located so as to point in different orientations away from the bridge. This may provide a benefit of ensuring some probes are spatially accessible once the bridge is in place. The spacing between such probes along the molecular bridge would in some embodiments be in the range of 1-10 nm, or in the range of 10 nm-100 nm. In some embodiments, the bridge molecule is fabricated by bottoms up synthesis to have these multiple probe conjugation sites at precisely defined locations in the chemical structure of the bridge.

Additional Embodiments of Molecular Electronic Primer Extension Sensors The sensor disclosed herein can have many embodiments. The following some embodiments illustrate some of these, and are not meant to be limiting. Many additional variations are implied from these disclosures as would be obvious to one skilled in the fields of molecular electronics and nanotechnology, and these are meant to encompassed by the present disclosures.

FIG. 36 shows an exemplary embodiment in which some of the dNTPs used in the extension carry a signal enhancing group, indicated by the shaded circle, and these are incorporated into the extended primer. Such groups may produce an enhanced distinguishable signal either during the extension process, as the polymerase is actively incorporating bases, or after the extension is complete, in a post-extension measurement phase. In such a post extension phase, a special measurement buffer may be introduced to enhance the signal production. Further embodiments of post-extension measurement, and advantages are disclosed below in reference to FIG. 37.

In some embodiments, such an enhanced signaling group is attached to the dNTPs through some form of chemical bond or conjugation, or produced as some modified or analog chemical form of the dNTPs. Polymerase enzymes can in general incorporate various such modified forms of dNTPs in extension reactions, and many dNTP analogs (and the compatible polymerases) are well known in molecular biology. Such modifications may include the addition of such signaling groups as in FIG. 36, to some or all of the dNTPs, to facilitate the observation of extension, or the addition of binding sites on some or all of the dNTPs, to allow for post-extension addition of such signaling groups, as indicated in FIG. 37. In some embodiments, such modified chemical structures may have an addition onto the base portion of the dNTPs, at locations on the base which are well known to be tolerated by various polymerase, such as the 5-sites on the pyrimidine rings (of C, T), or 7-sites on the purine rings (of A, G). Other modifications may be added at the alpha phosphate site the dNTPs. Such modifications on the bases or alpha phosphate are retained in the growing strand (i.e. as indicated in FIG. 36). In other some embodiments, the modified structures are additions onto the gamma-phosphate terminal of the dNTPs. In further some embodiments, such gamma-phosphate modifications may comprise a polyphosphate chain extending the existing triphosphate chain, such as to extend the chain length from 3 to 4, 5, 6, 7, 8, 9, or more phosphates. In some embodiments, additional, highly diverse chemical groups may be added onto these extended chains. Such longer chains may allow the enzyme to more efficiently incorporate the modified dNTPs. Additions to the gamma phosphate are cleaved away during extension, and do not persist in the growing strand (i.e. unlike what is indicated in FIG. 36).

In some embodiments, all dNTPs may have an attached group. In other some embodiments, there may be a mix of standard dNTPs and those with an attached group, which allows for a portion of the extended strand to be native form, as indicated in FIG. 36. This may have benefits of more efficient extension, or to achieve a desired lower density of signal groups in the strand.

FIG. 37 shows an exemplary embodiment in which some of the dNTPs used in the extension carry a conjugation site, indicated by the small shaded circle, and these are incorporated into the extended primer. Following extension, a labelling reaction takes place, in which a conjugatable signaling group is provided to conjugate to the conjugation sites. The result is that the extended strand is labelled with the signaling group. At that point, as indicated, a signal detection phase of the measurement takes place, wherein the signaling groups produce an enhanced detectable signal. In some embodiments, a special measurement buffer may be introduced to enhance the signal production. It is an advantage of this approach that signal groups can be attached, or signal measurement solutions may be used, which may not be directly compatible with polymerase activity. It is also an advantage that the duration of the measurement phase does not have to be constrained by the rate or time of extension, and could be either much shorter or much longer. It is also an advantage that acquisition of the signal does not require continuous monitoring, and can be done at any time after the reaction, without risk of missing signaling events, in contrast to direct real-time observation of extension. In particular, this is an advantage for large sensor arrays, since not all sensors need to be monitored at the same time, thereby reducing the required data acquisition rate, or allowing measurement circuitry to be shared among multiple sensors, such as in FIG. 7. In some embodiments, all dNTPs may have a conjugation site. In other some embodiments, there may be a mix of standard dNTPs and those with a conjugation site.

The modified dNTPs must have a conjugation site that is retained post incorporation in the growing strand. Some embodiments may have such a modification at the 5-sites on the pyrimidine rings (of C, T), or 7-sites on the purine rings (of A, G), or at the alpha phosphate site the dNTPs. Many possible conjugation mechanisms known to those skilled in the art of bioconjugation can be used for this purpose, wherein one of the conjugation partner groups is placed in the dNTP, and the other on the signaling group. In some embodiments, this conjugation could be a click chemistry coupling, such as DBCO-azide or TCO-tetrazine or other copper or non-copper click reactions, an APN-thiol coupling, an amine-NETS ester coupling, a biotin-avidin coupling, an antigen-antibody coupling. In one exemplary embodiment, biotinylated dNTPs are used, and the signal groups are complexed with avidin. In some embodiments, attached of the conjugation partners to the dNTPs and signal groups comprises linkers or spacers as well, such as PEG chains or Carbon chains, or other commonly used linkers and spacers well known in conjugation chemistry.

FIG. 39 illustrates that in various some embodiments the primer may be conjugated to the bridge anywhere along its length. As shown, this may be at or near the 5′ terminus, or near the middle of the primer, or at or near the 3′ end of the primer. In some embodiments, in order to have the extension process occurring near the bridge the conjugation should be near the 3′ end, such as at the 3′ end, or within 5 bases, or within 10 bases. Such conjugation should be done so as to not interfere with the primer binding and extension process.

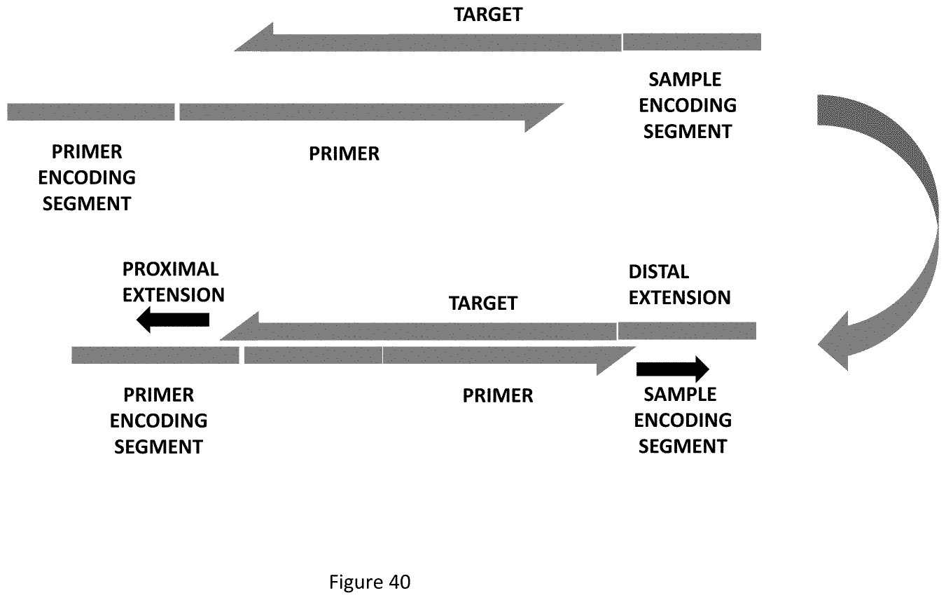

FIG. 40 illustrates that in various embodiments, the primer may bind the target template such that both the 3′ and of the primer and the 3′ end of the template could be used as sites for primer extension. These two sites are labeled there as proximal and distal relative to the bridge, for the purpose of distinguishing them, but in general the site of conjugation of the primer to bridge could be anywhere along the primer, as indicated in FIG. 39. Such dual extensions could occur at the same time, as would be the case in polymerase being allowed to bind and extend the upper configuration of FIG. 40. This may have the advantage of producing more extension signal. In some embodiments, there may be distinguished, selectively removable blocking groups, on either one or both ends, as shown in the lower configuration FIG. 40. In this case, selective removal can be used to extend a first and, and then in a later reaction, extend a second end. In one exemplary embodiment, there is one such group, the other 3′ end is free, and the first extension extends that end, and then the selective group is removed, and the other 3′ end is extended in the next extension reaction. In some embodiments, such a removable blocking group could be a cleavable chemical group protecting the 3′OH group of the 3′ end. In some embodiments, the block group could instead be a DNA oligo bound on the template and adjacent to the 3′ end, which can block extension by a polymerase that cannot displace strands. This block oligo can be removed by melting off thermally, or adding a denaturing chemical, such as betaine or DMSO.

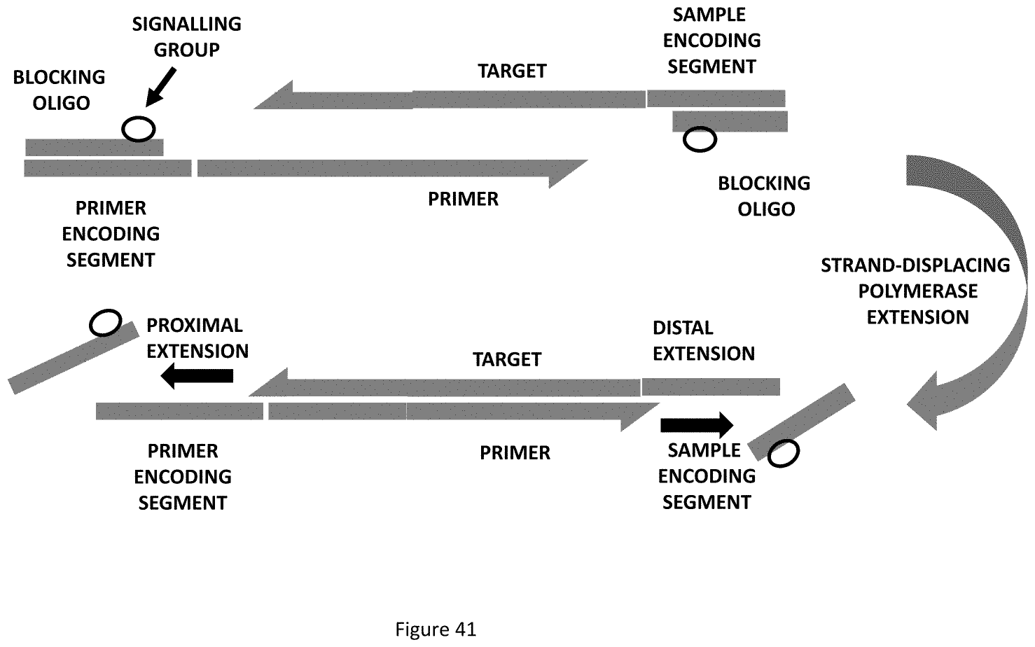

FIG. 41 illustrates various embodiment in which the sample is prepared such that the target segment 3′ end matches to the primer as indicated, and may also have a sample encoding segment on the 5′ end. The primer on the sensor has a binding site for the target 3′ end, and for a primer encoding segment. Upon hybridization of the primer to its target, the target 3′ end is extended to acquire a decoding signature from the encoding segment, that is used to determine the identity of the primer on the multiplex array of primers. In a separate extension reaction, the primer is extended, which also may generate a signature for the sample encoding segment that is used to identity the sample. In some embodiments, a blocking group or groups is used on one or both ends, to control which of these extension reactions occurs, and prevent them from occurring at the same time. The elements of this embodiment have the advantage of allowing for samples to be encoded and multiplexed onto one array sensor device for detection, or to allow for multiplex primers on the array to have encoded identities, that are decoded by extension of the 3′ end of the template. In referred embodiments, such templates that align to the sensor primer can be prepared by a target amplification reaction, that provides for a specific amplicon, which therefore has a specific sequence for which the primer can be chosen as the complement.

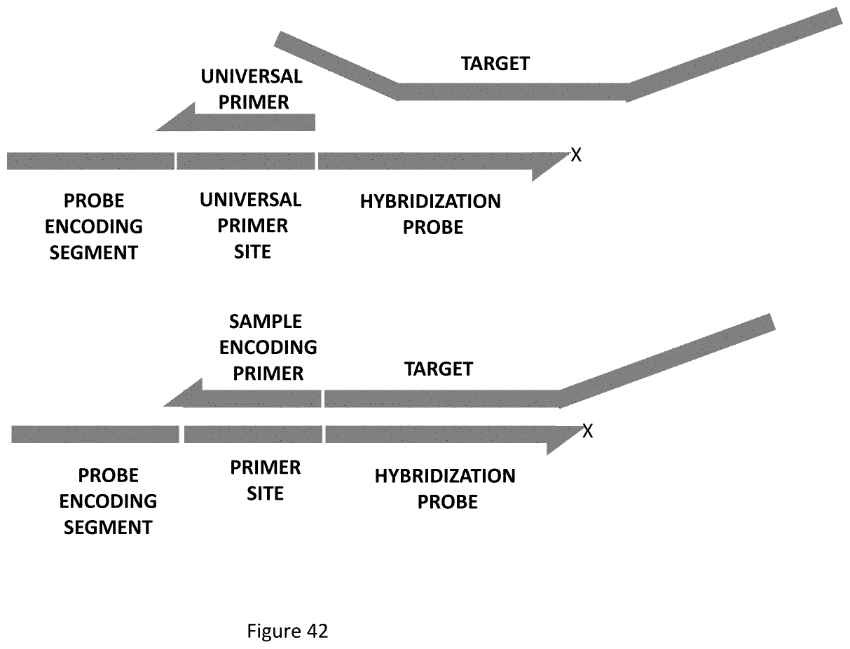

FIG. 42 Shows an embodiment the same as that disclosed in FIG. 41, except there are in addition oligos pre-bound to the primer encoding and sample encoding segments, and where such oligos are displaced during the respective extensions, by use of a strand-displacing polymerase, such as Klenow, Bst, Bsu, or Phi29. In some embodiments the displacement of these oligos produce a distinguishable signal, for use in the detection of the strand displacement, or in creating the signal signature of extension. Such oligos may further have attached to them, as indicated in FIG. 41, a signal generating group, that creates an enhanced signal when the strand is displaced. In some embodiments, there may be more than one such oligo bound along the strand on either end, and the corresponding series of strand displacements during the respective extension may provide for a unique signal signature of the extension.

As illustrated in FIG. 42, in some embodiments a signal of extension may be produced by the polymerase displacing one or more oligos that are pre-bound on the template, where the polymerase is a strand-displacing polymerase. Such oligos may have signal enhancing groups on them, that create an enhanced signal of displacement.

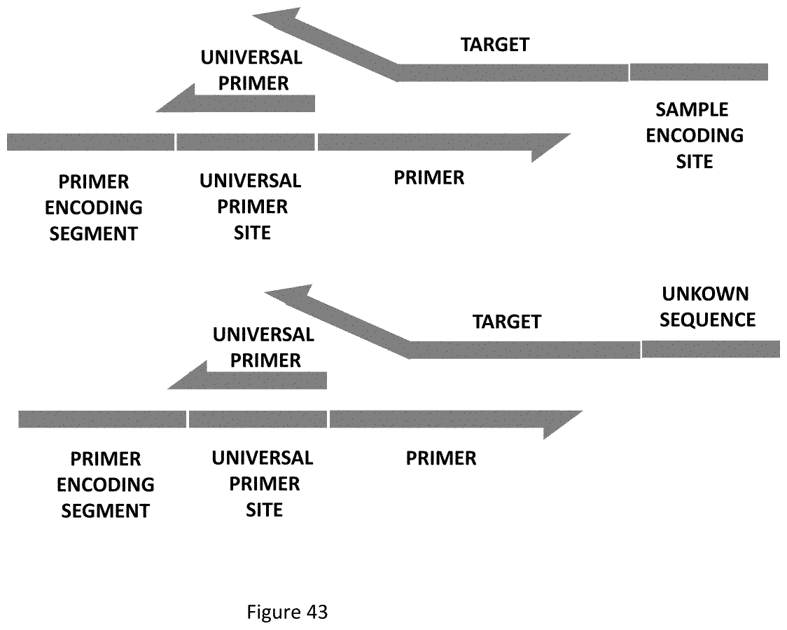

FIG. 43 illustrates two more some embodiments. In each of these, the 3′ end of the hybridization probe is blocked (X), so that the 3′ end cannot be extended. Such a block could be chemical group placed on the 3′OH, or the removal of the 3′OH group. In the upper embodiment shown, there is a sample encoding segment on the hybridization probe, flanked by a universal primer site that is common to multiple different probes on an array. The hybridization probe binds its target, and registers a hybridization probe binding detection signal. Separately, the universal primer is applied to the array, and its extension produces a signal signature that identifies the hybridization probe. This universal extension reaction could be done as a probe mapping step prior to use of the array in an assay, could be done within the same reaction as the hybridization (in which case the extension and hybridization signals must be distinguishable), or could be done in a separate decoding step to identify the probe after the hybridization reaction is already complete and detected. This provides for decoding and mapping of the hybridization probes by a primer extension reaction.

In the embodiment shown in the lower part of FIG. 43, the multiple samples are encoding by the targeted addition of a sample encoding 3′ end primer, such that N multiple samples can be applied on the array, and for each specific hybridization probe target segment, N instances of it occur on the array, with such a segment appended to the N different sample encoding primers. In this embodiment, a particular sample-target segment will hybridize to the appropriate complement on the array, and then primer extension generates a decoding signature from the probe encoding segment that decodes that specific probe, which included both the sample identity and the identity of the probe target. This has the advantage of allowing for both multiplexing of samples onto one array, and primer extension decoding and mapping of the array probes. One disadvantage of this approach is that to multiplex N samples, each target of interest has to be represented by N different probes on the array.

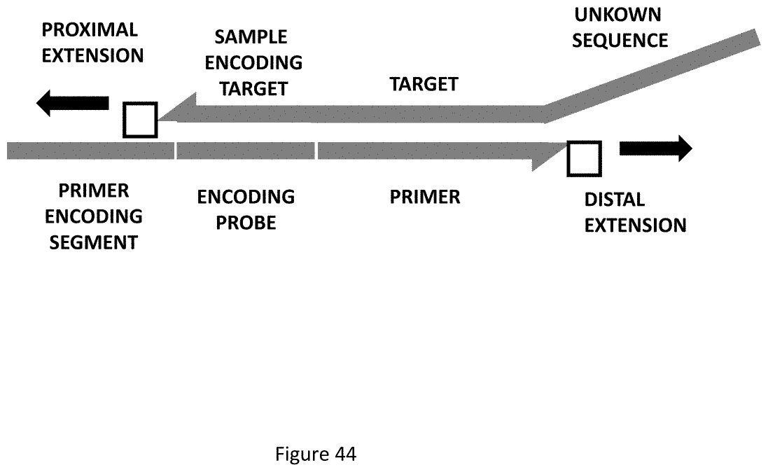

FIG. 44 illustrates embodiments where two primer extensions are used. In both embodiments shown, a universal primer is applied to an array, to read the primer identities, for the decoding and mapping of primers on the array. In the upper embodiment, the samples are prepared with a sample identifier segment attach to their 5′ ends, and multiple such samples are pooled and multiplexed on the sensor array, and then the second primer extension is used to decode these sample identifiers. This embodiment provides for efficient encoding and decoding of both the primers on the array, and the samples that are multiplexed together. In the lower embodiment shown, the specific primer extension is used to acquire sequence information about the unknown sequence segment. In one embodiment this could simply be a SNP genotyping at the location of the 3′ end of the primer, using the match/mismatch there to drive primer extension or non-extension. In other embodiments, in the case where sequence information can be recovered by processing the signal traces produced during incorporation, this can provide for sequencing of the unknown segment of the target. This provides for a method to use the primer sensor array for targeted sequencing of a sample, at multiple targets of interest.

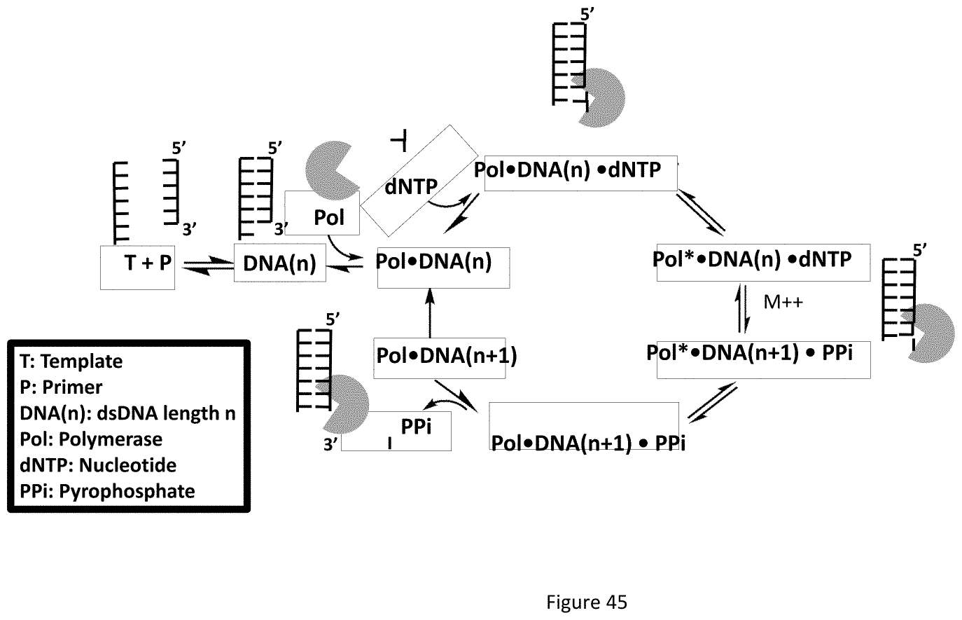

FIG. 45 illustrates on preferred embodiment, combining elements of disclosure above, to allow for primer extension decoding and mapping of primers on an array, multiplex encoding and decoding of samples, and targeted sequencing of the unknown sequence segment. One or two selective blocking groups are used to control which extension is performed in a given primer extension reaction. In one exemplary embodiment, there is one such group, the other 3′ end is free, and the first extension extends that end, and then the selective group is removed, and the other 3′ end is extended in the next extension reaction. The samples are prepared with a targets reaction that adds a sample encoding target on the 3′ end. The primer extension reaction in some embodiments in which the sensor is capable of extracting sequence information from the extension signals, provides for targeted sequencing on an array of sensors, with efficient decoding and mapping of probes, and multiplexing of sample.

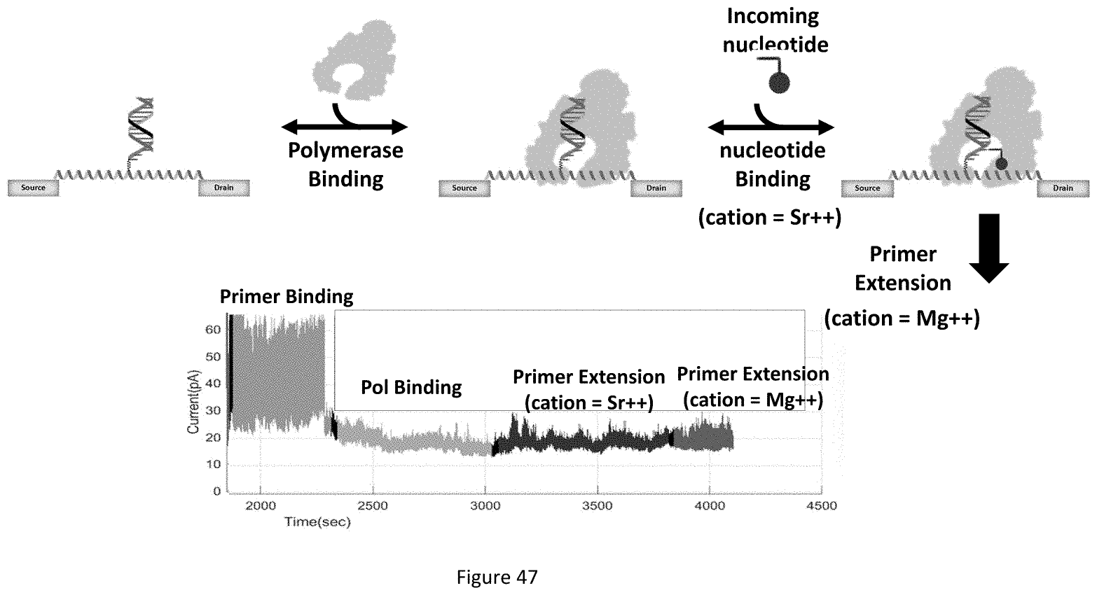

FIG. 47 illustrates an exemplary embodiment where instead the template strand is put onto the sensor on the array, and the primer for primers extension are provided in solution. This then allows for the remainder of the primer extension assay to occur, for allele specific detection, or for sequencing of the template in cases where the sensor recovers sequence information form the extension signals. In one exemplary embodiment, a set of target amplicons is produced, and bound to the array, and the allele-specific extension can be used to type these alleles. In another embodiment, such sample segments may be prepared with a universal priming site, and the fragments can be sequenced, if the signals of extension allow the recovery of sequence information. In this latter case, there is no need to otherwise decode the identity of the segment or primer—the sequence information also identifies the fragment in question.

In some embodiments, the application could be gene expression analysis of any cellular samples, and in general could be any application where methods such as DNA microarrays have been used for gene expression. In some embodiments, this would include gene expression applied to tumor tissue as may be used in cancer diagnostics.

In some embodiments, the application could be SNP genotyping in human, animal, or other cellular samples, and in general any application where methods such as DNA microarrays have been used for genotyping. In some embodiments, this would be applied to SNP genotyping in humans.

In some embodiments, the application is massively multiplexed primer extension detection and/or concentration measurement of targets in a complex pool. The level of multiplexing could be up to 100 different probes, 1000 probes, 10,000 probes, 100,000 probes, 1 million probes, 10 million probes, 100 million probes, or 1 billion probes. The provides an alternative to DNA microarrays for such high levels of multiplex detection, with the advantages of all-electronic, chip-based system, single molecule sensitivity, speed, low cost consumables and instrument, and compact mobile, portable, or point-of-use instruments.

In some embodiments, the application could be species identification, in particular determining what species a given tissue sample is taken from, or in the identification of which pathogens, such as a parasite, fungus, bacteria or viruses are present in a given sample. In some embodiments, this could be testing of environmental samples for the presence of a given virus, such as COVID-19. In an exemplary embodiment, the sample could be a tissue, or fluid, from any of the common vectors for viral transmission, such as bats, birds, rodents or mosquitoes. In preferred embodiment the sample could be material filtered from air or water, or material swabbed from a surface. In some embodiments the sample could be a biosample taken from a human or animal subject, such as saliva, mucous, buccal swab, blood, sweat, urine, stool, or exhaled air.

In some embodiments, the application could be strain identification, in particular determining what strain of a pathogen, such as a parasite, fungus, bacteria or virus, are present in a given sample. In some embodiments, this could be testing of environmental samples for the presence of a given strain of a virus, such as COVID-19. The samples for this, could be the same as for the previous species identification application.

Sensor Array Chips—In some embodiments, these molecular electronics hybridization sensors are deployed on integrated circuit semiconductor chip devices, where such chips include the circuitry to supply voltages to the sensors, measure currents in the sensors, and transfer such data off-chip, and to control such operations. In some embodiments, and as illustrated in FIG. 6, the architecture of such a chip is as an array of pixel, with such pixels providing the circuits needed to apply voltages and measure the currents. In the preferred embodiment shown in FIG. 6, the chip has a rectangular array of pixels circuits, each such pixel comprising a molecular electronics sensor which transduces molecular interaction events into the current signal, indicated schematically by the cartoon inset of the hybridization sensor, as well as having other major architectural blocks as labeled, that apply the needed voltages (Bias), manage the transfer of measurements from the array, and their conversion to digital form, (Row Decoder and ADC, or Analog-to-Digital Convertor), and where in particular the ADC converts each analog measured value to a binary digital value, with a bit resolution that in some embodiments may be 8 bits, 10 bits, 12 bits, or 16 bits, or bit a depth selected in the range of 1-32 bits. In addition, other blocks indicated is a local on chip memory buffer (Memory), and the control circuitry (Timing and Control), which in some embodiments comprises circuits to produce, synchronize and distribute clock signals, including PLL circuits. One exemplary embodiment of the pixel schematic circuit design is also indicated in FIG. 6, which here is a TIA circuit or Trans-Impedance-Amplifier. This circuit schematic shown allows indicates the application of Gate, Source and Drain Voltages, and the measurement of the resulting current by collection of charge onto a capacitor, which may be reset to an uncharged state or nominal state by closing the indicated “Reset” switch. Closing the “En” switch shown then outputs the measure data, i.e. The voltage across the capacitor, from the capacitor to the column bus and encoder.

These circuit blocks indicated in the schematic, as well as the blocks of the pixel, or the pixel circuit itself, can be fulfilled by many possible detailed circuit designs and IC layouts, well-known to those skilled in the art of VLSI Integrated Circuit Design, Digital Circuit Design, Mixed-Signal Circuit Design, and Analog Circuit Design. The architectures and schematics shown in FIG. 6 are not meant to be limiting on the use of such circuit designs or layouts that provide similar functionality.

In some embodiments, the molecular electronic sensor is deployed onto a CMOS chip device, which is a specific form of semiconductor chip and chip manufacturing process. The advantage of using CMOS chips is the very large manufacturing base for such chips, and related supply chains, as well as the aggressive scaling roadmap for such devices. The majority of chips presently made are of the CMOS type, including the common processors, memory, and digital imaging chips used in commercial products. Another advantage is that aggressive scaling has led to shrink the features on such chips down to the near the 1 nm scale, so that such processes are in principle capable of producing nano-electrodes needed for the present disclosed sensor, thereby enhancing the manufacturability of the devices disclosed herein.

In some embodiments, such a chip operates synchronously, by each pixel acquiring a single current measurement value, and then the array of such values are transferred off chip as a “frame” of digital data, in a row-by-row fashion as indicated in FIG. 6, at some total frame rate, which in some embodiments could be up to 10 frames per second, or up to 100 frames per second, or up to 1000 frames per second, or up to 10,000 or 100,000 frames per second. In some embodiments, the pixel array may contain up to 100 pixels, 1000 pixels, 10,000 pixels, 100,000 pixels, 1,000,000 pixels, 100,000,000 pixels, or up to 1 billion such pixels. The physical size of such a chip is related to the number of pixels, and in some embodiments may be as small as 1 square millimeter, and as large as a full reticle size used in for the photolithography, which may be up to approximately 30×30 millimeters squared.

In such sensor array chips, in some embodiments, as illustrated in FIG. 7, there are more than one pair of nanoelectrodes per measurement pixel, and therefore more than one molecular electronic sensor per pixel, so that one measurement pixel circuit may be used to monitor signals from multiple sensor devices (nanoelectrode pairs with bridge and hybridization probe molecule). In some embodiments of this, the measurement circuitry for applying voltages, measuring current, and reading out data in each pixel is applied serially in time to each sensor within the pixel, under control of transistor-based switches in the sensor circuit that can select any of the sensors for measurement. In FIG. 7, the addition of these selection switches is indicated by the switches labeled “Select” in the pixel schematic shown. By closing a single one of the available switches, the measurement circuit in the pixel is coupled in to just one sensor, and can be used to acquire data for that sensor. Such data can be transferred of the pixel in a frame transfer process, and then the pixel can be cycled on to the next bridge. In some embodiments this is done synchronously, so that all pixels acquire data with their local switch 1 closed, transfer a frame off the array, and hen all pixels move on to closing switch 2, etc. Cycling through all such switches in this way, frame by frame, if each pixel serves M sensors, a chip with N pixel circuits embodied on chip can acquire measurements from a larger (and potentially much larger) number, NM, of sensors. This in general has the potential to lower chip costs: the price of chips is based on area, and the area required for the chip is dominated by the area needed for pixel circuits, and this has the benefit of greatly lower the total pixel count and pixel circuit area required to serve the number of sensors of interest, versus having one pixel per sensor. In some embodiments, each pixel may have from 2-1024 sensors per pixel, and preferable in the range of 4-128 sensors per pixel. However, this form of multiplexing of sensors per pixel has the cost that measuring from all pixels takes longer, by the multiplier M, as it requires M times more frames, and also the disadvantage each sensor is offline and not measuring while its switch is open, so that the respective current goes un-observed for part of the time, creating the potential to miss signal features.

In some embodiments, the chip pixel array architecture is such that nearby pixels in the array share a common staging area, where the many nanoelectrode pairs for these neighbors are all located, and suitably electrically routed back to these adjacent pixel circuits. This applies to both cases where each pixel has one sensor affiliated with it, or cases of M multiple sensors per pixel. The use of such staging areas further improves the efficiency of circuit layout, and allows the staging area to have a larger opening, and better accessibility for nanofabrication or molecular assembly purposes or to facilitate wetting by the solution.

For arrays of sensor on chip, in one exemplary embodiment multiple probes with the same target are represented on all or part of the array. In some embodiments, these multiple measures of the same target can be aggregated or averaged together to produce a more robust detection of presence/absence of the target, or to provide more sensitive detection or lower detection limits, or to provide a measure of concentration. For example, in some embodiments, if N sensors have the same hybridization probe and target, and a fraction f of these register a detection event, within a measurement time T of exposure to the sample, then detection becomes more robust or sensitive or accurate if a minimum threshold, fmin, is required for detection, f>fmin. Or, in other some embodiments, the ratio of f/T, which is the rate of detection, provides a measure of the concentration of the target in the sample. In other some embodiments, more detailed analysis of the f(t) curve acquired during the time interval [0,T] could provide various robust fits to the slope of this curve, or this curve could be fit to characteristic profiles or measured calibration curves produced by known reference concentrations, to provide a measure of concentration from these multiple probe measurements on the chip array. Such aggregation measures also typically provide a related estimate of confidence or measurement uncertainty, such as a suitable mean and standard deviation. If such individual probes are otherwise directly providing concentration measures, for each probe, these can also be averaged together, by various well-known means of averaging measurements, to produce a more accurate estimate of concentration, as well as error bars or confidence intervals on the measurement, based on the spreads or standard deviations observing in the set of individual measurements. This provides benefits of greater accuracy and measurement confidence for the concentration estimate for the target of interest.

For arrays of sensor on chip, in another preferred embodiment different hybridization probes with different targets are represented on some or all of the array. In some embodiments, these multiple measures of different targets provide the ability for multiplex or in-parallel measurements of the set of targets of interest. This has the advantage of lower cost of testing the targets, and faster testing of the targets, or simpler testing of the targets, or the use of less sample material or less reagents, to test for all the targets, versus separately testing for such targets on separate devices. This is generally referred to as multiplex testing or parallel testing, and is widely appreciated as a potential benefit to testing systems.

In some embodiments of sensor array devices, both forms of multiple probes will be present, i.e. for the give set of hybridization probes with the respective targets of interest, each specific type of hybridization probe will be represented by multiple, replicated sensors on the array, proving the benefits of redundant, replicate measurement above, and the multiplex probes for the multiple targets will be represented on the array, to also proved the benefits of multiplexing. The resulting compound benefit is multiplex testing, with confident measurements for each target that have the benefits of statistical replication for accuracy and confidence interval estimation. For this purpose, it a benefit in some embodiments to allow for very large chip-based large arrays of probes, in some embodiments up to 100 probes, up to 1000 probes, up to up to 10,000 probes, up to 100,000 probes, up to 1 million probes, up to 10 million probes, up to 100 million probes, or up to 1 billion probes, or up to 10 billion probes.

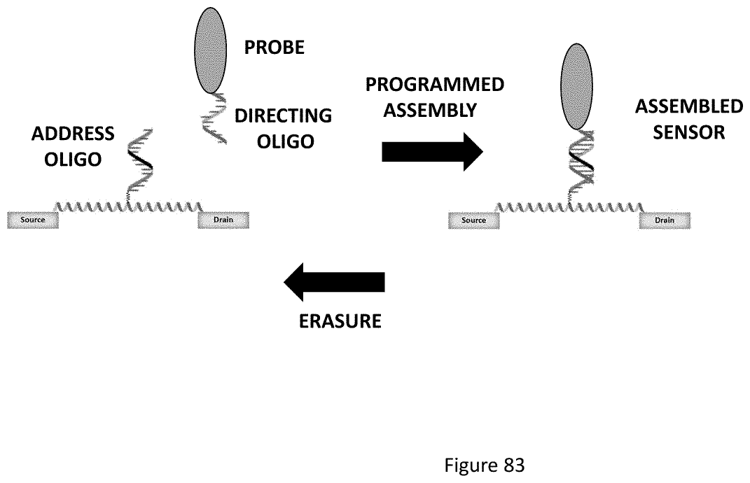

In some embodiments, the desired probe molecules on the sensor array can be deposited in a secondary reaction, in which probes of interest are directed to conjugate to existing bridge constructs. This provides for a convenient way to alter, or “program”, the targets of detection of a primary sensor chip device. FIG. 83 illustrates a programmable sensor probe for use in a personal virometer or pathometer device. In an exemplary embodiment, a DNA “address oligo” is attached to the bridge molecule in the circuit via a suitable conjugation. The probe of interest to be directed to the sensor site is conjugated to a complimentary oligo to the address oligo, referred to here as the directing oligo. This is placed into a hybridization reaction, which results in the desired probe being bound to the bridge via the hybridization, thus establishing the desired probe-bridge complex. The process constitutes programming the sensor with the desired probe content, using that address oligo. In the case where there is a multiplicity of such addressed sites, with distinct oligo sequence addresses, a multiplicity of probe molecules, conjugated to respective directing oligos, can be simultaneously hybridized to the sensor array, thereby simultaneously programming the array with the probe content of interest, with said probes going to the affiliated addressed sites. Assuming there has already been established a map of the pixel locations of the address oligos, this provides the map for the respective probe content programmed onto the array, so that the pixel response signals can be affiliated with the respective probes. In some embodiments, the content programmed in this method can be erased, by dissociating the hybridized strands. In various embodiments, this dissociation could be achieved by elevating the temperature to induce melting, or by adding chemical denaturing agents that disrupt the hybridized duplex, such as formamide or DMSO or betaine. In some embodiments, the directing oligo on the probe make be degradable, and the degrading agent may be added or applied. This could include the use of photocleavable bases (degraded by applied light), or the use of an RNA oligo for the address complement (degraded by an RNAse such as RNAse H), or the use of a nicking enzyme that makes a single stranded nick in the directing oligo. In some embodiments, the length of the address oligo may be reduced by using nucleic acid compositions (such as high GC content) analogs such as PNAs or LNAs, or other analogues known to raise the melting point of hybrid duplexes, for either of the address oligo or the directing oligo, or both. In some embodiments, using such means, the length of the address oligo could be less than 30 bases, less than 20 bases, less than 15 bases, less than 10 bases, or as few as 4, 5, 6, 7 or 8 bases. A shorter address oligo may in some embodiments allow for bringing the probe closer to the bridge, to enhance resulting signals. The distance from bridge to probe in these embodiments may be less than 10 nm, less than 5 nm, less than 3 nm, or less than 2 nm.

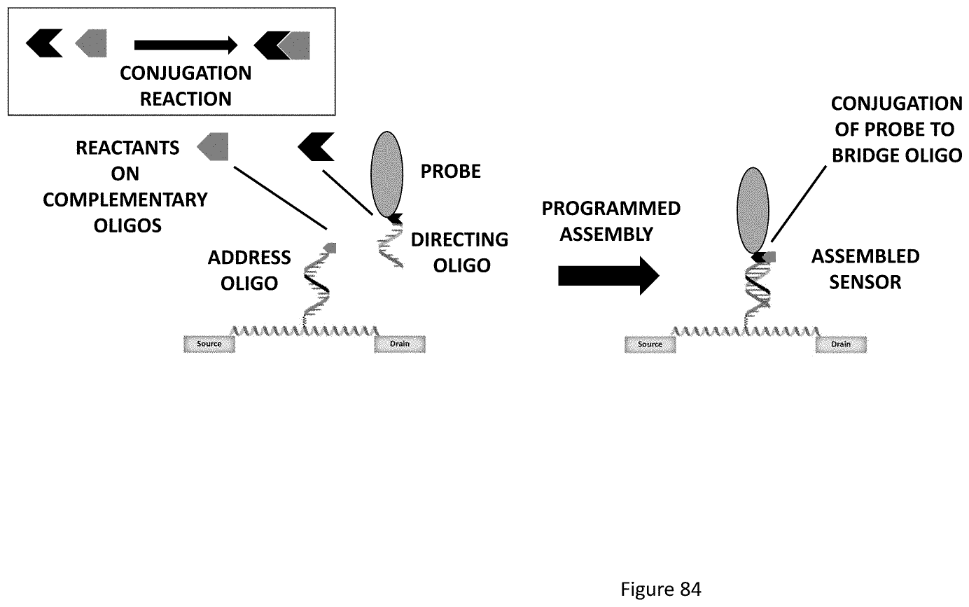

As illustrated in FIG. 84, in other some embodiments, the programming can be stabilized or made irreversible by the addition of a pair of conjugating groups to the address oligo and directing oligo, such that when these oligos hybridized, the conjugating groups are located so as to favor their conjugation, establishing the more durable or irreversible binding. The figure shows these groups as being located for binding at the terminal of the address oligo, but these conjugating groups in general may be positioned anywhere along the address oligo, and corresponding site on the directing oligo.

In some embodiments, these conjugation groups may be any of many bioconjugation groups well known to those skilled in conjugation chemistry, and most preferably utilizing small molecules, such as click chemistry conjugation, such as DBCO-azide or TCO-azide of copper click chemistry, or also such as NHS ester-amine conjugation.

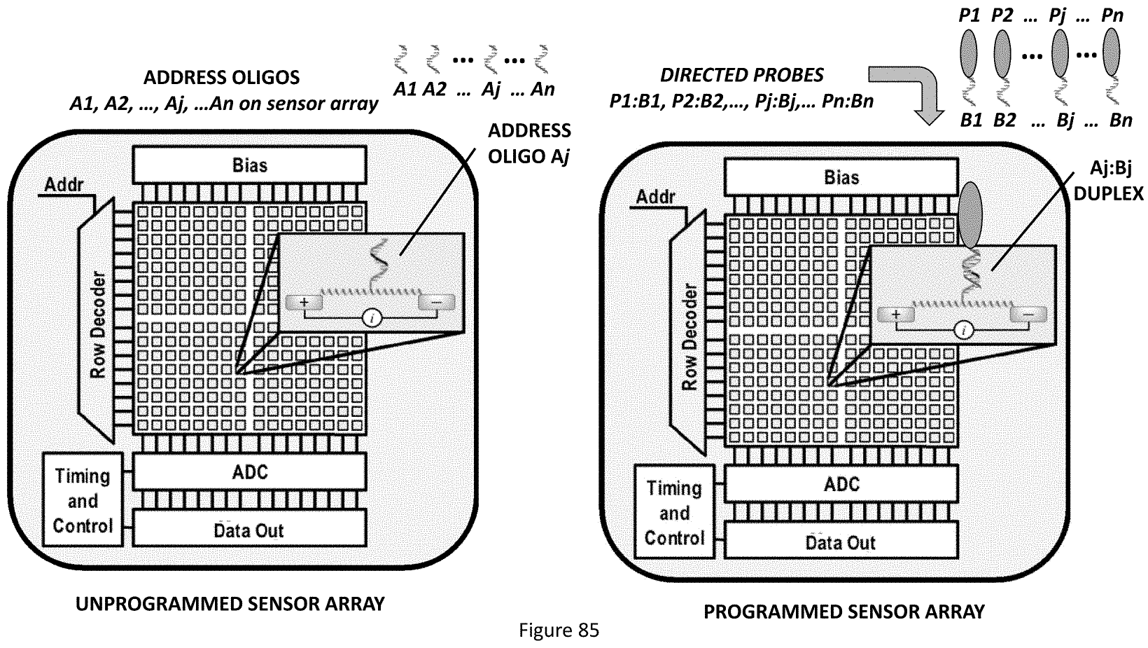

The programming of an entire sensor array chip by these means is shown in FIG. 85. Using an set of n address oligos, A1, . . . , An, and corresponding directing oligos B1, . . . , Bn, a set of n probes P1, . . . , Pn is programmed onto the array, with Pi conjugated to Bi, and directed to all sites on the array that hold the address oligo Ai. In some embodiments, each Ai address will occur at a multiplicity of pixels on the array. The pixel map stablished for the Ai, using the means decoding described herein, thereby provides the pixel map for the Pi probes, respectively.

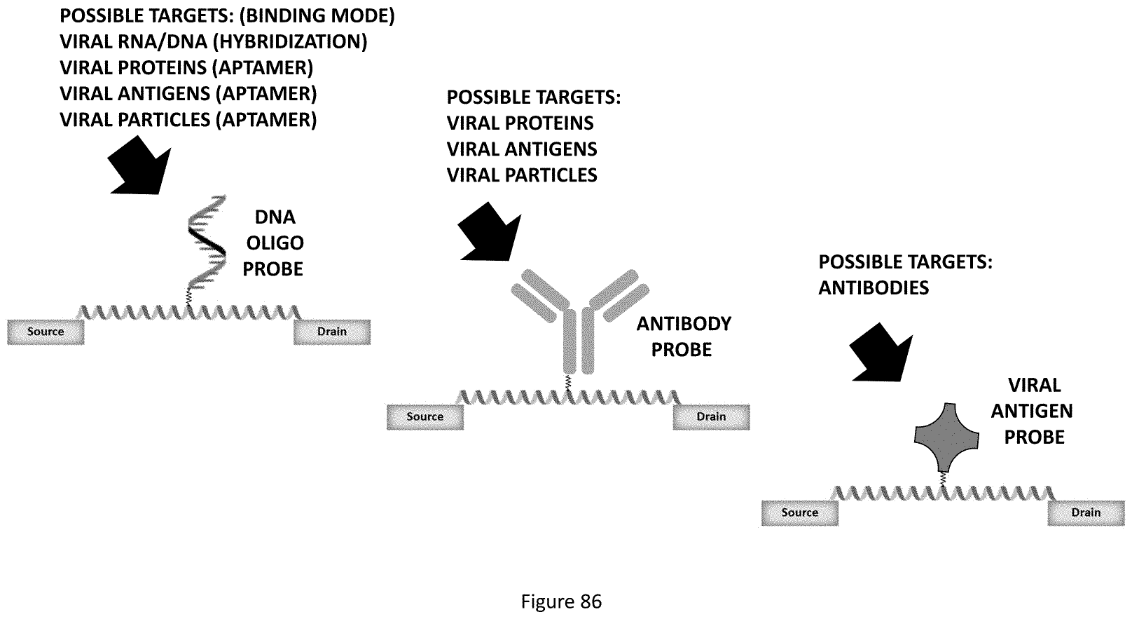

FIG. 86 illustrates classes of probe molecules that may be used in personal virometer and pathometer sensors. As shown at left, the case where the probe is a DNA oligo, can be used for RNA or DNA targets, or DNA oligo aptamers can be used for targets that are proteins, antigens, or viral particles. As shown in the middle, the case where the probe is an antibody can be used to target antigens, proteins, or viral particles. As shown at right, the case where the probe is a viral antigen (such as a viral protein or portion of a viral coat), can be used to target antibodies for such antigens.

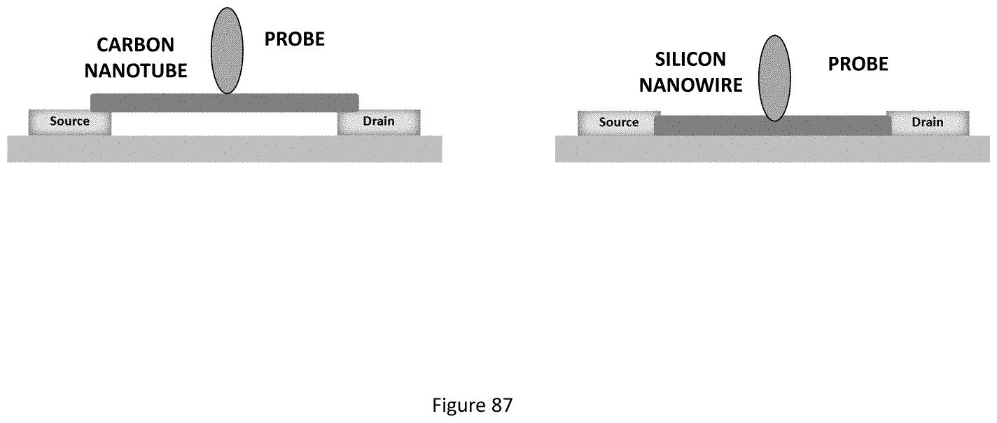

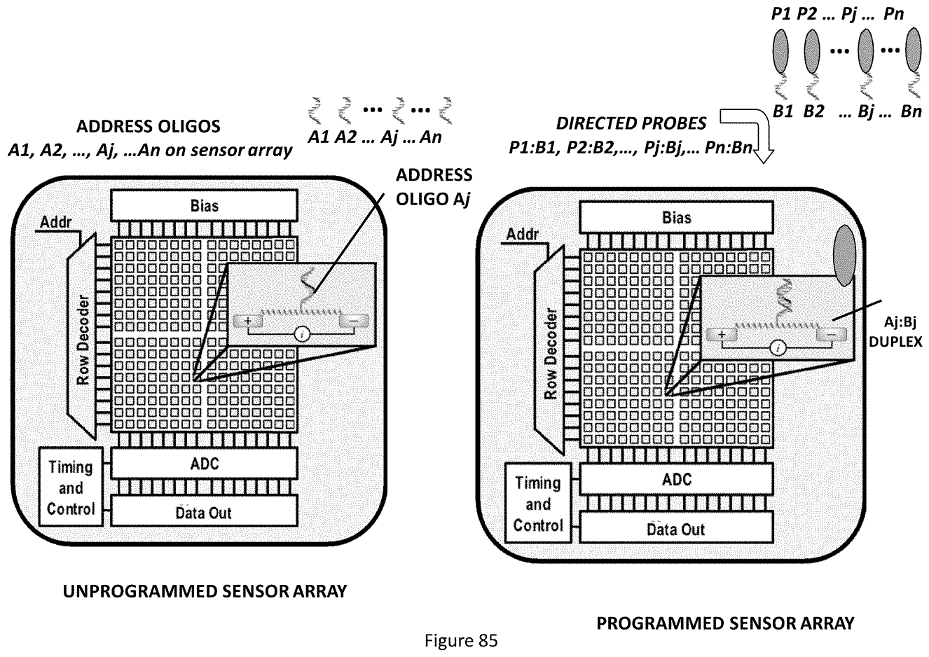

FIG. 87 illustrates that in some embodiments, the bridge between electrodes may be a carbon nanotube, or a silicon nanowire, with probes bound to these by suitable conjugation reactions.

Multiplex Probe Maps and Decoding Methods In such some embodiments of sensor arrays in which multiple distinct hybridization probe sensors are deployed on one chip for the purpose of multiplex testing, there is a map provided that specifies what probe type is present at each different pixel location or sensor location (if multiple sensors are affiliate with each pixel). This allows the measured sensor data readout from the pixel array to be related to which probe target was being assessed at each sensor. Such a map may be produced by various techniques, based on how the probe molecules are prepared and applied to assemble into the array. This map is referred to as the probe map for the sensor array or pixel array.

In one some embodiments for establishing this map, spatially controlled exposure of the pixels to the different solutions containing the different probe types for assembly (or, instead of just the probe molecule, in some embodiments it is the probe-bridge molecular complex pre-formed which is assembled into the sensors) during sensor assembly, so that the probe map is known from which solutions were applied to which pixels. In some embodiments, such spatial control can be achieved by mechanically applying solution only to certain regions of the chip, instead of applying solution to the entire chip pixel array. In other some embodiments, this can be achieved by applying a probe assembly solution to the entire chip array, but using a voltage driven assembly processes such that only electrically activated pixels will assembly probe molecules into the sensor nanoelectrodes. In some embodiments, this could be done by applying a voltage to electrodes that either attracts or repels the probes or probe-bridge complexes, such as using a positive voltage to attract the negative chard on DNA in solution, or a negative voltage to repel such DNA. This relies on the well-known process of electrophoresis, in which an applied voltage on an electrode causes charged molecules in solution to be attracted to an electrode that has the opposite charge, or repelled from one of same charge. In other some embodiments, an applied AC voltage may be used to selectively attract or repel the probe or probe-bridge molecules, using the well-known process of dielectrophoretic forcing. In particular, in one exemplary embodiment, the particular probe type or probe-bridge type for a particular target is applied to the solution, for a short period of time, and in a low concentration, such that diffusive transport is unlikely to deliver these molecules to bind to nanoelectrodes on the chip array. However, for the desired nanoelectrode locations of the molecules, an AC voltage of proper frequency and amplitude to create a dielectrophoretic force that will drive these molecules to concentrate near the electrode gaps of the intended sites, allowing them to selectively bind the intended probes or probe-bridge complexes. The solution is then flushed away, and the next probe may be introduced, similarly target sites for it. This may be done for individual probes, or pools of distinct probe types, in which case their locations are restricted to a much smaller set of possible sites, but probe type from the pool is still randomly dotributed across electrodes within those site sets, and further location information would be required to complete the map to the individual sensor level. In some embodiments, the low concentrations of the probes used may be in the range of 1 pM (pico-Molar) to 100 nM (nano-molar), the exposure time used may be in the range of 0.1 s to 100 s, and amplitude of voltage used maybe be in the range of 0.1V to 10V, and the frequency of AC modulation used may be in the range of 1 kHz to 100 MHz.

In other some embodiments, the probe map may be constructed by a process of decoding hybridization probe locations using the result of special binding reactions, with special detectable probes (not necessarily DNA hybridization probes) that are designed to be able to locate or localize each specific hybridization probes type on the array. In this approach, each distinct hybridization probe type is provided with one or more binding sites, directly coupled to or integrated into the probe molecule (or, in some embodiments, to the probe-bridge molecular complex, pre-formed), and where such sites are capable of producing an observable binding signature in response to binding with the corresponding decoding probe molecules, and with such a signature being localizable to the resolution of a specific probe site (nanoelectrode pair) or pixel, as required for the complete hybridization probe map. One or a series of these decoding signatures can thereby be affiliated to a specific probe site on the array, and these may be used to decode which particular type of hybridization probe is present at the site. In this method, the hybridization probes (or probe-bridge complexes) are applied as a pool to the pixel array, allowing them to randomly assemble into the nanoelectrodes on the pixels, and after they are so assembled, in a series of subsequent reactions, known decoding probe molecules are applied to the array and observing for the production of their unique binding signatures, localized to sensor sites. For each site, there is at the end of this procedure a resulting series signals localized to that sight, that are sufficient in combination to determine confidently which hybridization probe must be present at the site.

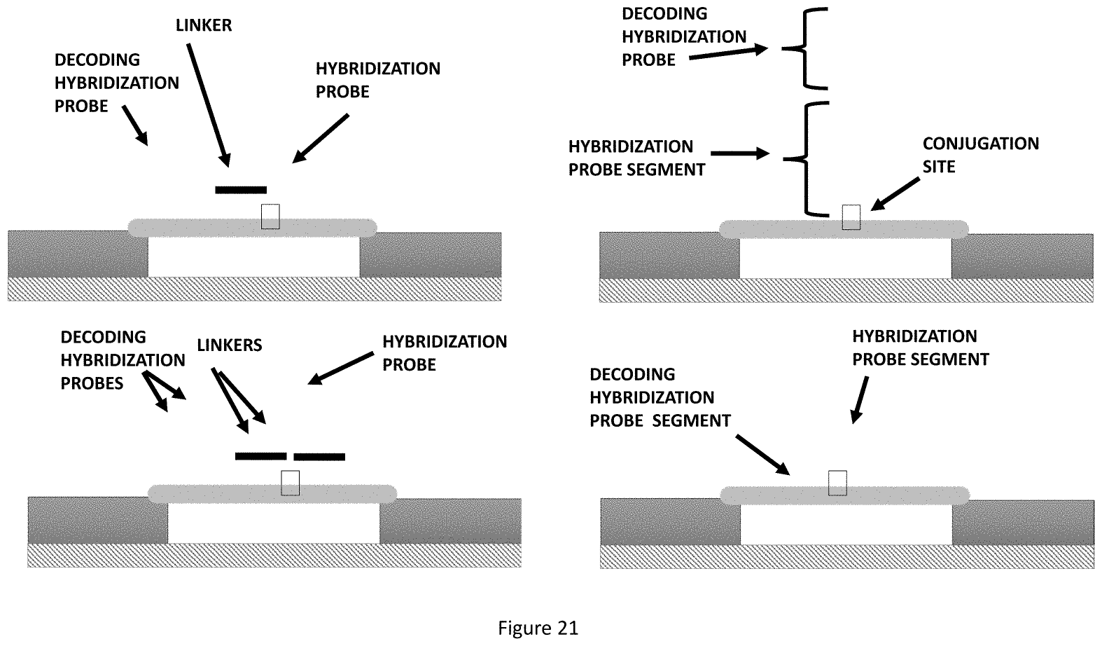

Such observation of a decoding signature in some embodiments may be done by monitoring electrical signals from the pixels and respective sensor sites, that are produced by the binding of the decoding probe. In some embodiments, these decoding probes are themselves DNA hybridization probes, whose targets are affiliated with the hybridization probes, and their hybridization events also can produce detectable signals in the sensor. The decoding scheme may require one or multiple such decoding hybridization targets affiliated with the hybridization probe. There are many ways such targets can be affiliated with a hybridization probe, FIG. 21 shows four representative examples, and many other schemes can be readily extrapolated from these specific examples. For example, the upper left embodiment in FIG. 21 shows that the DNA target of a decoding probe is linked to the primary hybridization probe DNA by a linker molecule, and the lower left figure illustrates that there could be two or more such linked targets. Generalizing this, in that way, any number of decoding targets can be affiliated with the probe to be encoded. For another example, the embodiment in the upper right of FIG. 21 shows that a segment of the DNA strand that contains the hybridization probe, could also include another contiguous segment that is a target of the decoding probe and, by generalization, and number of such targets could be present in series, all encompassed in a single DNA strand. The figure in the lower right illustrates that the conjugation of such a strand to the bride could be at an interior part of the strand, in order to allow both the hybridization probe segment, and the decoding target segments, to be similarly close to the bridge, for more sensitive detection of the decoding targets.

One exemplary embodiment of a decoding method to produce the probe map is “direct decoding”, in which individual decoding probes specific to the individual hybridization probes are used to directly locate the sites of each probe type on the array. In an exemplary embodiment, the decoding probes are hybridization probes. Assuming there are N hybridization probes types H1, H2, . . . Hi, . . . HN, in this method there are provided N decoding hybridization probes D1, D2, . . . , Di, . . . , DN. These should have distinct sequence targets, that have very low cross-hybridization between them. A target oligo for Di is physically affiliated to probe Pi, for I=1 . . . N, such as by any of the means represented in FIG. 21. These probes are assembled randomly onto the sites on the chip array. Then, a series of N hybridization reactions is performed on the chip. In reaction i, decoding probe Di is hybridized to the array, and the detection readout of each sensor is recorded Sensors where Di finds it target produce a detection signal, and this directly identifies which sensors have probe Pi. Do this for reactions I=1 . . . N decodes the locations of all probes, and thus provides the probe map. In some embodiments, this same method works if instead of electrical readout, optical labeling and imaging location is used to localize the decoding probes. In one exemplary embodiment of this, the decoding probes could simply be taken to be the targets of the hybridization probes in question, i.e. Di is the target of HI. The has the advantage that not extra target sequences need to be added to the hybridization probes to achieve this decoding. However, in other some embodiments, the decoding probes Di may be chosen to have better physical hybridization properties, than that simple choice affords, such as stronger on-target binding and weaker cross-hybridization with other decoding probe targets, or more uniform such performance across the set.

Another family of some embodiments for decoding methods to produce the probe map is generally termed “combinatorial encoding and decoding”. In these embodiments, a series of decoding probe reactions are applied, and for each given probe site on the array, the series of detection/non-detection results from these reactions provides enough information in aggregate to uniquely determine the identity of the probe at the site. Several canonical exemplar embodiments of such combinatorial methods are given here. It is understood that there are many variations, reformulations, and combinations of these provided canonical exemplars that can be used as alternative decoding schemes for building the probe map, and which would also be obvious, from the canonical examples, to one skilled in the theory of codes. All such obvious variations, reformulations, and combinations are meant to be encompassed by these canonical exemplar embodiments.

The canonical combinatorial decoding embodiments provided may be described succinctly and efficiently as follows, wherein to achieve this, the assay to be performed, their order, and their outcomes are arranged and represented with 0/1 in way that allows the direct relation of decoding probe assay results to probe identification codes. Assume there are N hybridization probes types H1, H2, . . . Hi, . . . HN for which a location map is desired. In various some embodiments of this method, there is provided a set of N distinct K-bit binary code strings {B1, B2, . . . Bi, . . . , BN}, where these Bi are various strings of length K, composed of the symbols “0” and “1”, such as such as for example might be the string B=“1001011”, in a case where the length is K=7. The code Bi is assigned to probe Hi, for i=1, . . . , N, and these codes will be used in physical encoding and decoding process to identify this probe for the probe map on the array. Note that any such set of N such strings will provide a valid encoding for the methods that follow, although special sets of such strings, as described in some embodiments below, can also provide for the additional feature of error detection and correction in the decoding measurement process used in array assays. Also, note that as there are exactly 2\hat{ }K distinct strings of length K, so it is required, in order to have enough such binary codes, that that 2\hat{ }K≥N. Indeed, for any K satisfying this, some embodiments include the choice of any subset set of N strings from the master set of 2\hat{ }K possibilities, and if N=2\hat{ }K, an exemplary embodiment is simply to use all K-bit strings, listed in any order. Note that in these code assignments, if all the code strings {B1, B2, . . . Bi, . . . BN} have the same binary digit in position j (i.e. the jth digit is always 0, or always 1), this position is uninformative and can be eliminated from the strings, reducing their length K to K−1. This can be repeated to remove all such uninformative positions in the strings, so as to reduce the number of physical encoding probes required in the methods below.

In one family some embodiments, there is further provided a set of 2K decoding probes that are hybridization probes denoted as Dij, where i=0 or 1, and j=1 . . . K. These decoding probes should have distinct target sequences, and preferably low potential for cross-hybridization. For the probe Hi, the associated physical encoding targets are taken to be the target DNA oligos of the encoding probes D(b1)1, D(b2)2, . . . , D(bK)K, where here b1, b2, . . . , bK are the binary digits of the encoding string Bi, i.e. bj is the jth digit of string Bi. These encoding probe target DNA oligos are then to be physically linked or affiliated with the physical hybridization probe oligo, such as by the methods illustrated in FIG. 21, or other means. Note that with the probes so encoded, that for any probe, Hi, and a pair of encoding probes D0j and D1j, precisely one of these two probes will have its target on the physically encoded probe for Hi. To achieve decoding of probe locations on the arrays, the series of 2K reactions trying to hybridize the individual encoding probes D01, D11, D02, D12, . . . D0j, D1j, . . . , D0K, D1K is performed, and for any probe site of the array, the outcome of these reactions is recorded by taking the trial of both D0j and D1j, and recording the outcome of these two reactions for the site as trialj=0 if D0j bound or trialj=1 if D1j bound, at the site in question. Then the complete binary string of trial outcomes for the site in question is succinctly written as (trial1)(trial2) . . . (trialk). As constructed in this process, this string will be identical to the code Bi assigned to the hybridization probe Hi that is in fact located at the site in question. In this way, for each site, the reaction results from the decoding probes are decoded to some precise Bi code and related Hi probe from the probe set. Therefore, the outcomes of this series of 2K decoding hybridizations across all sites on the array provides the code strings that identify and localizes in the array all occurrences of every probe H1 . . . HN. Thus, the probe map is constructed.

In another family of some embodiments, the situation is as in the above embodiments, but the physical encoding is done in a more compact form: For the probe Hi, the associated physical encoding targets are taken to be the target DNA oligos of the encoding probes D(b1)1, D(b2)2, . . . , D(bK)K, but only tag the probe physically with the D1x targets, ie. Do not tag them with any of the D0x targets, and when doing the decoding above, apply only the K reactions of the probe D1x probes, D11, D12, . . . D1j, . . . , D1K. The results of these trial assays can be recorded as testj=1 if D1j binds at a probe site, and testj=0 if it does not bind. In this case, the result string (test1)(test2) . . . (testj) . . . (testK) is the same binary string as recovered above in the previous embodiment, because above, if D1j did bind, testj=1, as in the present method, and if D1j did not bind, this is the same as D0j binding, which also recorded as 0 above and in the present method. Thus, the same probe map decodind is achieved. It is a benefit of this embodiment that fewer physical target oligos need to be linked to each hybridization probes, and overall the method requires only half as many physical encoding probes to be produced, and their associated targets to be produced and linked to probes.

Another family of some embodiments of methods for making probe location maps may be described efficiently and succinctly as follows. Again, reaction procedures and outcomes are efficiently encoded by 0/1 indicators that allows direct interpretation of decoding assay results for an unknown probe as the binary code identifying the probe. for This method relies on reacting pools of decoding probes, rather than individual probe reactions, within otherwise a similar logical framework. Assume there are hybridization probe types H1, . . . , HN, with assigned K-bit binary codes {B1, . . . , BN}. There are then further provided the same number of N decoding probes that are hybridization probes denoted as D1, . . . , DN. These decoding probes should have distinct target sequences, and preferably low potential for cross-hybridization. The target of each Di is to be physically linked to the corresponding probe Hi, such as by the means illustrated in FIG. 21. A total of 2K Pools of decoding probes P01, P11, P02, P12, . . . P0i, P1i, . . . , P0K, P1K, are defined as follows: The members of pool P0j are all probes Di for which bit j of code Bi is 1, and similarly, the members of pool P1j are all probes Di for which bit j of code Bi is 0. Given these pool memberships, the corresponding physical probe pooled are produce, as equimolar mixtures of the decoding probe oligoes for the pool. Under this construction, note the result of reacting the physical pool of probes P1j against a probe Hi on the array will be a match if code Bi has 1 in position j, and this outcome is to be recorded as trailj=1, while otherwise, if there is a 0 in position j off code Bi, the match will instead occur for pool P0j, and this outcome is denoted by trialj=0. Let the outcome of all the K pooled reactions against the array be recorded by the string (trial1)(trial2) . . . (trialK). Then this string matches the code Bi of the probe in question Hi, and these series of reaction outcomes there provides the decoding of the probe identity. The results of reacting the 2K pools to the array, therefore, decodes all occurrences of all probes on the array, and provides the required probe map. Note in one exemplary embodiment above, the Di could be taken as the targets of the Hi, in which case no special linkage of targets to the Hi is required. However, in general, other sets of {Di} could have more desirable hybridization properties of uniformity of Tm and low cross-hybridization potential, and better discrimination of perfect match signals from background.

In another family of some embodiments, the situation is as in the above embodiments, but the physical encoding is done in a more compact form as follows. Only the K pools P11, P12, . . . , P1j, . . . P1K are physically constructed, and these are reacted to the array, in a series of K reactions, and for each site on the array, the result is recorded as trialj=1 if hybridization was observed with pool P1j, other 0 if it was not observed. The resulting string (trial1)(trial2) . . . (trialK) that encodes this outcome, is identical to the string in the above embodiments, and therefore this string provides the code string Bi that identifies the probe Hi. The results of reacting these K pools to the array, therefore, decodes all occurrences of all probes on the array, and provides the required probe map. This requires half as many pool constructions and hybridizations as the previous embodiments.

Some embodiments for Error Detection and Correction in Probe Mapping. As noted, in the above embodiments of decoding methods, any set of N binary K-bit strings {B1, . . . , BN} provides an encoding and decoding method, as so a great number of possible methodologies are outline above. Within this framework, the specification of specific code word sets for some embodiments can provide substantial benefits. For illustration of this point, note that in the combinatorial decoding schemes above, if the number of probes is N=2\hat{ }K, each and every K-bit binary string is then necessarily assigned a probe, in 1-to-1 fashion. However, in this minimal code length K scenario, if an error were made in measuring the code of a probe in the above methods, it would produce the code of a different probe, since all codes are used, and thus the result in incorrect decoding. Allowing a larger binary coding string length K than the minimum required allows for robustness against such errors. Specifically, it is possible that the set of binary codes {B1, B2, . . . , BK} is chosen as a set that allows for error correction or detection, such that if a code string from this set were corrupted by one or more bit flipping errors, it is possible to determine such corruption has occurred, and with some encodings, also to correct it back to the uncorrupted state, error free. This will provide for protection against errors that could be made in the decoding measurement process outlined above, in the form of an false detection of hybridization (error of 0→1), or missing a true hybridization (error of 1→0), so that such errors do not lead to incorrect or indeterminate decoding of probe identity. Many such error correction or error detection encodings are known to those skilled in the art of error correction methods for binary data. In some preference embodiments, one such method is the use of binary strings that add one or more parity bits add the end of an initial given string, which provide power to detect or correct certain errors. Another preferred embodiment is the use of Hamming Codes and Hamming distance to detect and correct errors. In this class of methods, the assigned number N of code words must be only a small fraction of all possible binary codes of length K, and the precise code words are taken to have highly distinct bit sequences, such as, for example, this could be N randomly selected code words from all 2\hat{ }K>>N. In such a case, if there is a corrupted code, it may be detected because it does not match any of the assigned codes, and it can be corrected back to the closest of the allowed assigned code strings, with closeness measured by the Hamming Distance (number of mismatches between the digits of two binary strings). This general technique always affords some power for error correction of at least limited number of bit errors, and for any proposed set of code words, {B1, . . . , BN}, the error correction properties of this can be directly and exactly assessed by brute for examination of all possible corrupted versions of each Bi, and noting for which corruptions this process corrects them. Some embodiments of such methods are provided by specific Hamming Codes, which are strings sets {B1, . . . BN} that have optimal or highly effective and uniform error correction by this means of correcting to the Hamming distance closest allowed code. In general, many other error correction encoding schemes are known to those skilled in the art of coding theory, and any of these schemes defined for K-bit strings can be used to provide K-mer code word sets that also have powerful error correction capabilities, and which can be used here to correct for possible decoding hybridization errors. In general, this provides a mechanism with arbitrarily good power to correct errors, at the cost of larger K—and therefore more physical decoding probes and more decoding reactions).

In some embodiments, the decoding probes used in the above decoding methods, electrical or optical, are shorter oligos, such as in the range of 8-25 bases, and any two such targets have multiple mismatches between them, to reduce cross-hybridization, preferable 2 or more, and preferably 4 or more. In some embodiments, they may be PNA probes, so that a short probe can have stronger binding and higher Tm, and the impact of single mismatches can be greater on reducing cross-hybridization. In some embodiments, all of the methods disclosed above can be used with electronic detection of decoding probe hybridization provided by the sensor chip array, or, in other some embodiments, using optically labeled decoding probes—such as a dye label or Quantum dot label, or gold nanoparticle label, or any other label detectable by microscopy and compatible with attachment to a single molecule DNA oligo—and localization of probe binding by microscopic imaging.

In another preferred embodiment of the above decoding methodologies, the objective and benefit is to have a decoding method in which the number of decoding targets added to each probe is a number J that can be specified as desired, so as to control the amount or of hybridization target added to the probes for decoding purposes. This can be achieved as follows, using the compact form of the first family of preferred methods above: the binary codes string {B1, . . . , BN} are defined as follows: for the set of numbers {1, . . . , L}, for some L, represent a subset S of this set by the K-bit string (b1)(b2) . . . (bK), where bi=1 in i is in the subset S, and 0 if not. This is the sometimes called the indicator function for the subset. E.g., the subset {2,4} would have indicator string 0101000 . . . 0. There are 2\hat{ }K such strings, corresponding to the membership indicator strings of all 2\hat{ }K subsets of S. In the setting, define as the codes the set of all strings that have exactly J 1's in them. The number of such strings is known in combinatorics as “L choose J”, and is N=L!/(J! (L−J)!), where “n!” denotes n factorial=n×n−1× . . . ×2×1. When this set of code strings is used in specified “compact” forms of the methods above, this has the advantage that for the physical encoding, wherein a target is added for every 1 occurring in the encoding string Bi, there are always exactly J such 1's, and so exactly J hybridization targets are added to encode each hybridization probe. This therefore has the advantage of controlling the amount of target material added for decoding, to be J oligo targets. For any desired number of hybridization probes N to be encoded, and any desired J>1, L can be chosen large enough to that L!/(J! (L−J)!) in >=N, and therefore provides enough such codes. The cost of achieving this as that L encoding probes are required. For example, suppose there were N=1024 hybridization probe types. One option would be to take all K=10-bit binary strings, and assigns all these as codes. However, in the above methods, each probe would get linked to either 10 targets (in the non-compact scheme), or a variable number of targets between 0 and 10 in the compact schemes. The decoding would require 20 reactions in the full scheme, or 10 in the compact scheme. However, restricted to linking to J=2 targets per probe, L=46 encoding probes and reactions are required, but allowing J=3 reduces this to L=20, and J=4 allows L=15. These are generally more desirable, such as required 15 probes and reactions, but only needing to add 4 decoding oligo targets to each probe. However, these do not provide any error correction capability, as a single bit error would produce a 3 element or 5 element subset indicator string, which does not have a unique Hamming distance closest string in 4 element set indicators.

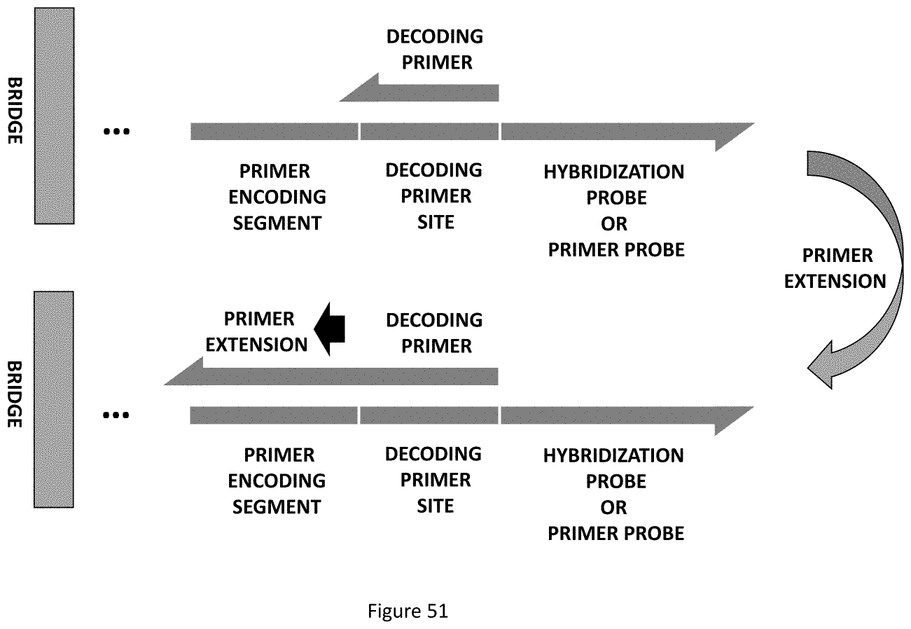

Decoding by Primer Extension in some embodiments, using the sensor to detect primer extension can be used in the decoding process. In this method, the primer probes on the array contain independent targets for decoding primers, such as is shown in the top of FIG. 51. In this configuration, the probe on the array—whether a hybridization probe or a primer extension probe—has affiliated with it a primer binding site for a decoding primer. To decode, such a primer is bound to the target on the probe, primer extension is performed as indicated in the bottom of FIG. 51, and the sensor detects this primer extension. This form of detection can be used as an alternative to the hybridization probe detection of all decoding methods disclosed above. In referred embodiments, the primer extension may produce distinguishable extension signals depending on the sequence of the extended segment. In this case, a universal decoding primer could be used, and the different probe identities could be encoded in these different primer extension signatures that are acquired during/after the primer extension. This has the advantage that multiple hybridizations are not required to decode all probes on the array. Examples of such signatures could be the different signatures produced by block homopolymer templates, which are sequences where each base occurs in repeat form, i.e. in the patterns [n1×B1][n2×B2][n3×B3] . . . [nk×Bk], where B1-B2-B3- . . . Bk could be any sequence of the bases {A,C,G,T}, and the ni, i=1, . . . , k are repeat counts for each bases, preferably each ni is in the range of 1-10 repeats. More generally and set of sequences S1, . . . , SN that produce distinguishable signal traces during extension can be used for such encoding.

In some embodiments, decoding probe binding/not binding and extension signature may both be used in combination to encode the probe identities on the array.

Chip-Based Systems—In some embodiments, the disclosed chip-based multiplex hybridization probe sensor devices are deployed in a compact, low cost electronic instrument that is suitable for distributed use, field use, or point-of-care use. Such instrument architectures in some embodiments comprise a chip board that mates to the chip, motherboard that hosts the chip, and FPGA-based control and data transfer subsystem, a data processing subsystem, which may comprise CPUs, GPUs, FPGAs or other signal processing hardware, a fluidics subsystem, on instrument data storage, and of-instrument data transfer systems.

In some embodiments, this chip device is deployed into a cartridge that in preferred embodiments also allows for some or all other liquid reagents or input sample required for operation to be on-cartridge, to allow for a partial or fully dry instrument platform. In some embodiments, this cartridge is run on a desktop instrument that provides for a user interface, a control computer controlling chip and system functions, control of any on-board fluidics or actuators that control on-cartridge fluidics to supply sample and reagents to the chip, transfer of data from the chip to internal storage or data processors, such as FPGA, GPU or CPU data processors, and transfer of data off instrument via direct internet or wireless connectivity to remote or cloud-based data centers, and such system also provides a sample prep system, internally or as a companion instrument, that takes biosamples of interest and coverts them to the form for on-chip application. In some embodiments, such a system can have a compact form factor suitable for mobile use. In other some embodiments, such a system can have a highly compact form factor suitable for point-of-use or point-of-care or in the field deployment. Such point-of-use applications in some embodiments would include testing stations deployed at airports, transportation hubs, hospitals, schools, stadiums, cruise ships, transport chips, or other major sites of congregation, or deployed at site of business or commercial activity. In some embodiments, such testing stations would be deployed for use in the home, for personal testing and monitoring. In some embodiments, point-of-use systems may be deployed in the field for military, police, customs or border control point-of-contact testing, or other in-the field testing and monitoring applications, such as testing of commercial vehicles, trains or aircraft for presence of pathogens.

Experimental Demonstrations of Molecular Electronic Hybridization Sensors and Chips. Experiments that reduce these devices, methods and apparatus to practice are presented here.

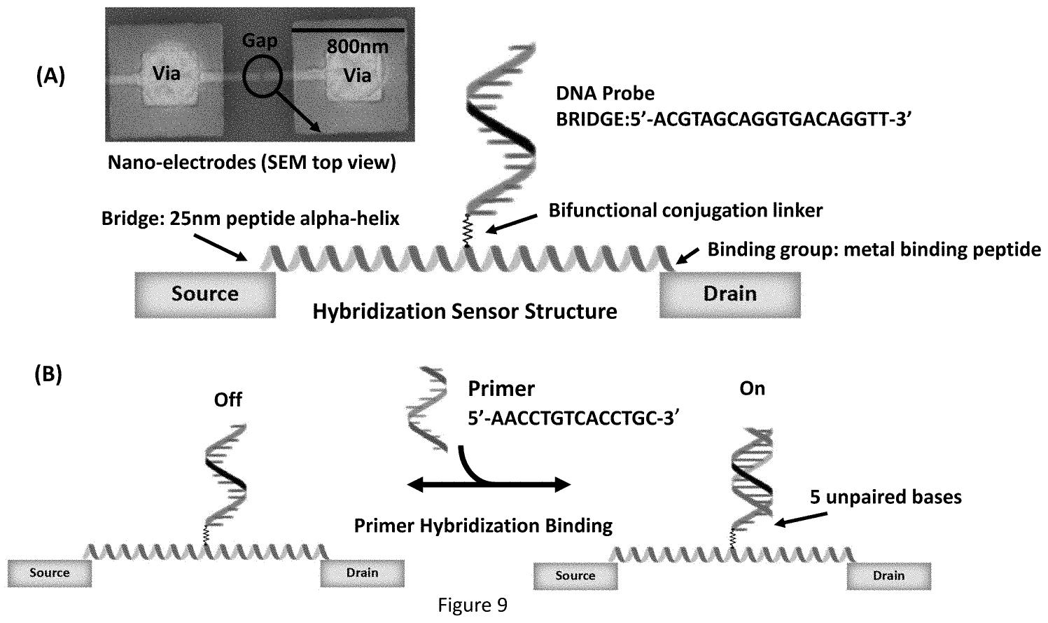

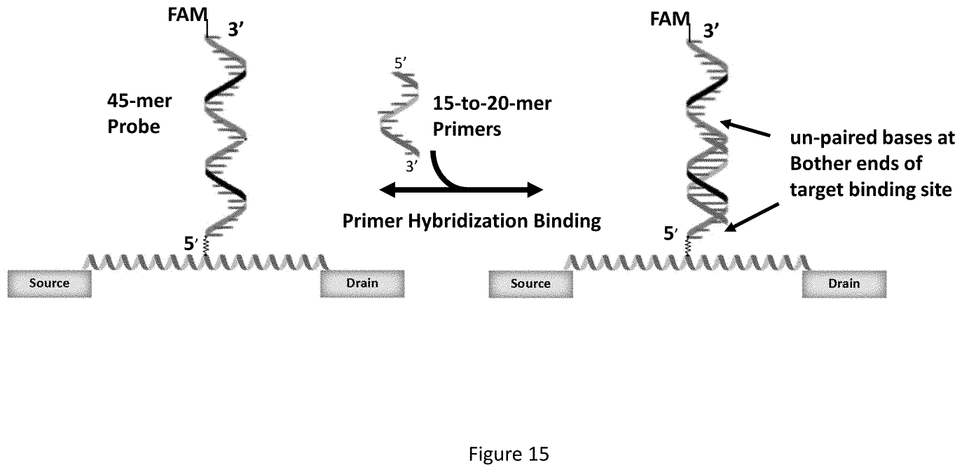

The sensor embodiments used for these experiments are shown in FIGS. 9 and 15. As shown, these consist of a DNA oligo probe, conjugated to an alpha-helical peptide bride molecule, which is bound to Ruthenium nano-electrodes. These devices are deployed on a 16k pixel array CMOS chip device, with a 20-micron pixel pitch, fabricated in a 180 nm CMOD node, and which is a specific embodiment of the chip and pixel architecture shown in FIG. 6. Nanoelectrodes, shown in FIG. 9 in a top SEM view, were fabricated on the chip using standard methods of Electron beam Lithography, sputtering deposition of metal electrodes, and lift-off. The nanoelectrode geometry near the gap is a gap of 15 nm-20 nm, height of 20 nm, and width (looking down from above) of 50 nm. The nanoelectrodes connect to exposed Vias on the chip surface that connect the sensor into the pixel amplifier circuit as in the schematic of FIG. 6, and as shown in the SEM image in FIG. 9, showing the vias. The chip produces 8-bit digital data, at a frame rate of 1000 frames per second. The chip is mounted in a flow cell that expose a 10 uL solution volume to sensor array. the These devices are run on a custom desktop instrument platform, such as indicated be the sensor measurement instrument of FIG. 8, and data are collected on instrument, and transferred to a centralized private server, as well as the Amazon and Google clouds, for Analysis and storage, using internet and broadband wireless connections.

The specifics of the probe-bridge complex for these experiments is as follows. In all cases, the bridge molecule is a peptide that forms an alpha-helical protein structure, with primary 227 amino acid sequence SEQ1

SEQ ID NO: 1:
QQSWPISGSGQQSWPISGSGQQSWPISGSGAEAAAREAAAREA
AAREAAAREAAAREAAAREAAAREAAAREAAAREAAAREAAAR
EAAAREAAAREAAAREAAAREAAAREACAREAAAREAAAREAA
AREAAAREAAAREAAAREAAAREAAAREAAAREAAAREAAARE
AAAREAAAREAAAREAAAREAAARAGSGQQSWPISGSGQQSWP
ISGSGQQSWPIS

The structure of this peptide is that of a repeat of the helix-promoting motif EAAAR (SEQ ID NO: 11), and where a central amino acid is replaced by a C to allow for Cysteine-mediated conjugation to the probe. The termini of this peptide consist of the repeats QQSWPISGSGQQSWPISGSGQQSWPISGSG (SEQ ID NO: 12), which is three repetitions of the metal binding peptide QQSWPIS (SEQ ID NO: 10), separated by short GSG spacers, and which provides for binding to the metal electrodes. In helical form, the length of this peptide is approximated 25 nm. It is used with nanoelectrode gaps in the range of 15-20 nm. This peptide was produced by bacterial protein expression of a synthetic gene encoding the peptide.

The conjugation of the hybridization probe to the bridge is done using a bifunctional cross-linker APN-BCN (Bicyclo[6.1.0]non-4-yn-9-ylmethyl (4-(cyanoethynyl)phenyl)carbamate; Sigma-Aldrich). The conjugation product is purified using a desalting spin-column, and the product peptide reacted with the hybridization probe oligonucleotide having an azide at the 5′ end, such that the 5′ end is conjugated proximal to the bridge. The resulting peptide/DNA complex was purified by size-exclusion chromatography and verified by SDS Gel electrophoresis. The relative quantities of fluorescein (for the labeled SEQ4), DNA and tryptophan were checked by UV-vis spectroscopy. In the case of the probe in FIG. 9, the attached single stranded oligo is a shorter oligo, a 19-mer DNA indicated as SEQ2:

SEQ ID NO: 2:
5′-ACGTAGCAGGTGACAGGTT-3′

The target for this hybridization probe in experiments is he 14-mer SEQ3, which binds leaving a 5-base gap at the Bridge-end of the probe strand:

SEQ ID NO: 3:
5′-AACCTGTCACCTGC-3

In the case of the longer probe indicated in FIG. 15, this also has a fluorescein attached at the 3′ hydroxyl group, for optical detection, and the sequence is the 45-mer (also indicating the 5′ azide and 3′ FAM dye attached):

SEQ ID NO: 4:
/5AzideN/CGATCAGGCCTTCACAGAGGAAGT
ATCCTGTCGTTTAGCATACCC/36-FAM/

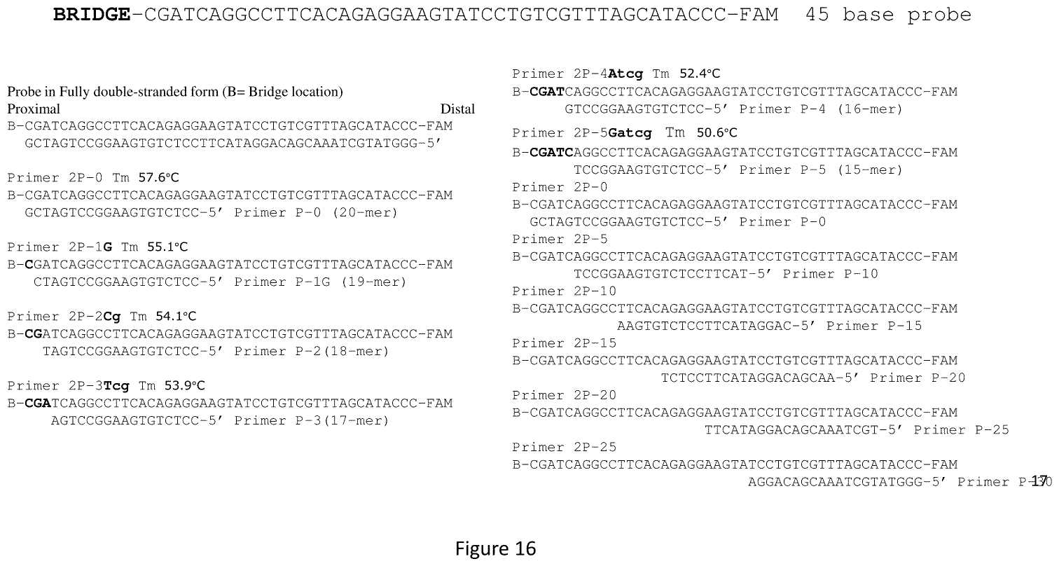

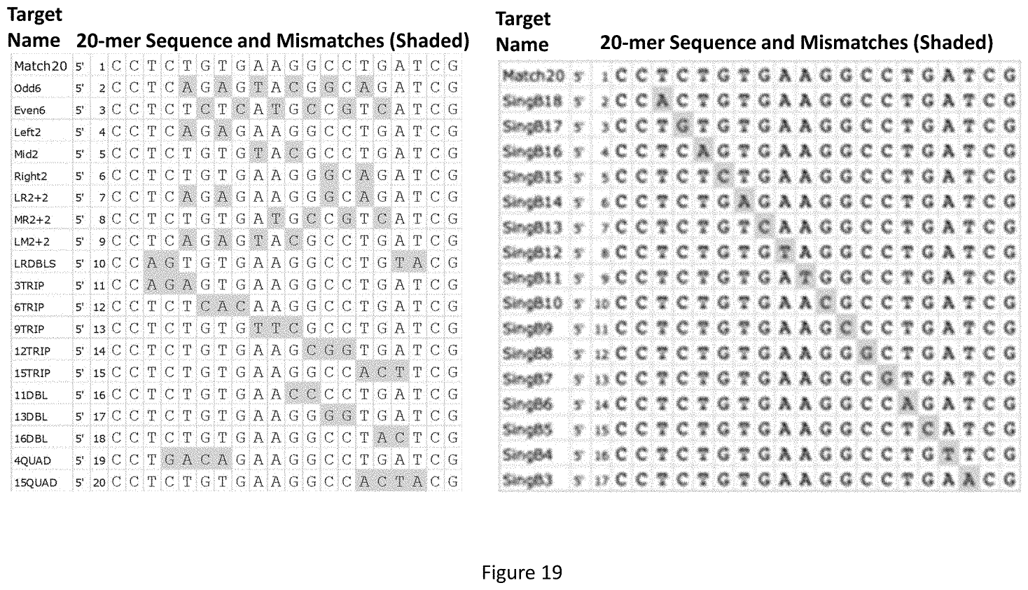

The various different primers (perfect match and with mismatches) bound to this sequence in various experiments are indicated in the table shown below and in FIG. 16, and FIG. 19, and the names of these many primers are indicated here.

| | |
|---|---|
| GCAATGGTACGGTACTTCCGGGATGC | SEQ ID NO: 5 |
| GGAAACTGGCTAATTGGTGAGGCTGG | |
| GGCGGTCGTGCAGCAAAAGTGCACGC | |
| TACTTGCTAA | |
| GACCGCCCCAGCCT | SEQ ID NO: 6 |
| CAGCACCGACCTTGTGCTTTGGGAGT | SEQ ID NO: 7 |
| GCTGGTCCAAGGGCGTTAATGGACA | |
| CCCGCAATTACCTGT | SEQ ID NO: 8 |

The experimental results shown were run at room temperature, in a standard molecular biology buffer, with composition:

| | | |
|---|---|---|
| 50 | mM | NaCl |
| 10 | mM | Tris-HCl |
| 10 | mM | MgCl2 |
| 1 | mM | DTT |
| (pH 7.9 at 25° C.) | | |

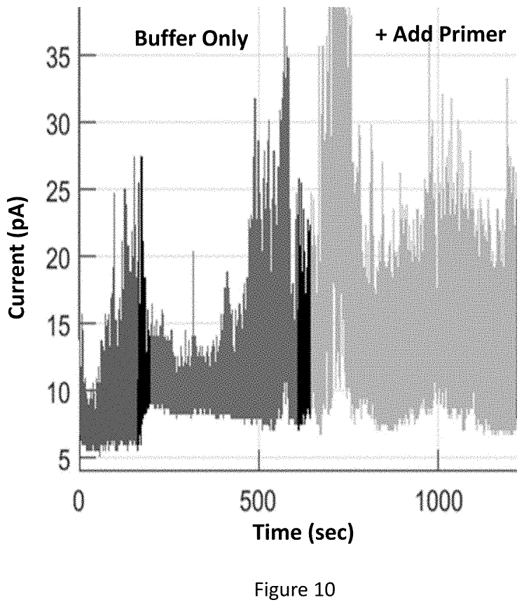

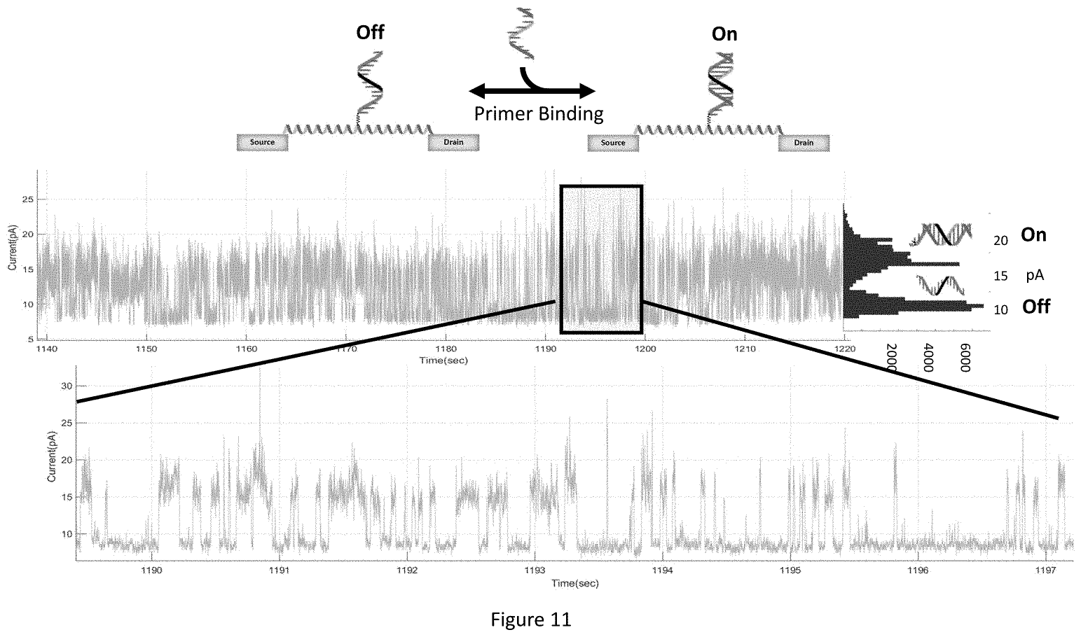

FIG. 10 shows an example signal pixel trace from the short SEQ2 probe sensor deployed on the 16k pixel chip. In the indicated Buffer Only phase, the hybridization target SEQ3 is not present, and this is then added into the solution, typically at a 1 uM (micro-Molar) concentration. The signa trace shown covers approximately 1000 seconds of observation time. Viewed at this high level, it is clear that more and greater amplitude signal spikes occur once the primer is added. The current levels and fluctuations observed are on the scale of 20-30 pA. FIG. 11 shows a close of the signal from the pixel in the hybridization (or “primer binding”) phase, over an 8 second interval. In the close-up, two-level step ups to ˜15 pA are observed, that are interpreted as the primer-bound or “on” states indicated in the cartoon of FIG. 11, while the baseline lower current state at ˜10 pA is interpreted as the off state indicated in the cartoon. This histogram of measured current values, from the 1 kHz sampling, shows the current is distributed around these on and off levels, and provides a view of the relative time spent in each state. This trace illustrates that the sensor detects the basic hybridization of the target, distinguishable as this on-off signal train, which differs from the noise seen in the Buffer-Only phase. This demonstrates the basic functionality of this device as a target detector when viewed as a hybridization sensor.

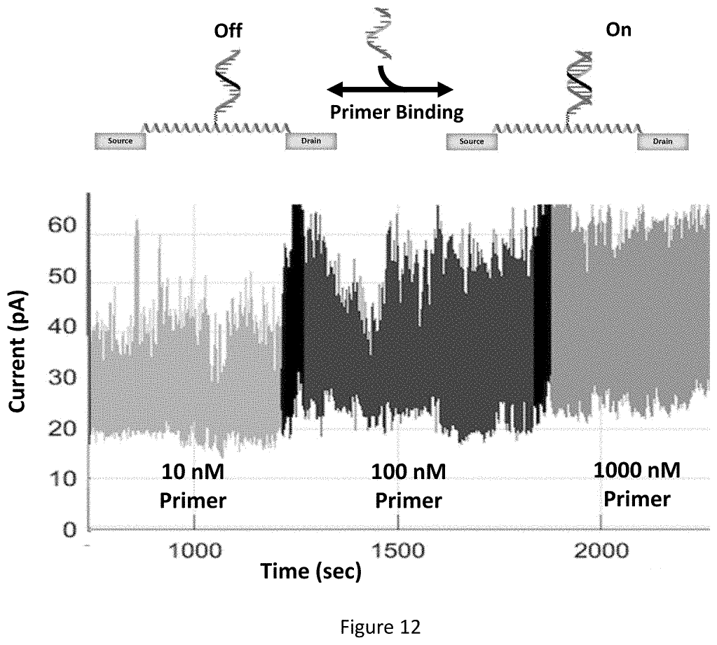

FIG. 12 shows the high-level trace response of the sensor on the chip, when the concentration of target is raised from 10 nM to 100 nM to 1000 nM (1 uM) over the course of 1500 seconds. It is clear from this high-level trace the nature of the signal is changes as concentration increases.

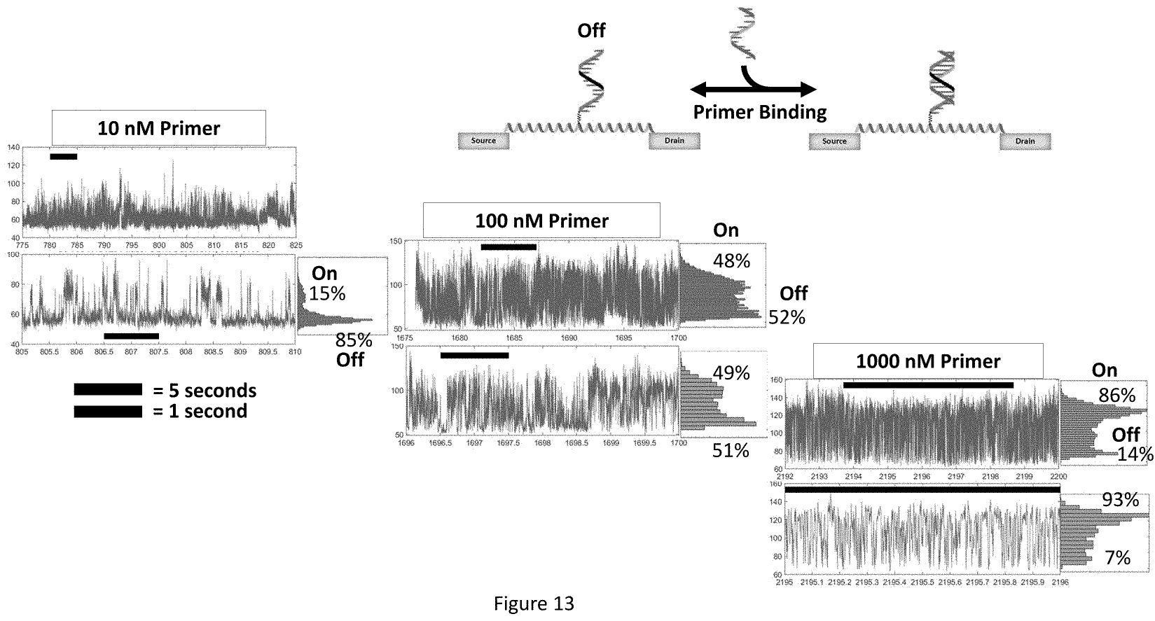

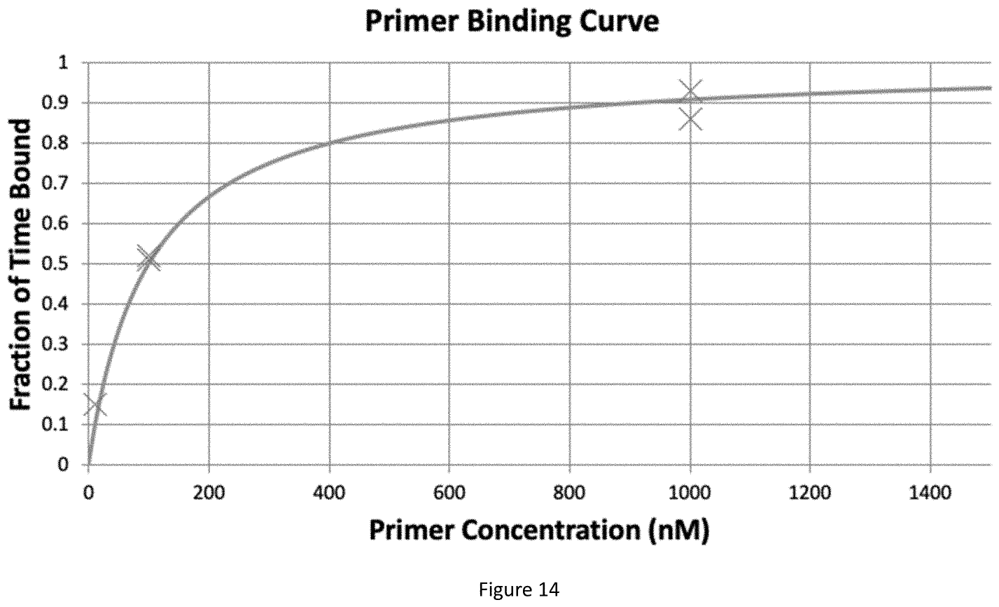

FIG. 13 shows close-ups of the signal trains in each of these phases, as well as histograms that show their relative time spent in the On and Off states. From the close-up signals, it is clear the rate of signal spikes increases in frequency as concentration increases, and also the relative proportion of on time increases, while the amplitude remains nearly constant. Thus, the variables such as pulse rate and on fraction correlate with concentration, and provide the demonstration that such a hybridization sensor provides a measure of target concentration. This demonstrates the basic functionality of this device to measure target concentration when viewed as a hybridization sensor. FIG. 14 shows the data points for on fraction versus concentration (marked as “X” in plot), and the fit of a standard exponential decay curve to such data. The curve fit shows how this sensor can be calibrated to provide an actual measure of concentration for a primary measurement such as on fraction.

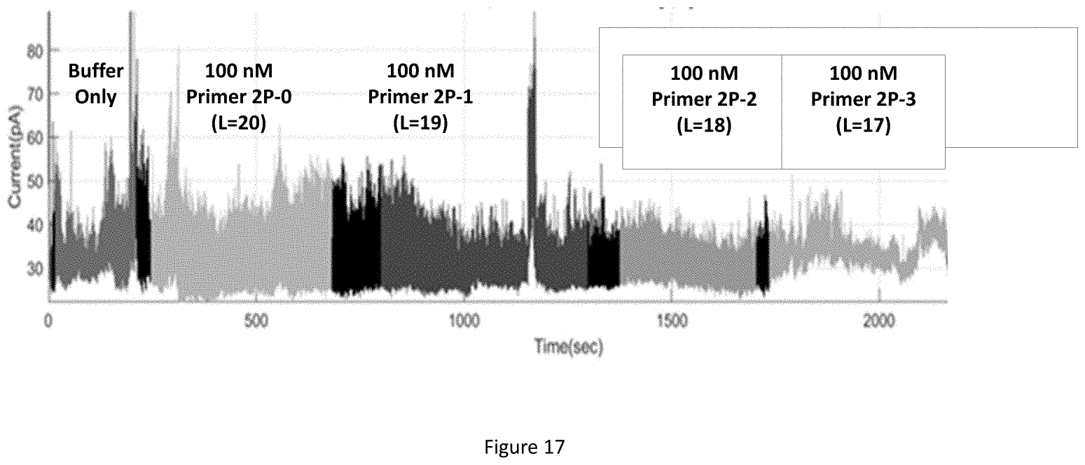

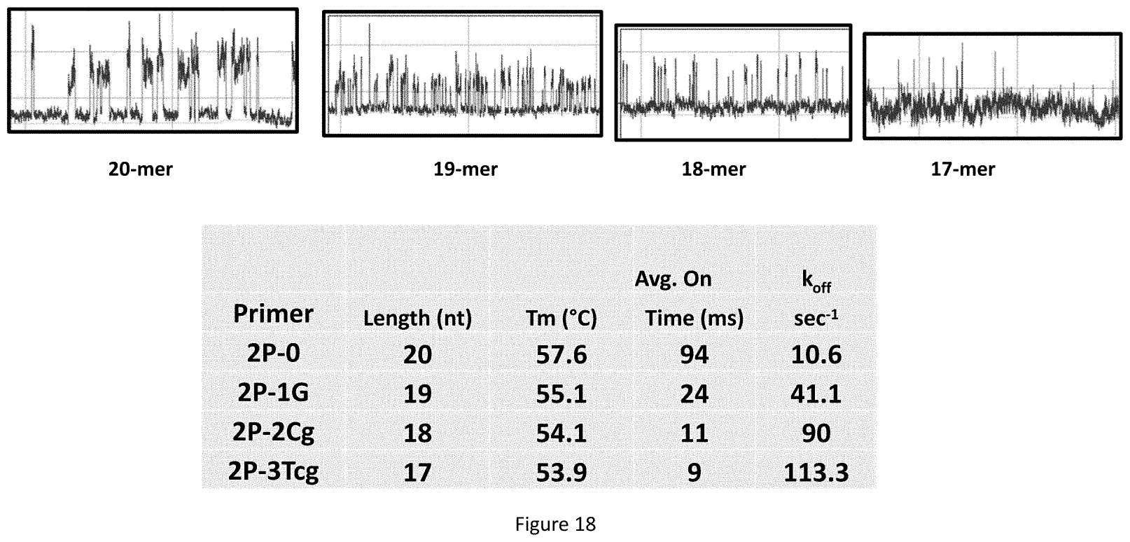

FIG. 17 shows a signal trace from one sensor on the 16k chip, in an experiment in which the hybridization probe binds a series of progressive shorter targets, of length 20, 19, 18 and 17 bases. The targets are anchored to the same distal site, so the 3′ end of the target recedes away from the bridge as they are shortened. It can be seen in the high-level trace that the signals are diminishing as the length of target (and hence Tm) are reduced. FIG. 18 show close-ups of the signal trace in each phase, showing that as the target shortens, the amplitude of pulse drops slowly, but the average “on” time drops off more substantially, and net effect on signal trace is very clear and distinguishable. The table shown in FIG. 18 presents the length and Tm of the targets, and the average on time and corresponding off-rate. Overall, this demonstrated good sensor sensitivity, as the sensor clear detects 1-base level differences in the target (here, 1-base length differences).

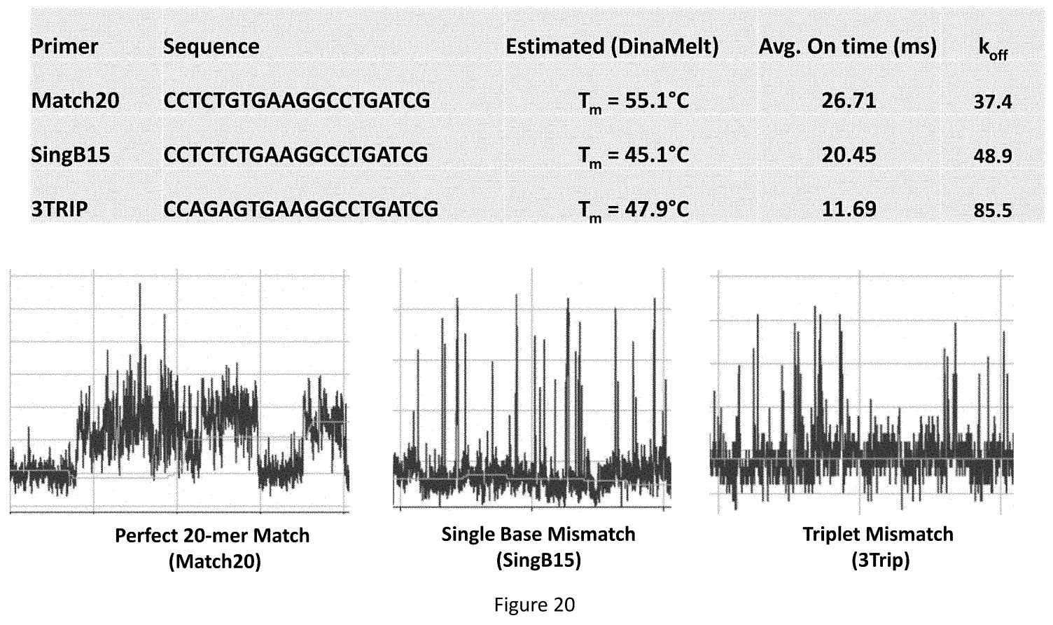

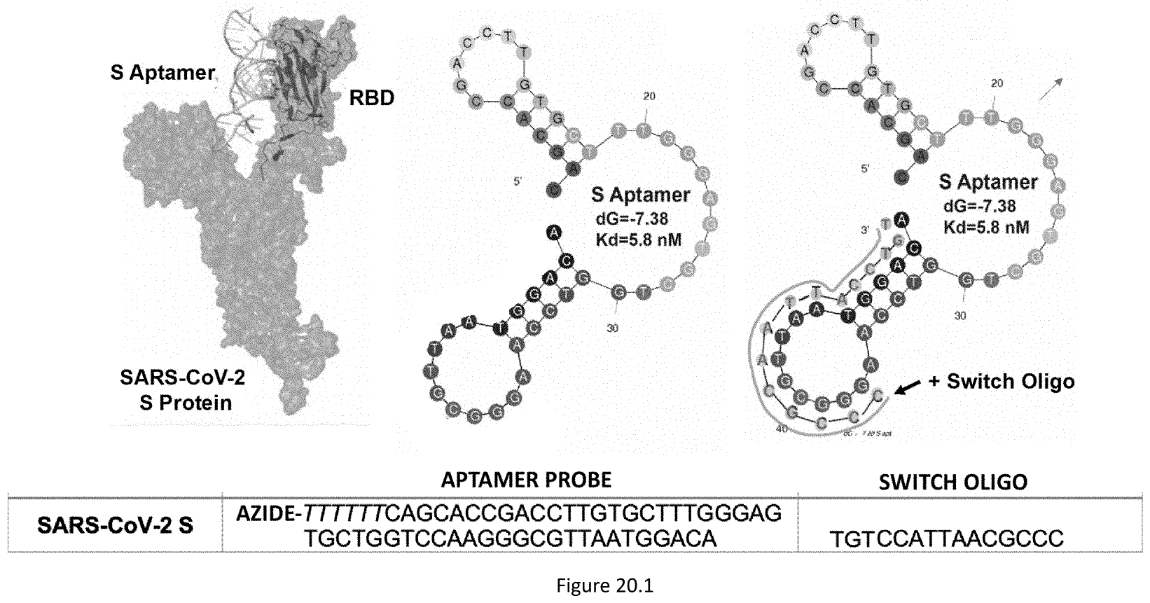

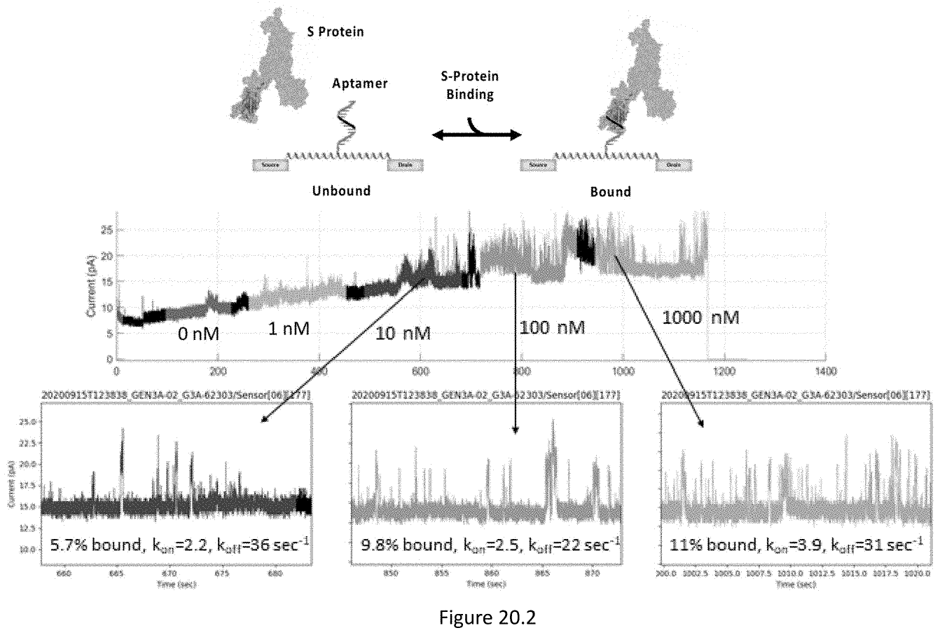

FIG. 19 shows a collection of mismatched targets, that also have 1-base differences, as well as multiple base differences, from the perfect match target shown (named as Match20). On the right side of FIG. 18 are a series of 20-mer targets with a single mismatch scanning across the length. On the left side are various 20-mer targets with multiple mismatches, from 2 to 6, spaced out or adjacent. FIG. 20 shows signal results from one base and 3 base mismatched targets relative to the signal for the perfect matched target, along with the table of respective Tm melting points, average on time, and corresponding off rate. Note that the single base mismatch makes a clear change to the signal trace, with lower “on” time as expected, as well as reduced amplitude, and the triple mismatch is even further substantially reduced in on time and amplitude. These results show that a single bas mismatch makes a substantial and detectible difference from a perfect match. This demonstrates the ability of this sensor to discriminate proper hybridization from cross-hybridization (e.g. the triple match target), which is critical to using this to sense targets in complex pools, and the single base discrimination enables applications such as SNP genotyping and strain determination, that depend on single base differences in targets.

FIG. 15 shows the primer binding process used for the primer extension experiments shown here. The primer binding indicated there provides for a primer extension experimental as indicted in FIG. 46, where the template DNA strand is on the array, bound to the bridge at the 5′ end, the primer binding site is in the interior of this segment, and binds with its 3′ end oriented towards the bridge. A maximum extension of 10 bases is possible before the polymerase reaches the end of the template. As indicated in the trace shown in FIG. 46, In the experiment, there is a primer binding phase, which generates signals of primer binding, a polymerase binding phase, which generates signals of the polymerase binding, and an extension phase which generates signals of extension.

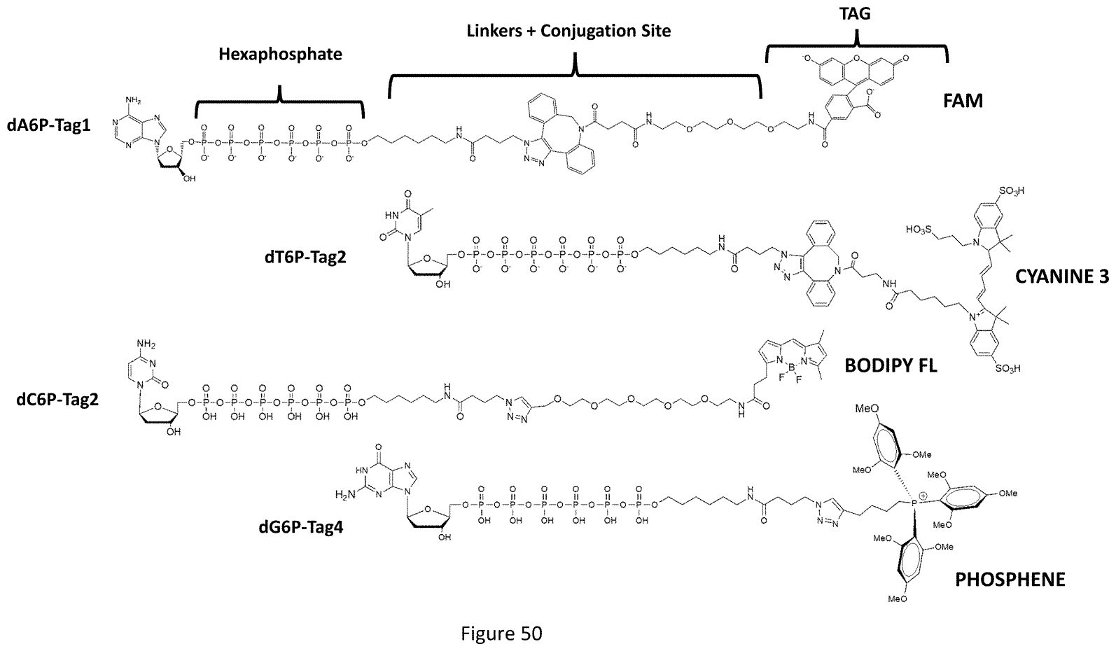

Experimental Conditions for Primer Extension. The primer concentration is 100 nano-Molar, for the priming of the targets on the sensors. The polymerase used is Klenow, used at 50 nano-Molar concentration unless otherwise indicated. Two different buffer compositions are used, one with Mg++ and Mn++ as the divalent metal cation co-factors for the enzyme, in which the enzyme is catalytic, and can extend, and a version where these are instead replaced by Strontium, at the same concentration, producing Sr++ as divalent metal cation co-factor for the enzyme, in which case the enzyme is not catalytic, and therefore will preferentially engage the dNTPs, but cannot incorporate and extend the primer. Instead, it stays in a dNTP sampling mode, with these transiting in and out of the enzyme binding pocket. dNTPs are added at a typical concentration of 20 micro-Molar concentration. In the present experiments, the dNTPs are modified forms, with an added signaling group. The chemical structures of the ones used in this experiment are shown in FIG. 50. These modified dNTPs have a hexaphosphate chain, followed by a linker to a conjugation site, followed by another linker to various signaling tags, hence the structure is dN6P-Tag, with specific tags as indicated: dAT6P-FAM, dT6P-Cyanine 3, dCTP-Bodipy FL, dG6P-Phosphine. Such modified forms require Manganese in the buffer for efficient incorporation. The assay buffer composition for primer extension is:

20 mM Tris

10 mM (NH4)2SO4

10 mM KCl

0.8 mM MnCl2

3.2 mM Magnesium Chloride

(ph7.5 at 25 C)

The Sr version of the buffer has the Mg component replaced by Sr.

FIG. 47 illustrates the phases of activity (top) and shows an actual experimental signal trace (bottom) over a 4000 second interval. As shows there, there is substantial signal produced in all phases of primer binding, polymerase binding, primer extension in the Strontium buffer (non-extending) and primer extending in the Magnesium buffer (extension).

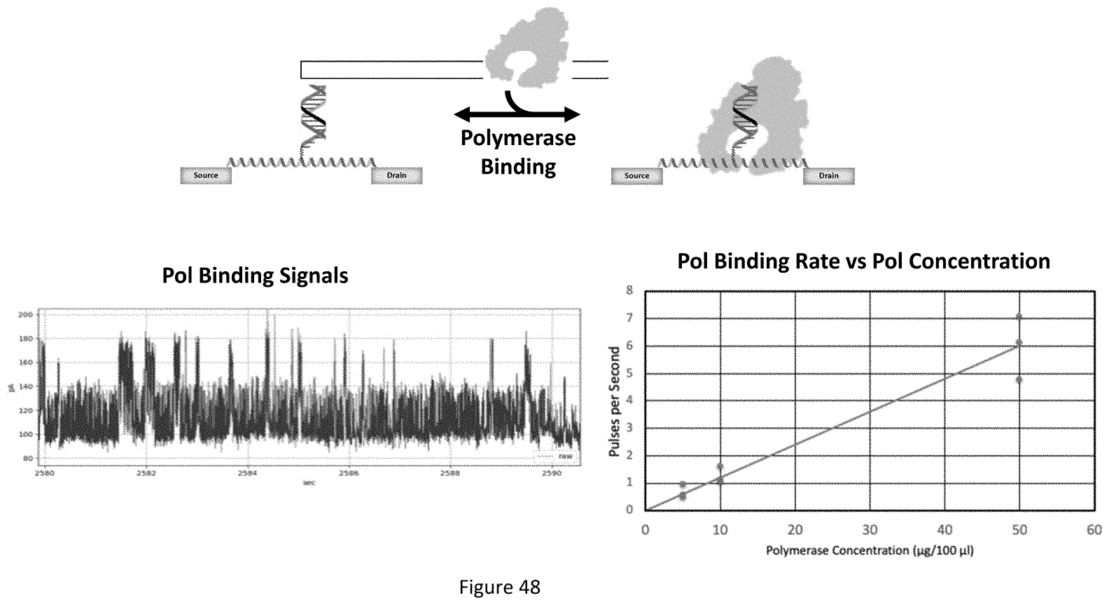

FIG. 48 shows a close up of the signal pulses produced by binding of the polymerase, as well as a graphic of pulse rate titrated against polymerase concentration, which shows that the rate of pulses increases approximately linearly with polymerase concentration.

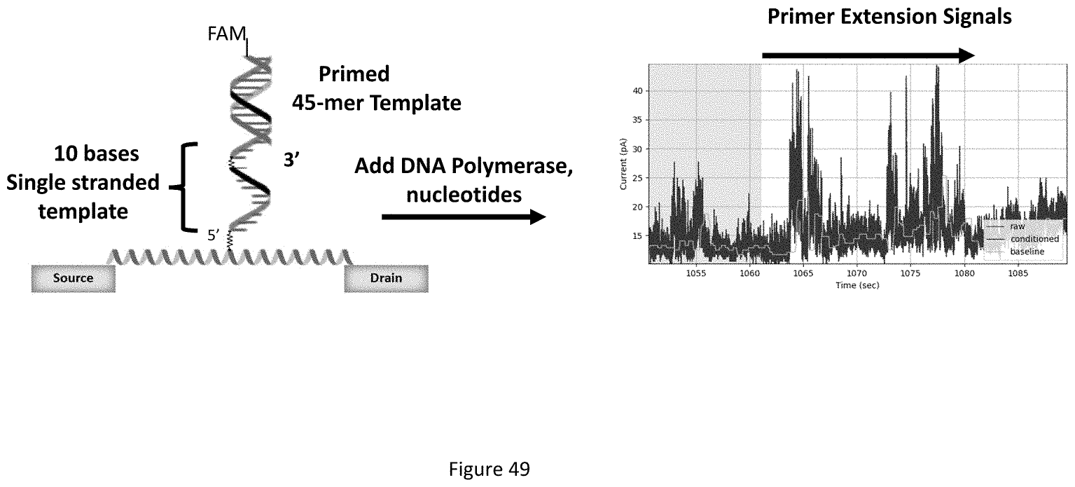

FIG. 49 shows the actual primer extension phase, the cartoon at left illustrating the primer template configuration, and the signal trace at right showing signals in the non-extending Sr++ buffer phase (shaded grey) and the extending Mg++ buffer phase, which shows substantial greater pulse activity as the enzyme extends the template for the up to 10 bases.

In particular, this extension phase data shown here illustrates reduction to practice of the type of primer extension signaling and primer extension signature traces that may be used to provide the distinguishable signals referred to in the disclosures herein, or the distinguishable signal signatures referred to in the disclosures herein.

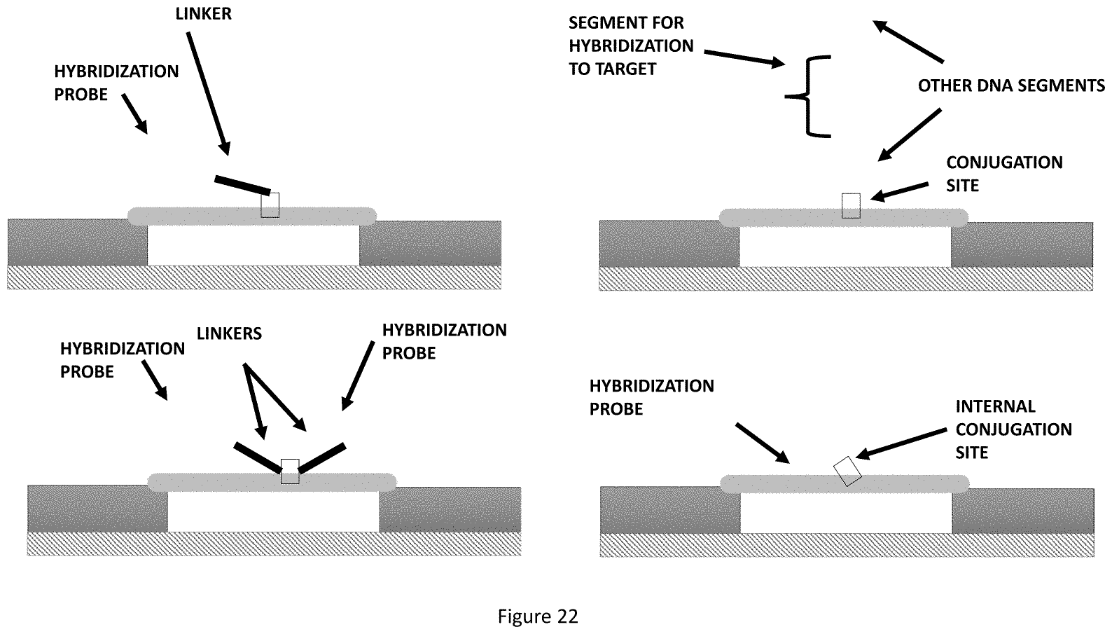

Additional Molecular Electronic Hybridization Sensor Embodiments In these experimental examples using the sensors shown in FIGS. 9 and 15, the hybridization probe oligo is attached to the bridge at its termini, as it also is in cartoon depictions of the sensor in FIGS. 2 through 5. However, these examples and depictions are not meant to be restrictive, and in some embodiments, the hybridization probe may be attached at either termini, or at an internal site, such as is indicated in FIG. 22. FIG. 22 illustrates examples where the attachment is by a long linker (upper left), or multiple such linked probes, identical or distinct (lower left), or where the probe of interest with sequence specific for a target is a sub-segment of a longer DNA strand, with additional bases at one or both ends (upper right) or attachment at an internal site of the probe strand (lower right).

A suggested by FIG. 22, in some embodiments, the hybridization probe-containing strand may be attached in diverse manners to the bridge, also including multiple point of attachment, or having a non-linear form, such as the branched forms in FIG. 21, or a circularized form, or other diverse forms obvious to those skilled in the arts of DNA conjugation and manipulation, as long as such forms allow the portion of the strand that complements the target to be available to form a hybridized duplex with the target strand.

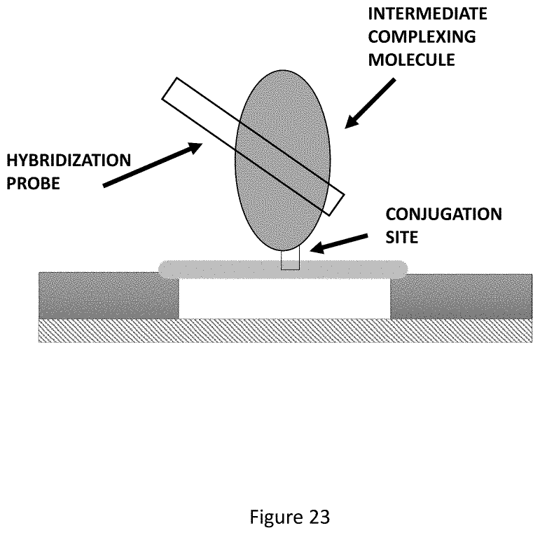

In other some embodiments, an additional intermediary molecule may be used to complex the hybridization probe with the bridge, such as shown in FIG. 23. Such a molecule could be a larger protein that complexes with DNA, such as a DNA binding protein, or a DNA binding enzyme such as a polymerase, ligase, restriction enzyme, CRISPR/CAS9 protein. Such an intermediate molecule may in some embodiments enhance the signal produced by the hybridization event.

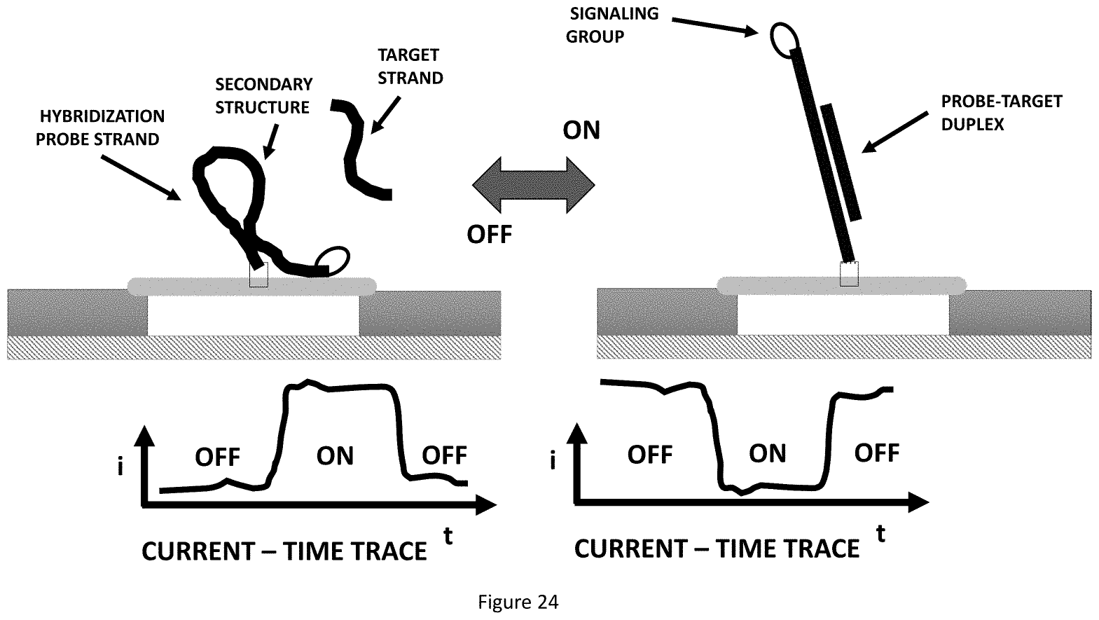

As shown in FIG. 24, in other some embodiments, the single stranded probe may have an exemplary secondary structure with itself and or with the bridge (shown at left), that unfolds to hybridize with the target (at right). In some embodiments, there may also be a signaling group on the probe, which may produce a detectible signal when it changes configuration between the on and off states. In general, the secondary structure present is intended to have a melting point Tm near or slightly below that of the exact match hybridization, so that the exact match can compete with some efficiency for binding, by displacement of the secondary structure, but other fragments that do not match, have greatly reduced ability to form stable interactions with the probe. The secondary structure changes, included optionally the change in signal group location, can be used to produce a detectible signal, or a larger signal, or greater signal to noise in detecting hybridization. The secondary structure may also decrease the signals or noise from off-target strands in solution. In some embodiments, the Tm of the secondary structure would be 5 C-10 C below the target-probe duplex Tm (C=degree Centigrade), or 1 C-5 C below or within 1 degree of, or 1 C-5 C degrees above this Tm, or 5 C-10 C above this Tm. In some embodiments, all or part of the secondary structure may be designed or induced by DNA hybridization bonding. In some embodiments, this is done through the use of matched and mismatched base pairing. Such secondary structure elements may include hairpin formation.

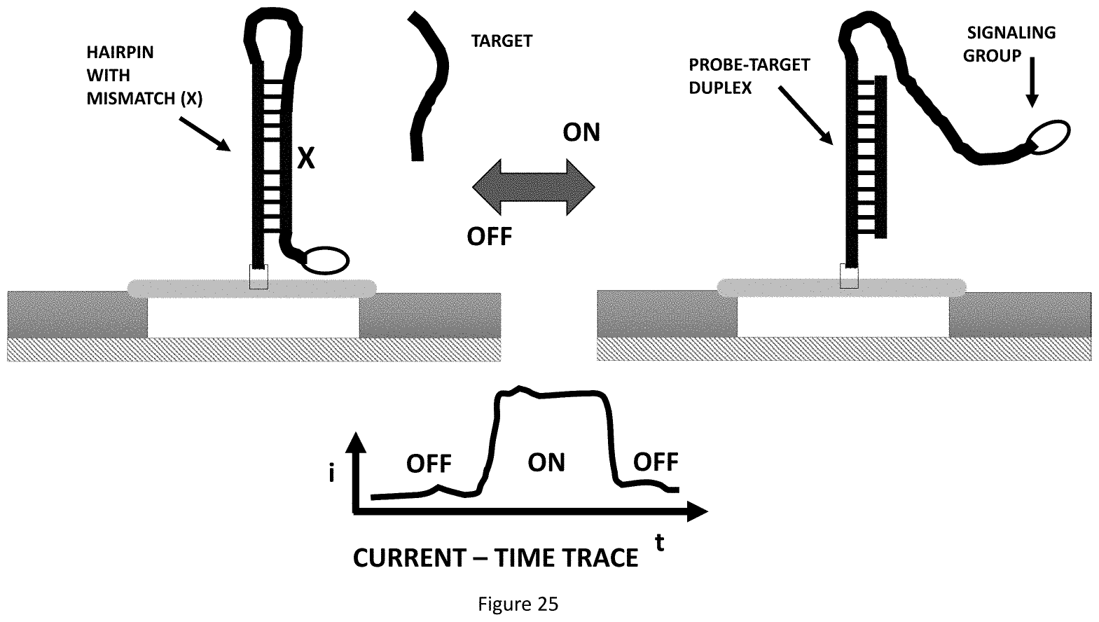

As indicated in FIG. 25, one exemplary embodiment extends the probe to include a segment that forms a hairpin with one or more mismatches to the probe segment specific to the target of the hybridization. In this setting, the target molecule can bind with high stringency against unwanted off-target hybridization. The hairpin also affords the option to put a signaling group on the hairpin, such that in the hairpin form, the signal group will be held near the bridge, and in the on form bound to target, this group will be relatively far from the bridge, and more mobile, both which may contribute to a signal.

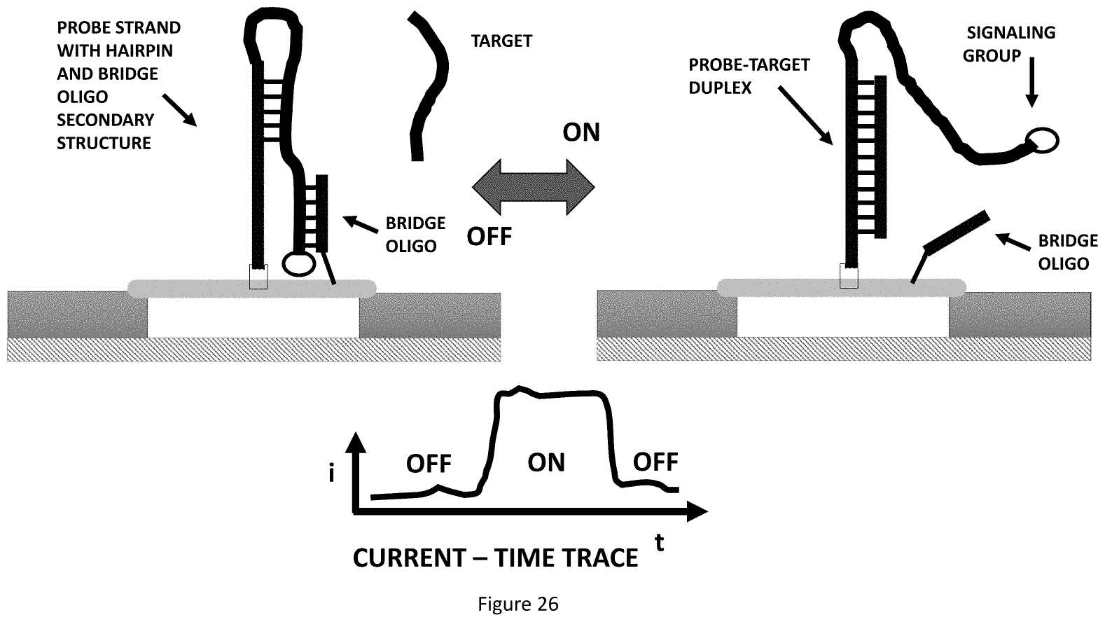

FIG. 26 shows another preferred embodiment, with a hairpin configuration and also binding to a secondary oligo attached to the bridge. This may also be used to enhance signal, either from the disruption of this secondary hybridization, or by changes in location of the signal group.

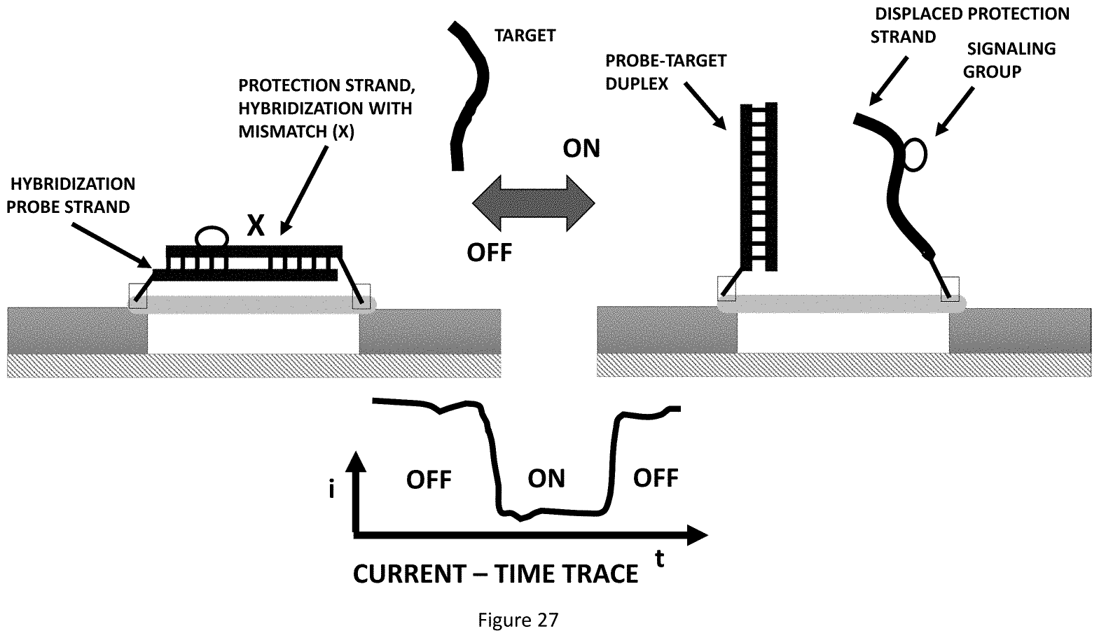

FIG. 27 shows another preferred embodiment, where a protection strand is used, which binds the target less effectively than the target, and in some embodiments differs by mismatches form the exact match, for finer control of this effect. The protection strand is attached and positioned as indicated, so that it creates a partial duplex with the probe that results in a parallel bridge structure to the primary bridge. This may also be enhanced by the presence of a signaling group, in some embodiments. Upon hybridization to target, the parallel bridge is disrupted, as is the signal group, both of which may produce a detectable signal.

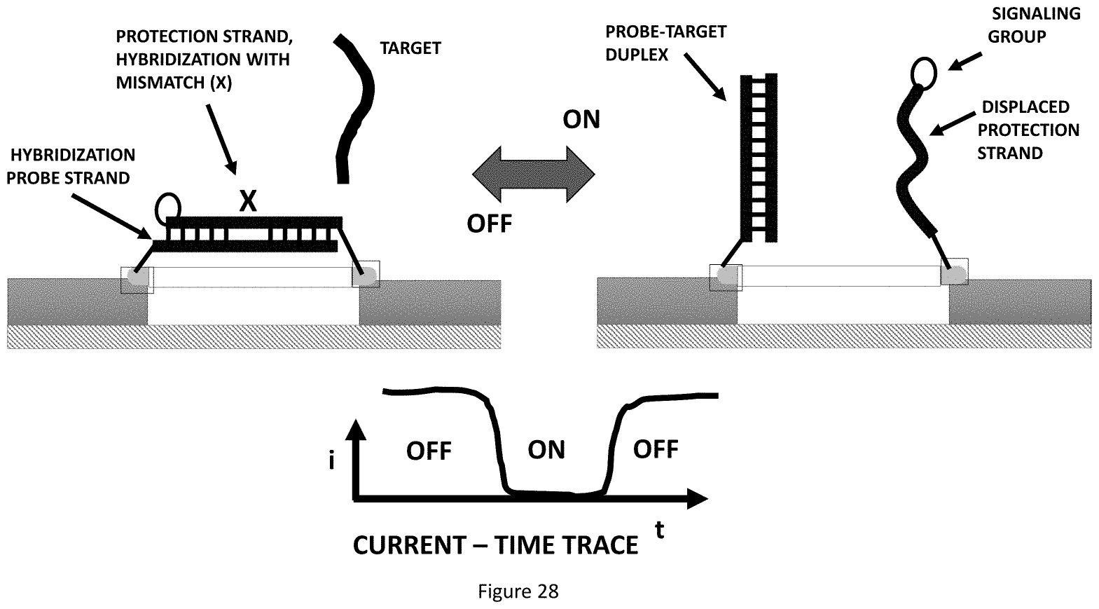

FIG. 28 shows an exemplary embodiment where the bridge in FIG. 27 is the sole bridge, and so in this case the hybridization to the target completes disconnects the electrodes. This may produce a large detectable drop in current.

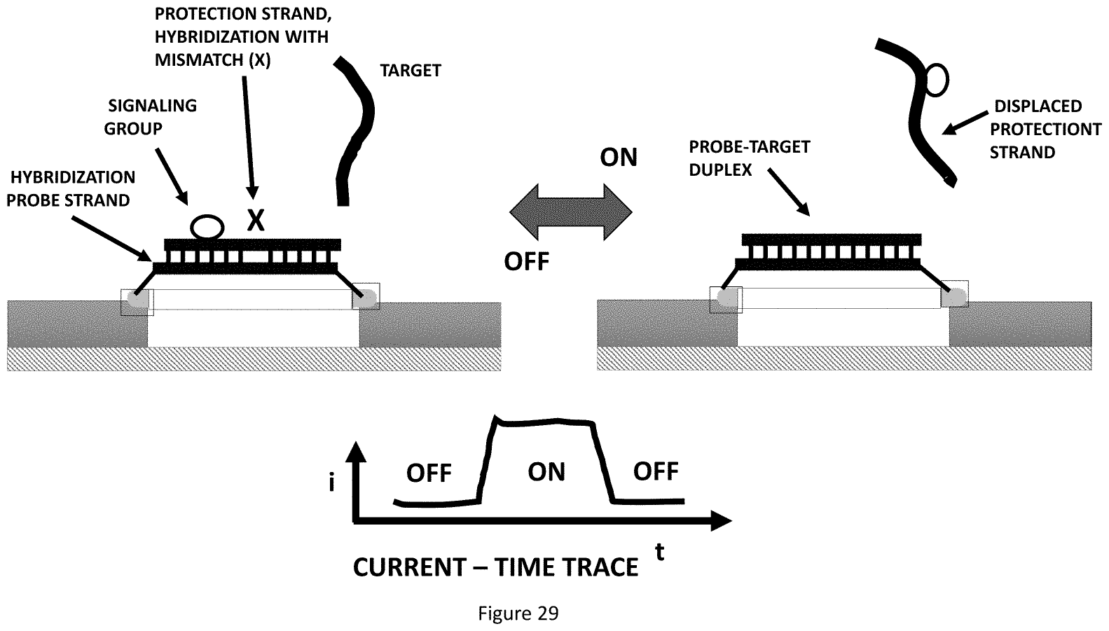

FIG. 29 shows an exemplary embodiment where the bridge is formed by the protecting strand in a mismatched duplex or otherwise lower Tm duplex (relative to target Tm), optionally with a signal group on the strand, and where the protecting strand is not otherwise attached. In this case, upon hybridization to the target, the protecting stand is displaced and lost (along with the signal group), and a complete duplex is formed. These processes may result in a detectible signal.

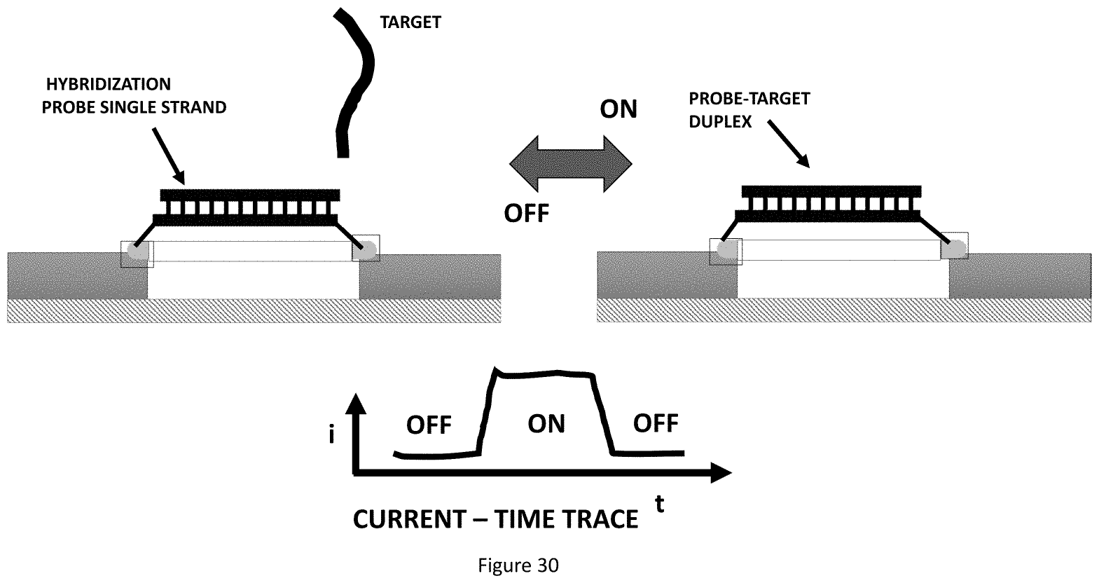

FIG. 30 shows an exemplary embodiment where the single stranded hybridization probe spans the electrodes and binding to the target creates a duplex DNA bridge, which may produce a signal of a large jump to much higher current levels.

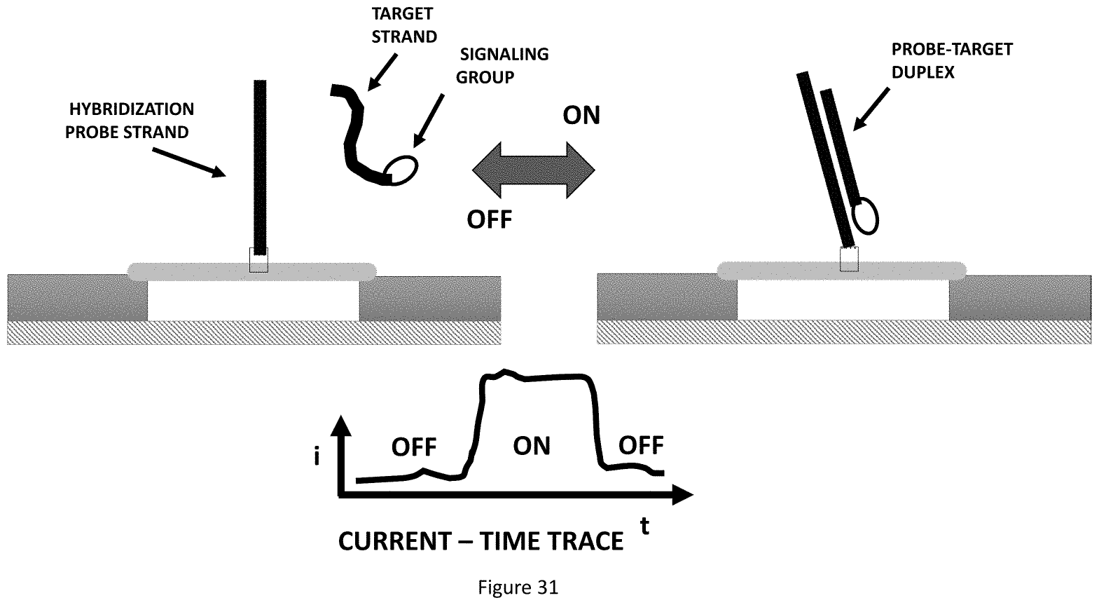

FIG. 31 shows an exemplary embodiment where the target strand has an added signaling group, resulting in larger signal from the hybridization binding this group near the bridge. Such a group may be added to the sample by standard sample labelling reactions that are commonly used to attach diverse labels to DNA or RNA, and many such reactions are widely used and well known to those skilled in molecular biology or conjugation chemistry.

Note that the embodiment shown in FIG. 31, of labelling the target with a signaling group, can apply broadly to all the hybridization sensor embodiments and applications disclosed here.

There are many variations and combinations of the embodiments disclosed above that may provide useful benefits to molecular electronics hybridization sensors, methods and applications, and to one skilled in the art of molecular biology, these would be obvious variations, and these are therefore also all encompassed by the disclosure here.

Experimental Demonstrations of Molecular Electronic Aptamer Sensors and Chips.

FIG. 20.1 shows the S protein of the SARS-CoV-2 virus (left), with the DNA aptamer shown bound. The sequence and secondary structure of this aptamer are shown (middle). Details on the development and properties of this aptamer were published in [1]. Song, Yanling; Song, Jia; Wei, Xinyu; Huang, Mengjiao; Sun, Miao; Zhu, Lin; et al. (2020): Discovery of Aptamers Targeting Receptor-Binding Domain of the SARS-CoV-2 Spike Glycoprotein. ChemRxiv. Preprint. https://doi.org/10.26434/chemrxiv.12053535.v1

In addition, the binding of this aptamer to a complementary “molecular switch oligo” is shown (right), this oligo is displaced by the S protein binding, and provides for a secondary detection mechanism for the binding. The sequence of the aptamer, along with the linker and conjugation group used to attach it to the molecular bridge is shown in FIG. 20.1 (bottom). (The sequence of the 15-mer switch is also shown there.) The attachment ground is an azide, with a “TTTTTT” linker to the aptamer, which is conjugated to a bridge molecule by the same process as described above for the hybridization probe.

FIG. 20.2 (top) depicts the S aptamer-based molecular electronics sensor and its binding kinetics with the S protein target. An exemplar signal trace is shown in FIG. 20.2 (bottom), for a sensor array chip experiment, with the aptamer sensor deployed on the same 16k sensor CMOS chip device as used in the hybridization experiments. The experiment is performed in a 0.5× concentration PBS buffer, and shows sensor response to a series of increasing nano-Molar concentrations of the S protein (0 nM, 1 nM, 10 nM, 100 nM, 1000 nM) are applied to the sensor array. The inset figures show close up of the current vs. time trace in these different concentration phases, illustrating the increasing rate of signal spikes (increasing on-rate, k_on) as the S concentration increases.

Assay Methods for the Personal Virometer—It is an element of this disclosure to describe the assay methods for the personal virometer. In some embodiments of the personal virometer, the primary biosample is a saliva sample, or a nasal swab, or buccal swab, or a stool sample, or a blood sample, or a sample of other bodily excretions. In other some embodiments, the primary biosample is taken from the environment, and may be a swab of a surface, material filtered from air, or a sample taken or filtered from water or wastewater.

Given such a sample, in some embodiments, there may be a primary sample prep step in which viral material, such as particles, proteins, or DNA or RNA, is extracted or isolated from the primary biosample. This may comprise steps for disrupting the viral particles, and freeing up proteins or nucleic acids, as well as comprise steps of targeted capture, elution, or purification.

The material from the primary sample prep step, in the case of DNA or RNA, may undergo an amplification process, such as PCR and related primer-mediated cyclical amplification procedures, or isothermal nucleic acid amplification. Such methods may be from the well-known methods widely used in rapid nucleic acid diagnostics, such as LAMP, RPA, RCA, NASBA, WGA, SDA, HADA, as well as oligo isothermal amplification methods such a Hybridization Chain Reaction (HCR) and Signal Amplification By Exchange Reactions (SABER). Such amplification methods may include reverse transcription, such that an RNA viral genome is amplified into DNA products. Alternatively, such methods may amplify RNA into RNA products.

In some embodiments, such amplification methods are targeted with specific primers, to amplify multiple target segments of interest, from one or multiple viral targets.

In some embodiments, such targets of interest may include targets in the human genome of the test subject, such as might be target segments for SNP genotyping. In such cases, the primary sample preparation would properly preserve the human genome content of the sample as well for this purpose.

The resulting products of the primary sample prep, and possible amplification and targeting, are then applied to the sensor array device for detection and readout. Such a sensor array will have suitable and corresponding hybridization probes, primer extension probes, or aptamer probes, targeting the desired targets in the material delivered to the chip, or, in other some embodiments, other properly matched probe types, such as antibodies.

Electronic Concentration In some embodiments, it is desirable to enhance the sensitivity of detection by using electrical forces generated on the sensor array chip, to concentrate the targets of interest in solution to the vicinity of the sensor pixel molecular probes. Such concentration ideally concentrates target molecules to within 10's or 100's of nanometers of the sensor probe molecules, and overall achieves up to 1 thousand fold, 1 million fold, up to 1 billion fold, or up to 1 trillion fold concentration enhancement in the region within 10's or 100's of nanometers of the probe molecules, relative to the ambient concentration of the target molecule as introduced to in the chip flow cell. The are two general methods of voltage driven concentration enhancement that can be applied for such electronic concentration, and which can be implemented on the sensor array chips disclosed, by providing the needed supporting circuitry:

Electrophoresis—When a constant voltage (DC) is applied to the sensor nanoelectrodes, targets in solution of the opposite charge will be attracted from the bulk solution to the electrodes, which is also known as the process of electrophoresis. This is useful in locally concentrating charged molecules.

Dielectrophoresis—When an alternating (AC) voltage is applied to the sensor nanoelectrodes, targets in solution that can be electrically polarized will be attracted from the bulk solution to the gap between electrodes, which is also known as the process of dielectrophoresis (or DEP) trapping of particles or molecules. This can be useful in locally concentrating most molecules, even if they have no net charge, as most molecules exhibit some degree of polarizability.

Indirect Aptamer Detection—In some embodiments in which aptamer probes are available for the detection binding reaction, such aptamers can be employed in a different off-chip, solution phase manner that allows for additional signal amplification and therefore additional sensitivity of the detection assay on chip. In such embodiment, a “molecular switch oligo” is also provided with each aptamer, such as the one illustrated in FIG. 20.1. Such switch oligos are displaced when the aptamer binds its specific target, and thereby become a free oligo molecule in solution upon the aptamer binding its target. In this preferred embodiment, the aptamers are deployed in solution phase, in multiplex fashion, and exposed to the prepared sample (off of the chip flow cell, or in the flow cell). The subsequent binding events results in the release of switch oligos. These can then be subject to a suitable nucleic acid amplification reaction of the types described above, so that each oligo results in a highly amplified number of cognate amplification products. On such embodiment is to have suitable primer pairs that target these oligos as templates for amplification, as per standard exponential amplification reactions. Such amplification could be up to 1000-fold, up to 1 million-fold, up to 1 billion-fold or up to 1 trillion-fold. These amplification products are then preferably detected by cognate hybridization probes on the sensor array chip, or similarly by primer extension probes and reactions.

Informatics Methods for the Personal Virometer—In some embodiments, after reading of the assay materials on the sensor array chip, the results are located reported in via possibly a user interface screen on the virometer instrument, as well as a wireless link to a local device such as a personal cell phone, smart watch, or personal computer, or uploaded to a cloud-based data repository. This information app supporting this storage, in some embodiments, provides for centralizing of such information across multiple test subjects, test devices, test times, and areas. This may include centralization of information for a household, or for larger collections of individuals, such as by geographical regions or government jurisdictions, including neighborhoods, communities, cities, states, countries, or globally.

In some embodiments, the virometer supports smart, remote, delayed notification, such that after a test is initiated, up completion it can forward the test results to another smart device, such as a cell-phone or personal computer or website. This provides for a use case where individuals are tested at a primary site, such as at home, and then notified while they may be in transit, such as to work or school or the airport. In the case of a positive result, they could abort their trip prior to reaching the destination, but otherwise not have to delay their travel to wait for test results. This means that the turn-around-time of the test does not cause delays for many forms of important daily activity.

In some embodiments, the results of the of the sensor assay, which comprise detection results on the viral targets, are affiliated with metadata which may comprise an automatically generated time stamp and GPS location information, as detected by the device, and subject or sample identifiers provided by or selected by the user. Such metadata may also include identifiers for the device, chip, and version of the assay.

In some embodiments, the sensor array chip assay will also determine a genetic fingerprint of the test subject, such as an identifier based on a set of SNP genotypes, which is affiliated with the test viral measurement results as a personal identifier. In some embodiments, this personal identifier may remain anonymous, or may be affiliated with other common personal identifiers such as subject legal names, user-provided nick-names, or issued ID numbers. In some embodiments, the genetic fingerprint can be used to affiliate distinct tests done at different times with the same individual, using a data aggregation app running on a cell phone, computer or in the cloud. In some embodiments, the virometer provides the means to register new users, and assay their genotype (in a separate assay, or with a viral assay), and affiliate this genotype with user-desired information to be stored in the information app.

In some embodiments, the data can be used to create a digital certificate of screening, which certifies the individual was tested, along with metadata such as the time, location, and identify of the testing device or chip or assay. Metadata may include the genetic fingerprint. Such a digital certificates could be used a rapid screening proxy, so as to allow individuals entry into areas that are being controlled for access to prevent spread of viral infections if they have certificate produced within a certain time window, that clears them from viral infection. This could be used for access control for schools, places of business or employment, hospitals, airports, on board air travel, other travel hubs or public transportation, large gatherings, entertainment or sporting events, hotels, cruise ships, taxis, hired ride vehicles, or any other areas where is it desirable to have screen individuals for possible infection before granting access in order to reduce the spread of viral diseases.

Such certificates could also be applied personally, using phone-based verification apps, to ensure that individuals are not infected prior to engaging in any forms of social or personal gatherings or meetings. In referred embodiments, this is combined with social networking sites, such as dating sites, to provide assurance that individuals are recently screened as virus-free prior to meeting in person.

In some embodiments, the information app supporting the virometer data automatically aggregates personal test results of an individual over time, so as to provide for monitoring of personal health states. This also provides for the ability to establish baseline levels of viral infection, over a broad range of viruses, so as to have precision understanding of how tests differ in time from individual baseline levels.

In some embodiments, the information app is cloud based and centralized, so as to aggregate test results from large populations of individuals, such as from communities, regions, or individuals who otherwise may be able to transmit viral infections to each other. In some embodiments, such aggregated data is monitored for signs of emerging outbreaks, and used to provide early warning notifications that viral infection is present in a certain location or potential point of contact. For example, such information could be combined with online mapping applications, so that local degree of detected infection is reported as a feature on online maps. In some embodiments, such information may be available as a subscription service.

In some embodiments, distributed test information residing on local information devices, such as cell-phones or personal computers, or personal accounts on a cloud, can be shared by secure private network means, such as block-chain databases and verification, so that such information can be shared for such purposes in a secure manner.

In an exemplary embodiment, the information application supports precision, automated, anonymous contact tracing. In this embodiment, personal testing sites are distributed throughout a population, via virometers that maybe at public sites, or private. An adjacent cell-phone based contract tracing app that checks in with GPS stamps at frequent times. A DNA fingerprint is acquired with the test, along with a time stamp and GPS location of the test. Individuals can fully register into the network by having the tracing app and taking a virometer test, which affiliates their DNA fingerprint with their app identifier. When positive tests are taken, they can be anonymously affiliated with individuals GPS traces via the DNA fingerprint, and then other members of the network can be notified if that have been in proximity to the individual, as per their GPS trace data, before or after the positive test. Such individuals can then be tested by any accessible virometer, and their information is then automatically fed back into the tacking network. This provides for a precision contact tracing network, with tracking of individual identity, but with personal identity remaining anonymous.

In one preferred embodiment, there is a testing device that is a smart device, which will only process a test if a suitable digit code or key is provided, which is unique to once specific instance of the test. In this scenario, the tests can be procured beforehand, for example purchased and stored at home, or at another field site, and this purchase can be made with no physician prescription. Without a digital key, the smart device will not run a test. Then, via a telemedicine session, when an examining Physician decides a test is medically justified for the patient, having access to previously procured tests, they provide the prescription in the form of a unique digital key that will unlock the test. For example, such a key could be a unique alpha-numeric string that may be 5, 10 or more characters long. Or the key could be an optical barcode or QR code. Or the key could be an electronically transmittable binary key.

Such a key, in preferred embodiments, in which the test device is also connected, could be transmitted from the Physician electronically, directly to the smart test device, to unlock the test. In other embodiments, the key is sent to the home/field user, and they enter the key into the smart test device locally. The digital key may be supplied entirely from a database of valid keys or valid key generator available to the physician, or may in other preferred embodiments also require information input from the smart device and test taker, that may relate to their identity and the location and time and type of test. In preferred embodiments, the key could encode the biometrics of the intended user, a time stamp that limits the time during which the test must be run, a location stamp that limits the location in which the test can be run, as well as the permission to run the test. The user in these embodiments must not only supply the key to the smart test device, but also supply the other required information and adhere to the constraints of the key, such as supplying the required biometrics, and doing so in the allowed time window and location range. The smart device may be equipped with GPS location capabilities to support such use cases with these constraints. The multiple constraints and permissions can be encoded into an electronic digital key by many means, such as the use of a hash function or other well-known data encoding and encryption methods.

In one preferred embodiment, in which there is a biometric requirement, the test device may acquire a DNA fingerprint form the test sample, which must match a previously determined DNA fingerprint of the individual, stored on the device or on an App connected to the device. In another preferred embodiment, other biometrics such as fingerprint, voice print, eye scan, or facial recognition may be used to ensure that the intended patient unlocks the test for use.

In one preferred embodiment, the smart testing device would be a molecular electronic virometer device, such as described in Applicant's prior provisional application No. 63/085,874 which is incorporated herein by reference in its entirety. Such a device could have tests for nucleic acids, antigens, or antibodies.

Such a device uses DNA oligo probes to target viral nucleic acids, wherein the oligo is used as a hybridization probe or a primer targeting the viral genome (or amplification products from this genome), or can use DNA oligo probes as aptamers that target proteins relevant to detecting the target the virus, such as the SARS-CoV-2 spike proteins in the case of COVID-19, or aptamers against any other viral antigens.

It is also an object of this disclosure that, in preferred embodiments, the telemedicine prescription may include entirely new content for the smart test device. In one preferred embodiment, this would be new sequences that define hybridization probes for new viruses or viral strains, based on genomic sequences to be detected by hybridization. In another preferred embodiment, this would be new sequences that define new aptamers that can detect novel viral proteins or antigens.

In preferred embodiments, this new content package provided by telemedicine is custom-tailored to have the targets relevant the precise symptoms and risk profile and concerns of the patient, to provide a Precision Medicine diagnostic, that may be uniplex or multiplex, and is targeting the precise set of targets of relevance to the patient. Such targets would be of highest diagnostic value to screen possible causes of the patient symptoms, or to screen possible diseases for which the patient is at high risk, or at a high risk for a negative outcome.

In such preferred embodiments, the smart device may be a molecular electronic ViroMeter in which it is further a field-programmable format, whereby supplying DNA oligos to the device, it can load these oligos as binding probes (in particular, as hybridization probes or primers for nucleic acid targets, or as aptamers for protein or antigen targets) for the molecular electronic sensors on the sensor chip. In this preferred embodiment, the patient or user is assumed to also have a home-use or field-deployed DNA oligo synthesizer, capable of taking a list of oligo sequences in a content package, and synthesizing the corresponding physical DNA molecules, at sufficient scale for programming of the sensor chip. In this use case, the telemedicine Physician not only provide the digital key activating a test, but also provides a content digital data file, that encodes for the production of the needed programming oligos via the at-home or field-use DNA synthesizer companion to the smart test. In this way, entirely new, prescribed content is transferred to the user via a digital information file, and the final diagnostic test is constituted on site using the synthesized DNA oligos and the oligo-programmable sensor chip. In preferred embodiments, this can be used to add content to the prescribed test, as per the judgements of the telemedicine physician, including content such as additional viral targets, novel viral targets, or novel strains or sequence variants of existing viral targets. In preferred embodiments, such content is added based on the precise needs of the patient, to provide a precision medicine diagnostic tailored to the patient.

Exemplary embodiments include but are not limited to the following:

Embodiment 1. A personal virometer, comprising
a. A compact instrument for rapid running of molecular electronics chip,
b. A molecular electronics sensor chip configured for detecting a multiplicity of viral targets
c. A sample applicator for acquiring a sample and transferring it to the chip
d. Data processing software and hardware providing a report out to the operator, on which viral targets were detected, and possibly also the level of detection.

2. The personal virometer of embodiment 1, wherein
a. The instrument has a form factor that is portable, mobile, handheld, or wearable
b. The rapid measurement time is less than an hour, 30 minutes, 15 minutes, 5 minutes, 3 minutes, 1 minute or 30 seconds.
c. The multiplex detection chip detects up 10 targets, up to 100 targets, up to 1000 targets, up to 10,000 targets, or more than 10,000 targets.

3. A personal virometer as in embodiments 1 or 2, where the molecular electronics chip uses a hybridization sensor to detect target viral DNA or RNA fragments, or uses a primer extension sensor to detect such viral DNA or RNA fragments, or uses a antibody or aptamer binding probe sensor to detect viral protein or antigen targets or uses such binding probe sensor to directly detect viral particles.

4. A method for personally testing a test subject or testing target, comprising
a. bringing a personal virometer device to the location of the test subject or testing target,
b. collecting the sample from the subject or target
c. immediately applying the sample to the molecular electronics chip,
d. obtaining the readout from the virometer of the test result

5. A field-programmable personal virometer, comprising a molecular electronics chip with addressable hybridization oligos at each sensor, and specific probe reagents that are conjugated to the complementary oligos of the hybridization oligos on the chip

6. A method of field-programming the molecular electronics chip, comprising:
a. Having a chip with addressable hybridization oligos at each sensor
b. Having specific probes conjugated to the complementary oligos
c. Having a software update specific the resulting specific probes that will reside at each sensor, and specifying any special analysis parameters required for such probes
d. Apply the specific probe reagent to the chip, and uploading the software update to the virometer,
e. Using the updated chip and virometer for virus measurements on a biosample

7. A personal virometer that comprises SARS-CoV-2 virus, strains and variants among its targets, to be used for testing for COVID-19.

8. A personal virometer that comprises SARS-CoV-2 virus, strains and variants among its targets, to be used for testing for COVID-19, used for testing in the home, in schools, in workplaces, in hotels, in restaurants, or in public places.

9. A personal virometer that comprises Influenza virus, strains and variants among its targets, to be used for testing for influenza, used for testing in the home, in schools, in workplaces, in hotels, in restaurants, or in public places.

10. A personal virometer that comprises cold virus, strains and variants among its targets, to be used for testing for influenza, used for testing in the home, in schools, in workplaces, in hotels, in restaurants, or in public places.

11. A method for screening for virus at airports using a personal virometer

12. A method for screening for virus in the home using a personal virometer

13. A network or personal virometers, connected to a common database of results.

14. A method for using Personal virometers to establish a database of viral infection, by uploading and aggregating detection results from many personal virometers, and use this data to track emerging infections, identify high risk areas, or identify emerging strains with different sequences or transmission properties.

15. A personal pathometer, similar to the virometer of the above embodiments, but having targets in a larger class of infectious disease pathogens, which may include viruses, bacteria, fungi or parasites.

16. A personal pathometer configured to test for common sexually transmitted diseases.

17. A personal pathometer configured to test for common food-borne illnesses.

18. Multimodal devices comprising a personal virometer.

19. Multimodal devices comprising a personal pathometer.

20. A method in which a personal virometer also measures a DNA fingerprint of the test subject.

21. A method of secure, verified and identified testing, in which the personal virometer also measures a DNA finger print of the test subject, and affiliates this with the test results, as well as optionally with other data such as time of test, GPS location of test, thereby providing an digital certificate of testing that can be used for verified test results, properly identified test result, or longitudinal affiliation of testing results with the same individual, or for tracing of infections in a population, or in biosecurity settings.

22. A method of access control, in which the personal virometer produces a time-stamped certificate of test results, which is carried on the personal cell phone or smart device of the test subject, and which is required for access control, allowing entry with only recent negative test results, by automated assessment of such certificates by other smart devices, for access to places at risk for the spread of viral infection, such as schools, hospitals, businesses, places of employment, restaurants, theatres, airports, airplanes, travel hubs, trains, sporting events, concerts, hotels, cruise ships, or other gathering sites.

23. A method of personal infection risk control, in which the personal virometer produces a time-stamped certificate of test results, which is carried on the personal cell phone or smart device of the test subject, and with the aid of associated apps, is used by individuals to mutually ensure negative test results prior to meeting in person.

24. A personal virometer wherein the detection assay comprises
a. Extraction of viral genetic material from a biosample
b. Option amplification of genetic material
c. production of single stranded DNA products
d. detection of such products on a hybridization array
e. optionally using electronic target concentration to increase sensitivity

25. A personal virometer wherein the detection assay comprises
a. Extraction of viral protein targets from a biosample
b. detection of such products on an aptamer array
c. optionally using electronic target concentration to increase sensitivity

26. A personal virometer wherein the detection assay comprises
a. Extraction of viral protein targets from a biosample
b. Exposure of such materials to a solution containing detection aptamers with molecular switches
c. Optional amplification of the free molecular switches
d. detection of such switches or such products on a hybridization array
e. optionally using electronic target concentration to increase sensitivity

27. A method for remote, delayed notification of personal virometer results, in which the virometer measurement is initiated, and results are sent to a cell-phone based or other remote app for notification of the test subject.

28. A method for early warning of viral infection outbreak in which personal virometer results are uploaded to a cloud-based database, and positive results are reported along with location information on viral tracking apps on cell phone devices or computers in the broader population.

29. A method for a contact tracing network, in which personal virometers automatically capture DNA fingerprints, and report test results and fingerprints to a central database, along with GPS and time stamp markers, and in which user cell-phone apps or smart device apps report user GPS and time stamps at frequent intervals, and such users are notified of a positive virometer result has been recorded by a user who has had proximate GPS and time stamp locations, and where notified users take a personal virometer reading, to increase the information content of the tracing network.

30. A personal virometer using electronic target concentration to increase sensitivity.

31. A personal virometer using an assay for aptamer targets, using a multiplicity of aptamers with switch oligos in solution, and detection of switch oligos on a hybridization on the sensor array chip, and which may comprise amplification of the switch oligos prior to detection.

32. A personal virometer for home use in which samples from a family are pooled and tested in a single test.

33. A personal virometer in which the sensor chip is flushed and re-used for multiple tests, with blank negative test results run to qualify the re-use.

34. A method of daily screening a family for viral infection, at reduced cost and time, in each morning, the family saliva samples are pooled into the virometer, and tested as a pool, providing back one test result for the entire family, and in the case of a negative, all family members are recorded as negative, while in the case of a positive, each individual is subsequently tested separately to identify which individuals are positive.

35. A method for immediate testing using a prescription diagnostic test, in which

a. A smart test device and consumable test kits are provided for pre-procurement by users, without prescription

b. Upon demonstration of medical need via a telemedicine session, a doctor provides the user a digital prescription for the kit, and a digital access key to activate the test, all transmitted via telemedicine means

c. The user unlocks the test, and performs the test on themselves, and receives an immediate answer, as rapidly as the test can provide.

36. The method of Embodiment 35, where the smart test device and kit comprise a molecular electronics chip and associated smart instrument platform.

37. The method of Embodiment 35, where the prescribed smart test device and kit are used to enable at-home testing for viral infections.

38. The method of Embodiment 35, where the prescribed smart test device and kit are used to enable at-home testing for COVID-19.

39. The method of laim 35, where the prescribed smart test device and kit are used to enable at-home multiplex testing for COVID-19, and also comprising multiple strains of COVID-19, tests for multiple strains of influenza, and tests for multiple cold viruses such as RSV.

40. A method for immediate testing using a prescription diagnostic test with new content, in which
a. A smart test device and consumable test kits that are DNA oligo-programmable, are provided for pre-procurement by users, without prescription
b. Users further have a suitable smart device for on-site synthesis of DNA oligos or oligo pools.
c. Upon demonstration of medical need via a telemedicine session, a doctor provides the user a digital prescription for the kit, and a digital access key to activate the test, and a digital sequence content file specific for the needs of the user, that is used to define the oligo synthesis, and all are transmitted via telemedicine means
d. The provided oligo sequences are synthesized and used to program the oligo-programmable test device
e. The user unlocks the new test, and performs the test on themselves, and receives an immediate answer, as rapidly as the test can provide.

41. The method of Embodiment 40, where the smart test device and kit comprise a molecular electronics chip and associated smart instrument platform, and such chips have oligo-programmable binding sensors, such that the programming oligos conjugate into the circuits, at known pixel locations on the chip, and provide the required binding probes.

42. The method of Embodiment 40, where the prescribed smart test device and kit are used to enable at-home testing for viral infections via nucleic acid or protein antigen other antigen targeting, using DNA oligos as binding probes for such targets/43.

43. The method of Embodiment 40, where the prescribed smart test device and kit are used to enable at-home testing for COVID-19, either based on nucleic acids or proteins, and emerging new strains or variants of virus, that require updated content.

44. The method of Embodiment 40, where the prescribed smart test device and kit are used to enable at-home multiplex testing for COVID-19, and also comprising multiple strains of COVID-19, tests for multiple strains of influenza, and tests for multiple cold viruses such as RSV, for tests that require new content to deal with the appropriate set of test targets for the patient, and where such targets are detected either through nucleic acid detection using hybridization DNA oligo probes, or through protein testing, such as the spike protein, o other viral antigens, via aptamer DNA oligo probes.

45. The method of 40, where the content package is selected based on the symptoms, risks and concerns of the patient, to provide a Precision Medicine Diagnostic test.

46. The method of embodiment 40, used to provide a Precision Medicine Diagnostic tailored to symptoms, risks and concerns of the patient.

47. A custom Precision Medicine Diagnostic, provided by such a programming oligo pool, and that has been specified via an oligo digital definition package tailored to symptoms, risks and concerns of the patient.

### Definitions and Interpretation

As used herein, the term “DNA” refers generally to not only to the formal meaning of deoxyribonucleic acid, but also in contexts where it would makes sense, this term also encompasses the well-known nucleic acid analogs of DNA that are used throughout molecular biology and biotechnology, such as RNA, or RNA or DNA that comprises modifications such as bases with chemical modifications, such as addition of conjugation groups at the 5′ or 3′ termini or on internal bases, or which includes nucleic acids analogues, such as PNA or LNA. DNA may generally refer to double stranded or single stranded forms as well in contexts where this makes sense, and unless specifically designated. In particular, when referring to hybridization and the probes and targets for this as DNA, they are interpreted in this broader sense of any of these analogs which undergo hybridization to form a bound duplex.

As used herein, the term “hybridization” or “DNA hybridization” refers to the process by which a single stranded segment of DNA in solution pairs with its reverse complement sequence to form a duplex molecule via Watson-Crick base pairing, and forming a double helical segment. It is understood here this includes the cases of DNA-RNA pairs forming, RNA-RNA pairs forming, and that such DNA could also include modified bases or nucleic acid analogs such as PNA or LNA. It is understood this pairing can occur between single strands of different length, such pairing occurring between the complementary segments of these longer sequences.

As used herein, the terms “complement”, “match”, “exact match” and “reverse complement” of a given segment of single stranded DNA or RNA all refer another single strand of DNA or RNA that will hybridize properly with this strand to form a duplex with Watson-Crick base pairings (and base pairing U-A for RNA-DNA or RNA-RNA pairings, as RNA has Uracil (U) instead of Thymine (T)) for the segment of interest.

As used herein, the term “hybridization probe” refers to a specific segment of DNA (or RNA) that is to be used to bind a complementary strand of interest. Such a strand of interest may exist within a sample or complex pool of known or unknown DNA or RNA fragments, or a diverse set of oligos presented in a solution environment that allows for the hybridization reaction. This term also may refer to the segment that will be anchored in place for exposure to the test sample solution. In context, the hybridization probe may refer to the single molecule of interest, or to a quantity of such molecules that all have the same sequence. A hybridization probe in many instances may be a short segment of DNA, in the range of 10-100 bases, but in general can be a DNA strand of any length. As used herein, the hybridization probe may generally refer to a DNA segment for which only a portion of it is used to hybridize to a target of interest, and other portions of it may serve difference purposes, such as spacers, segments comprising conjugation sites, segments intended to hybridize to other distinct targets, segments intended to bind DNA primers, or sites for binding of decoding probes use to produce location maps for the sensor on a chip, including segments that are sites for hybridization to targets that are decoding probes that are DNA hybridization oligos, including such oligos used for combinatorial decoding, which oligos which may be otherwise labelled or unlabeled with additional signaling groups to aid in decoding. As used herein, the primers for extension assays are also a type of hybridization probe, and in contexts where it makes sense, hybridization probe can be taken to include such primers as well.

As used herein, the term “decoding probe” generally refers to any molecule whose binding and subsequent detection is used for a process of determining the location map of where hybridization probes for different targets are located on a sensor pixel array. In this context, it is assumed there are a multiplicity of different types of DNA hybridization probes, having different target DNA as defined by the probe sequences, and that molecules of these types have been randomly assembled into a sensor pixel array, or otherwise placed in such a way that their location in the pixel array is unknown. It this context, each hybridization probe is assumed to have physically linked or connected to it, one or more binding sites that would bind to one or more of the decoding probe molecules. The series of decoded probes are applied to such an array in series or in pooled form, allowed to bind to their specific targets on the hybridization probes, and the bound state is read out using the detectable signal generated by the binding probes. Such binding probes in some embodiments are single stranded DNA oligo hybridization probes, with hybridization targets on or linker to the DNA hybridization probes on the array. In some embodiments, the detectable signal is the electrical hybridization signal measurable by the sensor. In other some embodiments, dye labels on such probes could be read out with an optical microscope imaging system. Other embodiments could use binding probes that are not based on DNA hybridization, such as using aptamers or antibodies or libraries of small molecules.

As used herein, the term “combinatorial decoding” generally refers to any process of decoding the location map of hybridization probes on an array, where a series of outcomes of multiple decoding probe binding reactions is used to generate a unique identifier “barcode” for the array probes, that determines the hybridization probe identity.

As used herein, the term “hybridization target” means DNA or RNA molecules which contain the complement of a DNA hybridization probe. Such a target could be the exact complementary strand, but in most cases it will be a longer strand that contains a segment exactly complementary to the probe. In the context of discussing a target with mismatches, it refers to molecules which match to the probe except at one or more bases, as indicated. In the context of hybridization, “perfect match” means a sequence that correctly hybridizes to the probe, with no mispaired bases, while a “mismatch” refers to a sequence that may bind to the probe, but has one or more mispaired bases, i.e. bases not engaged in the standard Watson-Crick bond found in natural double helix DNA-DNA or double helix DNA-RNA pairings. Such incomplete pairing will have reduced stability compared to the perfect match binding, which can be generally be used to discriminate perfect matches from mismatched forms—also known as cross-hybridization—in assay methodologies.

As used herein, the “hybridization assay” means any assay or test that comprises the process of hybridization.

As used herein, the term “sample” or “biosample” refers to any material that is intended for testing. Such material could be in solid or liquid form, and may also generally be in some form of container, such as a tube, and/or reside a carrier medium such as a swab or filter paper. Such material could comprise tissue, cells, bodily fluids, excrement, food products, portions of plants, of materials collected by a swab, air filter or water filter. Such material may also be maintained with some form of preservative or stabilizing agents. These terms may refer to the material in the state as initially collected, or materials that have undergone process steps, such as to extract or amplify DNA or RNA, prior to being in a form suitable to introduce to the sensor device.

As used herein, the term “PCR” refers broadly to any methods that use polymerase or reverse-transcriptase reactions to produce multiple copies of sequences from source DNA or RNA. In this context, the term “copies” may in general refer to single stranded reverse complements of segments of the source molecule, or single stranded exact copies of segments of the source molecule, or double stranded forms where one strand is identical to a segment of the source molecule. The term “copies” also may refer to the product of methods where an RNA template is converted to DNA molecules of the corresponding sequence, or a DNA template is converted to RNA molecules of the corresponding sequence. Such “PCR” methods in this context may include methods with linear amplification or exponential amplification, relative to time or cycle numbers. Such methods include those that use specific primers, or degenerate primers. Such methods also include isothermal reactions that occur in continuous time, or reactions that rely on thermal or chemical cycling. The “PCR” process may produce copies of specific target segments of the source DNA or RNA, as defined by specific primers, or may produce copies from many sites or random sites, as may result from degenerate primers. In particular, “PCR” in this connect may refer to isotheral amplification methods that can be used to rapidly produce large amounts of DNA copy fragments from a source genome, of RNA or DNA, using one of the many well-known methods, such as Rolling Circle or RCA, Genomify, or LAMP, and with such a method incorporating a reverse-transcriptase in the case of RNA starting material.

As used herein, “amplification” of DNA or RNA in a sample material refers to the use of PCR methods such as above, to make copies of the source DNA or RNA.

As used herein, “pathogen” refers to any disease-causing agent that has a genome, such as parasites, fungi, viruses, or bacteria, or other single or multicellular organisms that cause disease.

As used herein, “strain” refers the genetic variants within a species, i.e. members of the same species that have genomes that difference in sequence.

As used herein, “molecular electronics” refers to devices in which a single molecular or single molecular complex is integrated into an electronic circuit.

As used herein, a “molecular electronics sensor” is a device that transduces molecular interactions with target molecules in solution into electronic signals, using a single molecule or molecular complex integrated into an electrical circuit as the primary transduction mechanism.

As used herein, a “molecular complex” refers to small number of molecules that are held together by chemical conjugation, bioconjugation, or covalent or non-covalent bonds, such that the assembly is expected to retain this configuration or affiliation during the process of assembling it onto nanoelectrodes, and during use of the resulting sensor in assays. Such small number of molecules may be just two, such as a DNA oligo probe bound to a bridge molecule, but in other contexts may be in the range of 2-10, 10-100, or 100-1000.

As used herein, “nanoelectrodes” are conducting elements that define a nanometer scale gap, and have dimensions of nanometer scale height and width, and substantially longer length, which provide an electrical conducting connection into a circuit.

As used herein, “bridge” or “bridge molecule” refers to any type of molecular wire or conducting molecule than may be using to make a conducting connection across the gap between nanoelectrodes. Such molecules include double stranded DNA, peptide alpha helices, graphene nanoribbons, pilin filaments or bacterial nanowires, other multichain proteins or conjugates of multiple single-chain proteins, antibodies, Carbon nanotubes, or conducting polymers such as PDOT. Such molecules may include attachment groups that provide for specific attachment to, and/or self-assembly to, the nanoelectrode contacts.

As used herein, “semiconductor chip” refers to an integrated circuit chip comprising semiconductor materials such as Silicon or Gallium, and fabricated with techniques from the semiconductor industry.

As used herein, “CMOS chip” refers to an integrated circuit chip, fabricated using CMOS process techniques from the semiconductor industry. CMOS is an acronym for Complementary Metal-Oxide Semiconductor, and refers to a specific manufacturing process for making integrated circuit chips of the type most produced for processors, DRAM memory, and digital imager devices. As used herein, “CMOS chip” also refers to a device fabricated at the foundries that make such chips in industry, but which may also be postprocessed for purposes of the inventions disclosed here if, using processes to adding or exposing accessible nanoelectrodes, an suitably protecting such nanoelectrodes, for use in the molecule electronics sensors.

As used herein, the term “chip” used in isolation refers to a “semiconductor chip” or “CMOS chip”.

As used herein, the term “pixel” refers to a sensor and measurement circuit that is repeated throughout a regular rectangular array of such identical circuits on a chip. A pixel may in context refer to just the measurement circuit, which here is a form of current meter measuring circuit, or may also include the sensor transducer element or elements affiliated with the circuit, which here are the molecular electronic component, i.e. molecule attached to nanoelectrodes. For definiteness, the term “measurement pixel” as used herein refers to the measurement circuitry of the pixel, and the term “sensor pixel” refers to the pixel circuit affiliated with a given sensor element. The origins of this term come from image sensors, where such pixels contained light sensing elements and measurement circuitry, which captured an element of a picture, but in the present context, as used herein the term pixel is unrelated to light sensing or imaging, and the pixels disclosed herein are sensing chemical interactions, not light.

As used herein, the term “sensor” refers to the complex consisting of the nanoelectrodes, bridge molecule and hybridization probe, which is the primary transducer of interactions of the hybridization probes to electrical signals. In contexts where it makes sense, sensor could also refer to this plus the supporting current measurement circuitry, such as the including the pixel circuits. “Sensor pixel” refers to the pixel circuitry that provides measurements to a particular sensor.

As used herein, the term “primer sensor” or “primer extension sensor” refers to a molecular electronic sensor, with a primer attached to the bridge, suitable for performing primer extension detection.

As used herein, the term “signal group” or “signal enhancing group” refers to a chemical group that could be added to an oligo, and such that the presence of this group complexed into the probe-bridge complex, versus dissociation from this complex, produces a detectable signal. In particular, such a group may be displaced from the critical position by target probe binding, or may be brought into proximity as a label on the target strand.

As used herein, the term “secondary structure” refers to the physical conformation that a DNA strand takes in response to bonds it forms with itself or other molecules. In particular, this includes the structures that form from hybridization between portions of a DNA molecule, or between two DNA molecules. This also includes structure that may result from the DNA strand interacting with the bridge. Secondary structure can be induced by hybridization binding, and other forms of binding.

As used herein, the term “primer” refers to a DNA strand that hybridizes to another single strand, and provides a free 3′ end at a double-single strand junction that can serve as a site for polymerase extension to occur. A perfect-match refers to a primer that is properly based paired in biding a target, whereas a mismatched or off-target primer refers to one that is bound, but with one or more mis paired bases with the template.

As used herein, the term “primer probe” refers to a primer that is, or is intended to be, attached to a bridge in a molecular electronics sensor.

As used herein, the term “template” refers to a single stranded DNA that is to be used as the substrate for a polymerase extension reaction. It may refer in context to the entire physical strand, or the portion of the strand that directly engages in primer binding and extension.

As used herein, the term “primed template” refers to a single stranded DNA that has a primer bound to it.

As used herein, the term “primer binding” means the hybridization of a primer to its target site on a template DNA. In general, this may either refer to exact match binding, i.e. the primer binding to its reverse complement sequence on the template, or may mean binding with mismatched bases, which may still be stable if the melting point Tm is near or lower than the reaction temperature. In contexts where the distinction is important, such perfect match versus mismatch binding will be specified.

As used herein, the term “similar sequence” means two sequences with a high degree of DNA sequence homology or DNA sequence similarity, or which could hybridize with a melting point substantially higher than what would occur between to random sequences of the same lengths. In particular, in reference to a primer binding to target site versus similar off-target sites, it refers to similar sites where the primer could bind with a melting point much closer to the perfect match Tm than would occur at random sites, such as being within 2 C, 5 C, 10 C or 15 C of the perfect match Tm.

As used herein, the term “polymerase binding” means the attachment of a polymerase at the 3′ end of a primer bound to a template, resulting in the precursor state for primer extension.

As used herein, the term “primer extension” means the process in which a polymerase binds to a primer site, and synthesizes a strand complementary to the template strand.

As used herein, the term “primer extension reaction” or “primer extension assay” refers to a biochemical reaction or assay which comprises a primer extension process.

As used herein, the term “polymerase” refers broadly to any of the enzymes that can synthesize DNA or RNA segments, of length one or more bases, starting from primed DNA or RNA templates. This includes, in contexts where it makes sense, DNA polymerases that synthesize a DNA strand from a primed DNA template. Well known examples of this include DNA polymerases such as Taq, Bst, Bsu, Klenow, Pfu, Vent, Phi29, and Nine Degrees North, and genetically modified forms of these naturally enzymes, which also occur under a variety of commercial trademark names. This also includes, in contexts where it makes sense, Reverse Transcriptases that take an RNA substrate and synthesis DNA. Well known examples of these include Avian Myeloblastosis Virus (AMV) and Moloney Murine Leukemia Virus (MMLV) Reverse Transcriptases. This also includes, in contexts where it makes sense, enzymes that synthesize RNA strands from a DNA template, such as T7 RNA polymerase. This also includes, in contexts where it makes sense, enzymes that synthesize RNA strands from RNA templates. These are known as RNA dependent RNA polymerases (RdRp), and well know examples are polio virus 3DPol, and COVID-19 RdRp. It is also understood, in context, that in cases involving RNA, the U base occurs instead of T, U forms Watson-Crick base pairing with T, and the synthesis of RNA utilizes dUTP instead of dTTP. In particular, in discussion of polymerase priming and extension, reference to DNA, T and dTTP are meant to, in context where it would make sense, encompass RNA, U and dUTP.

As used herein, the term “allele” refers to a variant forms of a particular DNA segment that occur within in a species. This is a well-known term from genetics. As used herein, the term “locus” refers to a segment or location in the genome having multiple alleles, i.e. multiple different sequence variants that may be observed within a species.

As used herein, the terms “allele specific primer binding”, refers to the situation where there are a given set of two or more possible alleles at a given locus, and there is a primer that properly binds one of the alleles, and has a mis-match of its 3′ end at the similar sequence sites of all of the other alleles. For example, if the alleles were 5′-GATTACA-3′ and 5′-GACTACA-3′, then the primer 3′-AATGA-5′ would be specific for the first allele (binding site bases 3-7), while having the 3′ end mismatch at the T at base 3 in the second allele. The allele specific primer may have more mismatches than at its 3′ end, but the 3′ end mismatch is the essential one in this context.

As used herein, the term “allele specific extension”, refer to the situation where a polymerase extension is performed when priming is done with an allele specific primer, so that extension will only occur on a template that has the allele specifically matching the primer. Allele specific extension in particular refers to processes that rely on the mismatch at the 3′ end of the primer for specificity.

As used herein, the term “universal primer” means a single primer sequence that has targets on many different sequence fragments. For example, such a universal primer site may be added as a form of sample preparation, to diverse fragments in a sample.

As used herein, the term “Field-deployed” test or “Field” test means a test or testing device that is used outside of the a clinical or research testing laboratory setting. This includes use at sites such as in the home, businesses, workplaces, schools, restaurants, arenas, stadiums, concert venues, airports, transportation hubs, on board transportation, and all sites that are out of doors. It also includes sites to which the military are deployed, for use by military personnel. It includes border check-points, or point-of-entry or port-of-entry sites. Field deployment may also refer to all use cases in which the operator of the test is not a professional lab worker, or a professionally trained and certified testing technician.

As used herein, the term “At-home” test refers to a test that is done within a family home or residential setting, or in the vicinity of such places.

As used herein, the term “Point-of-use” test refers to a test that can be performed entirely at the location at which the primary sample is collected. This is in contrast to tests in which a sample is collected, and transported to a different location, typically a centralized lab facility, to perform the remainder of the test.

As used herein, the term “DNA fingerprint” refers to a set of genetic marker values that can distinguish between DNA samples from two individuals with high confidence. Typically, such marker sets are chosen so that the probability that two unrelated individuals would have the same marker values is extremely small, such as less than 1 in 1 million, 1 in 1 billion, or 1 in 1 trillion. By extension, these marker sets can distinguish individuals, by means of testing their DNA for their marker values. Such marker sets can also distinguish between closely related individuals, such as siblings or parents and their children, however they cannot distinguish between identical twins. Common marker sets are sets of variable length repeat sites in the genome, such as the widely known CODIS marker set, used by the FBI and law enforcement for human identification in criminal justice and forensics applications. This is a set of 13 tandem repeat markers. Other common marker sets are based on SNP markers. Panels of 10-100 or more such markers, distributed across the genome, and properly selected for being independent and variable in the population, can be highly informative as a DNA fingerprint. Many such SNP marker panels are possible, as there are millions of known SNP sites in the human genome.

As used herein, the term “aptamer” refers to a DNA or RNA oligomer, or peptide oligomer, which has specific, high binding affinity to a target molecule or molecular structure, and much lower affinity to the broader class of molecules to which it may be exposed. Such aptamers are typically chosen by screening a much larger and diverse set of oligomers against the target of interest, and against specific off-target materials, to select ones which preferentially bind to the target, and which have no, or much lower, binding affinity to the off-target materials. DNA or RNA aptamers are commonly in the range of 20 to 100 bases long. Peptide aptamers may typically be shorter, in the range of 5-20 amino acids long. As used herein, it is understood that in any context where an antibody is mentioned, to the extent it makes sense, the same comments are implied for aptamers as well.

As used herein, the term “antibody” refers to any of the Immunoglobin (Ig) recognition molecules produced by animal immune systems, to bind to specific foreign materials present in the blood of the animal. In particular this include molecules of the type IgG, IgM, IgA, IgD, or IgE. It includes such Ig molecules produced by any animals, such as mice, rabbits, rats, guinea pigs, sheep, or camels. It also includes such molecules that are produced by cell cultures derived from the immune cells of such animals. As used herein, “antibody” also generally refers includes synthetic versions or variations of such molecules, or any modified forms of any such molecules, including those made by genetic or chemical modification. As used herein, it is understood that in any context where an aptamer is mentioned, to the extent it makes sense, the same comments are implied for antibodies as well.

As used herein, the term “antigen” refers to the binding target of an antibody. As used herein, the term antigen may also be taken to refer to the target of an aptamer, to the extent this makes sense. In this general context, an “antigen test” is to be interpreted as a test using an antibody, or an aptamer, to target an antigen.

As used herein, the term “smart” or “smart device” refers to an electronic device that is capable of electronically receiving information, and of processing such information to make decisions such as whether or not to run a test.

As used herein, the term “connected” or “connected device” refers to an electronic device that is capable of electronically sending and receiving information, via a cell phone, the internet, or other electronic communication means.

As used herein, the term “test” may refer to a nucleic acid test, antigen test or antibody test, whenever such interpretations make sense. As used here, when it makes sense, the term test may refer to just the consumables for a single test, or the combination of these consumables and any associated device that runs the test.

As used herein, the term “at-home” refers to a test that can be run entirely from the home, from sample collection through return of results.

As used herein, the term “in the field” or “field deployed” refers to a test that can be run entirely from a site that is remote and need not be a clinical lab, such as a place of business, a school, an airport, on a plane, at a remote job o test site, or in a military battle theater.

As used herein, the term “telemedicine” refers accessing medical advice or guidance or evaluation from a medical professional, via a communications channel, such as a phone, cell phone, teleconference, or internet connection, and in contrast to being in close proximity to the medical professional and engaging by direct, in-person communication, such as in the same room, office, or clinic.

1. A portable virometer, comprising
a. a molecular electronics sensor comprising a first electrode; a second electrode spaced-apart from the first electrode by a nanogap; a bridge molecule having a first end and a second end, the first end coupled to the first electrode and the second end coupled to the second electrode; and a hybridization probe having an oligonucleotide sequence conjugated to the bridge molecule; wherein the oligonucleotide sequence is selected from SEQ ID NO:5, SEQ ID NO:6, SEQ ID NO:7, SEQ ID NO:8, or a hybridization probe having a sequence at least 90% sequence identity to one of the preceding;
b. a sample applicator for acquiring a sample and transferring it to the chip; and
c. data processing software and hardware for providing a report of detection of viral targets.

2. The portable virometer of claim 1, wherein the virometer comprises a plurality of sensor pixels configured in an array on a semiconductor chip and each sensor pixel in the plurality of sensor pixels comprises a molecular electronics sensor.

3. A portable virometer of claim 1, where the molecular electronics chip uses a hybridization sensor to detect target viral DNA or RNA fragments, or uses a primer extension sensor to detect such viral DNA or RNA fragments, or uses a antibody or aptamer binding probe sensor to detect viral protein or antigen targets or uses such binding probe sensor to directly detect viral particles.

4. The portable virometer of claim 1, wherein the virometer is field-programmable and comprises a molecular electronics chip with addressable hybridization oligos at each sensor, and specific probe reagents that are conjugated to the complementary oligos of the hybridization oligos on the chip.

5. The portable virometer of claim 1, wherein the hybridization probe has a sequence having at least 95% sequence identity to one or more of SEQ ID NO:5, SEQ ID NO:6, SEQ ID NO:7, and SEQ ID NO:8.

6. The portable virometer of claim 1, wherein the hybridization probe has a sequence having at least 98% sequence identity to one or more of SEQ ID NO:5, SEQ ID NO:6, SEQ ID NO:7, and SEQ ID NO:8.

7. The portable virometer of claim 1, wherein the hybridization probe comprises one or more of SEQ ID NO:5, SEQ ID NO:6, SEQ ID NO:7, and SEQ ID NO:8.

8. A portable virometer of claim 1, using electronic target concentration to increase sensitivity.

9. A portable virometer of claim 1, using an assay for aptamer targets, using a multiplicity of aptamers with switch oligos in solution, and detection of switch oligos on a hybridization on the sensor array chip, and optionally comprising amplification of the switch oligos prior to detection.

10. A portable smart device molecular electronics sensor device comprising: a first electrode; a second electrode spaced-apart from the first electrode by a nanogap; a bridge molecule having a first end and a second end, the first end coupled to the first electrode and the second end coupled to the second electrode; and a hybridization probe having an oligonucleotide sequence conjugated to the bridge molecule; wherein the oligonucleotide sequence comprises a target sequence of interest; a sample applicator for acquiring a sample and transferring it to the portable smart device molecular electronics sensor; and data processing software and hardware for determining and providing an immediate telemedicine session with a doctor or health care professional based upon a report generated by the portable smart device molecular electronics sensor based upon the of detection of a particular target sequence.

11. A portable smart device molecular electronics sensor device of claim 10, wherein the target sequence is a virus.

12. A portable smart device molecular electronics sensor device of claim 10, wherein the target sequence one or more strain of COVID-19, influenza, and cold viruses (e.g., RSV and rhinovirus).

13. A portable smart device molecular electronics sensor device of claim 10, wherein the target sequence is selected from SEQ ID NO:5, SEQ ID NO:6, SEQ ID NO:7, SEQ ID NO:8, a sequence at least 90% sequence identity to one of the preceding, or a complementary sequence to any of the preceding.

14. A portable smart device molecular electronics sensor device of claim 10 for home use.

15. A method for personally testing a test subject or testing target, comprising:
a. providing a portable smart molecular electronics sensor device of claim 10 to the location of the test subject or testing target;
b. collecting the sample from the subject or target;
c. immediately applying the sample to the sample applicator;
d. obtaining a readout, results, or recommendation from the portable smart molecular electronics sensor device of the test result; and
e. having a telemedicine session with a doctor or health care professional if the results or recommendation from step d) indicate a need.

16. A method according to claim 15, wherein the target sequence is a virus.

17. A method according to claim 15, wherein the target sequence one or more strain of COVID-19, influenza, and cold viruses (e.g., RSV and rhinovirus).

18. A method according to claim 15, wherein the target sequence one or more strain of COVID-19 and the method is for in home use.

19. A method according to claim 15, wherein the target sequence is selected from SEQ ID NO:5, SEQ ID NO:6, SEQ ID NO:7, SEQ ID NO:8, a sequence at least 90% sequence identity to one of the preceding, or a complementary sequence to any of the preceding.

20. A method according to claim 15, wherein the target sequence is selected from SEQ ID NO:5, SEQ ID NO:6, SEQ ID NO:7, SEQ ID NO:8, a sequence at least 95% sequence identity to one of the preceding, or a complementary sequence to any of the preceding.


SEQUENCE LISTING (71 sequences)

SEQ ID NO: 1 - protein, 227 aa, Artificial Sequence
  Description of Artificial Sequence Synthetic polypeptide
QQSWPISGSGQQSWPISGSGQQSWPISGSGAEAAAREAAAREAAAREAAAREAAAREAAAREAAAREAAAREAAAREAAAREAAAREAAAREAAAREAAAREAAAREAAAREACAREAAAREAAAREAAAREAAAREAAAREAAAREAAAREAAAREAAAREAAAREAAAREAAAREAAAREAAAREAAAREAAARAGSGQQSWPISGSGQQSWPISGSGQQSWPIS

SEQ ID NO: 2 - DNA, 19 bp, Artificial Sequence
  Description of Artificial Sequence Synthetic oligonucleotide
acgtagcaggtgacaggtt

SEQ ID NO: 3 - DNA, 14 bp, Artificial Sequence
  Description of Artificial Sequence Synthetic oligonucleotide
aacctgtcacctgc

SEQ ID NO: 4 - DNA, 45 bp, Artificial Sequence
  Description of Artificial Sequence Synthetic oligonucleotide
cgatcaggccttcacagaggaagtatcctgtcgtttagcataccc

SEQ ID NO: 5 - DNA, 88 bp, Artificial Sequence
  Description of Artificial Sequence Synthetic oligonucleotide
gcaatggtacggtacttccgggatgcggaaactggctaattggtgaggctggggcggtcgtgcagcaaaagtgcacgctacttgctaa

SEQ ID NO: 6 - DNA, 14 bp, Artificial Sequence
  Description of Artificial Sequence Synthetic oligonucleotide
gaccgccccagcct

SEQ ID NO: 7 - DNA, 51 bp, Artificial Sequence
  Description of Artificial Sequence Synthetic oligonucleotide
cagcaccgaccttgtgctttgggagtgctggtccaagggcgttaatggaca

SEQ ID NO: 8 - DNA, 15 bp, Artificial Sequence
  Description of Artificial Sequence Synthetic oligonucleotide
cccgcaattacctgt

SEQ ID NO: 9 - protein, 14 aa, Artificial Sequence
  Description of Artificial Sequence Synthetic peptide
MHGKTQATSGTIQS

SEQ ID NO: 10 - protein, 7 aa, Artificial Sequence
  Description of Artificial Sequence Synthetic peptide
QQSWPIS

SEQ ID NO: 11 - protein, 5 aa, Artificial Sequence
  Description of Artificial Sequence Synthetic peptide
EAAAR

SEQ ID NO: 12 - protein, 30 aa, Artificial Sequence
  Description of Artificial Sequence Synthetic polypeptide
QQSWPISGSGQQSWPISGSGQQSWPISGSG

SEQ ID NO: 13 - DNA, 13 bp, Artificial Sequence
  Description of Artificial Sequence Synthetic oligonucleotide
ataggacaccgtt

SEQ ID NO: 14 - DNA, 13 bp, Artificial Sequence
  Description of Artificial Sequence Synthetic oligonucleotide
aacggtgtcctat

SEQ ID NO: 15 - DNA, 13 bp, Artificial Sequence
  Description of Artificial Sequence Synthetic oligonucleotide
ttgccacaggata

SEQ ID NO: 16 - DNA, 13 bp, Artificial Sequence
  Description of Artificial Sequence Synthetic oligonucleotide
tatcctgtggcaa

SEQ ID NO: 17 - DNA, 45 bp, Artificial Sequence
  Description of Artificial Sequence Synthetic oligonucleotide
gggtatgctaaacgacaggatacttcctctgtgaaggcctgatcg

SEQ ID NO: 18 - DNA, 20 bp, Artificial Sequence
  Description of Artificial Sequence Synthetic oligonucleotide
cctctgtgaaggcctgatcg

SEQ ID NO: 19 - DNA, 19 bp, Artificial Sequence
  Description of Artificial Sequence Synthetic oligonucleotide
cctctgtgaaggcctgatc

SEQ ID NO: 20 - DNA, 18 bp, Artificial Sequence
  Description of Artificial Sequence Synthetic oligonucleotide
cctctgtgaaggcctgat

SEQ ID NO: 21 - DNA, 17 bp, Artificial Sequence
  Description of Artificial Sequence Synthetic oligonucleotide
cctctgtgaaggcctga

SEQ ID NO: 22 - DNA, 16 bp, Artificial Sequence
  Description of Artificial Sequence Synthetic oligonucleotide
cctctgtgaaggcctg

SEQ ID NO: 23 - DNA, 15 bp, Artificial Sequence
  Description of Artificial Sequence Synthetic oligonucleotide
cctctgtgaaggcct

SEQ ID NO: 24 - DNA, 20 bp, Artificial Sequence
  Description of Artificial Sequence Synthetic oligonucleotide
tacttcctctgtgaaggcct

SEQ ID NO: 25 - DNA, 20 bp, Artificial Sequence
  Description of Artificial Sequence Synthetic oligonucleotide
caggatacttcctctgtgaa

SEQ ID NO: 26 - DNA, 20 bp, Artificial Sequence
  Description of Artificial Sequence Synthetic oligonucleotide
aacgacaggatacttcctct

SEQ ID NO: 27 - DNA, 20 bp, Artificial Sequence
  Description of Artificial Sequence Synthetic oligonucleotide
tgctaaacgacaggatactt

SEQ ID NO: 28 - DNA, 20 bp, Artificial Sequence
  Description of Artificial Sequence Synthetic oligonucleotide
gggtatgctaaacgacagga

SEQ ID NO: 29 - DNA, 20 bp, Artificial Sequence
  Description of Artificial Sequence Synthetic oligonucleotide
cctcagagtacggcagatcg

SEQ ID NO: 30 - DNA, 20 bp, Artificial Sequence
  Description of Artificial Sequence Synthetic oligonucleotide
cctctctcatgccgtcatcg

SEQ ID NO: 31 - DNA, 20 bp, Artificial Sequence
  Description of Artificial Sequence Synthetic oligonucleotide
cctcagagaaggcctgatcg

SEQ ID NO: 32 - DNA, 20 bp, Artificial Sequence
  Description of Artificial Sequence Synthetic oligonucleotide
cctctgtgtacgcctgatcg

SEQ ID NO: 33 - DNA, 20 bp, Artificial Sequence
  Description of Artificial Sequence Synthetic oligonucleotide
cctctgtgaagggcagatcg

SEQ ID NO: 34 - DNA, 20 bp, Artificial Sequence
  Description of Artificial Sequence Synthetic oligonucleotide
cctcagagaagggcagatcg

SEQ ID NO: 35 - DNA, 20 bp, Artificial Sequence
  Description of Artificial Sequence Synthetic oligonucleotide
cctctgtgatgccgtcatcg

SEQ ID NO: 36 - DNA, 20 bp, Artificial Sequence
  Description of Artificial Sequence Synthetic oligonucleotide
cctcagagtacgcctgatcg

SEQ ID NO: 37 - DNA, 20 bp, Artificial Sequence
  Description of Artificial Sequence Synthetic oligonucleotide
ccagtgtgaaggcctgtacg

SEQ ID NO: 38 - DNA, 20 bp, Artificial Sequence
  Description of Artificial Sequence Synthetic oligonucleotide
ccagagtgaaggcctgatcg

SEQ ID NO: 39 - DNA, 20 bp, Artificial Sequence
  Description of Artificial Sequence Synthetic oligonucleotide
cctctcacaaggcctgatcg

SEQ ID NO: 40 - DNA, 20 bp, Artificial Sequence
  Description of Artificial Sequence Synthetic oligonucleotide
cctctgtgttcgcctgatcg

SEQ ID NO: 41 - DNA, 20 bp, Artificial Sequence
  Description of Artificial Sequence Synthetic oligonucleotide
cctctgtgaagcggtgatcg

SEQ ID NO: 42 - DNA, 20 bp, Artificial Sequence
  Description of Artificial Sequence Synthetic oligonucleotide
cctctgtgaaggccacttcg

SEQ ID NO: 43 - DNA, 20 bp, Artificial Sequence
  Description of Artificial Sequence Synthetic oligonucleotide
cctctgtgaacccctgatcg

SEQ ID NO: 44 - DNA, 20 bp, Artificial Sequence
  Description of Artificial Sequence Synthetic oligonucleotide
cctctgtgaaggggtgatcg

SEQ ID NO: 45 - DNA, 20 bp, Artificial Sequence
  Description of Artificial Sequence Synthetic oligonucleotide
cctctgtgaaggcctactcg

SEQ ID NO: 46 - DNA, 20 bp, Artificial Sequence
  Description of Artificial Sequence Synthetic oligonucleotide
cctgacagaaggcctgatcg

SEQ ID NO: 47 - DNA, 20 bp, Artificial Sequence
  Description of Artificial Sequence Synthetic oligonucleotide
cctctgtgaaggccactacg

SEQ ID NO: 48 - DNA, 20 bp, Artificial Sequence
  Description of Artificial Sequence Synthetic oligonucleotide
ccactgtgaaggcctgatcg

SEQ ID NO: 49 - DNA, 20 bp, Artificial Sequence
  Description of Artificial Sequence Synthetic oligonucleotide
cctgtgtgaaggcctgatcg

SEQ ID NO: 50 - DNA, 20 bp, Artificial Sequence
  Description of Artificial Sequence Synthetic oligonucleotide
cctcagtgaaggcctgatcg

SEQ ID NO: 51 - DNA, 20 bp, Artificial Sequence
  Description of Artificial Sequence Synthetic oligonucleotide
cctctctgaaggcctgatcg

SEQ ID NO: 52 - DNA, 20 bp, Artificial Sequence
  Description of Artificial Sequence Synthetic oligonucleotide
cctctgagaaggcctgatcg

SEQ ID NO: 53 - DNA, 20 bp, Artificial Sequence
  Description of Artificial Sequence Synthetic oligonucleotide
cctctgtcaaggcctgatcg

SEQ ID NO: 54 - DNA, 20 bp, Artificial Sequence
  Description of Artificial Sequence Synthetic oligonucleotide
cctctgtgtaggcctgatcg

SEQ ID NO: 55 - DNA, 20 bp, Artificial Sequence
  Description of Artificial Sequence Synthetic oligonucleotide
cctctgtgatggcctgatcg

SEQ ID NO: 56 - DNA, 20 bp, Artificial Sequence
  Description of Artificial Sequence Synthetic oligonucleotide
cctctgtgaacgcctgatcg

SEQ ID NO: 57 - DNA, 20 bp, Artificial Sequence
  Description of Artificial Sequence Synthetic oligonucleotide
cctctgtgaagccctgatcg

SEQ ID NO: 58 - DNA, 20 bp, Artificial Sequence
  Description of Artificial Sequence Synthetic oligonucleotide
cctctgtgaagggctgatcg

SEQ ID NO: 59 - DNA, 20 bp, Artificial Sequence
  Description of Artificial Sequence Synthetic oligonucleotide
cctctgtgaaggcgtgatcg

SEQ ID NO: 60 - DNA, 20 bp, Artificial Sequence
  Description of Artificial Sequence Synthetic oligonucleotide
cctctgtgaaggccagatcg

SEQ ID NO: 61 - DNA, 20 bp, Artificial Sequence
  Description of Artificial Sequence Synthetic oligonucleotide
cctctgtgaaggcctcatcg

SEQ ID NO: 62 - DNA, 20 bp, Artificial Sequence
  Description of Artificial Sequence Synthetic oligonucleotide
cctctgtgaaggcctgttcg

SEQ ID NO: 63 - DNA, 20 bp, Artificial Sequence
  Description of Artificial Sequence Synthetic oligonucleotide
cctctgtgaaggcctgaacg

SEQ ID NO: 64 - DNA, 15 bp, Artificial Sequence
  Description of Artificial Sequence Synthetic oligonucleotide
tgtccattaacgccc

SEQ ID NO: 65 - DNA, 57 bp, Artificial Sequence
  Description of Artificial Sequence Synthetic oligonucleotide
ttttttcagcaccgaccttgtgctttgggagtgctggtccaagggcgttaatggaca

SEQ ID NO: 66 - DNA, 24 bp, Artificial Sequence
  Description of Artificial Sequence Synthetic oligonucleotide
gattacaccaatgctatgcactcg

SEQ ID NO: 67 - DNA, 48 bp, Artificial Sequence
  Description of Artificial Sequence Synthetic oligonucleotide
ccctgctaaagtgcacgagtgcataggattggtgtaatcaaattggct

SEQ ID NO: 68 - DNA, 39 bp, Artificial Sequence
  Description of Artificial Sequence Synthetic oligonucleotide
gattacaccaatgctatgcactcgtggagtttaggaggg

SEQ ID NO: 69 - DNA, 24 bp, Artificial Sequence
  Description of Artificial Sequence Synthetic oligonucleotide
gattacaccaatgctatgcactct

SEQ ID NO: 70 - DNA, 48 bp, Artificial Sequence
  Description of Artificial Sequence Synthetic oligonucleotide
  modified_base (16)..(16): a, c, t, g, unknown or other
ccctgctaaagtgcangagtgcataggattggtgtaatcaaattggct

SEQ ID NO: 71 - DNA, 23 bp, Artificial Sequence
  Description of Artificial Sequence Synthetic oligonucleotide
gattacaccaatgctatgcactc